SEQUENCE LISTING (922 sequences)

SEQ ID NO: 1 - DNA, 74 bp, synthetic construct
  source 1..74: mol_type = other DNA
ggccggttagctcagttggttagagcgtggtgctctaaacgccaaggtcgcgggttcgatccccgtactggcca

SEQ ID NO: 2 - DNA, 74 bp, synthetic construct
  source 1..74: mol_type = other DNA
ggccggttagctcagttggttagagcgtggcgctctaaacgccaaggtcgcgggttcgatccccgtacgggcca

SEQ ID NO: 3 - DNA, 73 bp, synthetic construct
  source 1..73: mol_type = other DNA
gccgagatagctcagttgggagagcgttagactctagatctaaaggtccctggttcaatcccgggtttcggca

SEQ ID NO: 4 - DNA, 74 bp, synthetic construct
  source 1..74: mol_type = other DNA
ggctggttagctcagttggttagagcgtggtgctctaaacgccaaggtcgcgggttcgatccccgtactggcca

SEQ ID NO: 5 - DNA, 74 bp, synthetic construct
  source 1..74: mol_type = other DNA
ggccggttagctcagttggtcagagcgtggtgctctaaacgccaaggtcgcgggttcgatccccgtacgggcca

SEQ ID NO: 6 - DNA, 74 bp, synthetic construct; identical to SEQ ID NO: 4
  source 1..74: mol_type = other DNA

SEQ ID NO: 7 - DNA, 74 bp, synthetic construct
  source 1..74: mol_type = other DNA
ggccggttagctcagttggttagagcgtggtgctctaaacgccaaggtcgcgggttcgaaccccgtacgggcca

SEQ ID NO: 8 - DNA, 72 bp, synthetic construct
  source 1..72: mol_type = other DNA
ggggatgtagctcagtggtagagcgcgcgcttctaatgtgtgaggtcccgggttcaatccccggcatctcca

SEQ ID NO: 9 - DNA, 74 bp, synthetic construct
  source 1..74: mol_type = other DNA
ggccggttagctcagttggttagagcgtggtgctttaaacgctaaggtcgcgggttcgatccccgtactggcca

SEQ ID NO: 10 - DNA, 74 bp, synthetic construct
  source 1..74: mol_type = other DNA
ggccggttagctcagttggttagagcgtggtgcttcaaacgccaaggtcgcgggttcgatccccgtactggcca

SEQ ID NO: 11 - DNA, 73 bp, synthetic construct
  source 1..73: mol_type = other DNA
gccgaaatagctcagttgggagagcgttagacttcagatctaaaggtccctggttcgatcccgggtttcggca

SEQ ID NO: 12 - DNA, 72 bp, synthetic construct
  source 1..72: mol_type = other DNA
ggggatgtagctcagtggtagagcgcatgcttctaatgtatgaggccccgggttcgatccccggcatctcca

SEQ ID NO: 13 - DNA, 74 bp, synthetic construct
  source 1..74: mol_type = other DNA
ggccggttagctcagttggttagagcgtggtgcttcaaacgctaaggtcgcgggttcgatccccgtactggcca

SEQ ID NO: 14 - DNA, 73 bp, synthetic construct
  source 1..73: mol_type = other DNA
gccgaaatagctcagttgggagagcgttagactttagatctaaaggtccctggttcgatcccgggtttcggca

SEQ ID NO: 15 - DNA, 74 bp, synthetic construct
  source 1..74: mol_type = other DNA
ggctctgtggcttagttggctaaagcgcctgtctctaaaacaggagatcctgggttcgaatcccagcggggcct

SEQ ID NO: 16 - DNA, 74 bp, synthetic construct
  source 1..74: mol_type = other DNA
ggccggttagctcagttggttagagcgtggtgctctaaacgctaaggtcgcgggttcgatccccgtactggcca

SEQ ID NO: 17 - DNA, 73 bp, synthetic construct
  source 1..73: mol_type = other DNA
ggctccatagctcagtggttagagcactggtctctaaaaccaggggtcgcgagttcgatcctcgctggggcct

SEQ ID NO: 18 - DNA, 74 bp, synthetic construct
  source 1..74: mol_type = other DNA
ggctccgtagcttagttggttaaagcgcctgtctctaaaacaggagatcctgggttcgactcccagcggggcct

SEQ ID NO: 19 - DNA, 73 bp, synthetic construct
  source 1..73: mol_type = other DNA
gccgaaatagctcagttgggagagcgttagacttcagatctaaaggtccctggttcaatcccgggtttcggca

SEQ ID NO: 20 - DNA, 74 bp, synthetic construct
  source 1..74: mol_type = other DNA
ggccggttagctcagttggttagagcgtggtgctctaaacgccaaggtcgcgggttcgatccccgtacgggcca

SEQ ID NO: 21 - DNA, 74 bp, synthetic construct
  source 1..74: mol_type = other DNA
ggccggttagctcagttggttagagcgtggcgctttaaacgccaaggtcgcgggttcgatccccgtacgggcca

SEQ ID NO: 22 - DNA, 73 bp, synthetic construct
  source 1..73: mol_type = other DNA
gccgaaatagctcagttgggagagcgttagactttagatctaaaggtccctggttcaatcccgggtttcggca

SEQ ID NO: 23 - DNA, 72 bp, synthetic construct
  source 1..72: mol_type = other DNA
ggggatgtagctcagtggtagagcgcatgcttctaatgcatgaggtcccgggttcgatccccagcatctcca

SEQ ID NO: 24 - DNA, 72 bp, synthetic construct
  source 1..72: mol_type = other DNA
ggcgcggtggccaagtggtaaggcgtcggtctctaaaaccgaagatcacgggttcgaaccccgtccgtgcct

SEQ ID NO: 25 - DNA, 74 bp, synthetic construct
  source 1..74: mol_type = other DNA
ggccggttagctcagtcggctagagcgtggtgcttcaaacgccaaggtcgcgggttcgatccccgtacgggcca

SEQ ID NO: 26 - DNA, 74 bp, synthetic construct
  source 1..74: mol_type = other DNA
ggctctatggcttagttggttaaagcgcctgtctctaaaacaggagatcctgggttcgaatcccagtagagcct

SEQ ID NO: 27 - DNA, 72 bp, synthetic construct
  source 1..72: mol_type = other DNA
ggcgcggtggccaagtggtaaggcgtcggtcttcaaaaccgaagatcgcgggttcgaaccccgtccgtgcct

SEQ ID NO: 28 - DNA, 74 bp, synthetic construct
  source 1..74: mol_type = other DNA
ggccggttagctcagttggttagagcgtggtgctttaaacgccaaggtcgcgggttcgatccccgtactggcca

SEQ ID NO: 29 - DNA, 72 bp, synthetic construct
  source 1..72: mol_type = other DNA
gggggtatagctcagtggtagagcgcgtgcttctaatgcacgaggtcctgggttcgatccccagtacctcca

SEQ ID NO: 30 - DNA, 73 bp, synthetic construct
  source 1..73: mol_type = other DNA
gccgagatagctcagttgggagagcgttagacttcagatctaaaggtccctggttcaatcccgggtttcggca

SEQ ID NO: 31 - DNA, 74 bp, synthetic construct
  source 1..74: mol_type = other DNA
ggccggttagctcagtcggctagagcgtggtgctttaaacgccaaggtcgcgggttcgatccccgtacgggcca

SEQ ID NO: 32 - DNA, 74 bp, synthetic construct
  source 1..74: mol_type = other DNA
ggctccgtggcttagctggttaaagcgcctgtctctaaaacaggagatcctgggttcgaatcccagcggggcct

SEQ ID NO: 33 - DNA, 74 bp, synthetic construct
  source 1..74: mol_type = other DNA
ggctggttagctcagttggttagagcgtggtgctttaaacgccaaggtcgcgggttcgatccccgtactggcca

SEQ ID NO: 34 - DNA, 73 bp, synthetic construct
  source 1..73: mol_type = other DNA
ggggaattagctcaaatggtagagcgctcgcttctaatgcgagaggtagcgggatcgatgcccgcattctcca

SEQ ID NO: 35 - DNA, 74 bp, synthetic construct
  source 1..74: mol_type = other DNA
ggccggttagctcagttggtcagagcgtggtgctttaaacgccaaggtcgcgggttcgatccccgtacgggcca

SEQ ID NO: 36 - DNA, 72 bp, synthetic construct
  source 1..72: mol_type = other DNA
ggggatgtagctcagtggtagagcgcatgcttttaatgcatgaggtcccgggttcgatccccagcatctcca

SEQ ID NO: 37 - DNA, 72 bp, synthetic construct
  source 1..72: mol_type = other DNA
ggggatgtagctcagtggtagagcgcgcgcttttaatgtgtgaggtcccgggttcaatccccggcatctcca

SEQ ID NO: 38 - DNA, 72 bp, synthetic construct
  source 1..72: mol_type = other DNA
gggggtgtagctcagtggtagagcgcgtgcttttaatgcacgaggccccgggttcaatccctggcacctcca

SEQ ID NO: 39 - DNA, 72 bp, synthetic construct
  source 1..72: mol_type = other DNA
ggggatgtagctcagtggtagagcgcatgcttttaatgtatgaggccccgggttcgatccccggcatctcca

SEQ ID NO: 40 - DNA, 72 bp, synthetic construct
  source 1..72: mol_type = other DNA
gggggtgtagctcagtggtagagcgcgtgcttttaatgtacgaggtcccgggttcaatccccggcacctcca

SEQ ID NO: 41 - DNA, 72 bp, synthetic construct
  source 1..72: mol_type = other DNA
ggggatgtagctcagtggtagagcgcatgcttttaatgtatgaggtcccgggttcgatccccggcatctcca

SEQ ID NO: 42 - DNA, 72 bp, synthetic construct
  source 1..72: mol_type = other DNA
gggggtgtagctcagtggtagagcgcgtgcttttaatgcacgaggccccgggttcaatccccggcacctcca

SEQ ID NO: 43 - DNA, 73 bp, synthetic construct
  source 1..73: mol_type = other DNA
ggggaattagctcaaatggtagagcgctcgcttttaatgcgagaggtagcgggatcgatgcccgcattctcca

SEQ ID NO: 44 - DNA, 72 bp, synthetic construct
  source 1..72: mol_type = other DNA
gggggtatagctcagtggtagagcgcgtgcttttaatgcacgaggtcctgggttcgatccccagtacctcca

SEQ ID NO: 45 - DNA, 73 bp, synthetic construct
  source 1..73: mol_type = other DNA
gggggattagctcaaatggtagagcgctcgcttttaatgcgagaggtagcgggatcgatgcccgcatcctcca

SEQ ID NO: 46 - DNA, 73 bp, synthetic construct
  source 1..73: mol_type = other DNA
ggggaattagctcaagtggtagagcgctcgcttttaatgcgagaggtagtgggatcgatgcccgcattctcca

SEQ ID NO: 47 - DNA, 72 bp, synthetic construct
  source 1..72: mol_type = other DNA
gggggtgtagctcagtggtagagcgcgtgcttttaatgcacgaggccctgggttcaatccccagcacctcca

SEQ ID NO: 48 - DNA, 73 bp, synthetic construct
  source 1..73: mol_type = other DNA
ggggaattagctcaggcggtagagcgctcgcttttaatgcgagaggtagcgggatcgacgcccgcattctcca

SEQ ID NO: 49 - DNA, 72 bp, synthetic construct
  source 1..72: mol_type = other DNA
gggggtatagctcagcggtagagcgcgtgcttttaatgcacgaggtcctgggttcaatccccaatacctcca

SEQ ID NO: 50 - DNA, 73 bp, synthetic construct
  source 1..73: mol_type = other DNA
ggggaattagctcaagtggtagagcgcttgcttttaacgcaagaggtagtgggatcgatgcccacattctcca

SEQ ID NO: 51 - DNA, 73 bp, synthetic construct
  source 1..73: mol_type = other DNA
ggggaattagctcaagtggtagagcgcttgcttttaatgcaagaggtagtgggatcgatgcccacattctcca

SEQ ID NO: 52 - DNA, 73 bp, synthetic construct
  source 1..73: mol_type = other DNA
ggggaattagctcaagcggtagagcgcttgcttttaatgcaagaggtagtgggatcgatgcccacattctcca

SEQ ID NO: 53 - DNA, 73 bp, synthetic construct
  source 1..73: mol_type = other DNA
ggggaattagctcaagtggtagagcgcttgcttttaatgcaagaggtagtgggatcaatgcccacattctcca

SEQ ID NO: 54 - DNA, 73 bp, synthetic construct
  source 1..73: mol_type = other DNA
ggggaattagcccaagtggtagagcgcttgcttttaatgcaagaggtagtgggatcgatgcccacattctcca

SEQ ID NO: 55 - DNA, 72 bp, synthetic construct
  source 1..72: mol_type = other DNA
gggggtgtagctcagtggtagagcgcgtgcttttaatgtacgaggccccgggttcgacccccggctcctcca

SEQ ID NO: 56 - DNA, 72 bp, synthetic construct
  source 1..72: mol_type = other DNA
gggggtgtagctcagtggtagagcgcgtgcttctaatgtacgaggtcccgggttcaatccccggcacctcca

SEQ ID NO: 57 - DNA, 72 bp, synthetic construct
  source 1..72: mol_type = other DNA
gggggtgtagctcagtggtagagcgcgtgcttctaatgtacgaggccccgggttcgacccccggctcctcca

SEQ ID NO: 58 - DNA, 72 bp, synthetic construct
  source 1..72: mol_type = other DNA
gggggtgtagctcagtggtagagcgcgtgcttctaatgcacgaggccccgggttcaatccctggcacctcca

SEQ ID NO: 59 - DNA, 73 bp, synthetic construct
  source 1..73: mol_type = other DNA
gggggattagctcaaatggtagagcgctcgcttctaatgcgagaggtagcgggatcgatgcccgcatcctcca

SEQ ID NO: 60 - DNA, 73 bp, synthetic construct
  source 1..73: mol_type = other DNA
ggggaattagctcaggcggtagagcgctcgcttctaatgcgagaggtagcgggatcgacgcccgcattctcca

SEQ ID NO: 61 - DNA, 72 bp, synthetic construct
  source 1..72: mol_type = other DNA
gggggtgtagctcagtggtagagcgcgtgcttctaatgcacgaggccctgggttcaatccccagcacctcca

SEQ ID NO: 62 - DNA, 72 bp, synthetic construct
  source 1..72: mol_type = other DNA
gggggtgtagctcagtggtagagcgcgtgcttctaatgcacgaggccccgggttcaatccccggcacctcca

SEQ ID NO: 63 - DNA, 73 bp, synthetic construct
  source 1..73: mol_type = other DNA
ggggaattagctcaagtggtagagcgctcgcttctaatgcgagaggtagtgggatcgatgcccgcattctcca

SEQ ID NO: 64 - DNA, 73 bp, synthetic construct
  source 1..73: mol_type = other DNA
ggggaattagctcaagtggtagagcgcttgcttctaacgcaagaggtagtgggatcgatgcccacattctcca

SEQ ID NO: 65 - DNA, 73 bp, synthetic construct
  source 1..73: mol_type = other DNA
ggggaattagctcaagtggtagagcgcttgcttctaatgcaagaggtagtgggatcgatgcccacattctcca

SEQ ID NO: 66 - DNA, 73 bp, synthetic construct
  source 1..73: mol_type = other DNA
ggggaattagctcaagcggtagagcgcttgcttctaatgcaagaggtagtgggatcgatgcccacattctcca

SEQ ID NO: 67 - DNA, 73 bp, synthetic construct
  source 1..73: mol_type = other DNA
ggggaattagctcaagtggtagagcgcttgcttctaatgcaagaggtagtgggatcaatgcccacattctcca

SEQ ID NO: 68 - DNA, 73 bp, synthetic construct
  source 1..73: mol_type = other DNA
ggggaattagcccaagtggtagagcgcttgcttctaatgcaagaggtagtgggatcgatgcccacattctcca

SEQ ID NO: 69 - DNA, 72 bp, synthetic construct
  source 1..72: mol_type = other DNA
gggggtatagctcagcggtagagcgcgtgcttctaatgcacgaggtcctgggttcaatccccaatacctcca

SEQ ID NO: 70 - DNA, 72 bp, synthetic construct
  source 1..72: mol_type = other DNA
ggggatgtagctcagtggtagagcgcatgcttctaatgtatgaggtcccgggttcgatccccggcatctcca

SEQ ID NO: 71 - DNA, 72 bp, synthetic construct
  source 1..72: mol_type = other DNA
gggggtgtagctcagtggtagagcgcgtgctttcaatgtacgaggccccgggttcgacccccggctcctcca

SEQ ID NO: 72 - DNA, 72 bp, synthetic construct
  source 1..72: mol_type = other DNA
ggggatgtagctcagtggtagagcgcgcgctttcaatgtgtgaggtcccgggttcaatccccggcatctcca

SEQ ID NO: 73 - DNA, 72 bp, synthetic construct
  source 1..72: mol_type = other DNA
ggggatgtagctcagtggtagagcgcatgctttcaatgcatgaggtcccgggttcgatccccagcatctcca

SEQ ID NO: 74 - DNA, 72 bp, synthetic construct
  source 1..72: mol_type = other DNA
gggggtgtagctcagtggtagagcgcgtgctttcaatgcacgaggccccgggttcaatccctggcacctcca

SEQ ID NO: 75 - DNA, 72 bp, synthetic construct
  source 1..72: mol_type = other DNA
ggggatgtagctcagtggtagagcgcatgctttcaatgtatgaggccccgggttcgatccccggcatctcca

SEQ ID NO: 76 - DNA, 72 bp, synthetic construct
  source 1..72: mol_type = other DNA
gggggtgtagctcagtggtagagcgcgtgctttcaatgtacgaggtcccgggttcaatccccggcacctcca

SEQ ID NO: 77 - DNA, 73 bp, synthetic construct
  source 1..73: mol_type = other DNA
gggggattagctcaaatggtagagcgctcgctttcaatgcgagaggtagcgggatcgatgcccgcatcctcca

SEQ ID NO: 78 - DNA, 73 bp, synthetic construct
  source 1..73: mol_type = other DNA
ggggaattagctcaaatggtagagcgctcgctttcaatgcgagaggtagcgggatcgatgcccgcattctcca

SEQ ID NO: 79 - DNA, 72 bp, synthetic construct
  source 1..72: mol_type = other DNA
gggggtgtagctcagtggtagagcgcgtgctttcaatgcacgaggccctgggttcaatccccagcacctcca

SEQ ID NO: 80 - DNA, 72 bp, synthetic construct
  source 1..72: mol_type = other DNA
gggggtgtagctcagtggtagagcgcgtgctttcaatgcacgaggccccgggttcaatccccggcacctcca

SEQ ID NO: 81 - DNA, 72 bp, synthetic construct
  source 1..72: mol_type = other DNA
gggggtatagctcagtggtagagcgcgtgctttcaatgcacgaggtcctgggttcgatccccagtacctcca

SEQ ID NO: 82 - DNA, 72 bp, synthetic construct
  source 1..72: mol_type = other DNA
ggggatgtagctcagtggtagagcgcatgctttcaatgtatgaggtcccgggttcgatccccggcatctcca

SEQ ID NO: 83 - DNA, 73 bp, synthetic construct
  source 1..73: mol_type = other DNA
ggggaattagctcaagtggtagagcgctcgctttcaatgcgagaggtagtgggatcgatgcccgcattctcca

SEQ ID NO: 84 - DNA, 72 bp, synthetic construct
  source 1..72: mol_type = other DNA
gggggtatagctcagcggtagagcgcgtgctttcaatgcacgaggtcctgggttcaatccccaatacctcca

SEQ ID NO: 85 - DNA, 73 bp, synthetic construct
  source 1..73: mol_type = other DNA
ggggaattagctcaagcggtagagcgcttgctttcaatgcaagaggtagtgggatcgatgcccacattctcca

SEQ ID NO: 86 - DNA, 73 bp, synthetic construct
  source 1..73: mol_type = other DNA
ggggaattagctcaggcggtagagcgctcgctttcaatgcgagaggtagcgggatcgacgcccgcattctcca

SEQ ID NO: 87 - DNA, 73 bp, synthetic construct
  source 1..73: mol_type = other DNA
ggggaattagcccaagtggtagagcgcttgctttcaatgcaagaggtagtgggatcgatgcccacattctcca

SEQ ID NO: 88 - DNA, 73 bp, synthetic construct
  source 1..73: mol_type = other DNA
ggggaattagctcaagtggtagagcgcttgctttcaatgcaagaggtagtgggatcgatgcccacattctcca

SEQ ID NO: 89 - DNA, 73 bp, synthetic construct
  source 1..73: mol_type = other DNA
ggggaattagctcaagtggtagagcgcttgctttcaacgcaagaggtagtgggatcgatgcccacattctcca

SEQ ID NO: 90 - DNA, 73 bp, synthetic construct
  source 1..73: mol_type = other DNA
ggggaattagctcaagtggtagagcgcttgctttcaatgcaagaggtagtgggatcaatgcccacattctcca

SEQ ID NO: 91 - DNA, 73 bp, synthetic construct
  source 1..73: mol_type = other DNA
ggctccgtggcgcaatggatagcgcattggactttaaattcaaaggttccgggttcgagtcccggcggagtcg

SEQ ID NO: 92 - DNA, 73 bp, synthetic construct
  source 1..73: mol_type = other DNA
gacccagtggcctaatggataaggcatcagcctttagagctggggattgtgggttcgagtcccatctgggtcg

SEQ ID NO: 93 - DNA, 73 bp, synthetic construct
  source 1..73: mol_type = other DNA
gccccagtggcctaatggataaggcattggcctttaaagccagggattgtgggttcgagtcccatctggggtg

SEQ ID NO: 94 - DNA, 73 bp, synthetic construct
  source 1..73: mol_type = other DNA
gggccagtggcgcaatggataacgcgtctgactttagatcagaagattccaggttcgactcctggctggctcg

SEQ ID NO: 95 - DNA, 73 bp, synthetic construct
  source 1..73: mol_type = other DNA
gggccagtggcgcaatggataacgcgtctgactttagatcagaagattctaggttcgactcctggctggctcg

SEQ ID NO: 96 - DNA, 73 bp, synthetic construct
  source 1..73: mol_type = other DNA
gccccagtggcctaatggataaggcactggcctttaaagccagggattgtgggttcgagtcccacctggggtg

SEQ ID NO: 97 - DNA, 74 bp, synthetic construct
  source 1..74: mol_type = other DNA
gtctctgtggcgcaatggacgagcgcgctggactttaaatccagaggttccgggttcgagtcccggcagagatg

SEQ ID NO: 98 - DNA, 73 bp, synthetic construct
  source 1..73: mol_type = other DNA
ggctctgtggcgcaatggatagcgcattggactttaaattcaaaggttgtgggttcgagtcccaccagagtcg

SEQ ID NO: 99 - DNA, 73 bp, synthetic construct
  source 1..73: mol_type = other DNA
ggccgcgtggcctaatggataaggcgtctgactttagatcagaagattgcaggttcgagtcctgccgcggtcg

SEQ ID NO: 100 - DNA, 73 bp, synthetic construct
  source 1..73: mol_type = other DNA
gccccggtggcctaatggataaggcattggcctttaaagccagggattgtgggttcgagtcccacccggggta

SEQ ID NO: 101 - DNA, 73 bp, synthetic construct
  source 1..73: mol_type = other DNA
gaccgcgtggcctaatggataaggcgtctgactttagatcagaagattgagggttcgagtcccttcgtggtcg

SEQ ID NO: 102 - DNA, 73 bp, synthetic construct
  source 1..73: mol_type = other DNA
gccccagtggcctaatggataaggcactggcctttaaagccagggattgtgggttcgagtcccacctggggta

SEQ ID NO: 103 - DNA, 73 bp, synthetic construct; identical to SEQ ID NO: 98
  source 1..73: mol_type = other DNA

SEQ ID NO: 104 - DNA, 73 bp, synthetic construct
  source 1..73: mol_type = other DNA
ggctctgtggcgcaatggatagcgcattggactttaaattcaaaggttgtgggttcgaatcccaccagagtcg

SEQ ID NO: 105 - DNA, 73 bp, synthetic construct
  source 1..73: mol_type = other DNA
gaccacgtggcctaatggataaggcgtctgactttagatcagaagattgagggttcgaatccctccgtggtta

SEQ ID NO: 106 - DNA, 73 bp, synthetic construct
  source 1..73: mol_type = other DNA
gaccacgtggcctaatggataaggcgtctgactttagatcagaagattgagggttcgaatcccttcgtggtta

SEQ ID NO: 107 - DNA, 73 bp, synthetic construct
  source 1..73: mol_type = other DNA
gaccacgtggcctaatggataaggcgtctgactttagatcagaagattgagggttcgaatcccttcgtggttg

SEQ ID NO: 108 - DNA, 73 bp, synthetic construct
  source 1..73: mol_type = other DNA
ggctctgtggcgcaatggatagcgcattggactttaaattcaaaggttgcgggttcgagtccctccagagtcg

SEQ ID NO: 109 - DNA, 73 bp, synthetic construct
  source 1..73: mol_type = other DNA
ggccgcgtggcctaatggataaggcgtctgattttagatcagaagattgagggttcgagtcccttcgtggtcg

SEQ ID NO: 110 - DNA, 69 bp, synthetic construct
  source 1..69: mol_type = other DNA
ggcagcatagcagagtggttcaggttacaggtttaagatgtaaactgagttcaaatcccagttctgcca

SEQ ID NO: 111 - DNA, 73 bp, synthetic construct
  source 1..73: mol_type = other DNA
gccccagtggcctgatggataaggtactggcctttaaagccagggattgtgggttcgagttccacctggggta

SEQ ID NO: 112 - DNA, 73 bp, synthetic construct
  source 1..73: mol_type = other DNA
ggccgtgtggcctaatggataaggcgtctgactttagatcaaaagattgcaggtttgagttctgccacggtcg

SEQ ID NO: 113 - DNA, 73 bp, synthetic construct
  source 1..73: mol_type = other DNA
ggctccgtggcgcaatggatagcgcattggactctaaattcaaaggttccgggttcgagtcccggcggagtcg

SEQ ID NO: 114 - DNA, 73 bp, synthetic construct
  source 1..73: mol_type = other DNA
gccccagtggcctaatggataaggcattggcctctaaagccagggattgtgggttcgagtcccatctggggtg

SEQ ID NO: 115 - DNA, 73 bp, synthetic construct
  source 1..73: mol_type = other DNA
ggctctgtggcgcaatggatagcgcattggactctaaattcaaaggttgtgggttcgagtcccaccagagtcg

SEQ ID NO: 116 - DNA, 73 bp, synthetic construct
  source 1..73: mol_type = other DNA
gccccggtggcctaatggataaggcattggcctctaaagccagggattgtgggttcgagtcccacccggggta

SEQ ID NO: 117 - DNA, 73 bp, synthetic construct
  source 1..73: mol_type = other DNA
ggctctgtggcgcaatggatagcgcattggactctaaattcaaaggttgtgggttcgaatcccaccagagtcg

SEQ ID NO: 118 - DNA, 73 bp, synthetic construct
  source 1..73: mol_type = other DNA
gacccagtggcctaatggataaggcatcagcctctagagctggggattgtgggttcgagtcccatctgggtcg

SEQ ID NO: 119 - DNA, 73 bp, synthetic construct
  source 1..73: mol_type = other DNA
gaccacgtggcctaatggataaggcgtctgactctagatcagaagattgagggttcgaatcccttcgtggtta

SEQ ID NO: 120 - DNA, 73 bp, synthetic construct
  source 1..73: mol_type = other DNA
ggccgcgtggcctaatggataaggcgtctgactctagatcagaagattgcaggttcgagtcctgccgcggtcg

SEQ ID NO: 121 - DNA, 73 bp, synthetic construct
  source 1..73: mol_type = other DNA
gggccagtggcgcaatggataacgcgtctgactctagatcagaagattccaggttcgactcctggctggctcg

SEQ ID NO: 122 - DNA, 73 bp, synthetic construct
  source 1..73: mol_type = other DNA
ggctctgtggcgcaatggatagcgcattggactctaaattcaaaggttgcgggttcgagtccctccagagtcg

SEQ ID NO: 123 - DNA, 73 bp, synthetic construct
  source 1..73: mol_type = other DNA
gccccagtggcctaatggataaggcactggcctctaaagccagggattgtgggttcgagtcccacctggggta

SEQ ID NO: 124 - DNA, 73 bp, synthetic construct
  source 1..73: mol_type = other DNA
gaccgcgtggcctaatggataaggcgtctgactctagatcagaagattgagggttcgagtcccttcgtggtcg

SEQ ID NO: 125 - DNA, 73 bp, synthetic construct
  source 1..73: mol_type = other DNA
gccccagtggcctaatggataaggcactggcctctaaagccagggattgtgggttcgagtcccacctggggtg

SEQ ID NO: 126 - DNA, 73 bp, synthetic construct
  source 1..73: mol_type = other DNA
gaccacgtggcctaatggataaggcgtctgactctagatcagaagattgagggttcgaatcccttcgtggttg

SEQ ID NO: 127 - DNA, 73 bp, synthetic construct
  source 1..73: mol_type = other DNA
ggccgcgtggcctaatggataaggcgtctgattctagatcagaagattgagggttcgagtcccttcgtggtcg

SEQ ID NO: 128 - DNA, 73 bp, synthetic construct
  source 1..73: mol_type = other DNA
gggccagtggcgcaatggataacgcgtctgactctagatcagaagattctaggttcgactcctggctggctcg

SEQ ID NO: 129 - DNA, 74 bp, synthetic construct
  source 1..74: mol_type = other DNA
gtctctgtggcgcaatggacgagcgcgctggactctaaatccagaggttccgggttcgagtcccggcagagatg

SEQ ID NO: 130 - DNA, 73 bp, synthetic construct
  source 1..73: mol_type = other DNA
gaccacgtggcctaatggataaggcgtctgactctagatcagaagattgagggttcgaatccctccgtggtta

SEQ ID NO: 131 - DNA, 73 bp, synthetic construct
  source 1..73: mol_type = other DNA
gccccagtggcctgatggataaggtactggcctctaaagccagggattgtgggttcgagttccacctggggta

SEQ ID NO: 132 - DNA, 69 bp, synthetic construct
  source 1..69: mol_type = other DNA
ggcagcatagcagagtggttcaggttacaggtctaagatgtaaactgagttcaaatcccagttctgcca

SEQ ID NO: 133 - DNA, 73 bp, synthetic construct
  source 1..73: mol_type = other DNA
ggccgtgtggcctaatggataaggcgtctgactctagatcaaaagattgcaggtttgagttctgccacggtcg

SEQ ID NO: 134 - DNA, 73 bp, synthetic construct
  source 1..73: mol_type = other DNA
gaccacgtggcctaatggataaggcgtctgacttcagatcagaagattgagggttcgaatcccttcgtggttg

SEQ ID NO: 135 - DNA, 73 bp, synthetic construct
  source 1..73: mol_type = other DNA
ggctctgtggcgcaatggatagcgcattggacttcaaattcaaaggttgtgggttcgaatcccaccagagtcg

SEQ ID NO: 136 - DNA, 73 bp, synthetic construct
  source 1..73: mol_type = other DNA
gccccagtggcctaatggataaggcactggccttcaaagccagggattgtgggttcgagtcccacctggggta

SEQ ID NO: 137 - DNA, 73 bp, synthetic construct
  source 1..73: mol_type = other DNA
gccccggtggcctaatggataaggcattggccttcaaagccagggattgtgggttcgagtcccacccggggta

SEQ ID NO: 138 - DNA, 73 bp, synthetic construct
  source 1..73: mol_type = other DNA
gccccagtggcctaatggataaggcactggccttcaaagccagggattgtgggttcgagtcccacctggggtg

SEQ ID NO: 139 - DNA, 73 bp, synthetic construct
  source 1..73: mol_type = other DNA
ggctctgtggcgcaatggatagcgcattggacttcaaattcaaaggttgtgggttcgagtcccaccagagtcg

SEQ ID NO: 140 - DNA, 74 bp, synthetic construct
  source 1..74: mol_type = other DNA
gtctctgtggcgcaatggacgagcgcgctggacttcaaatccagaggttccgggttcgagtcccggcagagatg

SEQ ID NO: 141 - DNA, 73 bp, synthetic construct
  source 1..73: mol_type = other DNA
gacccagtggcctaatggataaggcatcagccttcagagctggggattgtgggttcgagtcccatctgggtcg

SEQ ID NO: 142 - DNA, 73 bp, synthetic construct
  source 1..73: mol_type = other DNA
gccccagtggcctgatggataaggtactggccttcaaagccagggattgtgggttcgagttccacctggggta

SEQ ID NO: 143 - DNA, 73 bp, synthetic construct
  source 1..73: mol_type = other DNA
ggctccgtggcgcaatggatagcgcattggacttcaaattcaaaggttccgggttcgagtcccggcggagtcg

SEQ ID NO: 144 - DNA, 73 bp, synthetic construct
  source 1..73: mol_type = other DNA
gaccgcgtggcctaatggataaggcgtctgacttcagatcagaagattgagggttcgagtcccttcgtggtcg

SEQ ID NO: 145 - DNA, 73 bp, synthetic construct
  source 1..73: mol_type = other DNA
ggctctgtggcgcaatggatagcgcattggacttcaaattcaaaggttgcgggttcgagtccctccagagtcg

SEQ ID NO: 146 - DNA, 73 bp, synthetic construct
  source 1..73: mol_type = other DNA
gggccagtggcgcaatggataacgcgtctgacttcagatcagaagattctaggttcgactcctggctggctcg

SEQ ID NO: 147 - DNA, 73 bp, synthetic construct; identical to SEQ ID NO: 139
  source 1..73: mol_type = other DNA

SEQ ID NO: 148 - DNA, 73 bp, synthetic construct
  source 1..73: mol_type = other DNA
gggccagtggcgcaatggataacgcgtctgacttcagatcagaagattccaggttcgactcctggctggctcg

SEQ ID NO: 149 - DNA, 73 bp, synthetic construct
  source 1..73: mol_type = other DNA
gaccacgtggcctaatggataaggcgtctgacttcagatcagaagattgagggttcgaatcccttcgtggtta

SEQ ID NO: 150 - DNA, 73 bp, synthetic construct
  source 1..73: mol_type = other DNA
ggccgcgtggcctaatggataaggcgtctgacttcagatcagaagattgcaggttcgagtcctgccgcggtcg

SEQ ID NO: 151 - DNA, 73 bp, synthetic construct
  source 1..73: mol_type = other DNA
gaccacgtggcctaatggataaggcgtctgacttcagatcagaagattgagggttcgaatccctccgtggtta

SEQ ID NO: 152 - DNA, 73 bp, synthetic construct
  source 1..73: mol_type = other DNA
ggccgtgtggcctaatggataaggcgtctgacttcagatcaaaagattgcaggtttgagttctgccacggtcg

SEQ ID NO: 153 - DNA, 73 bp, synthetic construct
  source 1..73: mol_type = other DNA
ggccgcgtggcctaatggataaggcgtctgatttcagatcagaagattgagggttcgagtcccttcgtggtcg

SEQ ID NO: 154 - DNA, 69 bp, synthetic construct
  source 1..69: mol_type = other DNA
ggcagcatagcagagtggttcaggttacaggttcaagatgtaaactgagttcaaatcccagttctgcca

SEQ ID NO: 155 - DNA, 73 bp, synthetic construct
  source 1..73: mol_type = other DNA
gccccagtggcctaatggataaggcattggccttcaaagccagggattgtgggttcgagtcccatctggggtg

SEQ ID NO: 156 - DNA, 74 bp, synthetic construct
  source 1..74: mol_type = other DNA
gtctctgtggcgcaatcggttagcgcgttcggctttaaaccgaaaggttggtggttcgagcccacccagggacg

SEQ ID NO: 157 - DNA, 74 bp, synthetic construct
  source 1..74: mol_type = other DNA
gtctctgtggcgcaatgggttagcgcgttcggctttaaaccgaaaggttggtggttcgagcccatccagggacg

SEQ ID NO: 158 - DNA, 74 bp, synthetic construct
  source 1..74: mol_type = other DNA
gtctctgtggcgcaatcggttagcgcgttcggctttaaaccgaaaggttggtggttcgatcccacccagggacg

SEQ ID NO: 159 - DNA, 74 bp, synthetic construct
  source 1..74: mol_type = other DNA
gtctctgtggcgcaatcggttagcgcattcggctttaaaccgaaaggttggtggttcgagcccacccagggacg

SEQ ID NO: 160 - DNA, 74 bp, synthetic construct
  source 1..74: mol_type = other DNA
gtctctgtggcgcaatcggttagcgcgttcggctttaaactgaaaggttggtggttcgagcccacccagggacg

SEQ ID NO: 161 - DNA, 74 bp, synthetic construct
  source 1..74: mol_type = other DNA
gtctctgtggcgcaatcggttagcgcgttcggctttaaaccgaaagattggtggttcgagcccacccagggacg

SEQ ID NO: 162 - DNA, 74 bp, synthetic construct
  source 1..74: mol_type = other DNA
gtctctgtggcgcaatcggctagcgcgtttggctttaaactaaaaggttggtggttcgaacccacccagaggcg

SEQ ID NO: 163 - DNA, 74 bp, synthetic construct
  source 1..74: mol_type = other DNA
gtctctgtggcgcaatcggctagcgcgtttggctttaaactaaaaggttggcggttcgaacccacccagaggcg

SEQ ID NO: 164 - DNA, 74 bp, synthetic construct
  source 1..74: mol_type = other DNA
gtctctgtggcgcaatcggctagcgcgtttggctttaaactaaaaagttggtggttcgaacacacccagaggcg

SEQ ID NO: 165 - DNA, 74 bp, synthetic construct
  source 1..74: mol_type = other DNA
gtctctgtggcgtagtcggttagcgcgttcggctttaaaccgaaaagttggtggttcgagcccacccaggaacg

SEQ ID NO: 166 - DNA, 74 bp, synthetic construct
  source 1..74: mol_type = other DNA
gtctctgtggtgcaatcggttagcgcgttccgctttaaaccgaaagcttggtggttcgagcccacccagggatg

SEQ ID NO: 167 - DNA, 74 bp, synthetic construct
  source 1..74: mol_type = other DNA
gtctctgtggcgcaatcggttagcgcgttcggctttaaactgaaaggttagtggttcgagcccacccggggacg

SEQ ID NO: 168 - DNA, 74 bp, synthetic construct
  source 1..74: mol_type = other DNA
gtctctgtggcgcaatgggttagcgcgttcggctctaaaccgaaaggttggtggttcgagcccatccagggacg

SEQ ID NO: 169 - DNA, 74 bp, synthetic construct
  source 1..74: mol_type = other DNA
gtctctgtggcgcaatcggctagcgcgtttggctctaaactaaaaggttggtggttcgaacccacccagaggcg

SEQ ID NO: 170 - DNA, 74 bp, synthetic construct
  source 1..74: mol_type = other DNA
gtctctgtggcgcaatcggttagcgcgttcggctctaaaccgaaaggttggtggttcgatcccacccagggacg

SEQ ID NO: 171 - DNA, 74 bp, synthetic construct
  source 1..74: mol_type = other DNA
gtctctgtggcgcaatcggttagcgcattcggctctaaaccgaaaggttggtggttcgagcccacccagggacg

SEQ ID NO: 172 - DNA, 74 bp, synthetic construct
  source 1..74: mol_type = other DNA
gtctctgtggcgcaatcggttagcgcgttcggctctaaaccgaaaggttggtggttcgagcccacccagggacg

SEQ ID NO: 173 - DNA, 74 bp, synthetic construct
  source 1..74: mol_type = other DNA
gtctctgtggcgcaatcggttagcgcgttcggctctaaactgaaaggttggtggttcgagcccacccagggacg

SEQ ID NO: 174 - DNA, 74 bp, synthetic construct
  source 1..74: mol_type = other DNA
gtctctgtggcgcaatcggttagcgcgttcggctctaaaccgaaagattggtggttcgagcccacccagggacg

SEQ ID NO: 175 - DNA, 74 bp, synthetic construct
  source 1..74: mol_type = other DNA
gtctctgtggcgcaatcggctagcgcgtttggctctaaactaaaaagttggtggttcgaacacacccagaggcg

SEQ ID NO: 176 - DNA, 74 bp, synthetic construct
  source 1..74: mol_type = other DNA
gtctctgtggcgcaatcggctagcgcgtttggctctaaactaaaaggttggcggttcgaacccacccagaggcg

SEQ ID NO: 177 - DNA, 74 bp, synthetic construct
  source 1..74: mol_type = other DNA
gtctctgtggcgtagtcggttagcgcgttcggctctaaaccgaaaagttggtggttcgagcccacccaggaacg

SEQ ID NO: 178 - DNA, 74 bp, synthetic construct
  source 1..74: mol_type = other DNA
gtctctgtggtgcaatcggttagcgcgttccgctctaaaccgaaagcttggtggttcgagcccacccagggatg

SEQ ID NO: 179 - DNA, 74 bp, synthetic construct
  source 1..74: mol_type = other DNA
gtctctgtggcgcaatcggttagcgcgttcggctctaaactgaaaggttagtggttcgagcccacccggggacg

SEQ ID NO: 180 - DNA, 74 bp, synthetic construct
  source 1..74: mol_type = other DNA
gtctctgtggcgcaatcggttagcgcgttcggcttcaaactgaaaggttggtggttcgagcccacccagggacg

SEQ ID NO: 181 - DNA, 74 bp, synthetic construct
  source 1..74: mol_type = other DNA
gtctctgtggcgcaatcggttagcgcgttcggcttcaaaccgaaagattggtggttcgagcccacccagggacg

SEQ ID NO: 182 - DNA, 74 bp, synthetic construct
  source 1..74: mol_type = other DNA
gtctctgtggcgcaatcggttagcgcattcggcttcaaaccgaaaggttggtggttcgagcccacccagggacg

SEQ ID NO: 183 - DNA, 74 bp, synthetic construct
  source 1..74: mol_type = other DNA
gtctctgtggcgcaatcggttagcgcgttcggcttcaaaccgaaaggttggtggttcgatcccacccagggacg

SEQ ID NO: 184 - DNA, 74 bp, synthetic construct
  source 1..74: mol_type = other DNA
gtctctgtggcgcaatgggttagcgcgttcggcttcaaaccgaaaggttggtggttcgagcccatccagggacg

SEQ ID NO: 185 - DNA, 74 bp, synthetic construct
  source 1..74: mol_type = other DNA
gtctctgtggcgcaatcggttagcgcgttcggcttcaaaccgaaaggttggtggttcgagcccacccagggacg

SEQ ID NO: 186 - DNA, 74 bp, synthetic construct
  source 1..74: mol_type = other DNA
gtctctgtggcgcaatcggctagcgcgtttggcttcaaactaaaaggttggtggttcgaacccacccagaggcg

SEQ ID NO: 187 - DNA, 74 bp, synthetic construct
  source 1..74: mol_type = other DNA
gtctctgtggcgcaatcggctagcgcgtttggcttcaaactaaaaagttggtggttcgaacacacccagaggcg

SEQ ID NO: 188 - DNA, 74 bp, synthetic construct
  source 1..74: mol_type = other DNA
gtctctgtggtgcaatcggttagcgcgttccgcttcaaaccgaaagcttggtggttcgagcccacccagggatg

SEQ ID NO: 189 - DNA, 74 bp, synthetic construct
  source 1..74: mol_type = other DNA
gtctctgtggcgcaatcggctagcgcgtttggcttcaaactaaaaggttggcggttcgaacccacccagaggcg

SEQ ID NO: 190 - DNA, 74 bp, synthetic construct
  source 1..74: mol_type = other DNA
gtctctgtggcgtagtcggttagcgcgttcggcttcaaaccgaaaagttggtggttcgagcccacccaggaacg

SEQ ID NO: 191 - DNA, 74 bp, synthetic construct
  source 1..74: mol_type = other DNA
gtctctgtggcgcaatcggttagcgcgttcggcttcaaactgaaaggttagtggttcgagcccacccggggacg

SEQ ID NO: 192 - DNA, 72 bp, synthetic construct
  source 1..72: mol_type = other DNA
tcctcgttagtatagtggttagtatccccgcctttaacgcgggagaccggggttcaattccccgacggggag

SEQ ID NO: 193 - DNA, 72 bp, synthetic construct
  source 1..72: mol_type = other DNA
tcctcgttagtatagtggtgagtatccccgcctttaacgcgggagaccggggttcgattccccgacggggag

SEQ ID NO: 194 - DNA, 72 bp, synthetic construct
  source 1..72: mol_type = other DNA
tcctcgttagtatagtggtgagtgtccccgtctttaacgcgggagaccggggttcgattccccgacggggag

SEQ ID NO: 195 - DNA, 72 bp, synthetic construct
  source 1..72: mol_type = other DNA
tcctcgttagtatagtggtgagtatccccgcctctaacgcgggagaccggggttcgattccccgacggggag

SEQ ID NO: 196 - DNA, 72 bp, synthetic construct
  source 1..72: mol_type = other DNA
tcctcgttagtatagtggttagtatccccgcctctaacgcgggagaccggggttcaattccccgacggggag

SEQ ID NO: 197 - DNA, 72 bp, synthetic construct
  source 1..72: mol_type = other DNA
tcctcgttagtatagtggtgagtgtccccgtctctaacgcgggagaccggggttcgattccccgacggggag

SEQ ID NO: 198 - DNA, 72 bp, synthetic construct
  source 1..72: mol_type = other DNA
tcctcgttagtatagtggtgagtgtccccgtcttcaacgcgggagaccggggttcgattccccgacggggag

SEQ ID NO: 199 - DNA, 72 bp, synthetic construct
  source 1..72: mol_type = other DNA
tcctcgttagtatagtggttagtatccccgccttcaacgcgggagaccggggttcaattccccgacggggag

SEQ ID NO: 200 - DNA, 72 bp, synthetic construct
  source 1..72: mol_type = other DNA
tcctcgttagtatagtggtgagtatccccgccttcaacgcgggagaccggggttcgattccccgacggggag

SEQ ID NO: 201 - DNA, 72 bp, synthetic construct
  source 1..72: mol_type = other DNA
gggggtatagctcagtggtagagcatttgactttagatcaagaggtccctggttcaaatccgggtgccccct

SEQ ID NO: 202 - DNA, 73 bp, synthetic construct
  source 1..73: mol_type = other DNA
gggggtatagctcagtgggtagagcatttgactttagatcaagaggtccccggttcaaatccgggtgccccct

SEQ ID NO: 203 - DNA, 72 bp, synthetic construct
  source 1..72: mol_type = other DNA
gggggtatagctcaggggtagagcatttgactttagatcaagaggtccccggttcaaatccgggtgccccct

SEQ ID NO: 204 - DNA, 72 bp, synthetic construct
  source 1..72: mol_type = other DNA
gggggtatagcttagcggtagagcatttgactttagatcaagaggtccccggttcaaatccgggtgccccct

SEQ ID NO: 205 - DNA, 72 bp, synthetic construct
  source 1..72: mol_type = other DNA
gggggtatagctcaggggtagagcatttgactttagatcaagaagtccccggttcaaatccgggtgccccct

SEQ ID NO: 206 - DNA, 72 bp, synthetic construct
  source 1..72: mol_type = other DNA
gggggtgtagctcagtggtagagcatttgactttagatcaagaggtccctggttcaaatccaggtgccccct

SEQ ID NO: 207 - DNA, 72 bp, synthetic construct
  source 1..72: mol_type = other DNA
gggggtatagctcaggggtagagcatttgactttagatcaagaggtccctggttcaaatccaggtgccccct

SEQ ID NO: 208 - DNA, 72 bp, synthetic construct
  source 1..72: mol_type = other DNA
gggggtatagctcaggggtagagcatttgactttagatcaagaggtctctggttcaaatccaggtgccccct

SEQ ID NO: 209 - DNA, 72 bp, synthetic construct
  source 1..72: mol_type = other DNA
gggggtatagctcaggggtagagcatttgactttagatcaagaggtccccagttcaaatctgggtgccccct

SEQ ID NO: 210 - DNA, 72 bp, synthetic construct
  source 1..72: mol_type = other DNA
gggggtatagctcaggggtagagcatttgactttagatcaagaggtccctggttcaaatccaggtgcccccc

SEQ ID NO: 211 - DNA, 72 bp, synthetic construct
  source 1..72: mol_type = other DNA
gggggtatagctcaggggtagagcacttgactttagatcaagaggtccctggttcaaatccaggtgccccct

SEQ ID NO: 212 - DNA, 72 bp, synthetic construct
  source 1..72: mol_type = other DNA
gggggtatagcttaggggtagagcatttgactttagatcaaaaggtccctggttcaaatccaggtgcccctt

SEQ ID NO: 213 - DNA, 72 bp, synthetic construct
  source 1..72: mol_type = other DNA
gggggtatagctcaggggtagagcacttgactttagatcaagaagtccttggttcaaatccaggtgccccct

SEQ ID NO: 214 - DNA, 66 bp, synthetic construct
  source 1..66: mol_type = other DNA
ggtatatctcagggggcagagcatttgactttagatcaagaggtccccggttgaaatccgggtgct

SEQ ID NO: 215 - DNA, 72 bp, synthetic construct
  source 1..72: mol_type = other DNA
gggggcatagctcagtggtagagcatttgactttagatcaagaggtccctggttcaaatccaggtgccccct

SEQ ID NO: 216 - DNA, 72 bp, synthetic construct
  source 1..72: mol_type = other DNA
gggggtatagctcacaggtagagcatttgactttagatcaagaggtccccggttcaaatccggttactccct

SEQ ID NO: 217 - DNA, 75 bp, synthetic construct
  source 1..75: mol_type = other DNA
ggcagaatggtgcagcggttcagcacccaggctctttagccagctgttgcctgggctcaaatcccagctctgcca

SEQ ID NO: 218 - DNA, 72 bp, synthetic construct
  source 1..72: mol_type = other DNA
gggggtatagctcacaggtagagcatttgactttagatcaagaggtccccggttcaaatctgggtgccccct

SEQ ID NO: 219 - DNA, 72 bp, synthetic construct
  source 1..72: mol_type = other DNA
gggggtagggctcagggatagagcatttgactttagatcaagaggtccccggttcgaatctaggtgccccct

SEQ ID NO: 220 - DNA, 72 bp, synthetic construct
  source 1..72: mol_type = other DNA
gggcgtatagctcaggggtagagcatttgactttagatcaagaggtccccagttcaaatctgggtgccccct

SEQ ID NO: 221 - DNA, 72 bp, synthetic construct
  source 1..72: mol_type = other DNA
gggggtatagttcaggggtagagcatttgactttagatcaagaggtccctggttcaaatccaggtgccccct

SEQ ID NO: 222 - DNA, 79 bp, synthetic construct
  source 1..79: mol_type = other DNA
ggctgtatagctcagtggtagagcatttgactttagctttaaagtcatatgtagctgggttcaaatcctgtttctgcca

SEQ ID NO: 223 - DNA, 72 bp, synthetic construct
  source 1..72: mol_type = other DNA
gggggtatagctcaggggtagagcatttgactttaaatcaagaggtccctgattcaaatccaggtgccccct

SEQ ID NO: 224 - DNA, 72 bp, synthetic construct
  source 1..72: mol_type = other DNA
ggggatatagctcaggggtagagcatttgactttagatcaagaggtccccggttcaaatccgggtgcccccc

SEQ ID NO: 225 - DNA, 70 bp, synthetic construct
  source 1..70: mol_type = other DNA
gggcgtatagctcaggggtagagcatttgactttagatcaagaggtccccagttcaaatctgggtgccca

SEQ ID NO: 226 - DNA, 72 bp, synthetic construct
  source 1..72: mol_type = other DNA
gggggtatagctcaggggtagagcatttgactctagatcaagaggtccctggttcaaatccaggtgcccccc

SEQ ID NO: 227 - DNA, 73 bp, synthetic construct
  source 1..73: mol_type = other DNA
gggggtatagctcagtgggtagagcatttgactctagatcaagaggtccccggttcaaatccgggtgccccct

SEQ ID NO: 228 - DNA, 72 bp, synthetic construct
  source 1..72: mol_type = other DNA
gggggtatagctcagtggtagagcatttgactctagatcaagaggtccctggttcaaatccgggtgccccct

SEQ ID NO: 229 - DNA, 72 bp, synthetic construct
  source 1..72: mol_type = other DNA
gggggtgtagctcagtggtagagcatttgactctagatcaagaggtccctggttcaaatccaggtgccccct

SEQ ID NO: 230 - DNA, 72 bp, synthetic construct
  source 1..72: mol_type = other DNA
gggggtatagctcaggggtagagcatttgactctagatcaagaggtccccggttcaaatccgggtgccccct

SEQ ID NO: 231 - DNA, 72 bp, synthetic construct
  source 1..72: mol_type = other DNA
gggggtatagctcaggggtagagcatttgactctagatcaagaggtccctggttcaaatccaggtgccccct

SEQ ID NO: 232 - DNA, 72 bp, synthetic construct
  source 1..72: mol_type = other DNA
gggggcatagctcagtggtagagcatttgactctagatcaagaggtccctggttcaaatccaggtgccccct

SEQ ID NO: 233 - DNA, 72 bp, synthetic construct
  source 1..72: mol_type = other DNA
gggggtatagctcaggggtagagcacttgactctagatcaagaggtccctggttcaaatccaggtgccccct

SEQ ID NO: 234 - DNA, 72 bp, synthetic construct
  source 1..72: mol_type = other DNA
gggggtatagctcaggggtagagcatttgactctagatcaagaggtccccagttcaaatctgggtgccccct

SEQ ID NO: 235 - DNA, 72 bp, synthetic construct
  source 1..72: mol_type = other DNA
gggggtatagctcaggggtagagcatttgactctagatcaagaagtccccggttcaaatccgggtgccccct

SEQ ID NO: 236 - DNA, 72 bp, synthetic construct
  source 1..72: mol_type = other DNA
gggggtatagcttagcggtagagcatttgactctagatcaagaggtccccggttcaaatccgggtgccccct

SEQ ID NO: 237 - DNA, 72 bp, synthetic construct
  source 1..72: mol_type = other DNA
gggggtatagctcaggggtagagcatttgactctagatcaagaggtctctggttcaaatccaggtgccccct

SEQ ID NO: 238 - DNA, 72 bp, synthetic construct
  source 1..72: mol_type = other DNA
gggggtatagctcaggggtagagcacttgactctagatcaagaagtccttggttcaaatccaggtgccccct

SEQ ID NO: 239 - DNA, 72 bp, synthetic construct
  source 1..72: mol_type = other DNA
gggggtagggctcagggatagagcatttgactctagatcaagaggtccccggttcgaatctaggtgccccct

SEQ ID NO: 240 - DNA, 72 bp, synthetic construct
  source 1..72: mol_type = other DNA
ggggatatagctcaggggtagagcatttgactctagatcaagaggtccccggttcaaatccgggtgcccccc

SEQ ID NO: 241 - DNA, 72 bp, synthetic construct
  source 1..72: mol_type = other DNA
gggggtatagttcaggggtagagcatttgactctagatcaagaggtccctggttcaaatccaggtgccccct

SEQ ID NO: 242 - DNA, 72 bp, synthetic construct
  source 1..72: mol_type = other DNA
gggggtatagctcacaggtagagcatttgactctagatcaagaggtccccggttcaaatccggttactccct

SEQ ID NO: 243 - DNA, 72 bp, synthetic construct
  source 1..72: mol_type = other DNA
gggggtatagctcaggggtagagcatttgactctaaatcaagaggtccctgattcaaatccaggtgccccct

SEQ ID NO: 244 - DNA, 70 bp, synthetic construct
  source 1..70: mol_type = other DNA
gggcgtatagctcaggggtagagcatttgactctagatcaagaggtccccagttcaaatctgggtgccca

SEQ ID NO: 245 - DNA, 72 bp, synthetic construct
  source 1..72: mol_type = other DNA
gggggtatagctcacaggtagagcatttgactctagatcaagaggtccccggttcaaatctgggtgccccct

SEQ ID NO: 246 - DNA, 72 bp, synthetic construct
  source 1..72: mol_type = other DNA
gggcgtatagctcaggggtagagcatttgactctagatcaagaggtccccagttcaaatctgggtgccccct

SEQ ID NO: 247 - DNA, 66 bp, synthetic construct
  source 1..66: mol_type = other DNA
ggtatatctcagggggcagagcatttgactctagatcaagaggtccccggttgaaatccgggtgct

SEQ ID NO: 248 - DNA, 79 bp, synthetic construct
  source 1..79: mol_type = other DNA
ggctgtatagctcagtggtagagcatttgactctagctttaaagtcatatgtagctgggttcaaatcctgtttctgcca

SEQ ID NO: 249 - DNA, 72 bp, synthetic construct
  source 1..72: mol_type = other DNA
gggggtatagcttaggggtagagcatttgactctagatcaaaaggtccctggttcaaatccaggtgcccctt

SEQ ID NO: 250 - DNA, 75 bp, synthetic construct
  source 1..75: mol_type = other DNA
ggcagaatggtgcagcggttcagcacccaggctctctagccagctgttgcctgggctcaaatcccagctctgcca

SEQ ID NO: 251 - DNA, 73 bp, synthetic construct
  source 1..73: mol_type = other DNA
gggggtatagctcagtgggtagagcatttgacttcagatcaagaggtccccggttcaaatccgggtgccccct

SEQ ID NO: 252 - DNA, 72 bp, synthetic construct
  source 1..72: mol_type = other DNA
gggggtatagcttagcggtagagcatttgacttcagatcaagaggtccccggttcaaatccgggtgccccct

SEQ ID NO: 253 - DNA, 72 bp, synthetic construct
  source 1..72: mol_type = other DNA
gggggtatagctcaggggtagagcatttgacttcagatcaagaggtccccggttcaaatccgggtgccccct

SEQ ID NO: 254 - DNA, 72 bp, synthetic construct
  source 1..72: mol_type = other DNA
gggggtatagctcaggggtagagcatttgacttcagatcaagaggtccccagttcaaatctgggtgccccct

SEQ ID NO: 255 - DNA, 72 bp, synthetic construct
  source 1..72: mol_type = other DNA
gggggtatagctcagtggtagagcatttgacttcagatcaagaggtccctggttcaaatccgggtgccccct

SEQ ID NO: 256 - DNA, 72 bp, synthetic construct
  source 1..72: mol_type = other DNA
gggggtatagctcaggggtagagcatttgacttcagatcaagaggtctctggttcaaatccaggtgccccct

SEQ ID NO: 257 - DNA, 72 bp, synthetic construct
  source 1..72: mol_type = other DNA
gggggcatagctcagtggtagagcatttgacttcagatcaagaggtccctggttcaaatccaggtgccccct

SEQ ID NO: 258 - DNA, 72 bp, synthetic construct
  source 1..72: mol_type = other DNA
gggggtatagctcaggggtagagcatttgacttcagatcaagaggtccctggttcaaatccaggtgccccct

SEQ ID NO: 259 - DNA, 72 bp, synthetic construct
  source 1..72: mol_type = other DNA
gggggtgtagctcagtggtagagcatttgacttcagatcaagaggtccctggttcaaatccaggtgccccct

SEQ ID NO: 260 - DNA, 72 bp, synthetic construct
  source 1..72: mol_type = other DNA
gggggtatagcttaggggtagagcatttgacttcagatcaaaaggtccctggttcaaatccaggtgcccctt

SEQ ID NO: 261 - DNA, 72 bp, synthetic construct
  source 1..72: mol_type = other DNA
gggggtatagctcaggggtagagcacttgacttcagatcaagaggtccctggttcaaatccaggtgccccct

SEQ ID NO: 262 - DNA, 72 bp, synthetic construct
  source 1..72: mol_type = other DNA
gggggtatagctcaggggtagagcatttgacttcagatcaagaagtccccggttcaaatccgggtgccccct

SEQ ID NO: 263 - DNA, 72 bp, synthetic construct
  source 1..72: mol_type = other DNA
gggggtatagctcaggggtagagcatttgacttcagatcaagaggtccctggttcaaatccaggtgcccccc

SEQ ID NO: 264 - DNA, 72 bp, synthetic construct
  source 1..72: mol_type = other DNA
gggggtatagctcaggggtagagcacttgacttcagatcaagaagtccttggttcaaatccaggtgccccct

SEQ ID NO: 265 - DNA, 66 bp, synthetic construct
  source 1..66: mol_type = other DNA
ggtatatctcagggggcagagcatttgacttcagatcaagaggtccccggttgaaatccgggtgct

SEQ ID NO: 266 - DNA, 79 bp, synthetic construct
  source 1..79: mol_type = other DNA
ggctgtatagctcagtggtagagcatttgacttcagctttaaagtcatatgtagctgggttcaaatcctgtttctgcca

SEQ ID NO: 267 - DNA, 75 bp, synthetic construct
  source 1..75: mol_type = other DNA
ggcagaatggtgcagcggttcagcacccaggctcttcagccagctgttgcctgggctcaaatcccagctctgcca

SEQ ID NO: 268 - DNA, 72 bp, synthetic construct
  source 1..72: mol_type = other DNA
gggggtatagctcacaggtagagcatttgacttcagatcaagaggtccccggttcaaatccggttactccct

SEQ ID NO: 269 - DNA, 72 bp, synthetic construct
  source 1..72: mol_type = other DNA
ggggatatagctcaggggtagagcatttgacttcagatcaagaggtccccggttcaaatccgggtgcccccc

SEQ ID NO: 270 - DNA, 72 bp, synthetic construct
  source 1..72: mol_type = other DNA
gggggtatagctcacaggtagagcatttgacttcagatcaagaggtccccggttcaaatctgggtgccccct

SEQ ID NO: 271 - DNA, 70 bp, synthetic construct
  source 1..70: mol_type = other DNA
gggcgtatagctcaggggtagagcatttgacttcagatcaagaggtccccagttcaaatctgggtgccca

SEQ ID NO: 272 - DNA, 72 bp, synthetic construct
  source 1..72: mol_type = other DNA
gggcgtatagctcaggggtagagcatttgacttcagatcaagaggtccccagttcaaatctgggtgccccct

SEQ ID NO: 273 - DNA, 72 bp, synthetic construct
  source 1..72: mol_type = other DNA
gggggtagggctcagggatagagcatttgacttcagatcaagaggtccccggttcgaatctaggtgccccct

SEQ ID NO: 274 - DNA, 72 bp, synthetic construct
  source 1..72: mol_type = other DNA
gggggtatagctcaggggtagagcatttgacttcaaatcaagaggtccctgattcaaatccaggtgccccct

SEQ ID NO: 275 - DNA, 72 bp, synthetic construct
  source 1..72: mol_type = other DNA
gggggtatagttcaggggtagagcatttgacttcagatcaagaggtccctggttcaaatccaggtgccccct

SEQ ID NO: 276 - DNA, 72 bp, synthetic construct
  source 1..72: mol_type = other DNA
ggtcccatggtgtaatggttagcactctggactttaaatccagcgatccgagttcaaatctcggtgggacct

SEQ ID NO: 277 - DNA, 72 bp, synthetic construct
  source 1..72: mol_type = other DNA
ggttccatggtgtaatggttagcactctggactttaaatccagcgatccgagttcaaatctcggtggaacct

SEQ ID NO: 278 - DNA, 72 bp, synthetic construct
  source 1..72: mol_type = other DNA
ggccccatggtgtaatggttagcactctggactttaaatccagcgatccgagttcaaatctcggtgggacct

SEQ ID NO: 279 - DNA, 72 bp, synthetic construct
  source 1..72: mol_type = other DNA
ggccccatggtgtaatggtcagcactctggactttaaatccagcgatccgagttcaaatctcggtgggaccc

SEQ ID NO: 280 - DNA, 72 bp, synthetic construct
  source 1..72: mol_type = other DNA
ggttccatggtgtaatggtaagcactctggactttaaatccagcgatccgagttcgagtctcggtggaacct

SEQ ID NO: 281 - DNA, 72 bp, synthetic construct
  source 1..72: mol_type = other DNA
ggtcccatggtgtaatggttagcactctggactttaaatccagcaatccgagttcgaatctcggtgggacct

SEQ ID NO: 282 - DNA, 72 bp, synthetic construct
  source 1..72: mol_type = other DNA
ggttccatggtgtaatggttagcactctggactttaaatccggtaatccgagttcaaatctcggtggaacct

SEQ ID NO: 283 - DNA, 72 bp, synthetic construct
  source 1..72: mol_type = other DNA
ggttccatggtgtaatggtgagcactctggactttaaatccagcgatccgagttcgagtctcggtggaacct

SEQ ID NO: 284 - DNA, 72 bp, synthetic construct
  source 1..72: mol_type = other DNA
ggttccatggtgtaatggttagcactctggactttaaatccagcgatccgagttcaagtctcggtggaacct

SEQ ID NO: 285 - DNA, 72 bp, synthetic construct
  source 1..72: mol_type = other DNA
ggtcccatggtgtaatggttagcactctgggctttaaatccagcaatccgagttcgaatcttggtgggacct

SEQ ID NO: 286 - DNA, 74 bp, synthetic construct
  source 1..74: mol_type = other DNA
ggtacagtgttaaaggggagaaaaattgctgactttaaatacagtagacctaggtttgaatcctggctttacca

SEQ ID NO: 287 - DNA, 73 bp, synthetic construct
  source 1..73: mol_type = other DNA
ggctgtgtacctcagtgggcaagggtatggactttaaagccagactatttgggttcaaatcccagcttggcct

SEQ ID NO: 288 - DNA, 72 bp, synthetic construct
  source 1..72: mol_type = other DNA
ggttccatggtgtaatggtgagggctttggactttaactacagtgatcagagttcaagtctcagtgggacct

SEQ ID NO: 289 - DNA, 72 bp, synthetic construct
  source 1..72: mol_type = other DNA
ggttccatggtgtaatggtgaccactttggactttaaatacagtgatcagagttcaagtctcactggaacct

SEQ ID NO: 290 - DNA, 73 bp, synthetic construct
  source 1..73: mol_type = other DNA
ggttccatggtgtaatggtaagcaccctggactttaaatccagcaaccagagttccagtctcagcgtggacct

SEQ ID NO: 291 - DNA, 68 bp, synthetic construct
  source 1..68: mol_type = other DNA
gtttccatggtgtaatggtgagcactctggactttaaatccagaaatacattcaaagaattaagaaca

SEQ ID NO: 292 - DNA, 72 bp, synthetic construct
  source 1..72: mol_type = other DNA
ggttccatggtgtaatggtgagcactttggactttaaatacagtgatcagagttcaagtctcactgggacct

SEQ ID NO: 293 - DNA, 72 bp, synthetic construct
  source 1..72: mol_type = other DNA
ggttccatgggttaatggtgagcaccctggactttaaatcaagcgatccgagttcaaatctcggtggtacct

SEQ ID NO: 294 - DNA, 72 bp, synthetic construct
  source 1..72: mol_type = other DNA
ggttccatggtgtaatggtaagcactctggactttaaatccagccatctgagttcgagtctctgtggaacct

SEQ ID NO: 295 - DNA, 72 bp, synthetic construct
  source 1..72: mol_type = other DNA
ggtagtgtagtctactggttaaacgcttgggctttaacattaacgtcctgggttcaaatcccagctttgtca

SEQ ID NO: 296 - DNA, 73 bp, synthetic construct
  source 1..73: mol_type = other DNA
gaccatgtggcctaagggaaaagacatctcactttaggtcagaagattgagggttcaagtcctttcatggtca

SEQ ID NO: 297 - DNA, 72 bp, synthetic construct
  source 1..72: mol_type = other DNA
ggttccatggtgtaatggtgagcactctggactctaaatccagcgatccgagttcgagtctcggtggaacct

SEQ ID NO: 298 - DNA, 72 bp, synthetic construct
  source 1..72: mol_type = other DNA
ggccccatggtgtaatggtcagcactctggactctaaatccagcgatccgagttcaaatctcggtgggaccc

SEQ ID NO: 299 - DNA, 72 bp, synthetic construct
  source 1..72: mol_type = other DNA
ggttccatggtgtaatggtaagcactctggactctaaatccagcgatccgagttcgagtctcggtggaacct

SEQ ID NO: 300 - DNA, 72 bp, synthetic construct
  source 1..72: mol_type = other DNA
ggttccatggtgtaatggttagcactctggactctaaatccggtaatccgagttcaaatctcggtggaacct

SEQ ID NO: 301 - DNA, 72 bp, synthetic construct
  source 1..72: mol_type = other DNA
ggttccatggtgtaatggttagcactctggactctaaatccagcgatccgagttcaagtctcggtggaacct

SEQ ID NO: 302 - DNA, 72 bp, synthetic construct
  source 1..72: mol_type = other DNA
ggccccatggtgtaatggttagcactctggactctaaatccagcgatccgagttcaaatctcggtgggacct

SEQ ID NO: 303 - DNA, 72 bp, synthetic construct
  source 1..72: mol_type = other DNA
ggtcccatggtgtaatggttagcactctggactctaaatccagcaatccgagttcgaatctcggtgggacct

SEQ ID NO: 304 - DNA, 72 bp, synthetic construct
  source 1..72: mol_type = other DNA
ggtcccatggtgtaatggttagcactctggactctaaatccagcgatccgagttcaaatctcggtgggacct

SEQ ID NO: 305 - DNA, 72 bp, synthetic construct
  source 1..72: mol_type = other DNA
ggttccatggtgtaatggttagcactctggactctaaatccagcgatccgagttcaaatctcggtggaacct

SEQ ID NO: 306 - DNA, 72 bp, synthetic construct
  source 1..72: mol_type = other DNA
ggtcccatggtgtaatggttagcactctgggctctaaatccagcaatccgagttcgaatcttggtgggacct

SEQ ID NO: 307 - DNA, 72 bp, synthetic construct
  source 1..72: mol_type = other DNA
ggttccatggtgtaatggtgagcactttggactctaaatacagtgatcagagttcaagtctcactgggacct

SEQ ID NO: 308 - DNA, 72 bp, synthetic construct
  source 1..72: mol_type = other DNA
ggttccatggtgtaatggtgaccactttggactctaaatacagtgatcagagttcaagtctcactggaacct

SEQ ID NO: 309 - DNA, 72 bp, synthetic construct
  source 1..72: mol_type = other DNA
ggttccatggtgtaatggtaagcactctggactctaaatccagccatctgagttcgagtctctgtggaacct

SEQ ID NO: 310 - DNA, 73 bp, synthetic construct
  source 1..73: mol_type = other DNA
ggttccatggtgtaatggtaagcaccctggactctaaatccagcaaccagagttccagtctcagcgtggacct

SEQ ID NO: 311 - DNA, 72 bp, synthetic construct
  source 1..72: mol_type = other DNA
ggttccatggtgtaatggtgagggctttggactctaactacagtgatcagagttcaagtctcagtgggacct

SEQ ID NO: 312 - DNA, 72 bp, synthetic construct
  source 1..72: mol_type = other DNA
ggtagtgtagtctactggttaaacgcttgggctctaacattaacgtcctgggttcaaatcccagctttgtca

SEQ ID NO: 313 - DNA, 72 bp, synthetic construct
  source 1..72: mol_type = other DNA
ggttccatgggttaatggtgagcaccctggactctaaatcaagcgatccgagttcaaatctcggtggtacct

SEQ ID NO: 314 - DNA, 73 bp, synthetic construct
  source 1..73: mol_type = other DNA
ggctgtgtacctcagtgggcaagggtatggactctaaagccagactatttgggttcaaatcccagcttggcct

SEQ ID NO: 315 - DNA, 74 bp, synthetic construct
  source 1..74: mol_type = other DNA
ggtacagtgttaaaggggagaaaaattgctgactctaaatacagtagacctaggtttgaatcctggctttacca

SEQ ID NO: 316 - DNA, 68 bp, synthetic construct
  source 1..68: mol_type = other DNA
gtttccatggtgtaatggtgagcactctggactctaaatccagaaatacattcaaagaattaagaaca

SEQ ID NO: 317 - DNA, 73 bp, synthetic construct
  source 1..73: mol_type = other DNA
gaccatgtggcctaagggaaaagacatctcactctaggtcagaagattgagggttcaagtcctttcatggtca

SEQ ID NO: 318 - DNA, 72 bp, synthetic construct
  source 1..72: mol_type = other DNA
ggccccatggtgtaatggttagcactctggacttcaaatccagcgatccgagttcaaatctcggtgggacct

SEQ ID NO: 319 - DNA, 72 bp, synthetic construct
  source 1..72: mol_type = other DNA
ggttccatggtgtaatggtaagcactctggacttcaaatccagcgatccgagttcgagtctcggtggaacct

SEQ ID NO: 320 - DNA, 72 bp, synthetic construct
  source 1..72: mol_type = other DNA
ggtcccatggtgtaatggttagcactctggacttcaaatccagcaatccgagttcgaatctcggtgggacct

SEQ ID NO: 321 - DNA, 72 bp, synthetic construct
  source 1..72: mol_type = other DNA
ggtcccatggtgtaatggttagcactctggacttcaaatccagcgatccgagttcaaatctcggtgggacct

SEQ ID NO: 322 - DNA, 72 bp, synthetic construct
  source 1..72: mol_type = other DNA
ggttccatggtgtaatggtgagcactctggacttcaaatccagcgatccgagttcgagtctcggtggaacct

SEQ ID NO: 323 - DNA, 72 bp, synthetic construct
  source 1..72: mol_type = other DNA
ggttccatggtgtaatggttagcactctggacttcaaatccggtaatccgagttcaaatctcggtggaacct

SEQ ID NO: 324 - DNA, 72 bp, synthetic construct
  source 1..72: mol_type = other DNA
ggccccatggtgtaatggtcagcactctggacttcaaatccagcgatccgagttcaaatctcggtgggaccc

SEQ ID NO: 325 - DNA, 72 bp, synthetic construct
  source 1..72: mol_type = other DNA
ggttccatggtgtaatggttagcactctggacttcaaatccagcgatccgagttcaaatctcggtggaacct

SEQ ID NO: 326 - DNA, 72 bp, synthetic construct
  source 1..72: mol_type = other DNA
ggtcccatggtgtaatggttagcactctgggcttcaaatccagcaatccgagttcgaatcttggtgggacct

SEQ ID NO: 327 - DNA, 73 bp, synthetic construct
  source 1..73: mol_type = other DNA
gaccatgtggcctaagggaaaagacatctcacttcaggtcagaagattgagggttcaagtcctttcatggtca

SEQ ID NO: 328 - DNA, 72 bp, synthetic construct
  source 1..72: mol_type = other DNA
ggttccatggtgtaatggttagcactctggacttcaaatccagcgatccgagttcaagtctcggtggaacct

SEQ ID NO: 329 - DNA, 72 bp, synthetic construct
  source 1..72: mol_type = other DNA
ggttccatggtgtaatggtgagcactttggacttcaaatacagtgatcagagttcaagtctcactgggacct

SEQ ID NO: 330 - DNA, 74 bp, synthetic construct
  source 1..74: mol_type = other DNA
ggtacagtgttaaaggggagaaaaattgctgacttcaaatacagtagacctaggtttgaatcctggctttacca

SEQ ID NO: 331 - DNA, 73 bp, synthetic construct
  source 1..73: mol_type = other DNA
ggctgtgtacctcagtgggcaagggtatggacttcaaagccagactatttgggttcaaatcccagcttggcct

SEQ ID NO: 332 - DNA, 72 bp, synthetic construct
  source 1..72: mol_type = other DNA
ggttccatggtgtaatggtaagcactctggacttcaaatccagccatctgagttcgagtctctgtggaacct

SEQ ID NO: 333 - DNA, 72 bp, synthetic construct
  source 1..72: mol_type = other DNA
ggttccatgggttaatggtgagcaccctggacttcaaatcaagcgatccgagttcaaatctcggtggtacct

SEQ ID NO: 334 - DNA, 72 bp, synthetic construct
  source 1..72: mol_type = other DNA
ggttccatggtgtaatggtgagggctttggacttcaactacagtgatcagagttcaagtctcagtgggacct

SEQ ID NO: 335 - DNA, 68 bp, synthetic construct
  source 1..68: mol_type = other DNA
gtttccatggtgtaatggtgagcactctggacttcaaatccagaaatacattcaaagaattaagaaca

SEQ ID NO: 336 - DNA, 73 bp, synthetic construct
  source 1..73: mol_type = other DNA
ggttccatggtgtaatggtaagcaccctggacttcaaatccagcaaccagagttccagtctcagcgtggacct

SEQ ID NO: 337 - DNA, 72 bp, synthetic construct
  source 1..72: mol_type = other DNA
ggttccatggtgtaatggtgaccactttggacttcaaatacagtgatcagagttcaagtctcactggaacct

SEQ ID NO: 338 - DNA, 72 bp, synthetic construct
  source 1..72: mol_type = other DNA
ggtagtgtagtctactggttaaacgcttgggcttcaacattaacgtcctgggttcaaatcccagctttgtca

SEQ ID NO: 339 - DNA, 72 bp, synthetic construct
  source 1..72: mol_type = other DNA
tcccacatggtctagcggttaggattcctggttttaacccaggcggcccgggttcgactcccggtgtgggaa

SEQ ID NO: 340 - DNA, 72 bp, synthetic construct
  source 1..72: mol_type = other DNA
tcccatatggtctagcggttaggattcctggttttaacccaggtggcccgggttcgactcccggtatgggaa

SEQ ID NO: 341 - DNA, 72 bp, synthetic construct
  source 1..72: mol_type = other DNA
tccctggtggtctagtggctaggattcggcgctttaaccgccgcggcccgggttcgattcccggtcagggaa

SEQ ID NO: 342 - DNA, 72 bp, synthetic construct
  source 1..72: mol_type = other DNA
tccctggtggtctagtggttaggattcggcgctttaaccgccgcggcccgggttcgattcccggtcagggaa

SEQ ID NO: 343 - DNA, 72 bp, synthetic construct
  source 1..72: mol_type = other DNA
tccctggtggtctagtggctaggattcggcgctttaaccgccgcggcccgggttcgattcccggccagggaa

SEQ ID NO: 344 - DNA, 71 bp, synthetic construct
  source 1..71: mol_type = other DNA
tccttgatgtctagtggttaggatttggtgctttaactgcagcagcctgggttcatttctcagtcagggaa

SEQ ID NO: 345 - DNA, 72 bp, synthetic construct
  source 1..72: mol_type = other DNA
tccttggtggtctagtggctaggattcggtgctttaacctgtgcggcccgggttcaattcccgatgaaggaa

SEQ ID NO: 346 - DNA, 73 bp, synthetic construct
  source 1..73: mol_type = other DNA
tccctggtggtctagtggctaggattcggcgctttaaccgcctgcagctcgagttcgattcctggtcagggaa

SEQ ID NO: 347 - DNA, 72 bp, synthetic construct
  source 1..72: mol_type = other DNA
gcgttggtggtgtagtggtgagcacagctgcctttaaagcagttaacgcgggttcgattcccgggtaacgaa

SEQ ID NO: 348 - DNA, 72 bp, synthetic construct
  source 1..72: mol_type = other DNA
tccctggtggtctagtggttaggattcggcgctttaaccgccgcggcccgggttcgattcccggtcaggaaa

SEQ ID NO: 349 - DNA, 71 bp, synthetic construct
  source 1..71: mol_type = other DNA
gcaatggtggttcagtggtagaattctcgcctttaacacaggagacccgggttcaattcctgacccatgta

SEQ ID NO: 350 - DNA, 72 bp, synthetic construct
  source 1..72: mol_type = other DNA
tgtctggtggtcaagtggctaggatttggcgctttaactgccgcggcccgcgttcgattcccggtcagggaa

SEQ ID NO: 351 - DNA, 69 bp, synthetic construct
  source 1..69: mol_type = other DNA
ccctgtggtctagtggctaagactttgtgctttaattgctgcatcctaggttcaattcccagtcaggga

SEQ ID NO: 352 - DNA, 72 bp, synthetic construct
  source 1..72: mol_type = other DNA
cccctggtggtctagtgcttaggattcggtgctttaaccgctgctgcctgcgttcgattcccggtcagggaa

SEQ ID NO: 353 - DNA, 73 bp, synthetic construct
  source 1..73: mol_type = other DNA
tccgtggtggtctagtggctaggattcggcgctttaaccgcctgcagctcgagttcgattcctggtcagggaa

SEQ ID NO: 354 - DNA, 73 bp, synthetic construct
  source 1..73: mol_type = other DNA
tccgtggtggtctagtggctaggattcggcgctctaaccgcctgcagctcgagttcgattcctggtcagggaa

SEQ ID NO: 355 - DNA, 72 bp, synthetic construct
  source 1..72: mol_type = other DNA
tcccacatggtctagcggttaggattcctggttctaacccaggcggcccgggttcgactcccggtgtgggaa

SEQ ID NO: 356 - DNA, 72 bp, synthetic construct
  source 1..72: mol_type = other DNA
tccctggtggtctagtggctaggattcggcgctctaaccgccgcggcccgggttcgattcccggtcagggaa

SEQ ID NO: 357 - DNA, 72 bp, synthetic construct
  source 1..72: mol_type = other DNA
tccctggtggtctagtggttaggattcggcgctctaaccgccgcggcccgggttcgattcccggtcagggaa

SEQ ID NO: 358 - DNA, 72 bp, synthetic construct
  source 1..72: mol_type = other DNA
tccctggtggtctagtggctaggattcggcgctctaaccgccgcggcccgggttcgattcccggccagggaa

SEQ ID NO: 359 - DNA, 72 bp, synthetic construct
  source 1..72: mol_type = other DNA
tcccatatggtctagcggttaggattcctggttctaacccaggtggcccgggttcgactcccggtatgggaa

SEQ ID NO: 360 - DNA, 72 bp, synthetic construct
  source 1..72: mol_type = other DNA
gcgttggtggtgtagtggtgagcacagctgcctctaaagcagttaacgcgggttcgattcccgggtaacgaa

SEQ ID NO: 361 - DNA, 72 bp, synthetic construct
  source 1..72: mol_type = other DNA
tccctggtggtctagtggttaggattcggcgctctaaccgccgcggcccgggttcgattcccggtcaggaaa

SEQ ID NO: 362 - DNA, 69 bp, synthetic construct
  source 1..69: mol_type = other DNA
ccctgtggtctagtggctaagactttgtgctctaattgctgcatcctaggttcaattcccagtcaggga

SEQ ID NO: 363 - DNA, 71 bp, synthetic construct
  source 1..71: mol_type = other DNA
tccttgatgtctagtggttaggatttggtgctctaactgcagcagcctgggttcatttctcagtcagggaa

SEQ ID NO: 364 - DNA, 71 bp, synthetic construct
  source 1..71: mol_type = other DNA
gcaatggtggttcagtggtagaattctcgcctctaacacaggagacccgggttcaattcctgacccatgta

SEQ ID NO: 365 - DNA, 72 bp, synthetic construct
  source 1..72: mol_type = other DNA
cccctggtggtctagtgcttaggattcggtgctctaaccgctgctgcctgcgttcgattcccggtcagggaa

SEQ ID NO: 366 - DNA, 72 bp, synthetic construct
  source 1..72: mol_type = other DNA
tccttggtggtctagtggctaggattcggtgctctaacctgtgcggcccgggttcaattcccgatgaaggaa

SEQ ID NO: 367 - DNA, 73 bp, synthetic construct
  source 1..73: mol_type = other DNA
tccctggtggtctagtggctaggattcggcgctctaaccgcctgcagctcgagttcgattcctggtcagggaa

SEQ ID NO: 368 - DNA, 72 bp, synthetic construct
  source 1..72: mol_type = other DNA
tgtctggtggtcaagtggctaggatttggcgctctaactgccgcggcccgcgttcgattcccggtcagggaa

SEQ ID NO: 369 - DNA, 72 bp, synthetic construct
  source 1..72: mol_type = other DNA
tcccatatggtctagcggttaggattcctggtttcaacccaggtggcccgggttcgactcccggtatgggaa

SEQ ID NO: 370 - DNA, 72 bp, synthetic construct
  source 1..72: mol_type = other DNA
tcccacatggtctagcggttaggattcctggtttcaacccaggcggcccgggttcgactcccggtgtgggaa

SEQ ID NO: 371 - DNA, 72 bp, synthetic construct
  source 1..72: mol_type = other DNA
tgtctggtggtcaagtggctaggatttggcgcttcaactgccgcggcccgcgttcgattcccggtcagggaa

SEQ ID NO: 372 - DNA, 71 bp, synthetic construct
  source 1..71: mol_type = other DNA
tccttgatgtctagtggttaggatttggtgcttcaactgcagcagcctgggttcatttctcagtcagggaa

SEQ ID NO: 373 - DNA, 71 bp, synthetic construct
  source 1..71: mol_type = other DNA
gcaatggtggttcagtggtagaattctcgccttcaacacaggagacccgggttcaattcctgacccatgta

SEQ ID NO: 374 - DNA, 73 bp, synthetic construct
  source 1..73: mol_type = other DNA
tccgtggtggtctagtggctaggattcggcgcttcaaccgcctgcagctcgagttcgattcctggtcagggaa

SEQ ID NO: 375 - DNA, 72 bp, synthetic construct
  source 1..72: mol_type = other DNA
tccctggtggtctagtggctaggattcggcgcttcaaccgccgcggcccgggttcgattcccggccagggaa

SEQ ID NO: 376 - DNA, 72 bp, synthetic construct
  source 1..72: mol_type = other DNA
tccctggtggtctagtggttaggattcggcgcttcaaccgccgcggcccgggttcgattcccggtcaggaaa

SEQ ID NO: 377 - DNA, 72 bp, synthetic construct
  source 1..72: mol_type = other DNA
tccttggtggtctagtggctaggattcggtgcttcaacctgtgcggcccgggttcaattcccgatgaaggaa

SEQ ID NO: 378 - DNA, 73 bp, synthetic construct
  source 1..73: mol_type = other DNA
tccctggtggtctagtggctaggattcggcgcttcaaccgcctgcagctcgagttcgattcctggtcagggaa

SEQ ID NO: 379 - DNA, 72 bp, synthetic construct
  source 1..72: mol_type = other DNA
cccctggtggtctagtgcttaggattcggtgcttcaaccgctgctgcctgcgttcgattcccggtcagggaa

SEQ ID NO: 380 - DNA, 72 bp, synthetic construct
  source 1..72: mol_type = other DNA
gcgttggtggtgtagtggtgagcacagctgccttcaaagcagttaacgcgggttcgattcccgggtaacgaa

SEQ ID NO: 381 - DNA, 72 bp, synthetic construct
  source 1..72: mol_type = other DNA
tccctggtggtctagtggctaggattcggcgcttcaaccgccgcggcccgggttcgattcccggtcagggaa

SEQ ID NO: 382 - DNA, 72 bp, synthetic construct
  source 1..72: mol_type = other DNA
tccctggtggtctagtggttaggattcggcgcttcaaccgccgcggcccgggttcgattcccggtcagggaa

SEQ ID NO: 383 - DNA, 69 bp, synthetic construct
  source 1..69: mol_type = other DNA
ccctgtggtctagtggctaagactttgtgcttcaattgctgcatcctaggttcaattcccagtcaggga

SEQ ID NO: 384 - DNA, 72 bp, synthetic construct
  source 1..72: mol_type = other DNA
gcgttggtggtatagtggttagcatagctgcctttaaagcagttgacccgggttcgattcccggccaacgca

SEQ ID NO: 385 - DNA, 72 bp, synthetic construct
  source 1..72: mol_type = other DNA
gcgttggtggtatagtggtgagcatagctgcctttaaagcagttgacccgggttcgattcccggccaacgca

SEQ ID NO: 386 - DNA, 72 bp, synthetic construct
  source 1..72: mol_type = other DNA
gcgttggtggtatagtggtaagcatagctgcctttaaagcagttgacccgggttcgattcccggccaacgca

SEQ ID NO: 387 - DNA, 71 bp, synthetic construct
  source 1..71: mol_type = other DNA
gcattggtggttcagtggtagaattctcgcctttaacgcgggagacccgggttcaattcccggccaatgca

SEQ ID NO: 388 - DNA, 71 bp, synthetic construct
  source 1..71: mol_type = other DNA
gccttggtggtgcagtggtagaattctcgcctttaacgtgggagacccgggttcaattcccggccaatgca

SEQ ID NO: 389 - DNA, 71 bp, synthetic construct
  source 1..71: mol_type = other DNA
gcgccgctggtgtagtggtatcatgcaagattttaattcttgcgacccgggttcgattcccgggcggcgca

SEQ ID NO: 390 - DNA, 71 bp, synthetic construct
  source 1..71: mol_type = other DNA
gcgtcagtggtttagtggtggaattcctgcctttaatgcacgagatccgtgttcaactcctggttggtgca

SEQ ID NO: 391 - DNA, 72 bp, synthetic construct
  source 1..72: mol_type = other DNA
gcgttggtggtatagtggtgagcatagttgcctttaaagcagttgacccgggctcgattcccggccaacgca

SEQ ID NO: 392 - DNA, 71 bp, synthetic construct
  source 1..71: mol_type = other DNA
gcatgggtgattcagtggtagaattttcacctttaatgcaggaggtccaggttcatttcctggcctatgca

SEQ ID NO: 393 - DNA, 72 bp, synthetic construct
  source 1..72: mol_type = other DNA
gcgtcagtggttttagtggtggaattcctgcctttaatgcacgagatccgtgttcaactcctggttggtgca

SEQ ID NO: 394 - DNA, 71 bp, synthetic construct
  source 1..71: mol_type = other DNA
gcattggtggttcagtggtagaattctcgcctttaacgcgggaggcccgggtttgattcccggccagtgca

SEQ ID NO: 395 - DNA, 61 bp, synthetic construct
  source 1..61: mol_type = other DNA
gtggtctagtggttaggattcagcgctttaaccgccgcagcccgggttcgattcccggtca

SEQ ID NO: 396 - DNA, 71 bp, synthetic construct
  source 1..71: mol_type = other DNA
gcgttggtggtttagtggtagaattctcgcctttaatgcgggagacccgggttcaattcccggccactgca

SEQ ID NO: 397 - DNA, 71 bp, synthetic construct
  source 1..71: mol_type = other DNA
gcattggtggttcagtggtagaattctcgcctttaatgcgggcggccgggcttcgattcctggccaatgca

SEQ ID NO: 398 - DNA, 71 bp, synthetic construct
  source 1..71: mol_type = other DNA
gcataggtggttcagtggtagaattcttgcctttaacgcaggaggcccaggtttgattcctggcccatgca

SEQ ID NO: 399 - DNA, 71 bp, synthetic construct
  source 1..71: mol_type = other DNA
gcgttggcagttcagtggtagaattctcgcctttaacccgggagacctggattccatttccggcaaatgca

SEQ ID NO: 400 - DNA, 72 bp, synthetic construct
  source 1..72: mol_type = other DNA
gcgttggtggtatagtggtgagcatagttgcctttaaagcagttgacccgggctcgattcccgcccaacgca

SEQ ID NO: 401 - DNA, 71 bp, synthetic construct
  source 1..71: mol_type = other DNA
gcatgggtggttcagtggtagaattctcgcctttaacgcgggaggcccgggttcgattcccggcccatgca

SEQ ID NO: 402 - DNA, 71 bp, synthetic construct
  source 1..71: mol_type = other DNA
gcattggtggttcagtggtagaattctcgcctttaacgcgggaggcccgggttcgattcccggccaatgca

SEQ ID NO: 403 - DNA, 71 bp, synthetic construct
  source 1..71: mol_type = other DNA
gcattggtggttcaatggtagaattctcgcctttaacgcaggagacccaggttcgattcctggccaatgca

SEQ ID NO: 404 - DNA, 61 bp, synthetic construct
  source 1..61: mol_type = other DNA
ggtggttcagtggtagaattctcgcctttaacgcgggagacccgggtttaattcccggtca

SEQ ID NO: 405 - DNA, 71 bp, synthetic construct
  source 1..71: mol_type = other DNA
gcgtcagtggtttagtggtggaattcctgcctctaatgcacgagatccgtgttcaactcctggttggtgca

SEQ ID NO: 406 - DNA, 72 bp, synthetic construct
  source 1..72: mol_type = other DNA
gcgttggtggtatagtggttagcatagctgcctctaaagcagttgacccgggttcgattcccggccaacgca

SEQ ID NO: 407 - DNA, 72 bp, synthetic construct
  source 1..72: mol_type = other DNA
gcgttggtggtatagtggtgagcatagctgcctctaaagcagttgacccgggttcgattcccggccaacgca

SEQ ID NO: 408 - DNA, 72 bp, synthetic construct
  source 1..72: mol_type = other DNA
gcgttggtggtatagtggtaagcatagctgcctctaaagcagttgacccgggttcgattcccggccaacgca

SEQ ID NO: 409 - DNA, 71 bp, synthetic construct
  source 1..71: mol_type = other DNA
gcattggtggttcagtggtagaattctcgcctctaacgcgggagacccgggttcaattcccggccaatgca

SEQ ID NO: 410 - DNA, 71 bp, synthetic construct
  source 1..71: mol_type = other DNA
gcgccgctggtgtagtggtatcatgcaagattctaattcttgcgacccgggttcgattcccgggcggcgca

SEQ ID NO: 411 - DNA, 71 bp, synthetic construct
  source 1..71: mol_type = other DNA
gcattggtggttcaatggtagaattctcgcctctaacgcaggagacccaggttcgattcctggccaatgca

SEQ ID NO: 412 - DNA, 71 bp, synthetic construct
  source 1..71: mol_type = other DNA
gcgttggtggtttagtggtagaattctcgcctctaatgcgggagacccgggttcaattcccggccactgca

SEQ ID NO: 413 - DNA, 71 bp, synthetic construct
  source 1..71: mol_type = other DNA
gccttggtggtgcagtggtagaattctcgcctctaacgtgggagacccgggttcaattcccggccaatgca

SEQ ID NO: 414 - DNA, 71 bp, synthetic construct
  source 1..71: mol_type = other DNA
gcatgggtggttcagtggtagaattctcgcctctaacgcgggaggcccgggttcgattcccggcccatgca

SEQ ID NO: 415 - DNA, 71 bp, synthetic construct
  source 1..71: mol_type = other DNA
gcattggtggttcagtggtagaattctcgcctctaacgcgggaggcccgggtttgattcccggccagtgca

SEQ ID NO: 416 - DNA, 72 bp, synthetic construct
  source 1..72: mol_type = other DNA
gcgttggtggtatagtggtgagcatagttgcctctaaagcagttgacccgggctcgattcccgcccaacgca

SEQ ID NO: 417 - DNA, 71 bp, synthetic construct
  source 1..71: mol_type = other DNA
gcattggtggttcagtggtagaattctcgcctctaacgcgggaggcccgggttcgattcccggccaatgca

SEQ ID NO: 418 - DNA, 71 bp, synthetic construct
  source 1..71: mol_type = other DNA
gcatgggtgattcagtggtagaattttcacctctaatgcaggaggtccaggttcatttcctggcctatgca

SEQ ID NO: 419 - DNA, 72 bp, synthetic construct
  source 1..72: mol_type = other DNA
gcgttggtggtatagtggtgagcatagttgcctctaaagcagttgacccgggctcgattcccggccaacgca

SEQ ID NO: 420 - DNA, 61 bp, synthetic construct
  source 1..61: mol_type = other DNA
gtggtctagtggttaggattcagcgctctaaccgccgcagcccgggttcgattcccggtca

SEQ ID NO: 421 - DNA, 71 bp, synthetic construct
  source 1..71: mol_type = other DNA
gcattggtggttcagtggtagaattctcgcctctaatgcgggcggccgggcttcgattcctggccaatgca

SEQ ID NO: 422 - DNA, 71 bp, synthetic construct
  source 1..71: mol_type = other DNA
gcataggtggttcagtggtagaattcttgcctctaacgcaggaggcccaggtttgattcctggcccatgca

SEQ ID NO: 423 - DNA, 71 bp, synthetic construct
  source 1..71: mol_type = other DNA
gcgttggcagttcagtggtagaattctcgcctctaacccgggagacctggattccatttccggcaaatgca

SEQ ID NO: 424 - DNA, 61 bp, synthetic construct
  source 1..61: mol_type = other DNA
ggtggttcagtggtagaattctcgcctctaacgcgggagacccgggtttaattcccggtca

SEQ ID NO: 425 - DNA, 72 bp, synthetic construct
  source 1..72: mol_type = other DNA
gcgtcagtggttttagtggtggaattcctgcctctaatgcacgagatccgtgttcaactcctggttggtgca

SEQ ID NO: 426 - DNA, 72 bp, synthetic construct
  source 1..72: mol_type = other DNA
gcgttggtggtatagtggtgagcatagctgccttcaaagcagttgacccgggttcgattcccggccaacgca

SEQ ID NO: 427 - DNA, 72 bp, synthetic construct
  source 1..72: mol_type = other DNA
gcgttggtggtatagtggtaagcatagctgccttcaaagcagttgacccgggttcgattcccggccaacgca

SEQ ID NO: 428 - DNA, 71 bp, synthetic construct
  source 1..71: mol_type = other DNA
gcgttggcagttcagtggtagaattctcgccttcaacccgggagacctggattccatttccggcaaatgca

SEQ ID NO: 429 - DNA, 61 bp, synthetic construct
  source 1..61: mol_type = other DNA
gtggtctagtggttaggattcagcgcttcaaccgccgcagcccgggttcgattcccggtca

SEQ ID NO: 430 - DNA, 72 bp, synthetic construct
  source 1..72: mol_type = other DNA
gcgttggtggtatagtggtgagcatagttgccttcaaagcagttgacccgggctcgattcccgcccaacgca

SEQ ID NO: 431 - DNA, 71 bp, synthetic construct
  source 1..71: mol_type = other DNA
gccttggtggtgcagtggtagaattctcgccttcaacgtgggagacccgggttcaattcccggccaatgca

SEQ ID NO: 432 - DNA, 71 bp, synthetic construct
  source 1..71: mol_type = other DNA
gcattggtggttcaatggtagaattctcgccttcaacgcaggagacccaggttcgattcctggccaatgca

SEQ ID NO: 433 - DNA, 71 bp, synthetic construct
  source 1..71: mol_type = other DNA
gcgccgctggtgtagtggtatcatgcaagatttcaattcttgcgacccgggttcgattcccgggcggcgca

SEQ ID NO: 434 - DNA, 71 bp, synthetic construct
  source 1..71: mol_type = other DNA
gcataggtggttcagtggtagaattcttgccttcaacgcaggaggcccaggtttgattcctggcccatgca

SEQ ID NO: 435 - DNA, 71 bp, synthetic construct
  source 1..71: mol_type = other DNA
gcatgggtgattcagtggtagaattttcaccttcaatgcaggaggtccaggttcatttcctggcctatgca

SEQ ID NO: 436 - DNA, 71 bp, synthetic construct
  source 1..71: mol_type = other DNA
gcattggtggttcagtggtagaattctcgccttcaacgcgggaggcccgggtttgattcccggccagtgca

SEQ ID NO: 437 - DNA, 61 bp, synthetic construct
  source 1..61: mol_type = other DNA
ggtggttcagtggtagaattctcgccttcaacgcgggagacccgggtttaattcccggtca

SEQ ID NO: 438 - DNA, 71 bp, synthetic construct
  source 1..71: mol_type = other DNA
gcattggtggttcagtggtagaattctcgccttcaatgcgggcggccgggcttcgattcctggccaatgca

SEQ ID NO: 439 - DNA, 71 bp, synthetic construct
  source 1..71: mol_type = other DNA
gcgtcagtggtttagtggtggaattcctgccttcaatgcacgagatccgtgttcaactcctggttggtgca

SEQ ID NO: 440 - DNA, 72 bp, synthetic construct
  source 1..72: mol_type = other DNA
gcgtcagtggttttagtggtggaattcctgccttcaatgcacgagatccgtgttcaactcctggttggtgca

SEQ ID NO: 441 - DNA, 72 bp, synthetic construct
  source 1..72: mol_type = other DNA
gcgttggtggtatagtggttagcatagctgccttcaaagcagttgacccgggttcgattcccggccaacgca

SEQ ID NO: 442 - DNA, 72 bp, synthetic construct
  source 1..72: mol_type = other DNA
gcgttggtggtatagtggtgagcatagttgccttcaaagcagttgacccgggctcgattcccggccaacgca

SEQ ID NO: 443 - DNA, 71 bp, synthetic construct
  source 1..71: mol_type = other DNA
gcattggtggttcagtggtagaattctcgccttcaacgcgggagacccgggttcaattcccggccaatgca

SEQ ID NO: 444 - DNA, 71 bp, synthetic construct
  source 1..71: mol_type = other DNA
gcattggtggttcagtggtagaattctcgccttcaacgcgggaggcccgggttcgattcccggccaatgca

SEQ ID NO: 445 - DNA, 71 bp, synthetic construct
  source 1..71: mol_type = other DNA
gcgttggtggtttagtggtagaattctcgccttcaatgcgggagacccgggttcaattcccggccactgca

SEQ ID NO: 446 - DNA, 71 bp, synthetic construct
  source 1..71: mol_type = other DNA
gcatgggtggttcagtggtagaattctcgccttcaacgcgggaggcccgggttcgattcccggcccatgca

SEQ ID NO: 447 - DNA, 72 bp, synthetic construct
  source 1..72: mol_type = other DNA
gccatgatcgtatagtggttagtactctgcgctttagccgcagcaacctcggttcgaatccgagtcacggca

SEQ ID NO: 448 - DNA, 72 bp, synthetic construct
  source 1..72: mol_type = other DNA
gccgtgatcgtatagtggttagtactctgcgttttagccgcagcaacctcggttcgaatccgagtcacggca

SEQ ID NO: 449 - DNA, 72 bp, synthetic construct
  source 1..72: mol_type = other DNA
gccatgatcgtatagtggttagtactctgcgctctagccgcagcaacctcggttcgaatccgagtcacggca

SEQ ID NO: 450 - DNA, 72 bp, synthetic construct
  source 1..72: mol_type = other DNA
gccgtgatcgtatagtggttagtactctgcgttctagccgcagcaacctcggttcgaatccgagtcacggca

SEQ ID NO: 451 - DNA, 72 bp, synthetic construct
  source 1..72: mol_type = other DNA
gccatgatcgtatagtggttagtactctgcgcttcagccgcagcaacctcggttcgaatccgagtcacggca

SEQ ID NO: 452 - DNA, 72 bp, synthetic construct
  source 1..72: mol_type = other DNA
gccgtgatcgtatagtggttagtactctgcgtttcagccgcagcaacctcggttcgaatccgagtcacggca

SEQ ID NO: 453 - DNA, 74 bp, synthetic construct
  source 1..74: mol_type = other DNA
ggccggttagctcagttggttagagcgtggtgctttaaacgccaaggtcgcgggttcgatccccgtacgggcca

SEQ ID NO: 454 - DNA, 74 bp, synthetic construct; identical to SEQ ID NO: 35
  source 1..74: mol_type = other DNA

SEQ ID NO: 455 - DNA, 74 bp, synthetic construct
  source 1..74: mol_type = other DNA
ggccggttagctcagttggttagagcgtggtgctttaaacgccaaggtcgcgggttcgaaccccgtacgggcca

SEQ ID NO: 456 - DNA, 74 bp, synthetic construct
  source 1..74: mol_type = other DNA
ggccggttagctcagttggtaagagcgtggtgctttaaacaccaaggtcgcgggctcgactcccgcaccggcca

SEQ ID NO: 457 - DNA, 94 bp, synthetic construct
  source 1..94: mol_type = other DNA
gctccagtggcgcaatcggttagcgcgcggtactttaatggcagtatgtgtgcgagtgatgccgaggttgtgagttcgagcctcacctggagca

SEQ ID NO: 458 - DNA, 93 bp, synthetic construct
  source 1..93: mol_type = other DNA
gctccagtggcgcaatcggttagcgcgcggtactttaacagcagtacatgcagagcaatgccgaggttgtgagttcgagcctcacctggagca

SEQ ID NO: 459 - DNA, 94 bp, synthetic construct
  source 1..94: mol_type = other DNA
gctccagtggcgcaatcggttagcgcgcggtactttaacaacagtatatgtgcgggtgatgccgaggttgtgagttcgagcctcacctggagca

SEQ ID NO: 460 - DNA, 93 bp, synthetic construct
  source 1..93: mol_type = other DNA
gctccagtggcgcaatcggttagcgcgcggtactttaatgacagtgcgagcggagcaatgccgaggttgtgagttcgatcctcacctggagca

SEQ ID NO: 461 - DNA, 94 bp, synthetic construct
  source 1..94: mol_type = other DNA
gctccagtggcgcaatcggttagcgcgcggtactttaaagacagtgcacctgtgagcaatgccgaggttgtgagttcaagcctcacctggagca

SEQ ID NO: 462 - DNA, 74 bp, synthetic construct
  source 1..74: mol_type = other DNA
ggctggttagttcagttggttagagcgtggtgctttaaacgccaaggtcgtgggttcgatccccatatcggcca

SEQ ID NO: 463 - DNA, 74 bp, synthetic construct
  source 1..74: mol_type = other DNA
ggccggttagctcagttggtaagagcgtggtgctctaaacaccaaggtcgcgggctcgactcccgcaccggcca

SEQ ID NO: 464 - DNA, 94 bp, synthetic construct
  source 1..94: mol_type = other DNA
gctccagtggcgcaatcggttagcgcgcggtactctaatggcagtatgtgtgcgagtgatgccgaggttgtgagttcgagcctcacctggagca

SEQ ID NO: 465 - DNA, 94 bp, synthetic construct
  source 1..94: mol_type = other DNA
gctccagtggcgcaatcggttagcgcgcggtactctaaagacagtgcacctgtgagcaatgccgaggttgtgagttcaagcctcacctggagca

SEQ ID NO: 466 - DNA, 94 bp, synthetic construct
  source 1..94: mol_type = other DNA
gctccagtggcgcaatcggttagcgcgcggtactctaacaacagtatatgtgcgggtgatgccgaggttgtgagttcgagcctcacctggagca

SEQ ID NO: 467 - DNA, 93 bp, synthetic construct
  source 1..93: mol_type = other DNA
gctccagtggcgcaatcggttagcgcgcggtactctaacagcagtacatgcagagcaatgccgaggttgtgagttcgagcctcacctggagca

SEQ ID NO: 468 - DNA, 74 bp, synthetic construct
  source 1..74: mol_type = other DNA
ggccggttagctcagtcggctagagcgtggtgctctaaacgccaaggtcgcgggttcgatccccgtacgggcca

SEQ ID NO: 469 - DNA, 93 bp, synthetic construct
  source 1..93: mol_type = other DNA
gctccagtggcgcaatcggttagcgcgcggtactctaatgacagtgcgagcggagcaatgccgaggttgtgagttcgatcctcacctggagca

SEQ ID NO: 470 - DNA, 74 bp, synthetic construct
  source 1..74: mol_type = other DNA
ggctggttagttcagttggttagagcgtggtgctctaaacgccaaggtcgtgggttcgatccccatatcggcca

SEQ ID NO: 471 - DNA, 74 bp, synthetic construct
  source 1..74: mol_type = other DNA
ggccggttagctcagttggtcagagcgtggtgcttcaaacgccaaggtcgcgggttcgatccccgtacgggcca

SEQ ID NO: 472 - DNA, 74 bp, synthetic construct
  source 1..74: mol_type = other DNA
ggccggttagctcagttggttagagcgtggtgcttcaaacgccaaggtcgcgggttcgaaccccgtacgggcca

SEQ ID NO: 473 - DNA, 74 bp, synthetic construct
  source 1..74: mol_type = other DNA
ggccggttagctcagttggttagagcgtggtgcttcaaacgccaaggtcgcgggttcgatccccgtacgggcca

SEQ ID NO: 474 - DNA, 74 bp, synthetic construct
  source 1..74: mol_type = other DNA
ggccggttagctcagttggttagagcgtggcgcttcaaacgccaaggtcgcgggttcgatccccgtacgggcca

SEQ ID NO: 475 - DNA, 74 bp, synthetic construct
  source 1..74: mol_type = other DNA
ggccggttagctcagttggtaagagcgtggtgcttcaaacaccaaggtcgcgggctcgactcccgcaccggcca

SEQ ID NO: 476 - DNA, 94 bp, synthetic construct
  source 1..94: mol_type = other DNA
gctccagtggcgcaatcggttagcgcgcggtacttcaatggcagtatgtgtgcgagtgatgccgaggttgtgagttcgagcctcacctggagca

SEQ ID NO: 477 - DNA, 93 bp, synthetic construct
  source 1..93: mol_type = other DNA
gctccagtggcgcaatcggttagcgcgcggtacttcaacagcagtacatgcagagcaatgccgaggttgtgagttcgagcctcacctggagca

SEQ ID NO: 478 - DNA, 94 bp, synthetic construct
  source 1..94: mol_type = other DNA
gctccagtggcgcaatcggttagcgcgcggtacttcaaagacagtgcacctgtgagcaatgccgaggttgtgagttcaagcctcacctggagca

SEQ ID NO: 479 - DNA, 74 bp, synthetic construct
  source 1..74: mol_type = other DNA
ggctggttagttcagttggttagagcgtggtgcttcaaacgccaaggtcgtgggttcgatccccatatcggcca

SEQ ID NO: 480 - DNA, 74 bp, synthetic construct
  source 1..74: mol_type = other DNA
ggctggttagctcagttggttagagcgtggtgcttcaaacgccaaggtcgcgggttcgatccccgtactggcca

SEQ ID NO: 481 - DNA, 93 bp, synthetic construct
  source 1..93: mol_type = other DNA
gctccagtggcgcaatcggttagcgcgcggtacttcaatgacagtgcgagcggagcaatgccgaggttgtgagttcgatcctcacctggagca

SEQ ID NO: 482 - DNA, 94 bp, synthetic construct
  source 1..94: mol_type = other DNA
gctccagtggcgcaatcggttagcgcgcggtacttcaacaacagtatatgtgcgggtgatgccgaggttgtgagttcgagcctcacctggagca

SEQ ID NO: 483 - DNA, 83 bp, synthetic construct
  source 1..83: mol_type = other DNA
accaggatggccgagtggttaaggcgttggactttagatccaatggacatatgtccgcgtgggttcgaaccccactcctggta

SEQ ID NO: 484 - DNA, 83 bp, synthetic construct
  source 1..83: mol_type = other DNA
accgggatggccgagtggttaaggcgttggactttagatccaatgggctggtgcccgcgtgggttcgaaccccactctcggta

SEQ ID NO: 485 - DNA, 84 bp, synthetic construct
  source 1..84: mol_type = other DNA
gtcaggatggccgagtggtctaaggcgccagactttagttctggtctccgcatggaggcgtgggttcgaatcccacttctgaca

SEQ ID NO: 486 - DNA, 84 bp, synthetic construct
  source 1..84: mol_type = other DNA
gtcaggatggccgagtggtctaaggcgccagactttagttctggtctccgtatggaggcgtgggttcgaatcccacttctgaca

SEQ ID NO: 487 - DNA, 83 bp, synthetic construct
  source 1..83: mol_type = other DNA
gtcaggatggccgagtggtctaaggcgccagactttagttctggtctccaatggaggcgtgggttcgaatcccacttctgaca

SEQ ID NO: 488 - DNA, 84 bp, synthetic construct
  source 1..84: mol_type = other DNA
accaggatggccaagtagttaaaggcactggactttagagccaatggacatatgtctgtgtgggtttgaaccccactcctggtg

SEQ ID NO: 489 - DNA, 83 bp, synthetic construct
  source 1..83: mol_type = other DNA
accgggatggctgagtggttaaggcgttggactttagatccaatggacaggtgtccgcgtgggttcgagccccactcccggta

SEQ ID NO: 490 - DNA, 82 bp, synthetic construct
  source 1..82: mol_type = other DNA
ggtagcgtggccgagcggtctaaggcgctggattttagctccagtctcttcggaggcgtgggttcgaatcccaccgctgcca

SEQ ID NO: 491 - DNA, 82 bp, synthetic construct
  source 1..82: mol_type = other DNA
ggtagcgtggccgagtggtctaaggcgctggattttagctccagtcatttcgatggcgtgggttcgaatcccaccgctgcca

SEQ ID NO: 492 - DNA, 82 bp, synthetic construct
  source 1..82: mol_type = other DNA
ggtagtgtggccgagcggtctaaggcgctggattttagctccagtctcttcgggggcgtgggttcgaatcccaccactgcca

SEQ ID NO: 493 - DNA, 82 bp, synthetic construct
  source 1..82: mol_type = other DNA
ggtagcgtggccgagcggtctaaggcgctggattttagctccagtctcttcgggggcgtgggttcaaatcccaccgctgcca

SEQ ID NO: 494 - DNA, 83 bp, synthetic construct
  source 1..83: mol_type = other DNA
accagaatggccgagtggttaaggcgttggactttagatccaatggattcatatccgcgtgggttcgaaccccacttctggta

SEQ ID NO: 495 - DNA, 82 bp, synthetic construct
  source 1..82: mol_type = other DNA
ggtagtgtggttgaatggtctaaggcactgaattttagctccagtctctttggggacgtgggtttaaatcccactgctgcaa

SEQ ID NO: 496 - DNA, 69 bp, synthetic construct
  source 1..69: mol_type = other DNA
gagaaagtcatcgtagttacgaagttggctttaacccagttttgggaggttcaattccttcctttctct

SEQ ID NO: 497 - DNA, 83 bp, synthetic construct
  source 1..83: mol_type = other DNA
actcatttggctgagtggttaaggcattggactttagatccaatggagtagtggctgtgtgggtttaaaccccactactggta

SEQ ID NO: 498 - DNA, 74 bp, synthetic construct
  source 1..74: mol_type = other DNA
gcctccttagtgcagtaggtagcgcatcagtctttaaatctgaatggtcctgagttcaagcctcagagggggca

SEQ ID NO: 499 - DNA, 82 bp, synthetic construct
  source 1..82: mol_type = other DNA
ggtagggtggccgagcggtctaaggcactgtattttaactccagtctcttcagaggcatgggtttgaatcccactgctgcca

SEQ ID NO: 500 - DNA, 60 bp, synthetic construct
  source 1..60: mol_type = other DNA
ggttccatggtgtaatggtgagcactctggactttaaatccagaagtagtgctggaacaa

SEQ ID NO: 501 - DNA, 84 bp, synthetic construct
  source 1..84: mol_type = other DNA
gtcaggatggccgagcagtcttaaggcgctgcgttttaatcgcaccctccgctggaggcgtgggttcgaatcccacttttgaca

SEQ ID NO: 502 - DNA, 65 bp, synthetic construct
  source 1..65: mol_type = other DNA
gccgagcggtctaaggctccggattttagcgccggtgtcttcggaggcatgggttcgaattccac

SEQ ID NO: 503 - DNA, 73 bp, synthetic construct
  source 1..73: mol_type = other DNA
gggccagtggctcaatggataatgcgtctgactttaaatcagaagattccagccttgactcctggctggctca

SEQ ID NO: 504 - DNA, 82 bp, synthetic construct
  source 1..82: mol_type = other DNA
ggtagcgtggccgagtggtctaagacgctggattttagctccagtctcttcgggggcgtgggtttgaatcccaccgctgcca

SEQ ID NO: 505 - DNA, 85 bp, synthetic construct
  source 1..85: mol_type = other DNA
gtcaggatggccgagtggtctaaggcgccagactttagtttctggtctccggatggaggcgtgggttcgaatcccacttctgaca

SEQ ID NO: 506 - DNA, 83 bp, synthetic construct
  source 1..83: mol_type = other DNA
gtcagggtggctgagcagtctgaggggctgcgttttagtcgcagtctgccctggaggcgtgggttcgaatcccactcctgaaa

SEQ ID NO: 507 - DNA, 82 bp, synthetic construct
  source 1..82: mol_type = other DNA
ggtagcgtggccgagcggtctaaggcgctggattctagctccagtctcttcgggggcgtgggttcaaatcccaccgctgcca

SEQ ID NO: 508 - DNA, 84 bp, synthetic construct
  source 1..84: mol_type = other DNA
gtcaggatggccgagtggtctaaggcgccagactctagttctggtctccgcatggaggcgtgggttcgaatcccacttctgaca

SEQ ID NO: 509 - DNA, 83 bp, synthetic construct
  source 1..83: mol_type = other DNA
accagaatggccgagtggttaaggcgttggactctagatccaatggattcatatccgcgtgggttcgaaccccacttctggta

SEQ ID NO: 510 - DNA, 83 bp, synthetic construct
  source 1..83: mol_type = other DNA
accaggatggccgagtggttaaggcgttggactctagatccaatggacatatgtccgcgtgggttcgaaccccactcctggta

SEQ ID NO: 511 - DNA, 83 bp, synthetic construct
  source 1..83: mol_type = other DNA
gtcaggatggccgagtggtctaaggcgccagactctagttctggtctccaatggaggcgtgggttcgaatcccacttctgaca

SEQ ID NO: 512 - DNA, 84 bp, synthetic construct
  source 1..84: mol_type = other DNA
gtcaggatggccgagtggtctaaggcgccagactctagttctggtctccgtatggaggcgtgggttcgaatcccacttctgaca

SEQ ID NO: 513 - DNA, 83 bp, synthetic construct
  source 1..83: mol_type = other DNA
accgggatggctgagtggttaaggcgttggactctagatccaatggacaggtgtccgcgtgggttcgagccccactcccggta

SEQ ID NO: 514 - DNA, 82 bp, synthetic construct
  source 1..82: mol_type = other DNA
ggtagcgtggccgagtggtctaaggcgctggattctagctccagtcatttcgatggcgtgggttcgaatcccaccgctgcca

SEQ ID NO: 515 - DNA, 83 bp, synthetic construct
  source 1..83: mol_type = other DNA
accgggatggccgagtggttaaggcgttggactctagatccaatgggctggtgcccgcgtgggttcgaaccccactctcggta

SEQ ID NO: 516 - DNA, 82 bp, synthetic construct
  source 1..82: mol_type = other DNA
ggtagtgtggccgagcggtctaaggcgctggattctagctccagtctcttcgggggcgtgggttcgaatcccaccactgcca

SEQ ID NO: 517 - DNA, 82 bp, synthetic construct
  source 1..82: mol_type = other DNA
ggtagcgtggccgagcggtctaaggcgctggattctagctccagtctcttcggaggcgtgggttcgaatcccaccgctgcca

SEQ ID NO: 518 - DNA, 84 bp, synthetic construct
  source 1..84: mol_type = other DNA
accaggatggccaagtagttaaaggcactggactctagagccaatggacatatgtctgtgtgggtttgaaccccactcctggtg

SEQ ID NO: 519 - DNA, 85 bp, synthetic construct
  source 1..85: mol_type = other DNA
gtcaggatggccgagtggtctaaggcgccagactctagtttctggtctccggatggaggcgtgggttcgaatcccacttctgaca

SEQ ID NO: 520 - DNA, 60 bp, synthetic construct
  source 1..60: mol_type = other DNA
ggttccatggtgtaatggtgagcactctggactctaaatccagaagtagtgctggaacaa

SEQ ID NO: 521 - DNA, 83 bp, synthetic construct
  source 1..83: mol_type = other DNA
gtcagggtggctgagcagtctgaggggctgcgttctagtcgcagtctgccctggaggcgtgggttcgaatcccactcctgaaa

SEQ ID NO: 522 - DNA, 84 bp, synthetic construct
  source 1..84: mol_type = other DNA
gtcaggatggccgagcagtcttaaggcgctgcgttctaatcgcaccctccgctggaggcgtgggttcgaatcccacttttgaca

SEQ ID NO: 523 - DNA, 83 bp, synthetic construct
  source 1..83: mol_type = other DNA
actcatttggctgagtggttaaggcattggactctagatccaatggagtagtggctgtgtgggtttaaaccccactactggta

SEQ ID NO: 524 - DNA, 82 bp, synthetic construct
  source 1..82: mol_type = other DNA
ggtagtgtggttgaatggtctaaggcactgaattctagctccagtctctttggggacgtgggtttaaatcccactgctgcaa

SEQ ID NO: 525 - DNA, 82 bp, synthetic construct
  source 1..82: mol_type = other DNA
ggtagcgtggccgagtggtctaagacgctggattctagctccagtctcttcgggggcgtgggtttgaatcccaccgctgcca

SEQ ID NO: 526 - DNA, 74 bp, synthetic construct
  source 1..74: mol_type = other DNA
gcctccttagtgcagtaggtagcgcatcagtctctaaatctgaatggtcctgagttcaagcctcagagggggca

SEQ ID NO: 527 - DNA, 69 bp, synthetic construct
  source 1..69: mol_type = other DNA
gagaaagtcatcgtagttacgaagttggctctaacccagttttgggaggttcaattccttcctttctct

SEQ ID NO: 528 - DNA, 73 bp, synthetic construct
  source 1..73: mol_type = other DNA
gggccagtggctcaatggataatgcgtctgactctaaatcagaagattccagccttgactcctggctggctca

SEQ ID NO: 529 - DNA, 65 bp, synthetic construct
  source 1..65: mol_type = other DNA
gccgagcggtctaaggctccggattctagcgccggtgtcttcggaggcatgggttcgaattccac

SEQ ID NO: 530 - DNA, 82 bp, synthetic construct
  source 1..82: mol_type = other DNA
ggtagggtggccgagcggtctaaggcactgtattctaactccagtctcttcagaggcatgggtttgaatcccactgctgcca

SEQ ID NO: 531 - DNA, 83 bp, synthetic construct
  source 1..83: mol_type = other DNA
accaggatggccgagtggttaaggcgttggacttcagatccaatggacatatgtccgcgtgggttcgaaccccactcctggta

SEQ ID NO: 532 - DNA, 83 bp, synthetic construct
  source 1..83: mol_type = other DNA
accagaatggccgagtggttaaggcgttggacttcagatccaatggattcatatccgcgtgggttcgaaccccacttctggta

SEQ ID NO: 533 - DNA, 83 bp, synthetic construct
  source 1..83: mol_type = other DNA
accgggatggccgagtggttaaggcgttggacttcagatccaatgggctggtgcccgcgtgggttcgaaccccactctcggta

SEQ ID NO: 534 - DNA, 83 bp, synthetic construct
  source 1..83: mol_type = other DNA
accgggatggctgagtggttaaggcgttggacttcagatccaatggacaggtgtccgcgtgggttcgagccccactcccggta

SEQ ID NO: 535 - DNA, 84 bp, synthetic construct
  source 1..84: mol_type = other DNA
gtcaggatggccgagtggtctaaggcgccagacttcagttctggtctccgtatggaggcgtgggttcgaatcccacttctgaca

SEQ ID NO: 536 - DNA, 84 bp, synthetic construct
  source 1..84: mol_type = other DNA
gtcaggatggccgagtggtctaaggcgccagacttcagttctggtctccgcatggaggcgtgggttcgaatcccacttctgaca

SEQ ID NO: 537 - DNA, 83 bp, synthetic construct
  source 1..83: mol_type = other DNA
gtcaggatggccgagtggtctaaggcgccagacttcagttctggtctccaatggaggcgtgggttcgaatcccacttctgaca

SEQ ID NO: 538 - DNA, 82 bp, synthetic construct
  source 1..82: mol_type = other DNA
ggtagtgtggccgagcggtctaaggcgctggatttcagctccagtctcttcgggggcgtgggttcgaatcccaccactgcca

SEQ ID NO: 539 - DNA, 82 bp, synthetic construct
  source 1..82: mol_type = other DNA
ggtagcgtggccgagcggtctaaggcgctggatttcagctccagtctcttcgggggcgtgggttcaaatcccaccgctgcca

SEQ ID NO: 540 - DNA, 82 bp, synthetic construct
  source 1..82: mol_type = other DNA
ggtagcgtggccgagcggtctaaggcgctggatttcagctccagtctcttcggaggcgtgggttcgaatcccaccgctgcca

SEQ ID NO: 541 - DNA, 82 bp, synthetic construct
  source 1..82: mol_type = other DNA
ggtagcgtggccgagtggtctaaggcgctggatttcagctccagtcatttcgatggcgtgggttcgaatcccaccgctgcca

SEQ ID NO: 542 - DNA, 84 bp, synthetic construct
  source 1..84: mol_type = other DNA
gtcaggatggccgagcagtcttaaggcgctgcgtttcaatcgcaccctccgctggaggcgtgggttcgaatcccacttttgaca

SEQ ID NO: 543 - DNA, 82 bp, synthetic construct
  source 1..82: mol_type = other DNA
ggtagtgtggttgaatggtctaaggcactgaatttcagctccagtctctttggggacgtgggtttaaatcccactgctgcaa

SEQ ID NO: 544 - DNA, 60 bp, synthetic construct
  source 1..60: mol_type = other DNA
ggttccatggtgtaatggtgagcactctggacttcaaatccagaagtagtgctggaacaa

SEQ ID NO: 545 - DNA, 74 bp, synthetic construct
  source 1..74: mol_type = other DNA
gcctccttagtgcagtaggtagcgcatcagtcttcaaatctgaatggtcctgagttcaagcctcagagggggca

SEQ ID NO: 546 - DNA, 84 bp, synthetic construct
  source 1..84: mol_type = other DNA
accaggatggccaagtagttaaaggcactggacttcagagccaatggacatatgtctgtgtgggtttgaaccccactcctggtg

SEQ ID NO: 547 - DNA, 83 bp, synthetic construct
  source 1..83: mol_type = other DNA
actcatttggctgagtggttaaggcattggacttcagatccaatggagtagtggctgtgtgggtttaaaccccactactggta

SEQ ID NO: 548 - DNA, 85 bp, synthetic construct
  source 1..85: mol_type = other DNA
gtcaggatggccgagtggtctaaggcgccagacttcagtttctggtctccggatggaggcgtgggttcgaatcccacttctgaca

SEQ ID NO: 549 - DNA, 82 bp, synthetic construct
  source 1..82: mol_type = other DNA
ggtagcgtggccgagtggtctaagacgctggatttcagctccagtctcttcgggggcgtgggtttgaatcccaccgctgcca

SEQ ID NO: 550 - DNA, 65 bp, synthetic construct
  source 1..65: mol_type = other DNA
gccgagcggtctaaggctccggatttcagcgccggtgtcttcggaggcatgggttcgaattccac

SEQ ID NO: 551 - DNA, 82 bp, synthetic construct
  source 1..82: mol_type = other DNA
ggtagggtggccgagcggtctaaggcactgtatttcaactccagtctcttcagaggcatgggtttgaatcccactgctgcca

SEQ ID NO: 552 - DNA, 69 bp, synthetic construct
  source 1..69: mol_type = other DNA
gagaaagtcatcgtagttacgaagttggcttcaacccagttttgggaggttcaattccttcctttctct

SEQ ID NO: 553 - DNA, 83 bp, synthetic construct
  source 1..83: mol_type = other DNA
gtcagggtggctgagcagtctgaggggctgcgtttcagtcgcagtctgccctggaggcgtgggttcgaatcccactcctgaaa

SEQ ID NO: 554 - DNA, 73 bp, synthetic construct
  source 1..73: mol_type = other DNA
gggccagtggctcaatggataatgcgtctgacttcaaatcagaagattccagccttgactcctggctggctca

SEQ ID NO: 555 - DNA, 73 bp, synthetic construct
  source 1..73: mol_type = other DNA
gcctgggtagctcagtcggtagagcatcagactttaaatctgagggtccagggttcaagtccctgtccaggcg

SEQ ID NO: 556 - DNA, 73 bp, synthetic construct
  source 1..73: mol_type = other DNA
gcctggatagctcagttggtagagcatcagactttaaatctgagggtccagggttcaagtccctgttcaggca

SEQ ID NO: 557 - DNA, 73 bp, synthetic construct
  source 1..73: mol_type = other DNA
gcctggatagctcagttggtagagcatcagactttaaatctgagggtccagggttcaagtccctgttcaggcg

SEQ ID NO: 558 - DNA, 73 bp, synthetic construct
  source 1..73: mol_type = other DNA
gcccggctagctcagtcggtagagcatgggactttaaatctcagggtcgtgggttcgagccccacgttgggcg

SEQ ID NO: 559 - DNA, 65 bp, synthetic construct
  source 1..65: mol_type = other DNA
gcgctagtcagtagagcatgagactttaaatctcagggtcgtgggttcgagccccacatcgggcg

SEQ ID NO: 560 - DNA, 73 bp, synthetic construct
  source 1..73: mol_type = other DNA
gcccggatagctcagtcggtagagcatcagactttaaatctgagggtccggggttcaagtccctgttcgggcg

SEQ ID NO: 561 - DNA, 73 bp, synthetic construct
  source 1..73: mol_type = other DNA
gcctggatagctcagtcggtagagcatcagactttaaatctgagggtccagggttcaagtccctgttcaggcg

SEQ ID NO: 562 - DNA, 73 bp, synthetic construct
  source 1..73: mol_type = other DNA
gcccggctagctcagtcggtagagcatgagaccttaaatctcagggtcgtgggttcgagccccacgttgggcg

SEQ ID NO: 563 - DNA, 73 bp, synthetic construct
  source 1..73: mol_type = other DNA
ccccggctggctcagtcagtagatcatgagactttaaatctcagggtcgtgggttcacgccccacactgggcg

SEQ ID NO: 564 - DNA, 73 bp, synthetic construct
  source 1..73: mol_type = other DNA
gcccgactacctcagtcggtggagcatgggactttacatcccagggttgtgggttcgagccccacattgggca

SEQ ID NO: 565 - DNA, 74 bp, synthetic construct
  source 1..74: mol_type = other DNA
gcctggctacctcagttggtagagcatgggactttaaatcccagagtcagtgggttcaagcctcacattgagtg

SEQ ID NO: 566 - DNA, 76 bp, synthetic construct
  source 1..76: mol_type = other DNA
gcctggatagctccttcggtagagcatcatcagactttaaatgtgagggtccagggttcaagttcctgtttgggcg

SEQ ID NO: 567 - DNA, 73 bp, synthetic construct
  source 1..73: mol_type = other DNA
gcctggctagctcagtcggcaaagcatgagactttaaatctcagggtcgtgggctcgagctccatgttgggcg

SEQ ID NO: 568 - DNA, 74 bp, synthetic construct
  source 1..74: mol_type = other DNA
gcctgggtagctcagtcggtagagctatcagactttaagcctgaggattcagggttcaatcccttgctggggcg

SEQ ID NO: 569 - DNA, 73 bp, synthetic construct
  source 1..73: mol_type = other DNA
gccaggatagttcaggtggtagagcatcagactttaaacctgagggttcagggttcaagtctctgtttgggcg

SEQ ID NO: 570 - DNA, 73 bp, synthetic construct
  source 1..73: mol_type = other DNA
acccagatagctcagtcagtagagcatcagactttaaatctgagggtccaaggttcatgtccctttttgggtg

SEQ ID NO: 571 - DNA, 73 bp, synthetic construct
  source 1..73: mol_type = other DNA
gcccagctagctcagtcggtagagcataagactttaaatctcagggttgtggattcgtgccccatgctgggtg

SEQ ID NO: 572 - DNA, 73 bp, synthetic construct
  source 1..73: mol_type = other DNA
gttggggtaactcagttggtagagtagcagactttacatctgagggtccagggtttaagtccatgtccaggca

SEQ ID NO: 573 - DNA, 73 bp, synthetic construct
  source 1..73: mol_type = other DNA
gcccagctagctcagtcggtagagcatgagactttaaatctcagggtcatgggtttgagccccacgtttggtg

SEQ ID NO: 574 - DNA, 73 bp, synthetic construct
  source 1..73: mol_type = other DNA
acctgggtagcttagttggtagagcattggactttaaatttgagggcccaggtttcaagtccctgtttgggtg

SEQ ID NO: 575 - DNA, 73 bp, synthetic construct
  source 1..73: mol_type = other DNA
gcccggagagctcagtgggtagagcatcagactttaaatctgagggtccagggttcaagtcctcgttcgggca

SEQ ID NO: 576 - DNA, 73 bp, synthetic construct
  source 1..73: mol_type = other DNA
gtctagctagatcagttggtagagcataagactttaaatctcagggtcatgggtttgagccctacgttgggcg

SEQ ID NO: 577 - DNA, 73 bp, synthetic construct
  source 1..73: mol_type = other DNA
gcctggatagctcagttggtagaacatcagactttaaatctgacggtgcagggttcaagtccctgttcaggcg

SEQ ID NO: 578 - DNA, 73 bp, synthetic construct
  source 1..73: mol_type = other DNA
gcccggctagctcagtcgatagagcatgagactttaaatctcagggtcgtgggttcgagccgcacgttgggcg

SEQ ID NO: 579 - DNA, 71 bp, synthetic construct
  source 1..71: mol_type = other DNA
ctgcagctagctcagtcggtagagcatgagactttaaatctcagggtcatgggttcgtgccccatgttggg

SEQ ID NO: 580 - DNA, 69 bp, synthetic construct
  source 1..69: mol_type = other DNA
ccgaatagcttagttgatgaagcgtgagactttaaatctcagggtagtgggttcaagccccacattgga

SEQ ID NO: 581 - DNA, 73 bp, synthetic construct
  source 1..73: mol_type = other DNA
gcccagctagctcagccggtagagcacaagactttaaatctcagggtcgtgggtttgagccctgtgttgagca

SEQ ID NO: 582 - DNA, 70 bp, synthetic construct
  source 1..70: mol_type = other DNA
ccagcatgtctcagtcggtatagtgtgagactttaaatctcagggtcgtgggttcaagccccacattggg

SEQ ID NO: 583 - DNA, 73 bp, synthetic construct
  source 1..73: mol_type = other DNA
acctgggtagctcagtaggtagaacatcagactttaaatctgagggtctagggttcaagtccctgtccaggcg

SEQ ID NO: 584 - DNA, 62 bp, synthetic construct
  source 1..62: mol_type = other DNA
gatagctcagttgatagagcatcagactttaaatctgagggtccagggttcatgtccctgtt

SEQ ID NO: 585 - DNA, 69 bp, synthetic construct
  source 1..69: mol_type = other DNA
ccgaatagcttagttgatgaagcgtgagactctaaatctcagggtagtgggttcaagccccacattgga

SEQ ID NO: 586 - DNA, 73 bp, synthetic construct
  source 1..73: mol_type = other DNA
acctgggtagcttagttggtagagcattggactctaaatttgagggcccaggtttcaagtccctgtttgggtg

SEQ ID NO: 587 - DNA, 73 bp, synthetic construct
  source 1..73: mol_type = other DNA
gcctgggtagctcagtcggtagagcatcagactctaaatctgagggtccagggttcaagtccctgtccaggcg

SEQ ID NO: 588 - DNA, 73 bp, synthetic construct
  source 1..73: mol_type = other DNA
gcctggatagctcagtcggtagagcatcagactctaaatctgagggtccagggttcaagtccctgttcaggcg

SEQ ID NO: 589 - DNA, 73 bp, synthetic construct
  source 1..73: mol_type = other DNA
gcctggatagctcagttggtagagcatcagactctaaatctgagggtccagggttcaagtccctgttcaggcg

SEQ ID NO: 590 - DNA, 73 bp, synthetic construct
  source 1..73: mol_type = other DNA
gcctggatagctcagttggtagagcatcagactctaaatctgagggtccagggttcaagtccctgttcaggca

SEQ ID NO: 591 - DNA, 73 bp, synthetic construct
  source 1..73: mol_type = other DNA
gcccggatagctcagtcggtagagcatcagactctaaatctgagggtccggggttcaagtccctgttcgggcg

SEQ ID NO: 592 - DNA, 73 bp, synthetic construct
  source 1..73: mol_type = other DNA
gcccggctagctcagtcggtagagcatgggactctaaatctcagggtcgtgggttcgagccccacgttgggcg

SEQ ID NO: 593 - DNA, 73 bp, synthetic construct
  source 1..73: mol_type = other DNA
gcccggctagctcagtcggtagagcatgagaccctaaatctcagggtcgtgggttcgagccccacgttgggcg

SEQ ID NO: 594 - DNA, 70 bp, synthetic construct
  source 1..70: mol_type = other DNA
ccagcatgtctcagtcggtatagtgtgagactctaaatctcagggtcgtgggttcaagccccacattggg

SEQ ID NO: 595 - DNA, 74 bp, synthetic construct
  source 1..74: mol_type = other DNA
gcctgggtagctcagtcggtagagctatcagactctaagcctgaggattcagggttcaatcccttgctggggcg

SEQ ID NO: 596 - DNA, 73 bp, synthetic construct
  source 1..73: mol_type = other DNA
gcccgactacctcagtcggtggagcatgggactctacatcccagggttgtgggttcgagccccacattgggca

SEQ ID NO: 597 - DNA, 73 bp, synthetic construct
  source 1..73: mol_type = other DNA
gcccagctagctcagtcggtagagcataagactctaaatctcagggttgtggattcgtgccccatgctgggtg

SEQ ID NO: 598 - DNA, 76 bp, synthetic construct
  source 1..76: mol_type = other DNA
gcctggatagctccttcggtagagcatcatcagactctaaatgtgagggtccagggttcaagttcctgtttgggcg

SEQ ID NO: 599 - DNA, 65 bp, synthetic construct
  source 1..65: mol_type = other DNA
gcgctagtcagtagagcatgagactctaaatctcagggtcgtgggttcgagccccacatcgggcg

SEQ ID NO: 600 - DNA, 73 bp, synthetic construct
  source 1..73: mol_type = other DNA
gcctggatagctcagttggtagaacatcagactctaaatctgacggtgcagggttcaagtccctgttcaggcg

SEQ ID NO: 601 - DNA, 73 bp, synthetic construct
  source 1..73: mol_type = other DNA
gtctagctagatcagttggtagagcataagactctaaatctcagggtcatgggtttgagccctacgttgggcg

SEQ ID NO: 602 - DNA, 73 bp, synthetic construct
  source 1..73: mol_type = other DNA
gcccagctagctcagccggtagagcacaagactctaaatctcagggtcgtgggtttgagccctgtgttgagca

SEQ ID NO: 603 - DNA, 71 bp, synthetic construct
  source 1..71: mol_type = other DNA
ctgcagctagctcagtcggtagagcatgagactctaaatctcagggtcatgggttcgtgccccatgttggg

SEQ ID NO: 604 - DNA, 73 bp, synthetic construct
  source 1..73: mol_type = other DNA
gcccggagagctcagtgggtagagcatcagactctaaatctgagggtccagggttcaagtcctcgttcgggca

SEQ ID NO: 605 - DNA, 73 bp, synthetic construct
  source 1..73: mol_type = other DNA
ccccggctggctcagtcagtagatcatgagactctaaatctcagggtcgtgggttcacgccccacactgggcg

SEQ ID NO: 606 - DNA, 73 bp, synthetic construct
  source 1..73: mol_type = other DNA
acctgggtagctcagtaggtagaacatcagactctaaatctgagggtctagggttcaagtccctgtccaggcg

SEQ ID NO: 607 - DNA, 62 bp, synthetic construct
  source 1..62: mol_type = other DNA
gatagctcagttgatagagcatcagactctaaatctgagggtccagggttcatgtccctgtt

SEQ ID NO: 608 - DNA, 73 bp, synthetic construct
  source 1..73: mol_type = other DNA
gcctggctagctcagtcggcaaagcatgagactctaaatctcagggtcgtgggctcgagctccatgttgggcg

SEQ ID NO: 609 - DNA, 74 bp, synthetic construct
  source 1..74: mol_type = other DNA
gcctggctacctcagttggtagagcatgggactctaaatcccagagtcagtgggttcaagcctcacattgagtg

SEQ ID NO: 610 - DNA, 73 bp, synthetic construct
  source 1..73: mol_type = other DNA
acccagatagctcagtcagtagagcatcagactctaaatctgagggtccaaggttcatgtccctttttgggtg

SEQ ID NO: 611 - DNA, 73 bp, synthetic construct
  source 1..73: mol_type = other DNA
gcccagctagctcagtcggtagagcatgagactctaaatctcagggtcatgggtttgagccccacgtttggtg

SEQ ID NO: 612 - DNA, 73 bp, synthetic construct
  source 1..73: mol_type = other DNA
gcccggctagctcagtcgatagagcatgagactctaaatctcagggtcgtgggttcgagccgcacgttgggcg

SEQ ID NO: 613 - DNA, 73 bp, synthetic construct
  source 1..73: mol_type = other DNA
gccaggatagttcaggtggtagagcatcagactctaaacctgagggttcagggttcaagtctctgtttgggcg

SEQ ID NO: 614 - DNA, 73 bp, synthetic construct
  source 1..73: mol_type = other DNA
gttggggtaactcagttggtagagtagcagactctacatctgagggtccagggtttaagtccatgtccaggca

SEQ ID NO: 615 - DNA, 73 bp, synthetic construct
  source 1..73: mol_type = other DNA
gcccgactacctcagtcggtggagcatgggacttcacatcccagggttgtgggttcgagccccacattgggca

SEQ ID NO: 616 - DNA, 73 bp, synthetic construct
  source 1..73: mol_type = other DNA
gcctgggtagctcagtcggtagagcatcagacttcaaatctgagggtccagggttcaagtccctgtccaggcg

SEQ ID NO: 617 - DNA, 73 bp, synthetic construct
  source 1..73: mol_type = other DNA
gcctggctagctcagtcggcaaagcatgagacttcaaatctcagggtcgtgggctcgagctccatgttgggcg

SEQ ID NO: 618 - DNA, 73 bp, synthetic construct
  source 1..73: mol_type = other DNA
gcccggctagctcagtcgatagagcatgagacttcaaatctcagggtcgtgggttcgagccgcacgttgggcg

SEQ ID NO: 619 - DNA, 73 bp, synthetic construct
  source 1..73: mol_type = other DNA
gcccggctagctcagtcggtagagcatgggacttcaaatctcagggtcgtgggttcgagccccacgttgggcg

SEQ ID NO: 620 - DNA, 73 bp, synthetic construct
  source 1..73: mol_type = other DNA
gcccggatagctcagtcggtagagcatcagacttcaaatctgagggtccggggttcaagtccctgttcgggcg

SEQ ID NO: 621 - DNA, 73 bp, synthetic construct
  source 1..73: mol_type = other DNA
gcctggatagctcagtcggtagagcatcagacttcaaatctgagggtccagggttcaagtccctgttcaggcg

SEQ ID NO: 622 - DNA, 73 bp, synthetic construct
  source 1..73: mol_type = other DNA
gcccggagagctcagtgggtagagcatcagacttcaaatctgagggtccagggttcaagtcctcgttcgggca

SEQ ID NO: 623 - DNA, 73 bp, synthetic construct
  source 1..73: mol_type = other DNA
gcctggatagctcagttggtagagcatcagacttcaaatctgagggtccagggttcaagtccctgttcaggca

SEQ ID NO: 624 - DNA, 73 bp, synthetic construct
  source 1..73: mol_type = other DNA
gcccagctagctcagtcggtagagcataagacttcaaatctcagggttgtggattcgtgccccatgctgggtg

SEQ ID NO: 625 - DNA, 73 bp, synthetic construct
  source 1..73: mol_type = other DNA
gtctagctagatcagttggtagagcataagacttcaaatctcagggtcatgggtttgagccctacgttgggcg

SEQ ID NO: 626 - DNA, 73 bp, synthetic construct
  source 1..73: mol_type = other DNA
gcctggatagctcagttggtagaacatcagacttcaaatctgacggtgcagggttcaagtccctgttcaggcg

SEQ ID NO: 627 - DNA, 71 bp, synthetic construct
  source 1..71: mol_type = other DNA
ctgcagctagctcagtcggtagagcatgagacttcaaatctcagggtcatgggttcgtgccccatgttggg

SEQ ID NO: 628 - DNA, 73 bp, synthetic construct
  source 1..73: mol_type = other DNA
acctgggtagcttagttggtagagcattggacttcaaatttgagggcccaggtttcaagtccctgtttgggtg

SEQ ID NO: 629 - DNA, 76 bp, synthetic construct
  source 1..76: mol_type = other DNA
gcctggatagctccttcggtagagcatcatcagacttcaaatgtgagggtccagggttcaagttcctgtttgggcg

SEQ ID NO: 630 - DNA, 73 bp, synthetic construct
  source 1..73: mol_type = other DNA
acctgggtagctcagtaggtagaacatcagacttcaaatctgagggtctagggttcaagtccctgtccaggcg

SEQ ID NO: 631 - DNA, 70 bp, synthetic construct
  source 1..70: mol_type = other DNA
ccagcatgtctcagtcggtatagtgtgagacttcaaatctcagggtcgtgggttcaagccccacattggg

SEQ ID NO: 632 - DNA, 69 bp, synthetic construct
  source 1..69: mol_type = other DNA
ccgaatagcttagttgatgaagcgtgagacttcaaatctcagggtagtgggttcaagccccacattgga

SEQ ID NO: 633 - DNA, 74 bp, synthetic construct
  source 1..74: mol_type = other DNA
gcctgggtagctcagtcggtagagctatcagacttcaagcctgaggattcagggttcaatcccttgctggggcg

SEQ ID NO: 634 - DNA, 73 bp, synthetic construct
  source 1..73: mol_type = other DNA
gcctggatagctcagttggtagagcatcagacttcaaatctgagggtccagggttcaagtccctgttcaggcg

SEQ ID NO: 635 - DNA, 65 bp, synthetic construct
  source 1..65: mol_type = other DNA
gcgctagtcagtagagcatgagacttcaaatctcagggtcgtgggttcgagccccacatcgggcg

SEQ ID NO: 636 - DNA, 74 bp, synthetic construct
  source 1..74: mol_type = other DNA
gcctggctacctcagttggtagagcatgggacttcaaatcccagagtcagtgggttcaagcctcacattgagtg

SEQ ID NO: 637 - DNA, 73 bp, synthetic construct
  source 1..73: mol_type = other DNA
gttggggtaactcagttggtagagtagcagacttcacatctgagggtccagggtttaagtccatgtccaggca

SEQ ID NO: 638 - DNA, 73 bp, synthetic construct
  source 1..73: mol_type = other DNA
acccagatagctcagtcagtagagcatcagacttcaaatctgagggtccaaggttcatgtccctttttgggtg

SEQ ID NO: 639 - DNA, 62 bp, synthetic construct
  source 1..62: mol_type = other DNA
gatagctcagttgatagagcatcagacttcaaatctgagggtccagggttcatgtccctgtt

SEQ ID NO: 640 - DNA, 73 bp, synthetic construct
  source 1..73: mol_type = other DNA
gccaggatagttcaggtggtagagcatcagacttcaaacctgagggttcagggttcaagtctctgtttgggcg

SEQ ID NO: 641 - DNA, 73 bp, synthetic construct
  source 1..73: mol_type = other DNA
gcccagctagctcagccggtagagcacaagacttcaaatctcagggtcgtgggtttgagccctgtgttgagca

SEQ ID NO: 642 - DNA, 73 bp, synthetic construct
  source 1..73: mol_type = other DNA
gcccggctagctcagtcggtagagcatgagacctcaaatctcagggtcgtgggttcgagccccacgttgggcg

SEQ ID NO: 643 - DNA, 73 bp, synthetic construct
  source 1..73: mol_type = other DNA
gcccagctagctcagtcggtagagcatgagacttcaaatctcagggtcatgggtttgagccccacgtttggtg

SEQ ID NO: 644 - DNA, 73 bp, synthetic construct
  source 1..73: mol_type = other DNA
ccccggctggctcagtcagtagatcatgagacttcaaatctcagggtcgtgggttcacgccccacactgggcg

SEQ ID NO: 645 - DNA, 73 bp, synthetic construct
  source 1..73: mol_type = other DNA
gcctcgttagcgcagtaggtagcgcgtcagtctttaaatctgaaggtcgtgagttcgatcctcacacggggca

SEQ ID NO: 646 - DNA, 73 bp, synthetic construct
  source 1..73: mol_type = other DNA
gcctcgttagcgcagtaggcagcgcgtcagtctttaaatctgaaggtcgtgagttcgagcctcacacggggca

SEQ ID NO: 647 - DNA, 73 bp, synthetic construct
  source 1..73: mol_type = other DNA
gccctcttagcgcagctggcagcgcgtcagtctttaaatctgaaggtcctgagttcaagcctcagagagggca

SEQ ID NO: 648 - DNA, 73 bp, synthetic construct
  source 1..73: mol_type = other DNA
gccctcttagcgcagtgggcagcgcgtcagtctttaaatctgaaggtcctgagttcgagcctcagagagggca

SEQ ID NO: 649 - DNA, 73 bp, synthetic construct
  source 1..73: mol_type = other DNA
gccctcttagcgcagcgggcagcgcgtcagtctttaaatctgaaggtcctgagttcgagcctcagagagggca

SEQ ID NO: 650 - DNA, 73 bp, synthetic construct
  source 1..73: mol_type = other DNA
gcctccttagcgcagtaggcagcgcgtcagtctttaaatctgaaggtcctgagttcgaacctcagagggggca

SEQ ID NO: 651 - DNA, 73 bp, synthetic construct
  source 1..73: mol_type = other DNA
gccctcttagtgcagctggcagcgcgtcagtttttaaatctgaaagtcctgagttcaagcctcagagagggca

SEQ ID NO: 652 - DNA, 73 bp, synthetic construct
  source 1..73: mol_type = other DNA
gcctcgttagcgcagtaggcagcgcgtcagtctctaaatctgaaggtcgtgagttcgagcctcacacggggca

SEQ ID NO: 653 - DNA, 73 bp, synthetic construct
  source 1..73: mol_type = other DNA
gcctcgttagcgcagtaggtagcgcgtcagtctctaaatctgaaggtcgtgagttcgatcctcacacggggca

SEQ ID NO: 654 - DNA, 73 bp, synthetic construct
  source 1..73: mol_type = other DNA
gccctcttagcgcagtgggcagcgcgtcagtctctaaatctgaaggtcctgagttcgagcctcagagagggca

SEQ ID NO: 655 - DNA, 73 bp, synthetic construct
  source 1..73: mol_type = other DNA
gccctcttagcgcagcgggcagcgcgtcagtctctaaatctgaaggtcctgagttcgagcctcagagagggca

SEQ ID NO: 656 - DNA, 73 bp, synthetic construct
  source 1..73: mol_type = other DNA
gccctcttagtgcagctggcagcgcgtcagtttctaaatctgaaagtcctgagttcaagcctcagagagggca

SEQ ID NO: 657 - DNA, 73 bp, synthetic construct
  source 1..73: mol_type = other DNA
gcctccttagcgcagtaggcagcgcgtcagtctctaaatctgaaggtcctgagttcgaacctcagagggggca

SEQ ID NO: 658 - DNA, 73 bp, synthetic construct
  source 1..73: mol_type = other DNA
gccctcttagcgcagctggcagcgcgtcagtctctaaatctgaaggtcctgagttcaagcctcagagagggca

SEQ ID NO: 659 - DNA, 73 bp, synthetic construct
  source 1..73: mol_type = other DNA
gccctcttagtgcagctggcagcgcgtcagttttcaaatctgaaagtcctgagttcaagcctcagagagggca

SEQ ID NO: 660 - DNA, 73 bp, synthetic construct
  source 1..73: mol_type = other DNA
gccctcttagcgcagtgggcagcgcgtcagtcttcaaatctgaaggtcctgagttcgagcctcagagagggca

SEQ ID NO: 661 - DNA, 73 bp, synthetic construct
  source 1..73: mol_type = other DNA
gcctcgttagcgcagtaggcagcgcgtcagtcttcaaatctgaaggtcgtgagttcgagcctcacacggggca

SEQ ID NO: 662 - DNA, 73 bp, synthetic construct
  source 1..73: mol_type = other DNA
gccctcttagcgcagctggcagcgcgtcagtcttcaaatctgaaggtcctgagttcaagcctcagagagggca

SEQ ID NO: 663 - DNA, 73 bp, synthetic construct
  source 1..73: mol_type = other DNA
gccctcttagcgcagcgggcagcgcgtcagtcttcaaatctgaaggtcctgagttcgagcctcagagagggca

SEQ ID NO: 664 - DNA, 73 bp, synthetic construct
  source 1..73: mol_type = other DNA
gcctccttagcgcagtaggcagcgcgtcagtcttcaaatctgaaggtcctgagttcgaacctcagagggggca

SEQ ID NO: 665 - DNA, 73 bp, synthetic construct
  source 1..73: mol_type = other DNA
gcctcgttagcgcagtaggtagcgcgtcagtcttcaaatctgaaggtcgtgagttcgatcctcacacggggca

SEQ ID NO: 666 - DNA, 73 bp, synthetic construct
  source 1..73: mol_type = other DNA
gccgagatagctcagttgggagagcgttagactttagatctaaaggtccctggttcaatcccgggtttcggca

SEQ ID NO: 667 - DNA, 74 bp, synthetic construct
  source 1..74: mol_type = other DNA
gccgaaatagctcagttgggagagcgttagaccttagatcttaaaggtccctggttcaatcccgggtttcggca

SEQ ID NO: 668 - DNA, 74 bp, synthetic construct
  source 1..74: mol_type = other DNA
gctgaaatagctcagttgggagagcgttagactttagatcttaaagttccctggttcaaccctgggtttcagcc

SEQ ID NO: 669 - DNA, 73 bp, synthetic construct
  source 1..73: mol_type = other DNA
gccgaaatagctcagttgggagagcgttagactctagatctaaaggtccctggttcgatcccgggtttcggca

SEQ ID NO: 670 - DNA, 73 bp, synthetic construct
  source 1..73: mol_type = other DNA
gccgaaatagctcagttgggagagcgttagactctagatctaaaggtccctggttcaatcccgggtttcggca

SEQ ID NO: 671 - DNA, 74 bp, synthetic construct
  source 1..74: mol_type = other DNA
gctgaaatagctcagttgggagagcgttagactctagatcttaaagttccctggttcaaccctgggtttcagcc

SEQ ID NO: 672 - DNA, 74 bp, synthetic construct
  source 1..74: mol_type = other DNA
gccgaaatagctcagttgggagagcgttagaccctagatcttaaaggtccctggttcaatcccgggtttcggca

SEQ ID NO: 673 - DNA, 74 bp, synthetic construct
  source 1..74: mol_type = other DNA
gctgaaatagctcagttgggagagcgttagacttcagatcttaaagttccctggttcaaccctgggtttcagcc

SEQ ID NO: 674 - DNA, 74 bp, synthetic construct
  source 1..74: mol_type = other DNA
gccgaaatagctcagttgggagagcgttagacctcagatcttaaaggtccctggttcaatcccgggtttcggca

SEQ ID NO: 675 - DNA, 72 bp, synthetic construct
  source 1..72: mol_type = other DNA
ggctcgttggtctaggggtatgattctcggttttagtccgagaggtcccgggttcaaatcccggacgagccc

SEQ ID NO: 676 - DNA, 72 bp, synthetic construct
  source 1..72: mol_type = other DNA
ggctcgttggtctaggggtatgattctcgcttttagtgtgagaggtcccgggttcaaatcccggacgagccc

SEQ ID NO: 677 - DNA, 72 bp, synthetic construct
  source 1..72: mol_type = other DNA
ggctcgttggtctagtggtatgattctcgcttttagtgcgagaggtcccgggttcaaatcccggacgagccc

SEQ ID NO: 678 - DNA, 72 bp, synthetic construct
  source 1..72: mol_type = other DNA
ggctcgttggtctaggggtatgattctcgcttttagtgcgagaggtcccgggttcaaatcccggacgagccc

SEQ ID NO: 679 - DNA, 72 bp, synthetic construct
  source 1..72: mol_type = other DNA
ggctcgttggtctaggggtatgattctcgcttttaatgcgagaggtcccgggttcaaatcccggacgagccc

SEQ ID NO: 680 - DNA, 72 bp, synthetic construct
  source 1..72: mol_type = other DNA
ggctcgttggtctaggggtatgattctcggttctagtccgagaggtcccgggttcaaatcccggacgagccc

SEQ ID NO: 681 - DNA, 72 bp, synthetic construct
  source 1..72: mol_type = other DNA
ggctcgttggtctaggggtatgattctcgcttctagtgtgagaggtcccgggttcaaatcccggacgagccc

SEQ ID NO: 682 - DNA, 72 bp, synthetic construct
  source 1..72: mol_type = other DNA
ggctcgttggtctagtggtatgattctcgcttctagtgcgagaggtcccgggttcaaatcccggacgagccc

SEQ ID NO: 683 - DNA, 72 bp, synthetic construct
  source 1..72: mol_type = other DNA
ggctcgttggtctaggggtatgattctcgcttctagtgcgagaggtcccgggttcaaatcccggacgagccc

SEQ ID NO: 684 - DNA, 72 bp, synthetic construct
  source 1..72: mol_type = other DNA
ggctcgttggtctaggggtatgattctcgcttctaatgcgagaggtcccgggttcaaatcccggacgagccc

SEQ ID NO: 685 - DNA, 72 bp, synthetic construct
  source 1..72: mol_type = other DNA
ggctcgttggtctaggggtatgattctcgctttcagtgcgagaggtcccgggttcaaatcccggacgagccc

SEQ ID NO: 686 - DNA, 72 bp, synthetic construct
  source 1..72: mol_type = other DNA
ggctcgttggtctaggggtatgattctcgctttcagtgtgagaggtcccgggttcaaatcccggacgagccc

SEQ ID NO: 687 - DNA, 72 bp, synthetic construct
  source 1..72: mol_type = other DNA
ggctcgttggtctaggggtatgattctcgctttcaatgcgagaggtcccgggttcaaatcccggacgagccc

SEQ ID NO: 688 - DNA, 72 bp, synthetic construct
  source 1..72: mol_type = other DNA
ggctcgttggtctagtggtatgattctcgctttcagtgcgagaggtcccgggttcaaatcccggacgagccc

SEQ ID NO: 689 - DNA, 72 bp, synthetic construct
  source 1..72: mol_type = other DNA
ggctcgttggtctaggggtatgattctcggtttcagtccgagaggtcccgggttcaaatcccggacgagccc

SEQ ID NO: 690 - DNA, 82 bp, synthetic construct
  source 1..82: mol_type = other DNA
gctgtgatggccgagtggttaaggcgttggactttaaatccaatggggtctccccgcgcaggttcgaatcctgctcacagcg

SEQ ID NO: 691 - DNA, 82 bp, synthetic construct
  source 1..82: mol_type = other DNA
gtcacggtggccgagtggttaaggcgttggactttaaatccaatggggtttccccgcacaggttcgaatcctgttcgtgacg

SEQ ID NO: 692 - DNA, 82 bp, synthetic construct
  source 1..82: mol_type = other DNA
gacgaggtggccgagtggttaaggcgatggactttaaatccattgtgctctgcacgcgtgggttcgaatcccaccttcgtcg

SEQ ID NO: 693 - DNA, 82 bp, synthetic construct
  source 1..82: mol_type = other DNA
gcagcgatggccgagtggttaaggcgttggactttaaatccaatggggtctccccgcgcaggttcgaaccctgctcgctgcg

SEQ ID NO: 694 - DNA, 82 bp, synthetic construct
  source 1..82: mol_type = other DNA
gtagtcgtggccgagtggttaaggcgatggactttaaatccattggggtctccccgcgcaggttcgaatcctgccgactacg

SEQ ID NO: 695 - DNA, 82 bp, synthetic construct
  source 1..82: mol_type = other DNA
gtagtcgtggccgagtggttaaggcgatggactttaaatccattggggtttccccgcgcaggttcgaatcctgtcggctacg

SEQ ID NO: 696 - DNA, 82 bp, synthetic construct
  source 1..82: mol_type = other DNA
gctgtgatggccgagtggttaaggcgttggactttaaatccaatggggtctccccgcgcaggttcaaatcctgctcacagcg

SEQ ID NO: 697 - DNA, 82 bp, synthetic construct
  source 1..82: mol_type = other DNA
gacgaggtggccgagtggttaaggcgatggactttaaatccattgtgctctgcacacgtgggttcgaatcccatcctcgtcg

SEQ ID NO: 698 - DNA, 82 bp, synthetic construct
  source 1..82: mol_type = other DNA
gtagtcgtggccgagtggttaaggcgatggactttaaatccattggggtttccccacgcaggttcgaatcctgccgactacg

SEQ ID NO: 699 - DNA, 82 bp, synthetic construct
  source 1..82: mol_type = other DNA
gctgtgatggccgagtggttaaggtgttggactttaaatccaatgggggttccccgcgcaggttcaaatcctgctcacagcg

SEQ ID NO: 700 - DNA, 82 bp, synthetic construct
  source 1..82: mol_type = other DNA
gacgaggtggccgagtggttaaggcgatggactttaaatccattgtgctctgcacgcgtgggttcgaatcccaccctcgtcg

SEQ ID NO: 701 - DNA, 82 bp, synthetic construct
  source 1..82: mol_type = other DNA
gtagtcgtggccgagtggttaaggtgatggactttaaacccattggggtctccccgcgcaggttcgaatcctgccgactacg

SEQ ID NO: 702 - DNA, 74 bp, synthetic construct
  source 1..74: mol_type = other DNA
ggccggttagctcagttggttagagcgtgctttaactaatgccagggtcgaggtttcgatccccgtacgggcct

SEQ ID NO: 703 - DNA, 70 bp, synthetic construct
  source 1..70: mol_type = other DNA
caagaaattcatagaggttatgggattggctttaaaccagtttcaggaggttcgattccttcctttttgg

SEQ ID NO: 704 - DNA, 78 bp, synthetic construct
  source 1..78: mol_type = other DNA
gaggcctggccgagtggttaaggcgatggactttaaatccattgtgctctgcacgcgtgggttcgaatcccatcctcg

SEQ ID NO: 705 - DNA, 74 bp, synthetic construct
  source 1..74: mol_type = other DNA
ggccggttagctcagttggttagagcgtgctgctttaaatgccagggtcgaggtttcgatccccgtacgggcct

SEQ ID NO: 706 - DNA, 78 bp, synthetic construct
  source 1..78: mol_type = other DNA
agttgtagctgagtggttaaggcaacgagctttaaattcgttggtttctctctgtgcaggtttgaatcctgctaatta

SEQ ID NO: 707 - DNA, 72 bp, synthetic construct
  source 1..72: mol_type = other DNA
gggtgtatggctcaggggtagagaatttgactttagatcaagaggtccctggttcaaatccaggtgccccct

SEQ ID NO: 708 - DNA, 82 bp, synthetic construct
  source 1..82: mol_type = other DNA
gtagtcgtggccgagtggttaaggcgatggactctaaatccattggggtttccccgcgcaggttcgaatcctgtcggctacg

SEQ ID NO: 709 - DNA, 82 bp, synthetic construct
  source 1..82: mol_type = other DNA
gctgtgatggccgagtggttaaggcgttggactctaaatccaatggggtctccccgcgcaggttcaaatcctgctcacagcg

SEQ ID NO: 710 - DNA, 82 bp, synthetic construct
  source 1..82: mol_type = other DNA
gcagcgatggccgagtggttaaggcgttggactctaaatccaatggggtctccccgcgcaggttcgaaccctgctcgctgcg

SEQ ID NO: 711 - DNA, 82 bp, synthetic construct
  source 1..82: mol_type = other DNA
gacgaggtggccgagtggttaaggcgatggactctaaatccattgtgctctgcacacgtgggttcgaatcccatcctcgtcg

SEQ ID NO: 712 - DNA, 82 bp, synthetic construct
  source 1..82: mol_type = other DNA
gacgaggtggccgagtggttaaggcgatggactctaaatccattgtgctctgcacgcgtgggttcgaatcccaccttcgtcg

SEQ ID NO: 713 - DNA, 82 bp, synthetic construct
  source 1..82: mol_type = other DNA
gtcacggtggccgagtggttaaggcgttggactctaaatccaatggggtttccccgcacaggttcgaatcctgttcgtgacg

SEQ ID NO: 714 - DNA, 82 bp, synthetic construct
  source 1..82: mol_type = other DNA
gacgaggtggccgagtggttaaggcgatggactctaaatccattgtgctctgcacgcgtgggttcgaatcccaccctcgtcg

SEQ ID NO: 715 - DNA, 82 bp, synthetic construct
  source 1..82: mol_type = other DNA
gtagtcgtggccgagtggttaaggcgatggactctaaatccattggggtctccccgcgcaggttcgaatcctgccgactacg

SEQ ID NO: 716 - DNA, 74 bp, synthetic construct
  source 1..74: mol_type = other DNA
ggccggttagctcagttggttagagcgtgctctaactaatgccagggtcgaggtttcgatccccgtacgggcct

SEQ ID NO: 717 - DNA, 82 bp, synthetic construct
  source 1..82: mol_type = other DNA
gctgtgatggccgagtggttaaggtgttggactctaaatccaatgggggttccccgcgcaggttcaaatcctgctcacagcg

SEQ ID NO: 718 - DNA, 82 bp, synthetic construct
  source 1..82: mol_type = other DNA
gtagtcgtggccgagtggttaaggcgatggactctaaatccattggggtttccccacgcaggttcgaatcctgccgactacg

SEQ ID NO: 719 - DNA, 82 bp, synthetic construct
  source 1..82: mol_type = other DNA
gctgtgatggccgagtggttaaggcgttggactctaaatccaatggggtctccccgcgcaggttcgaatcctgctcacagcg

SEQ ID NO: 720 - DNA, 82 bp, synthetic construct
  source 1..82: mol_type = other DNA
gtagtcgtggccgagtggttaaggtgatggactctaaacccattggggtctccccgcgcaggttcgaatcctgccgactacg

SEQ ID NO: 721 - DNA, 78 bp, synthetic construct
  source 1..78: mol_type = other DNA
gaggcctggccgagtggttaaggcgatggactctaaatccattgtgctctgcacgcgtgggttcgaatcccatcctcg

SEQ ID NO: 722 - DNA, 74 bp, synthetic construct
  source 1..74: mol_type = other DNA
ggccggttagctcagttggttagagcgtgctgctctaaatgccagggtcgaggtttcgatccccgtacgggcct

SEQ ID NO: 723 - DNA, 72 bp, synthetic construct
  source 1..72: mol_type = other DNA
gggtgtatggctcaggggtagagaatttgactctagatcaagaggtccctggttcaaatccaggtgccccct

SEQ ID NO: 724 - DNA, 70 bp, synthetic construct
  source 1..70: mol_type = other DNA
caagaaattcatagaggttatgggattggctctaaaccagtttcaggaggttcgattccttcctttttgg

SEQ ID NO: 725 - DNA, 78 bp, synthetic construct
  source 1..78: mol_type = other DNA
agttgtagctgagtggttaaggcaacgagctctaaattcgttggtttctctctgtgcaggtttgaatcctgctaatta

SEQ ID NO: 726 - DNA, 82 bp, synthetic construct
  source 1..82: mol_type = other DNA
gctgtgatggccgagtggttaaggtgttggacttcaaatccaatgggggttccccgcgcaggttcaaatcctgctcacagcg

SEQ ID NO: 727 - DNA, 82 bp, synthetic construct
  source 1..82: mol_type = other DNA
gtagtcgtggccgagtggttaaggcgatggacttcaaatccattggggtttccccacgcaggttcgaatcctgccgactacg

SEQ ID NO: 728 - DNA, 82 bp, synthetic construct
  source 1..82: mol_type = other DNA
gctgtgatggccgagtggttaaggcgttggacttcaaatccaatggggtctccccgcgcaggttcgaatcctgctcacagcg

SEQ ID NO: 729 - DNA, 82 bp, synthetic construct
  source 1..82: mol_type = other DNA
gctgtgatggccgagtggttaaggcgttggacttcaaatccaatggggtctccccgcgcaggttcaaatcctgctcacagcg

SEQ ID NO: 730 - DNA, 82 bp, synthetic construct
  source 1..82: mol_type = other DNA
gcagcgatggccgagtggttaaggcgttggacttcaaatccaatggggtctccccgcgcaggttcgaaccctgctcgctgcg

SEQ ID NO: 731 - DNA, 82 bp, synthetic construct
  source 1..82: mol_type = other DNA
gacgaggtggccgagtggttaaggcgatggacttcaaatccattgtgctctgcacgcgtgggttcgaatcccaccttcgtcg

SEQ ID NO: 732 - DNA, 82 bp, synthetic construct
  source 1..82: mol_type = other DNA
gtagtcgtggccgagtggttaaggcgatggacttcaaatccattggggtctccccgcgcaggttcgaatcctgccgactacg

SEQ ID NO: 733 - DNA, 82 bp, synthetic construct
  source 1..82: mol_type = other DNA
gacgaggtggccgagtggttaaggcgatggacttcaaatccattgtgctctgcacgcgtgggttcgaatcccaccctcgtcg

SEQ ID NO: 734 - DNA, 82 bp, synthetic construct
  source 1..82: mol_type = other DNA
gtcacggtggccgagtggttaaggcgttggacttcaaatccaatggggtttccccgcacaggttcgaatcctgttcgtgacg

SEQ ID NO: 735 - DNA, 82 bp, synthetic construct
  source 1..82: mol_type = other DNA
gtagtcgtggccgagtggttaaggcgatggacttcaaatccattggggtttccccgcgcaggttcgaatcctgtcggctacg

SEQ ID NO: 736 - DNA, 82 bp, synthetic construct
  source 1..82: mol_type = other DNA
gacgaggtggccgagtggttaaggcgatggacttcaaatccattgtgctctgcacacgtgggttcgaatcccatcctcgtcg

SEQ ID NO: 737 - DNA, 82 bp, synthetic construct
  source 1..82: mol_type = other DNA
gtagtcgtggccgagtggttaaggtgatggacttcaaacccattggggtctccccgcgcaggttcgaatcctgccgactacg

SEQ ID NO: 738 - DNA, 78 bp, synthetic construct
  source 1..78: mol_type = other DNA
agttgtagctgagtggttaaggcaacgagcttcaaattcgttggtttctctctgtgcaggtttgaatcctgctaatta

SEQ ID NO: 739 - DNA, 72 bp, synthetic construct
  source 1..72: mol_type = other DNA
gggtgtatggctcaggggtagagaatttgacttcagatcaagaggtccctggttcaaatccaggtgccccct

SEQ ID NO: 740 - DNA, 78 bp, synthetic construct
  source 1..78: mol_type = other DNA
gaggcctggccgagtggttaaggcgatggacttcaaatccattgtgctctgcacgcgtgggttcgaatcccatcctcg

SEQ ID NO: 741 - DNA, 70 bp, synthetic construct
  source 1..70: mol_type = other DNA
caagaaattcatagaggttatgggattggcttcaaaccagtttcaggaggttcgattccttcctttttgg

SEQ ID NO: 742 - DNA, 74 bp, synthetic construct
  source 1..74: mol_type = other DNA
ggccggttagctcagttggttagagcgtgcttcaactaatgccagggtcgaggtttcgatccccgtacgggcct

SEQ ID NO: 743 - DNA, 74 bp, synthetic construct
  source 1..74: mol_type = other DNA
ggccggttagctcagttggttagagcgtgctgcttcaaatgccagggtcgaggtttcgatccccgtacgggcct

SEQ ID NO: 744 - DNA, 73 bp, synthetic construct
  source 1..73: mol_type = other DNA
ggctccatagctcagtggttagagcactggtctttaaaaccaggggtcgcgagttcgatcctcgctggggcct

SEQ ID NO: 745 - DNA, 74 bp, synthetic construct
  source 1..74: mol_type = other DNA
ggcgccgtggcttagctggttaaagcgcctgtctttaaaacaggagatcctgggttcgaatcccagcggtgcct

SEQ ID NO: 746 - DNA, 74 bp, synthetic construct
  source 1..74: mol_type = other DNA
ggctccgtggcttagctggttaaagcgcctgtctttaaaacaggagatcctgggttcgaatcccagcggggcct

SEQ ID NO: 747 - DNA, 74 bp, synthetic construct
  source 1..74: mol_type = other DNA
ggctccgtagcttagttggttaaagcgcctgtctttaaaacaggagatcctgggttcgactcccagcggggcct

SEQ ID NO: 748 - DNA, 74 bp, synthetic construct
  source 1..74: mol_type = other DNA
ggctctatggcttagttggttaaagcgcctgtctttaaaacaggagatcctgggttcgactcccagtggggcct

SEQ ID NO: 749 - DNA, 74 bp, synthetic construct
  source 1..74: mol_type = other DNA
ggccctgtggcttagctggtcaaagcgcctgtctttaaaacaggagatcctgggttcgaatcccagcggggcct

SEQ ID NO: 750 - DNA, 74 bp, synthetic construct
  source 1..74: mol_type = other DNA
ggctctatggcttagttggttaaagcgcctgtctttaaaacaggagatcctgggttcgaatcccagtagagcct

SEQ ID NO: 751 - DNA, 74 bp, synthetic construct
  source 1..74: mol_type = other DNA
ggcgccgtggcttagttggttaaagcgcctgtctttaaaacaggagatcctgggttcgaatcccagcggtgcct

SEQ ID NO: 752 - DNA, 73 bp, synthetic construct
  source 1..73: mol_type = other DNA
ggctccatagctcaggggttagagcactggtctttaaaaccaggggtcgcgagttcaaatctcgctggggcct

SEQ ID NO: 753 - DNA, 74 bp, synthetic construct
  source 1..74: mol_type = other DNA
ggctctgtggcttagttggctaaagcgcctgtctttaaaacaggagatcctgggttcgaatcccagcggggcct

SEQ ID NO: 754 - DNA, 74 bp, synthetic construct
  source 1..74: mol_type = other DNA
ggcttcgtggcttagctggttaaagcgcctgtctttaaaacaggagatcctgggttcgaatcccagcgaggcct

SEQ ID NO: 755 - DNA, 72 bp, synthetic construct
  source 1..72: mol_type = other DNA
ggcgcggtggccaagtggtaaggcgtcggtctttaaaaccgaagatcgcgggttcgaaccccgtccgtgcct

SEQ ID NO: 756 - DNA, 72 bp, synthetic construct
  source 1..72: mol_type = other DNA
ggcgcggtggccaagtggtaaggcgtcggtctttaaaaccgaagatcacgggttcgaaccccgtccgtgcct

SEQ ID NO: 757 - DNA, 73 bp, synthetic construct
  source 1..73: mol_type = other DNA
ggccctatagctcaggggttagagcactggtctttaaaaccaggggtcgcgagttcaaatctcgctggggcct

SEQ ID NO: 758 - DNA, 73 bp, synthetic construct
  source 1..73: mol_type = other DNA
ggctccatagctcaggggttagagcgctggtctttaaaaccaggggtcgcgagttcaattctcgctggggcct

SEQ ID NO: 759 - DNA, 72 bp, synthetic construct
  source 1..72: mol_type = other DNA
ggctccatagctcaggggttagagcactggtctttaaaaccagggtcgcgagttcaaatctcgctggggcct

SEQ ID NO: 760 - DNA, 74 bp, synthetic construct
  source 1..74: mol_type = other DNA
ggccctgtagctcagcggttggagcgctggtctttaaaacctaggggtcgtgagttcaaatctcaccagggcct

SEQ ID NO: 761 - DNA, 73 bp, synthetic construct
  source 1..73: mol_type = other DNA
ggctccatagctcaggggttagagcactggtctctaaaaccaggggtcgcgagttcaaatctcgctggggcct

SEQ ID NO: 762 - DNA, 73 bp, synthetic construct; identical to SEQ ID NO: 17
  source 1..73: mol_type = other DNA

SEQ ID NO: 763 - DNA, 73 bp, synthetic construct
  source 1..73: mol_type = other DNA
ggccctatagctcaggggttagagcactggtctctaaaaccaggggtcgcgagttcaaatctcgctggggcct

SEQ ID NO: 764 - DNA, 72 bp, synthetic construct
  source 1..72: mol_type = other DNA
ggcgcggtggccaagtggtaaggcgtcggtctctaaaaccgaagatcgcgggttcgaaccccgtccgtgcct

SEQ ID NO: 765 - DNA, 74 bp, synthetic construct
  source 1..74: mol_type = other DNA
ggccctgtggcttagctggtcaaagcgcctgtctctaaaacaggagatcctgggttcgaatcccagcggggcct

SEQ ID NO: 766 - DNA, 74 bp, synthetic construct
  source 1..74: mol_type = other DNA
ggcgccgtggcttagttggttaaagcgcctgtctctaaaacaggagatcctgggttcgaatcccagcggtgcct

SEQ ID NO: 767 - DNA, 74 bp, synthetic construct
  source 1..74: mol_type = other DNA
ggctctatggcttagttggttaaagcgcctgtctctaaaacaggagatcctgggttcgactcccagtggggcct

SEQ ID NO: 768 - DNA, 74 bp, synthetic construct
  source 1..74: mol_type = other DNA
ggcttcgtggcttagctggttaaagcgcctgtctctaaaacaggagatcctgggttcgaatcccagcgaggcct

SEQ ID NO: 769 - DNA, 74 bp, synthetic construct
  source 1..74: mol_type = other DNA
ggcgccgtggcttagctggttaaagcgcctgtctctaaaacaggagatcctgggttcgaatcccagcggtgcct

SEQ ID NO: 770 - DNA, 73 bp, synthetic construct
  source 1..73: mol_type = other DNA
ggctccatagctcaggggttagagcgctggtctctaaaaccaggggtcgcgagttcaattctcgctggggcct

SEQ ID NO: 771 - DNA, 74 bp, synthetic construct
  source 1..74: mol_type = other DNA
ggccctgtagctcagcggttggagcgctggtctctaaaacctaggggtcgtgagttcaaatctcaccagggcct

SEQ ID NO: 772 - DNA, 72 bp, synthetic construct
  source 1..72: mol_type = other DNA
ggctccatagctcaggggttagagcactggtctctaaaaccagggtcgcgagttcaaatctcgctggggcct

SEQ ID NO: 773 - DNA, 72 bp, synthetic construct
  source 1..72: mol_type = other DNA
ggcgcggtggccaagtggtaaggcgtcggtcttcaaaaccgaagatcacgggttcgaaccccgtccgtgcct

SEQ ID NO: 774 - DNA, 74 bp, synthetic construct
  source 1..74: mol_type = other DNA
ggctccgtggcttagctggttaaagcgcctgtcttcaaaacaggagatcctgggttcgaatcccagcggggcct

SEQ ID NO: 775 - DNA, 73 bp, synthetic construct
  source 1..73: mol_type = other DNA
ggctccatagctcagtggttagagcactggtcttcaaaaccaggggtcgcgagttcgatcctcgctggggcct

SEQ ID NO: 776 - DNA, 74 bp, synthetic construct
  source 1..74: mol_type = other DNA
ggccctgtggcttagctggtcaaagcgcctgtcttcaaaacaggagatcctgggttcgaatcccagcggggcct

SEQ ID NO: 777 - DNA, 74 bp, synthetic construct
  source 1..74: mol_type = other DNA
ggctctgtggcttagttggctaaagcgcctgtcttcaaaacaggagatcctgggttcgaatcccagcggggcct

SEQ ID NO: 778 - DNA, 73 bp, synthetic construct
  source 1..73: mol_type = other DNA
ggccctatagctcaggggttagagcactggtcttcaaaaccaggggtcgcgagttcaaatctcgctggggcct

SEQ ID NO: 779 - DNA, 74 bp, synthetic construct
  source 1..74: mol_type = other DNA
ggcgccgtggcttagctggttaaagcgcctgtcttcaaaacaggagatcctgggttcgaatcccagcggtgcct

SEQ ID NO: 780 - DNA, 74 bp, synthetic construct
  source 1..74: mol_type = other DNA
ggctctatggcttagttggttaaagcgcctgtcttcaaaacaggagatcctgggttcgactcccagtggggcct

SEQ ID NO: 781 - DNA, 74 bp, synthetic construct
  source 1..74: mol_type = other DNA
ggctctatggcttagttggttaaagcgcctgtcttcaaaacaggagatcctgggttcgaatcccagtagagcct

SEQ ID NO: 782 - DNA, 74 bp, synthetic construct
  source 1..74: mol_type = other DNA
ggcgccgtggcttagttggttaaagcgcctgtcttcaaaacaggagatcctgggttcgaatcccagcggtgcct

SEQ ID NO: 783 - DNA, 74 bp, synthetic construct
  source 1..74: mol_type = other DNA
ggcttcgtggcttagctggttaaagcgcctgtcttcaaaacaggagatcctgggttcgaatcccagcgaggcct

SEQ ID NO: 784 - DNA, 74 bp, synthetic construct
  source 1..74: mol_type = other DNA
ggctccgtagcttagttggttaaagcgcctgtcttcaaaacaggagatcctgggttcgactcccagcggggcct

SEQ ID NO: 785 - DNA, 73 bp, synthetic construct
  source 1..73: mol_type = other DNA
ggctccatagctcaggggttagagcactggtcttcaaaaccaggggtcgcgagttcaaatctcgctggggcct

SEQ ID NO: 786 - DNA, 73 bp, synthetic construct
  source 1..73: mol_type = other DNA
ggctccatagctcaggggttagagcgctggtcttcaaaaccaggggtcgcgagttcaattctcgctggggcct

SEQ ID NO: 787 - DNA, 74 bp, synthetic construct
  source 1..74: mol_type = other DNA
ggccctgtagctcagcggttggagcgctggtcttcaaaacctaggggtcgtgagttcaaatctcaccagggcct

SEQ ID NO: 788 - DNA, 72 bp, synthetic construct
  source 1..72: mol_type = other DNA
ggctccatagctcaggggttagagcactggtcttcaaaaccagggtcgcgagttcaaatctcgctggggcct

SEQ ID NO: 789 - DNA, 72 bp, synthetic construct
  source 1..72: mol_type = other DNA
gacctcgtggcgcaatggtagcgcgtctgactttagatcagaaggttgcgtgttcaagtcacgtcggggtca

SEQ ID NO: 790 - DNA, 72 bp, synthetic construct
  source 1..72: mol_type = other DNA
gacctcgtggcgcaacggcagcgcgtctgactttagatcagaaggttgcgtgttcaaatcacgtcggggtca

SEQ ID NO: 791 - DNA, 72 bp, synthetic construct
  source 1..72: mol_type = other DNA
gacctcgtggcgcaacggtagcgcgtctgactttagatcagaaggttgcgtgttcaaatcacgtcggggtca

SEQ ID NO: 792 - DNA, 72 bp, synthetic construct
  source 1..72: mol_type = other DNA
gacctcgtggcgcaacggtagcgcgtctgactttagatcagaaggctgcgtgttcgaatcacgtcggggtca

SEQ ID NO: 793 - DNA, 72 bp, synthetic construct
  source 1..72: mol_type = other DNA
ggcctcgtggcgcaacggtagcgcgtctgactttagatcagaaggttgcgtgttcaaatcacgtcggggtca

SEQ ID NO: 794 - DNA, 72 bp, synthetic construct
  source 1..72: mol_type = other DNA
ggcctcatggtgcaacagtagtgtgtctgactttagatcagaaggttgtatgttcaaatcacgtaggggtca

SEQ ID NO: 795 - DNA, 72 bp, synthetic construct
  source 1..72: mol_type = other DNA
ggcctcatggtgcaacagtagtgtgtctgactctagatcagaaggttgtatgttcaaatcacgtaggggtca

SEQ ID NO: 796 - DNA, 72 bp, synthetic construct
  source 1..72: mol_type = other DNA
gacctcgtggcgcaacggtagcgcgtctgactctagatcagaaggctgcgtgttcgaatcacgtcggggtca

SEQ ID NO: 797 - DNA, 72 bp, synthetic construct
  source 1..72: mol_type = other DNA
ggcctcgtggcgcaacggtagcgcgtctgactctagatcagaaggttgcgtgttcaaatcacgtcggggtca

SEQ ID NO: 798 - DNA, 72 bp, synthetic construct
  source 1..72: mol_type = other DNA
gacctcgtggcgcaatggtagcgcgtctgactctagatcagaaggttgcgtgttcaagtcacgtcggggtca

SEQ ID NO: 799 - DNA, 72 bp, synthetic construct
  source 1..72: mol_type = other DNA
gacctcgtggcgcaacggtagcgcgtctgactctagatcagaaggttgcgtgttcaaatcacgtcggggtca

SEQ ID NO: 800 - DNA, 72 bp, synthetic construct
  source 1..72: mol_type = other DNA
gacctcgtggcgcaacggcagcgcgtctgactctagatcagaaggttgcgtgttcaaatcacgtcggggtca

SEQ ID NO: 801 - DNA, 72 bp, synthetic construct
  source 1..72: mol_type = other DNA
ggcctcgtggcgcaacggtagcgcgtctgacttcagatcagaaggttgcgtgttcaaatcacgtcggggtca

SEQ ID NO: 802 - DNA, 72 bp, synthetic construct
  source 1..72: mol_type = other DNA
gacctcgtggcgcaatggtagcgcgtctgacttcagatcagaaggttgcgtgttcaagtcacgtcggggtca

SEQ ID NO: 803 - DNA, 72 bp, synthetic construct
  source 1..72: mol_type = other DNA
gacctcgtggcgcaacggtagcgcgtctgacttcagatcagaaggctgcgtgttcgaatcacgtcggggtca

SEQ ID NO: 804 - DNA, 72 bp, synthetic construct
  source 1..72: mol_type = other DNA
gacctcgtggcgcaacggtagcgcgtctgacttcagatcagaaggttgcgtgttcaaatcacgtcggggtca

SEQ ID NO: 805 - DNA, 72 bp, synthetic construct
  source 1..72: mol_type = other DNA
gacctcgtggcgcaacggcagcgcgtctgacttcagatcagaaggttgcgtgttcaaatcacgtcggggtca

SEQ ID NO: 806 - DNA, 72 bp, synthetic construct
  source 1..72: mol_type = other DNA
ggcctcatggtgcaacagtagtgtgtctgacttcagatcagaaggttgtatgttcaaatcacgtaggggtca

SEQ ID NO: 807 - DNA, 73 bp, synthetic construct
  source 1..73: mol_type = other DNA
ccttcgatagctcagttggtagagcggaggactttagatccttaggtcgctggttcgaatccggctcgaagga

SEQ ID NO: 808 - DNA, 73 bp, synthetic construct
  source 1..73: mol_type = other DNA
ccttcgatagctcagctggtagagcggaggactttagatccttaggtcgctggttcgaatccggctcggagga

SEQ ID NO: 809 - DNA, 73 bp, synthetic construct
  source 1..73: mol_type = other DNA
ccttcgatagctcagttggtagagcggaggactttagatccttaggtcgctggttcgaatccggctcggagga

SEQ ID NO: 810 - DNA, 73 bp, synthetic construct
  source 1..73: mol_type = other DNA
ccttcgatagctcagttggtagagcggaggactttagatccttaggtcgctggttcgattccggctcgaagga

SEQ ID NO: 811 - DNA, 73 bp, synthetic construct
  source 1..73: mol_type = other DNA
ccttcgatagctcagctggtagagcggaggactttagatccttaggtcgctggttcgattccagctcgaagga

SEQ ID NO: 812 - DNA, 73 bp, synthetic construct
  source 1..73: mol_type = other DNA
ccttcgatagctcagctggtagagcggaggactttagatccttaggtcgctggttcaattccggctcgaagga

SEQ ID NO: 813 - DNA, 73 bp, synthetic construct
  source 1..73: mol_type = other DNA
ctttcgatagctcagttggtagagcggaggactttagatccttaggtcgctggttcgaatccggctcgaagga

SEQ ID NO: 814 - DNA, 66 bp, synthetic construct
  source 1..66: mol_type = other DNA
ggtaaaatgactgagtaagcattagactttaaatctaaagacagaggtcaagacctctttttacca

SEQ ID NO: 815 - DNA, 66 bp, synthetic construct
  source 1..66: mol_type = other DNA
ggtaaaatggctgagtaagcattagactttaaatctaaagacagaggtcaaggcctcttttttcct

SEQ ID NO: 816 - DNA, 73 bp, synthetic construct
  source 1..73: mol_type = other DNA
gtcagtgttgcacaacggttaagtgaagaggctttaaacccagactggatgggttcaattcccatctctgccg

SEQ ID NO: 817 - DNA, 66 bp, synthetic construct
  source 1..66: mol_type = other DNA
ggtaaaatggctgagtaaagcattagactttaaatctaaggacagaggctaaacctctttttacca

SEQ ID NO: 818 - DNA, 66 bp, synthetic construct
  source 1..66: mol_type = other DNA
ggtaaaatggctgagtgaagcattggactttaaatctaaagacaggggttaagcctctttttacca

SEQ ID NO: 819 - DNA, 61 bp, synthetic construct
  source 1..61: mol_type = other DNA
tatagctcagtggtagagcatttaactttagatcaagaggtccctggatcaactctgggtg

SEQ ID NO: 820 - DNA, 64 bp, synthetic construct
  source 1..64: mol_type = other DNA
ggtaaaatggctgagtaagcattagactttaaatctaaagacagaggtcaaggcctttttacca

SEQ ID NO: 821 - DNA, 66 bp, synthetic construct
  source 1..66: mol_type = other DNA
ggtaaaatggctgagcaagcattggactttaaatctaaagacagatgttgagccatctttttagca

SEQ ID NO: 822 - DNA, 67 bp, synthetic construct
  source 1..67: mol_type = other DNA
ggtaaaatggctgagcaagcattagactttaaatctgaaaacagaggtcaaaggtctctttttacca

SEQ ID NO: 823 - DNA, 66 bp, synthetic construct
  source 1..66: mol_type = other DNA
ggtaaaatggctgagcaagcattagactttaaatctaaagacagaggttaaggcctctttttacca

SEQ ID NO: 824 - DNA, 66 bp, synthetic construct
  source 1..66: mol_type = other DNA
ggtaaaatggctgagtaagcattggactttaaatctaaagacagaggtcaagacctctttttacca

SEQ ID NO: 825 - DNA, 66 bp, synthetic construct
  source 1..66: mol_type = other DNA
ggtaaaatgactgaataagccttagactttaaatctgaagacagaggtcaaggcctctttttacca

SEQ ID NO: 826 - DNA, 64 bp, synthetic construct
  source 1..64: mol_type = other DNA
ggtaaaatggctgagtaagctttagactttaaatctaaagagagattgagctctctttttacca

SEQ ID NO: 827 - DNA, 73 bp, synthetic construct
  source 1..73: mol_type = other DNA
gggggtatagctcagggctagagctttttgactttagagcaagaggtccctggttcaaatccaggttctccct

SEQ ID NO: 828 - DNA, 73 bp, synthetic construct
  source 1..73: mol_type = other DNA
ccttcaatagttcagctggtagagcagaggactttaggtccttaggttgctggttcgattccagcttgaagga

SEQ ID NO: 829 - DNA, 66 bp, synthetic construct
  source 1..66: mol_type = other DNA
ggtaaaatggctgagtgaagcattggactttaaatctaaagacaggggctaagcctctttttacca

SEQ ID NO: 830 - DNA, 72 bp, synthetic construct
  source 1..72: mol_type = other DNA
tcttcaatagctcagctggtagagcggaggactttagattcttaggtgctggtttgattccgacttggagag

SEQ ID NO: 831 - DNA, 73 bp, synthetic construct
  source 1..73: mol_type = other DNA
ccttcgatagctcagttggtagagcggaggactctagatccttaggtcgctggttcgaatccggctcggagga

SEQ ID NO: 832 - DNA, 73 bp, synthetic construct
  source 1..73: mol_type = other DNA
ccttcgatagctcagctggtagagcggaggactctagatccttaggtcgctggttcgattccagctcgaagga

SEQ ID NO: 833 - DNA, 73 bp, synthetic construct
  source 1..73: mol_type = other DNA
ccttcgatagctcagctggtagagcggaggactctagatccttaggtcgctggttcgaatccggctcggagga

SEQ ID NO: 834 - DNA, 73 bp, synthetic construct
  source 1..73: mol_type = other DNA
ccttcgatagctcagctggtagagcggaggactctagatccttaggtcgctggttcaattccggctcgaagga

SEQ ID NO: 835 - DNA, 73 bp, synthetic construct
  source 1..73: mol_type = other DNA
ccttcgatagctcagttggtagagcggaggactctagatccttaggtcgctggttcgattccggctcgaagga

SEQ ID NO: 836 - DNA, 73 bp, synthetic construct
  source 1..73: mol_type = other DNA
ccttcgatagctcagttggtagagcggaggactctagatccttaggtcgctggttcgaatccggctcgaagga

SEQ ID NO: 837 - DNA, 73 bp, synthetic construct
  source 1..73: mol_type = other DNA
ctttcgatagctcagttggtagagcggaggactctagatccttaggtcgctggttcgaatccggctcgaagga

SEQ ID NO: 838 - DNA, 66 bp, synthetic construct
  source 1..66: mol_type = other DNA
ggtaaaatggctgagtgaagcattggactctaaatctaaagacaggggctaagcctctttttacca

SEQ ID NO: 839 - DNA, 66 bp, synthetic construct
  source 1..66: mol_type = other DNA
ggtaaaatggctgagcaagcattagactctaaatctaaagacagaggttaaggcctctttttacca

SEQ ID NO: 840 - DNA, 66 bp, synthetic construct
  source 1..66: mol_type = other DNA
ggtaaaatggctgagtgaagcattggactctaaatctaaagacaggggttaagcctctttttacca

SEQ ID NO: 841 - DNA, 61 bp, synthetic construct
  source 1..61: mol_type = other DNA
tatagctcagtggtagagcatttaactctagatcaagaggtccctggatcaactctgggtg

SEQ ID NO: 842 - DNA, 66 bp, synthetic construct
  source 1..66: mol_type = other DNA
ggtaaaatggctgagtaagcattagactctaaatctaaagacagaggtcaaggcctcttttttcct

SEQ ID NO: 843 - DNA, 66 bp, synthetic construct
  source 1..66: mol_type = other DNA
ggtaaaatgactgaataagccttagactctaaatctgaagacagaggtcaaggcctctttttacca

SEQ ID NO: 844 - DNA, 66 bp, synthetic construct
  source 1..66: mol_type = other DNA
ggtaaaatggctgagcaagcattggactctaaatctaaagacagatgttgagccatctttttagca

SEQ ID NO: 845 - DNA, 66 bp, synthetic construct
  source 1..66: mol_type = other DNA
ggtaaaatggctgagtaagcattggactctaaatctaaagacagaggtcaagacctctttttacca

SEQ ID NO: 846 - DNA, 66 bp, synthetic construct
  source 1..66: mol_type = other DNA
ggtaaaatggctgagtaaagcattagactctaaatctaaggacagaggctaaacctctttttacca

SEQ ID NO: 847 - DNA, 72 bp, synthetic construct
  source 1..72: mol_type = other DNA
tcttcaatagctcagctggtagagcggaggactctagattcttaggtgctggtttgattccgacttggagag

SEQ ID NO: 848 - DNA, 67 bp, synthetic construct
  source 1..67: mol_type = other DNA
ggtaaaatggctgagcaagcattagactctaaatctgaaaacagaggtcaaaggtctctttttacca

SEQ ID NO: 849 - DNA, 73 bp, synthetic construct
  source 1..73: mol_type = other DNA
gtcagtgttgcacaacggttaagtgaagaggctctaaacccagactggatgggttcaattcccatctctgccg

SEQ ID NO: 850 - DNA, 73 bp, synthetic construct
  source 1..73: mol_type = other DNA
ccttcaatagttcagctggtagagcagaggactctaggtccttaggttgctggttcgattccagcttgaagga

SEQ ID NO: 851 - DNA, 66 bp, synthetic construct
  source 1..66: mol_type = other DNA
ggtaaaatgactgagtaagcattagactctaaatctaaagacagaggtcaagacctctttttacca

SEQ ID NO: 852 - DNA, 73 bp, synthetic construct
  source 1..73: mol_type = other DNA
gggggtatagctcagggctagagctttttgactctagagcaagaggtccctggttcaaatccaggttctccct

SEQ ID NO: 853 - DNA, 64 bp, synthetic construct
  source 1..64: mol_type = other DNA
ggtaaaatggctgagtaagcattagactctaaatctaaagacagaggtcaaggcctttttacca

SEQ ID NO: 854 - DNA, 64 bp, synthetic construct
  source 1..64: mol_type = other DNA
ggtaaaatggctgagtaagctttagactctaaatctaaagagagattgagctctctttttacca

SEQ ID NO: 855 - DNA, 73 bp, synthetic construct
  source 1..73: mol_type = other DNA
ccttcgatagctcagttggtagagcggaggacttcagatccttaggtcgctggttcgaatccggctcgaagga

SEQ ID NO: 856 - DNA, 73 bp, synthetic construct
  source 1..73: mol_type = other DNA
ccttcgatagctcagttggtagagcggaggacttcagatccttaggtcgctggttcgattccggctcgaagga

SEQ ID NO: 857 - DNA, 73 bp, synthetic construct
  source 1..73: mol_type = other DNA
ccttcgatagctcagctggtagagcggaggacttcagatccttaggtcgctggttcgaatccggctcggagga

SEQ ID NO: 858 - DNA, 73 bp, synthetic construct
  source 1..73: mol_type = other DNA
ccttcgatagctcagctggtagagcggaggacttcagatccttaggtcgctggttcgattccagctcgaagga

SEQ ID NO: 859 - DNA, 73 bp, synthetic construct
  source 1..73: mol_type = other DNA
ccttcgatagctcagttggtagagcggaggacttcagatccttaggtcgctggttcgaatccggctcggagga

SEQ ID NO: 860 - DNA, 73 bp, synthetic construct
  source 1..73: mol_type = other DNA
ccttcgatagctcagctggtagagcggaggacttcagatccttaggtcgctggttcaattccggctcgaagga

SEQ ID NO: 861 - DNA, 66 bp, synthetic construct
  source 1..66: mol_type = other DNA
ggtaaaatggctgagtaagcattggacttcaaatctaaagacagaggtcaagacctctttttacca

SEQ ID NO: 862 - DNA, 73 bp, synthetic construct
  source 1..73: mol_type = other DNA
ctttcgatagctcagttggtagagcggaggacttcagatccttaggtcgctggttcgaatccggctcgaagga

SEQ ID NO: 863 - DNA, 66 bp, synthetic construct
  source 1..66: mol_type = other DNA
ggtaaaatgactgagtaagcattagacttcaaatctaaagacagaggtcaagacctctttttacca

SEQ ID NO: 864 - DNA, 64 bp, synthetic construct
  source 1..64: mol_type = other DNA
ggtaaaatggctgagtaagctttagacttcaaatctaaagagagattgagctctctttttacca

SEQ ID NO: 865 - DNA, 66 bp, synthetic construct
  source 1..66: mol_type = other DNA
ggtaaaatggctgagtaagcattagacttcaaatctaaagacagaggtcaaggcctcttttttcct

SEQ ID NO: 866 - DNA, 64 bp, synthetic construct
  source 1..64: mol_type = other DNA
ggtaaaatggctgagtaagcattagacttcaaatctaaagacagaggtcaaggcctttttacca

SEQ ID NO: 867 - DNA, 72 bp, synthetic construct
  source 1..72: mol_type = other DNA
tcttcaatagctcagctggtagagcggaggacttcagattcttaggtgctggtttgattccgacttggagag

SEQ ID NO: 868 - DNA, 66 bp, synthetic construct
  source 1..66: mol_type = other DNA
ggtaaaatggctgagcaagcattggacttcaaatctaaagacagatgttgagccatctttttagca

SEQ ID NO: 869 - DNA, 66 bp, synthetic construct
  source 1..66: mol_type = other DNA
ggtaaaatggctgagtgaagcattggacttcaaatctaaagacaggggttaagcctctttttacca

SEQ ID NO: 870 - DNA, 73 bp, synthetic construct
  source 1..73: mol_type = other DNA
gtcagtgttgcacaacggttaagtgaagaggcttcaaacccagactggatgggttcaattcccatctctgccg

SEQ ID NO: 871 - DNA, 67 bp, synthetic construct
  source 1..67: mol_type = other DNA
ggtaaaatggctgagcaagcattagacttcaaatctgaaaacagaggtcaaaggtctctttttacca

SEQ ID NO: 872 - DNA, 66 bp, synthetic construct
  source 1..66: mol_type = other DNA
ggtaaaatggctgagtaaagcattagacttcaaatctaaggacagaggctaaacctctttttacca

SEQ ID NO: 873 - DNA, 66 bp, synthetic construct
  source 1..66: mol_type = other DNA
ggtaaaatgactgaataagccttagacttcaaatctgaagacagaggtcaaggcctctttttacca

SEQ ID NO: 874 - DNA, 66 bp, synthetic construct
  source 1..66: mol_type = other DNA
ggtaaaatggctgagcaagcattagacttcaaatctaaagacagaggttaaggcctctttttacca

SEQ ID NO: 875 - DNA, 73 bp, synthetic construct
  source 1..73: mol_type = other DNA
gggggtatagctcagggctagagctttttgacttcagagcaagaggtccctggttcaaatccaggttctccct

SEQ ID NO: 876 - DNA, 73 bp, synthetic construct
  source 1..73: mol_type = other DNA
ccttcaatagttcagctggtagagcagaggacttcaggtccttaggttgctggttcgattccagcttgaagga

SEQ ID NO: 877 - DNA, 61 bp, synthetic construct
  source 1..61: mol_type = other DNA
tatagctcagtggtagagcatttaacttcagatcaagaggtccctggatcaactctgggtg

SEQ ID NO: 878 - DNA, 66 bp, synthetic construct
  source 1..66: mol_type = other DNA
ggtaaaatggctgagtgaagcattggacttcaaatctaaagacaggggctaagcctctttttacca

SEQ ID NO: 879 - DNA, 73 bp, synthetic construct
  source 1..73: mol_type = other DNA
ggttccatagtgtagcggttatcacgtctgcttttaacgcagaaggtcctgggttcgagccccagtggaacca

SEQ ID NO: 880 - DNA, 73 bp, synthetic construct
  source 1..73: mol_type = other DNA
ggttccatagtgtagtggttatcacgtctgcttttaacgcagaaggtcctgggttcgagccccagtggaacca

SEQ ID NO: 881 - DNA, 73 bp, synthetic construct
  source 1..73: mol_type = other DNA
gtttccgtagtgtagtggttatcacgttcgcctttaacgcgaaaggtccccggttcgaaaccgggcggaaaca

SEQ ID NO: 882 - DNA, 73 bp, synthetic construct
  source 1..73: mol_type = other DNA
gtttccgtggtgtagtggttatcacattcgcctttaacgcgaaaggtcctcgggtcgaaaccgagcggaaaca

SEQ ID NO: 883 - DNA, 73 bp, synthetic construct
  source 1..73: mol_type = other DNA
ggttccatagtgtagtggttatcacatctgcttttaacgcagaaggtcctgggttcaagccccagtggaacca

SEQ ID NO: 884 - DNA, 73 bp, synthetic construct
  source 1..73: mol_type = other DNA
gtttccgtagtgtagtggtcatcacgttcgcctttaacgcgaaaggtccccggttcgaaaccgggcggaaaca

SEQ ID NO: 885 - DNA, 73 bp, synthetic construct
  source 1..73: mol_type = other DNA
gtttccgtagtgtagcggttatcacattcgcctttaacgcgaaaggtccccggttcgatcccgggcggaaaca

SEQ ID NO: 886 - DNA, 73 bp, synthetic construct
  source 1..73: mol_type = other DNA
gtttccgtagtgtagtggttatcacgttcgcctttaacgcgaaaggtccccggttcgaaactgggcggaaaca

SEQ ID NO: 887 - DNA, 73 bp, synthetic construct
  source 1..73: mol_type = other DNA
gcttctgtagtgtagtggttatcacgttcgcctttaacgcgaaaggtccccggttcgaaaccgggcagaagca

SEQ ID NO: 888 - DNA, 73 bp, synthetic construct
  source 1..73: mol_type = other DNA
gtttccgtagtgtagtggttatcacgtttgcctttaacgcgaaaggtccccggttcgaaaccgggcagaaaca

SEQ ID NO: 889 - DNA, 74 bp, synthetic construct
  source 1..74: mol_type = other DNA
gtttccgtagtgtagtggttatcacgttcgcctttaacgcgtaaaggtccccggttcgaaaccgggcggaaaca

SEQ ID NO: 890 - DNA, 73 bp, synthetic construct
  source 1..73: mol_type = other DNA
gtttccgtagtgtagtggttatcacgttcgcctttaacgcgaaaggtccctggatcaaaaccaggcggaaaca

SEQ ID NO: 891 - DNA, 73 bp, synthetic construct
  source 1..73: mol_type = other DNA
gtttccgtagtggagtggttatcacgttcgcctttaacgcgaaaggtccccggtttgaaaccaggcggaaaca

SEQ ID NO: 892 - DNA, 73 bp, synthetic construct
  source 1..73: mol_type = other DNA
gtttccgtagtgtagtggttatcacgttcgcctttaacgcgaaaggtccgcggttcgaaaccgggcggaaaca

SEQ ID NO: 893 - DNA, 73 bp, synthetic construct
  source 1..73: mol_type = other DNA
ggttccatagtgtagtggttatcacgtctgcttctaacgcagaaggtcctgggttcgagccccagtggaacca

SEQ ID NO: 894 - DNA, 73 bp, synthetic construct
  source 1..73: mol_type = other DNA
ggttccatagtgtagcggttatcacgtctgcttctaacgcagaaggtcctgggttcgagccccagtggaacca

SEQ ID NO: 895 - DNA, 73 bp, synthetic construct
  source 1..73: mol_type = other DNA
ggttccatagtgtagtggttatcacatctgcttctaacgcagaaggtcctgggttcaagccccagtggaacca

SEQ ID NO: 896 - DNA, 73 bp, synthetic construct
  source 1..73: mol_type = other DNA
gtttccgtagtgtagtggttatcacgttcgcctctaacgcgaaaggtccccggttcgaaaccgggcggaaaca

SEQ ID NO: 897 - DNA, 73 bp, synthetic construct
  source 1..73: mol_type = other DNA
gcttctgtagtgtagtggttatcacgttcgcctctaacgcgaaaggtccccggttcgaaaccgggcagaagca

SEQ ID NO: 898 - DNA, 73 bp, synthetic construct
  source 1..73: mol_type = other DNA
gtttccgtggtgtagtggttatcacattcgcctctaacgcgaaaggtcctcgggtcgaaaccgagcggaaaca

SEQ ID NO: 899 - DNA, 73 bp, synthetic construct
  source 1..73: mol_type = other DNA
gtttccgtagtgtagtggtcatcacgttcgcctctaacgcgaaaggtccccggttcgaaaccgggcggaaaca

SEQ ID NO: 900 - DNA, 73 bp, synthetic construct
  source 1..73: mol_type = other DNA
gtttccgtagtgtagcggttatcacattcgcctctaacgcgaaaggtccccggttcgatcccgggcggaaaca

SEQ ID NO: 901 - DNA, 73 bp, synthetic construct
  source 1..73: mol_type = other DNA
gtttccgtagtgtagtggttatcacgttcgcctctaacgcgaaaggtccgcggttcgaaaccgggcggaaaca

SEQ ID NO: 902 - DNA, 74 bp, synthetic construct
  source 1..74: mol_type = other DNA
gtttccgtagtgtagtggttatcacgttcgcctctaacgcgtaaaggtccccggttcgaaaccgggcggaaaca

SEQ ID NO: 903 - DNA, 73 bp, synthetic construct
  source 1..73: mol_type = other DNA
gtttccgtagtggagtggttatcacgttcgcctctaacgcgaaaggtccccggtttgaaaccaggcggaaaca

SEQ ID NO: 904 - DNA, 73 bp, synthetic construct
  source 1..73: mol_type = other DNA
gtttccgtagtgtagtggttatcacgttcgcctctaacgcgaaaggtccctggatcaaaaccaggcggaaaca

SEQ ID NO: 905 - DNA, 73 bp, synthetic construct
  source 1..73: mol_type = other DNA
gtttccgtagtgtagtggttatcacgttcgcctctaacgcgaaaggtccccggttcgaaactgggcggaaaca

SEQ ID NO: 906 - DNA, 73 bp, synthetic construct
  source 1..73: mol_type = other DNA
gtttccgtagtgtagtggttatcacgtttgcctctaacgcgaaaggtccccggttcgaaaccgggcagaaaca

SEQ ID NO: 907 - DNA, 73 bp, synthetic construct
  source 1..73: mol_type = other DNA
gtttccgtagtgtagtggtcatcacgttcgccttcaacgcgaaaggtccccggttcgaaaccgggcggaaaca

SEQ ID NO: 908 - DNA, 73 bp, synthetic construct
  source 1..73: mol_type = other DNA
gtttccgtagtgtagtggttatcacgttcgccttcaacgcgaaaggtccccggttcgaaactgggcggaaaca

SEQ ID NO: 909 - DNA, 73 bp, synthetic construct
  source 1..73: mol_type = other DNA
gtttccgtagtgtagtggttatcacgttcgccttcaacgcgaaaggtccccggttcgaaaccgggcggaaaca

SEQ ID NO: 910 - DNA, 73 bp, synthetic construct
  source 1..73: mol_type = other DNA
gtttccgtagtggagtggttatcacgttcgccttcaacgcgaaaggtccccggtttgaaaccaggcggaaaca

SEQ ID NO: 911 - DNA, 73 bp, synthetic construct
  source 1..73: mol_type = other DNA
gtttccgtagtgtagtggttatcacgttcgccttcaacgcgaaaggtccgcggttcgaaaccgggcggaaaca

SEQ ID NO: 912 - DNA, 73 bp, synthetic construct
  source 1..73: mol_type = other DNA
ggttccatagtgtagcggttatcacgtctgctttcaacgcagaaggtcctgggttcgagccccagtggaacca

SEQ ID NO: 913 - DNA, 74 bp, synthetic construct
  source 1..74: mol_type = other DNA
gtttccgtagtgtagtggttatcacgttcgccttcaacgcgtaaaggtccccggttcgaaaccgggcggaaaca

SEQ ID NO: 914 - DNA, 73 bp, synthetic construct
  source 1..73: mol_type = other DNA
gtttccgtggtgtagtggttatcacattcgccttcaacgcgaaaggtcctcgggtcgaaaccgagcggaaaca

SEQ ID NO: 915 - DNA, 73 bp, synthetic construct
  source 1..73: mol_type = other DNA
gtttccgtagtgtagtggttatcacgttcgccttcaacgcgaaaggtccctggatcaaaaccaggcggaaaca

SEQ ID NO: 916 - DNA, 73 bp, synthetic construct
  source 1..73: mol_type = other DNA
ggttccatagtgtagtggttatcacatctgctttcaacgcagaaggtcctgggttcaagccccagtggaacca

SEQ ID NO: 917 - DNA, 73 bp, synthetic construct
  source 1..73: mol_type = other DNA
gtttccgtagtgtagtggttatcacgtttgccttcaacgcgaaaggtccccggttcgaaaccgggcagaaaca

SEQ ID NO: 918 - DNA, 73 bp, synthetic construct
  source 1..73: mol_type = other DNA
gtttccgtagtgtagcggttatcacattcgccttcaacgcgaaaggtccccggttcgatcccgggcggaaaca

SEQ ID NO: 919 - DNA, 73 bp, synthetic construct
  source 1..73: mol_type = other DNA
ggttccatagtgtagtggttatcacgtctgctttcaacgcagaaggtcctgggttcgagccccagtggaacca

SEQ ID NO: 920 - DNA, 73 bp, synthetic construct
  source 1..73: mol_type = other DNA
gcttctgtagtgtagtggttatcacgttcgccttcaacgcgaaaggtccccggttcgaaaccgggcagaagca

SEQ ID NO: 921 - RNA, 76 nt, synthetic construct
  source 1..76: mol_type = other RNA
  modified_base 1: mod_base = OTHER; note = Phosphorylated nucleotide
  modified_base 10: mod_base = OTHER; note = N2-methylguanosine
  modified_base 16: mod_base = OTHER; note = Dihydrouridine
  modified_base 17: mod_base = OTHER; note = Dihydrouridine
  modified_base 26: mod_base = OTHER; note = N2-methylguanosine
  modified_base 32: mod_base = OTHER; note = 2'-O-methyl modified nucleotide
  modified_base 34: mod_base = OTHER; note = 2'-O-methyl modified nucleotide
  modified_base 37: mod_base = OTHER; note = Wybutosine
  modified_base 39: mod_base = OTHER; note = Pseudouridine
  modified_base 40: mod_base = OTHER; note = 5-methylcytosine
  modified_base 46: mod_base = OTHER; note = N7-methylguanosine
  modified_base 49: mod_base = OTHER; note = 5-methylcytosine
  modified_base 54: mod_base = OTHER; note = Thymine
  modified_base 55: mod_base = OTHER; note = Pseudouridine
  modified_base 58: mod_base = OTHER; note = N1-methyladenosine
gcggatttagctcagnngggagagcgccagactgaananctggaggtcctgtgtncgatccacagaattcgcacca

SEQ ID NO: 922 - RNA, 73 nt, Homo sapiens
  source 1..73: mol_type = genomic RNA
  modified_base 34: mod_base = OTHER; note = Thymine
gacctcgtggcgcaacggttagcgcgtctgacttcagatcagaaggctgcgtgttcgaatcacgtcggggtca

(19) United States
(12) Patent Application Publication
(10) Pub. No.: US 2026/0317765 A1
(43) Pub. Date: Oct. 15, 2026

(54) ANTI-CODON ENGINEERED SUPPRESSOR TRANSFER RNAS

(71) Applicant: UNIVERSITY OF IOWA RESEARCH FOUNDATION, Iowa City, IA (US)

(72) Inventors: Christopher Ahern, Iowa City, IA (US); Lionel Gissot, Iowa City, IA (US)

(73) Assignee: UNIVERSITY OF IOWA RESEARCH FOUNDATION, Iowa City, IA (US)

(21) Appl. No.: 19/485,732

(22) PCT Filed: May 17, 2024
(86) PCT No.: PCT/US2024/030055

§ 371 Date: Nov. 19, 2025

Related U.S. Application Data
(60) Provisional application No. 63/467,828, filed on May 19, 2023.

(51) Int. Cl. C12N 15/113; C12N 15/88

(52) U.S. Cl. CPC C12N 15/113; C12N 15/88

(57) ABSTRACT

In certain aspects, provided herein is a transfer RNA (tRNA) comprising a T-arm, a D-arm, an anticodon-arm and an acceptor arm, wherein the anticodon-arm comprises a tri-nucleotide anticodon, wherein the tRNA is encoded by a sequence provided herein. In certain aspects, provided herein are oligonucleotide sequences and vectors that encode the tRNAs described herein. In certain aspects, provided herein are methods of use of the tRNAs, nucleic acids and vectors.

### CROSS-REFERENCE TO RELATED APPLICATION

This application claims priority to U.S. Provisional Application No. 63/467,828 that was filed on May 19, 2023, the entire contents of which are hereby incorporated by reference herein.

### STATEMENT REGARDING FEDERALLY SPONSORED RESEARCH

This invention was made with government support under Grant No. HG010824 Grant No. awarded by the National Institutes of Health. The U.S. government has certain rights in the invention.

### BACKGROUND

DNA molecules carry genetic information in the form of the sequence of the nucleotide bases that make up the DNA polymer. Only four nucleotide bases are utilized in DNA: adenine, guanine, cytosine, and thymine. This information, in the form of codons of three contiguous bases is transcribed into messenger RNA (mRNA), and then translated by transfer RNA (tRNA) and ribosomes to form proteins. Four nucleotide bases are utilized in RNA: adenine, guanine, cytosine, and uracil. The genetic code is the relation between a triplet codon and a particular amino acid. Sixty-four possible codon triplets form the genetic code, where three stop (also called terminating) codons, which provide a signal to the translation machinery (cellular ribosomes) to stop protein production at the particular codon. The other sixty-one triplets in the code correspond to one of the 20 standard amino acids. See FIG. 1.

DNA is translated by ribosomes, causing each amino acid to be linked together one by one to form polypeptides, according to the genetic instructions specifically provided by the DNA. When the ribosome reaches a stop codon, the elongation of the protein terminates. The three stop codons are UAG (amber), UAA (ochre) and UGA (opal). Mutations that occur that change an amino acid-encoding codon to stop codon are called “nonsense mutations.” These nonsense mutations can result in a significant truncation/shortening of the polypeptide sequence and can cause a profound change in genetic phenotype. Thus, even though a gene directing expression may be present, a crucial protein may not be produced because when the ribosome reaches the mutant stop signal, it terminates translation resulting in an unfinished protein.

Transfer RNAs translate mRNA into a protein on a ribosome. Each tRNA contains an “anti-codon” region that hybridizes with a complementary codon on the mRNA. A tRNA that carries its designated amino acid is called a “charged” tRNA. If the tRNA is one of the 61 amino-acid-associated (i.e., not a stop-signal-associated) tRNAs, it will normally attach its amino acid to the growing peptide. The structural gene of tRNA is about 72-90 nucleotides long and folds into a cloverleaf structure. tRNAs are transcribed by RNA polymerase III and contain their own intragenic split promoters that become a part of the mature tRNA coding sequence.

“Nonsense suppressors” are alleles of tRNA genes that contain an altered anticodon, such that instead of triggering a “stop” signal, they insert an amino acid in response to a termination codon. For example, an ochre mutation results in the creation of a UAA codon in an mRNA. An ochre suppressor gene produces tRNA with an AUU anticodon that inserts an amino acid at the UAA site, which permits the continued translation of the mRNA despite the presence of a codon that would normally trigger a stop in translation.

A number of nonsense suppressor tRNA alleles have been identified in prokaryotes and eukaryotes such as yeast and C. elegans. The different suppressor tRNAs vary in their suppression efficiency. In E. coli and other systems, the amber suppressors are relatively more efficient, ochre suppressors are less efficient while opal are the least, this suggests that the amber codons are used infrequently to terminate protein synthesis, while ochre and opal codons are more frequently used as natural termination signals.

Unwanted errors in the DNA blueprint can cause disease. For example, the occurrence of an unexpected “stop” signal in the middle of the protein, rather than at the end of the blueprint, results in the production of a truncated or shortened protein that has an altered function, or no function at all. Many human diseases, such as cystic fibrosis, muscular dystrophy, β-thalassemia and Liddle's syndrome result from unwanted stop signals in DNA reading frames for proteins that are important for proper lung, blood, muscle or kidney function, respectively.

Accordingly, there is a need to provide novel nonsense suppressor tRNAs and nonsense suppressor tRNAs that have activity effective to suppress termination of genes associated with diseases.

### SUMMARY

In one aspect, provided herein is a transfer RNA (tRNA) comprising a T-arm, a D-arm, an anticodon arm and an acceptor arm. In one aspect, the tRNA recognizes an undesirable stop codon or a premature termination codon (PTC).

In one aspect, provided herein is a transfer RNA (tRNA) comprising a T-arm, D-arm, and acceptor arm, wherein the anticodon arm recognizes a stop codon. In certain embodiments, the anticodon recognizes any one of the stops provided herein.

In one aspect, a transfer RNA (tRNA) comprising a T-arm, D-arm, and acceptor arm, wherein the sequence of the T-arm, D-arm and acceptor arm are of any one of the sequences provided herein.

In one aspect, provided herein is a transfer RNA (tRNA) of any one of the sequences provided herein.

In one aspect, provided herein is a transfer RNA (tRNA) that is an Alanine tRNA for use in any one of the compositions or methods provided herein.

In one aspect, provided herein is a transfer RNA (tRNA) that is an Arginine tRNA for use in any one of the compositions or methods provided herein.

In one aspect, provided herein is a transfer RNA (tRNA) that is an Asparagine tRNA for use in any one of the compositions or methods provided herein.

In one aspect, provided herein is a transfer RNA (tRNA) that is an Aspartic Acid tRNA for use in any one of the compositions or methods provided herein.

In one aspect, provided herein is a transfer RNA (tRNA) that is a Cysteine tRNA for use in any one of the compositions or methods provided herein.

In one aspect, provided herein is a transfer RNA (tRNA) that is a Glutamate tRNA for use in any one of the compositions or methods provided herein.

In one aspect, provided herein is a transfer RNA (tRNA) that is a Glutamine tRNA for use in any one of the compositions or methods provided herein.

In one aspect, provided herein is a transfer RNA (tRNA) that is a Glycine tRNA for use in any one of the compositions or methods provided herein.

In one aspect, provided herein is a transfer RNA (tRNA) that is a Histidine tRNA for use in any one of the compositions or methods provided herein.

In one aspect, provided herein is a transfer RNA (tRNA) that is an Isoleucine tRNA for use in any one of the compositions or methods provided herein.

In one aspect, provided herein is a transfer RNA (tRNA) that is a Leucine tRNA for use in any one of the compositions or methods provided herein.

In one aspect, provided herein is a transfer RNA (tRNA) that is a Lysine tRNA for use in any one of the compositions or methods provided herein.

In one aspect, provided herein is a transfer RNA (tRNA) that is a Methionine tRNA for use in any one of the compositions or methods provided herein.

In one aspect, provided herein is a transfer RNA (tRNA) that is a Phenylalanine tRNA for use in any one of the compositions or methods provided herein.

In one aspect, provided herein is a transfer RNA (tRNA) that is a Proline tRNA for use in any one of the compositions or methods provided herein.

In one aspect, provided herein is a transfer RNA (tRNA) that is a Serine tRNA for use in any one of the compositions or methods provided herein.

In one aspect, provided herein is a transfer RNA (tRNA) that is a Threonine tRNA for use in any one of the compositions or methods provided herein.

In one aspect, provided herein is a transfer RNA (tRNA) that is a Tryptophan tRNA for use in any one of the compositions or methods provided herein.

In one aspect, provided herein is a transfer RNA (tRNA) that is a Tyrosine tRNA for use in any one of the compositions or methods provided herein.

In one aspect, provided herein is a transfer RNA (tRNA) that is a Valine tRNA for use in any one of the compositions or methods provided herein.

In an embodiment, any one of the transfer RNAs (tRNAs) provided herein recognizes any one of the stops provided herein.

In one aspect, provided herein is a transfer RNA (tRNA) of any one of the sequences provided herein, wherein the anticodon-arm comprises a tri-nucleotide anticodon, wherein the anticodon is 5′-CUA-3′ and recognizes TAG-stop codons.

In one aspect, provided herein is a transfer RNA (tRNA) of any one of the sequences provided herein, wherein the anticodon-arm comprises a tri-nucleotide anticodon, wherein the anticodon is 5′-UCA-3′ and recognizes TGA stop codons.

In one aspect, provided herein is a transfer RNA (tRNA) of any one of the sequences provided herein, wherein the anticodon-arm comprises a tri-nucleotide anticodon, wherein the anticodon is 5′-UAA-3′ and recognizes TTA-stop codons.

In one aspect, provided herein is a transfer RNA (tRNA) of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 113, 114, 115, 116, 117, 118, 119,120, 121, 122, 226, 227, 229, 230, 231, 232, 233, 318, 354, 405, 453, 454, 507, 557, 669, 795, 857 or 858.

In one aspect, provided herein is a transfer RNA (tRNA) comprising a T-arm, a D-arm, an anticodon-arm and an acceptor arm, wherein the anticodon-arm comprises a tri-nucleotide anticodon, wherein the anticodon is 5′-CUA-3′ and recognizes TAG-stop codons, wherein the tRNA is encoded by a sequence comprising a sequence selected from the group consisting of: SEQ ID NOs: 1-8, 12, 15-18, 20, 23, 24, 26, 29, 32, 34, 113-122, 226, 227, 229-233, 354, 405, 507, 669, and 795.

In one aspect, provided herein is a transfer RNA (tRNA) comprising a T-arm, a D-arm, an anticodon-arm and an acceptor arm, wherein the anticodon-arm comprises a tri-nucleotide anticodon, wherein the anticodon is 5′-UCA-3′ and recognizes TGA stop codons, wherein the tRNA is encoded by a sequence comprising a sequence selected from the group consisting of: SEQ ID NOs: 10, 11, 13, 19, 25, 27, 30, 318, 857 and 858.

In one aspect, provided herein is a transfer RNA (tRNA) comprising a T-arm, a D-arm, an anticodon-arm and an acceptor arm, wherein the anticodon-arm comprises a tri-nucleotide anticodon, wherein the anticodon is 5′-UAA-3′ and recognizes TTA-stop codons, wherein the tRNA is encoded by a sequence comprising a sequence selected from the group consisting of: SEQ ID NOs: 22.

In one aspect, provided herein is a tRNA is SEQ ID NO: 9, 14, 21, 28, 31, 33, 35, 453, 454, or 557 wherein the anticodon is UUA.

In one aspect, a transfer RNA (tRNA) comprising a T-arm, a D-arm, an anticodon-arm and an acceptor arm, wherein the anticodon-arm comprises a tri-nucleotide anticodon, wherein the anticodon is 5′-UUA-3′ and recognizes TAA stop codons, wherein the tRNA is encoded by a sequence comprising a sequence selected from the group consisting of: SEQ ID NOs: 9, 14, 21, 28, 31, 33, 35, 453, 454, or 557.

In an embodiment of any one of the transfer RNAs (tRNAs) provided herein, the acceptor arm is not charged with an amino acid.

In certain aspects, in the tRNA described the acceptor arm is operably linked to an amino acid. In certain aspects, the amino acid is an amino acid listed in FIG. 1 or is a non-canonical amino acid. As used herein, a “non-canonical amino acid” is an unnatural amino acids that are are non-proteinogenic amino acids that are either found naturally in organisms or are synthetically made in a laboratory. They are amino acids that are not located in the genetic code of naturally occurring organisms. In certain aspects, the amino acid is arginine.

In certain embodiments, the present invention provides an oligonucleotide sequence that encodes any one of tRNAs as described described herein, such as, in an embodiment, wherein the oligonucleotide has a total length of less than 150 nucleotides. In certain embodiments, the oligonucleotide is DNA.

In certain embodiments, the present invention provides an oligonucleotide comprising a first oligonucleotide sequence and a second oligonucleotide sequence, wherein the first and second oligonucleotide sequences independently encode a tRNA as described above, wherein the first and second oligonucleotides independently have a total length of less than 150 nucleotides, and wherein the two sequences are in tandem.

In certain embodiments, the present invention provides an expression cassette comprising a promoter and a nucleic acid encoding a tRNA or oligonucleotides as described above.

In certain embodiments, the present invention provides a vector comprising the oligonucleotide or the expression cassette described above.

In certain embodiments, the vector is a viral or plasmid vector.

In certain embodiments, the present invention provides a composition comprising a tRNA, an oligonucleotide, or a vector described above, and a pharmaceutically acceptable carrier.

In certain embodiments, the carrier is a liposome.

In certain embodiments, the invention provides a cell comprising the vector described above.

The present invention provides a method of treating a stop-codon-associated genetic disease, comprising administering a tRNA composition described above to a patient in need thereof.

In certain embodiments, the genetic disease associated with a premature stop codon is cystic fibrosis, muscular dystrophy, β-thalassemia or Liddle's syndrome.

In certain embodiments, the present invention provides a method of restoring translation to a nucleotide sequence that includes a nonsense mutation in a cell, comprising introducing to the cell the composition described above.

In certain embodiments, the present invention provides a method of identifying tRNAs by high-throughput cloning and screening using suppression of a nonsense codon in luciferase enzymes including NanoLuc.

In one aspect, provided herein is a method of restoring translation to a nucleotide sequence that includes a nonsense mutation in a cell, comprising introducing to the cell a tRNA as described herein, wherein the tRNA restores translation to the nucleotide sequence that includes a nonsense mutation.

In one aspect, provided herein are nucleic acids that encode the tRNAs disclosed herein.

### BRIEF DESCRIPTION OF DRAWINGS

FIG. 1. Table of the Genetic Code.

FIG. 2. tRNAs have a general four-arm structure comprising a T-arm, a D-arm, an anticodon-arm, and an acceptor arm. These arms are also referred to as ‘loops’ throughout.

FIG. 3. An exemplary tRNA for nonsense suppression (H. sapiens tRNA^{Trp}_{TGA}).

FIG. 4. Anti-codon edited tRNA encoded in a vector used to identify functional tRNA sequences. This vector sequence includes a Nanoluciferase reporter system. The exemplary depicted vector was used to identify tRNA with TGA suppression.

FIG. 5. Arg tRNA sequences ranked by TAG rescue activity.

FIG. 6. Cys tRNA sequences ranked by TAG rescue activity.

FIG. 7. Gln tRNA sequence ranked by TGA rescue activity.

FIG. 8. Glu tRNA sequence ranked by TAG rescue activity.

FIG. 9. GlytRNA sequence ranked by TAG rescue activity.

FIG. 10. Ile tRNA sequences ranked by TAA rescue activity.

FIG. 11. Ile tRNA sequences ranked by TAG rescue activity.

FIG. 12. Leu tRNA sequences ranked by TAG rescue activity.

FIG. 13. Lys tRNA sequence ranked by TAA rescue activity.

FIG. 14. Phe tRNA sequences ranked by TAA rescue activity.

FIG. 15. Phe tRNA sequences ranked by TAG rescue activity.

FIG. 16. Trp tRNA sequences ranked by TAG rescue activity.

FIG. 17. Tyr tRNA sequences ranked by TGA rescue activity.

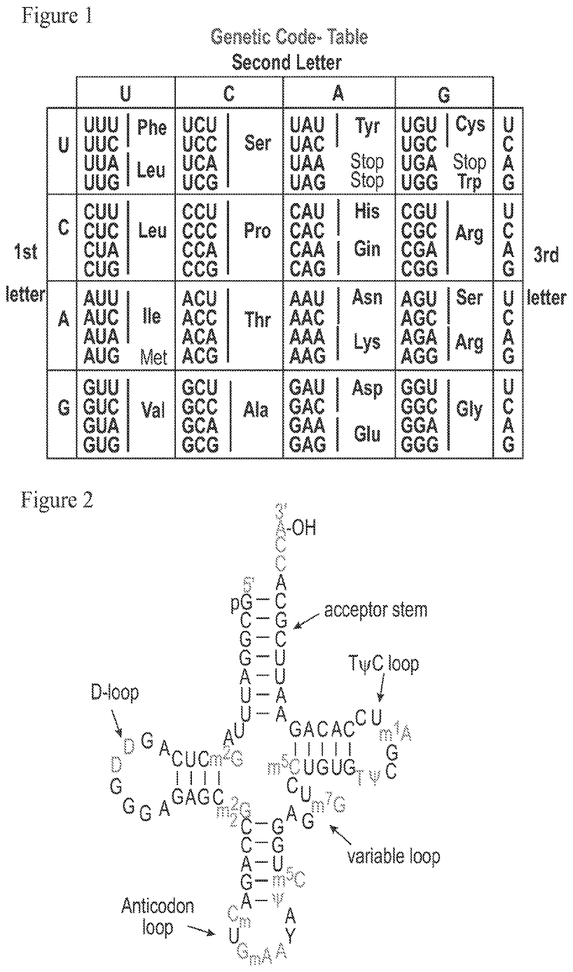

### DETAILED DESCRIPTION

Over the years, researchers have identified hundreds of unique point mutations that resulted in nonsense codons being established in human genes. These types of mutations result, for example, in muscular dystrophy, xeroderma pigmentosum, cystic fibrosis, hemophilia, anemia, hypothyroidism, p53 squamal cell carcinoma, p53 hepatocellular carcinoma, p53 ovarian carcinoma, esophageal carcinoma, osteocarcinoma, ovarian carcinoma, esophageal carcinoma, hepatocellular carcinoma, breast cancer, hepatocellular carcinoma, fibrous histiocytoma, ovarian carcinoma, SRY sex reversal, triosephosphate isomerase-anemia, diabetes and rickets. The BRACA-1 and BRACA-2 genes associated with breast cancer also have similar mutations.

The nucleotide sequences encoding several hundred human tRNAs are known and generally available to those of skill in the art through sources such as Genbank. The structure of tRNAs is highly conserved and tRNAs are often functional across species. Thus, bacterial or other eukaryotic tRNA sequences are also potential sources for the oligonucleotides for the stabilized tRNAs of the invention. The determination of whether a particular tRNA sequence is functional in a desired mammalian cell can be ascertained through routine experimentation.

tRNA genes generally have strong promoters that are active in all cell types. The promoters for eukaryotic tRNA genes are contained within the structural sequences encoding the tRNA molecule itself. Although there are elements that regulate transcriptional activity within the 5′ upstream region, the length of an active transcriptional unit may be considerably less than 500 base pairs and thus accommodation within a delivery vector can be straightforward. Once they have been transcribed and processed, tRNAs have low rates of degradation. Finally, gene therapy with a nonsense suppressor can maintain the endogenous physiological controls over the target gene that contains the nonsense codon.

### Nonsense Mutations

Transfer RNA (tRNA) is a type of RNA molecule that functions in the decoding of a messenger RNA (mRNA) sequence into a protein. tRNAs function at specific sites in the ribosome during translation, which synthesizes a protein from an mRNA molecule. Nonsense mutations, also called Premature Termination Codons (PTCs), make up ~10-15% of the single base pair mutations that cause human disease, and cystic fibrosis follows suit. In general, nonsense mutations have serious ramifications because of the almost complete loss of gene expression and activity and with the possibility of dominant negative effects of truncated products. PTCs result in premature translation termination and accelerated mRNA transcript decay through the Nonsense Mediated Decay (NMD) pathway.

The current studies show that the specific site within an RNA transcript to which a tRNA delivers its amino acid can be changed through molecular editing of the anti-codon sequence within the tRNA. This approach allowed for a premature termination codon (PTC) to be effectively and therapeutically reverted back into the originally lost amino acid.

Engineered tRNAs allow for “re-editing” of a disease-causing nonsense codon to a specific amino acid. These engineered tRNAs target only one type of stop codon, such as TGA over TAC or TAA. The small size of these tRNA molecules makes them amenable to ready expression, as the tRNA+the promoter is only ~300 bp. Briefly, in certain embodiments, an oligonucleotide is synthesized that comprises the structural component of a tRNA gene functional in human cells. In certain embodiments, the sequence of this oligonucleotide is designed based upon the known sequence with substitutions made in the anticodon region of the tRNA causing the specific tRNA to recognize a nonsense or other specific mutation.

Several small molecule screens have been performed to suppress nonsense stop codons through interactions with the ribosome, the most outstanding molecules being G418, Gentamicin and PTC124. PTC124 or Ataluren has recently been relieved from Phase 3 clinical trials as use for a cystic fibrosis therapeutic. Ataluren and aminoglycosides promote read-through of each of the three nonsense codons by putting in a near cognate amino acid that turn a nonsense mutation into a missense mutation. (Roy et al., PNAS 2016 Nov. 1; 113(44):12508-12513)

Anticodon-Targeting tRNA

tRNAs have a general four-arm structure comprising a T-arm, a D-arm, an anticodon-arm, and an acceptor arm (FIG. 2).

The T-arm is made up of a “T-stem” and a “TΨPC loop.” In certain embodiments, the T-stem is modified to increase the stability of the tRNA. In certain embodiments, the tRNA has a modified T-stem that increases the biological activity to suppress stop sites relative to the endogenous T-stem sequence.

Presented herein is a library of tRNA sequences with broad and unexpected potency for the therapeutic rescue of premature termination codons associated with 1000s of genetic diseases. Some of these tRNA sequences worked better than expected, e.g., isoleucine suppressor tRNA, while others also exhibit activity, e.g., serine. This dataset of the activity of over 900 tRNA sequences is useful in engineering effective tRNA molecules.

The present invention in one embodiment includes compositions comprising tRNAs that can be used with higher effectiveness in order to treat a wide variety of nonsense mutation-associated diseases. In certain aspects, the tRNAs are stabilized. The following sequences in Table 1 is written as DNA, but as RNA (transcribed DNA) the “T:thymidine” is “U:uracil.” Therefore, tRNAs transcribed from the following sequences all contain uracils in place of the thymidines.

TABLE 1

| | Anti- | tRNA | | SEQ ID |
|---|---|---|---|---|
| Ranking | codon | Identifier | tRNA Sequence | NO |
| 1 | CUA | tRNA-Ile- | GGCCGGTTAGCTCAGTTGGTtAGAGCGTGGTG | 1 |
| | | AAT-2-1 | CTctaAACGCCAAGGtCGCGGGTTCGATCCCC | |
| | | | GTACTGGCCA | |
| 2 | CUA | tRNA-Ile- | GGCCGGTTAGCTCAGTTGGTtAGAGCGTGGCG | 2 |
| | | AAT-1-1 | CTctaAACGCCAAGGtCGCGGGTTCGATCCCC | |
| | | | GTACGGGCCA | |
| 4 | CUA | tRNA- | GCCGAGATAGCTCAGTTGGGAGAGCGTTAGAC | 3 |
| | | Phe-GAA- | TctaGATCTAAAGGtCCCTGGTTCAATCCCGG | |
| | | 3-1 | GTTTCGGCA | |
| 6 | CUA | tRNA-Ile- | GGCTGGTTAGCTCAGTTGGTtAGAGCGTGGTG | 4 |
| | | AAT-3-1 | CTctaAACGCCAAGGtCGCGGGTTCGATCCCC | |
| | | | GTACTGGCCA | |
| 9 | CUA | tRNA-Ile- | GGCCGGTTAGCTCAGTTGGTCAGAGCGTGGTG | 5 |
| | | AAT-7-1 | CTctaAACGCCAAGGtCGCGGGTTCGATCCCC | |
| | | | GTACGGGCCA | |
| 12 | CUA | tRNA- | GGCTGGTTAGCTCAGTTGGTtAGAGCGTGGTG | 6 |
| | | Phe-GAA- | CTctaAACGCCAAGGtCGCGGGTTCGATCCCC | |
| | | 1-1 | GTACTGGCCA | |
| 16 | CUA | tRNA-Ile- | GGCCGGTTAGCTCAGTTGGTtAGAGCGTGGTG | 7 |
| | | AAT-4-1 | CTctaAACGCCAAGGtCGCGGGTTCGAACCCC | |
| | | | GTACGGGCCA | |
| 17 | CUA | tRNA- | GGGGATGTAGCTCAGTGGTAGAGCGCGCGCTT | 8 |
| | | Ala-CGC- | ctaATGTGTGAGGtCCCGGGTTCAATCCCCGG | |
| | | 3-1 | CATCTCCA | |
| 26 | UAA | tRNA-Ile- | GGCCGGTTAGCTCAGTTGGTtAGAGCGTGGTG | 9 |
| | | AAT-6-1 | CTttaAACGCTAAGGtCGCGGGTTCGATCCCC | |
| | | | GTACTGGCCA | |
| 27 | UCA | tRNA-Ile- | GGCCGGTTAGCTCAGTTGGTtAGAGCGTGGTG | 10 |
| | | AAT-2-1 | CTtcaAACGCCAAGGtCGCGGGTTCGATCCCC | |
| | | | GTACTGGCCA | |
| 35 | UCA | tRNA- | GCCGAAATAGCTCAGTTGGGAGAGCGTTAGAC | 11 |
| | | Phe-GAA- | TtcaGATCTAAAGGtCCCTGGTTCGATCCCGG | |
| | | 1-1 | GTTTCGGCA | |
| 39 | CUA | tRNA- | GGGGATGTAGCTCAGTGGTAGAGCGCATGCTT | 12 |
| | | Ala-CGC- | ctaATGTATGAGGCCCCGGGTTCGATCCCCGG | |
| | | 2-1 | CATCTCCA | |
| 40 | UCA | tRNA-Ile- | GGCCGGTTAGCTCAGTTGGTtAGAGCGTGGTG | 13 |
| | | AAT-6-1 | CTtcaAACGCTAAGGtCGCGGGTTCGATCCCC | |
| | | | GTACTGGCCA | |
| 47 | UUA | tRNA- | GCCGAAATAGCTCAGTTGGGAGAGCGTTAGAC | 14 |
| | | Phe-GAA- | TttaGATCTAAAGGtCCCTGGTTCGATCCCGG | |
| | | 1-1 | GTTTCGGCA | |
| 51 | CUA | tRNA- | GGCTCTGTGGCTTAGTTGGCtAAAGCGCCTGT | 15 |
| | | Thr-CGT- | CTctaAAACAGGAGAtCCTGGGTTCGAATCCC | |
| | | 3-1 | AGCGGGGCCT | |
| 57 | CUA | tRNA-Ile- | GGCCGGTTAGCTCAGTTGGTtAGAGCGTGGTG | 16 |
| | | AAT-6-1 | CTctaAACGCTAAGGtCGCGGGTTCGATCCCC | |
| | | | GTACTGGCCA | |
| 58 | CUA | tRNA- | GGCTCCATAGCTCAGTGGTtAGAGCACTGGTC | 17 |
| | | Thr-TGT- | TctaAAACCAGGGGtCGCGAGTTCGATCCTCG | |
| | | 2-1 | CTGGGGCCT | |
| 68 | CUA | tRNA- | GGCTCCGTAGCTTAGTTGGTtAAAGCGCCTGT | 18 |
| | | Thr-AGT- | CTctaAAACAGGAGAtCCTGGGTTCGACTCCC | |
| | | 3-1 | AGCGGGGCCT | |
| 71 | UCA | tRNA- | GCCGAAATAGCTCAGTTGGGAGAGCGTTAGAC | 19 |
| | | Phe-GAA- | TtcaGATCTAAAGGtCCCTGGTTCAATCCCGG | |
| | | 2-1 | GTTTCGGCA | |
| 74 | CUA | tRNA-Ile- | GGCCGGTTAGCTCAGTTGGTtAGAGCGTGGTG | 20 |
| | | AAT-5-1 | CTctaAACGCCAAGGtCGCGGGTTCGATCCCC | |
| | | | GTACGGGCCA | |
| 80 | UUA | tRNA-Ile- | GGCCGGTTAGCTCAGTTGGTtAGAGCGTGGCG | 21 |
| | | AAT-1-1 | CTttaAACGCCAAGGtCGCGGGTTCGATCCCC | |
| | | | GTACGGGCCA | |
| 84 | UUA | tRNA- | GCCGAAATAGCTCAGTTGGGAGAGCGTTAGAC | 22 |
| | | Phe-GAA- | TttaGATCTAAAGGtCCCTGGTTCAATCCCGG | |
| | | 2-1 | GTTTCGGCA | |
| 86 | CUA | tRNA- | GGGGATGTAGCTCAGTGGTAGAGCGCATGCTT | 23 |
| | | Ala-AGC- | ctaATGCATGAGGtCCCGGGTTCGATCCCCAG | |
| | | 4-1 | CATCTCCA | |
| 90 | CUA | tRNA- | GGCGCGGTGGCCAAGTGGTAAGGCGTCGGTCT | 24 |
| | | Thr-CGT- | ctaAAACCGAAGAtCACGGGTTCGAACCCCGT | |
| | | 2-1 | CCGTGCCT | |
| 94 | UCA | tRNA-Ile- | GGCCGGTTAGCTCAGTCGGCtAGAGCGTGGTG | 25 |
| | | AAT-8-1 | CTtcaAACGCCAAGGtCGCGGGTTCGATCCCC | |
| | | | GTACGGGCCA | |
| 95 | CUA | tRNA- | GGCTCTATGGCTTAGTTGGTLAAAGCGCCTGT | 26 |
| | | Thr-TGT- | CTctaAAACAGGAGAtCCTGGGTTCGAATCCC | |
| | | 1-1 | AGTAGAGCCT | |
| 96 | UCA | tRNA- | GGCGCGGTGGCCAAGTGGTAAGGCGTCGGTCT | 27 |
| | | Thr-CGT- | tcaAAACCGAAGAtCGCGGGTTCGAACCCCGT | |
| | | 4-1 | CCGTGCCT | |
| 103 | UUA | tRNA-Ile- | GGCCGGTTAGCTCAGTTGGTtAGAGCGTGGTG | 28 |
| | | AAT-2-1 | CTttaAACGCCAAGGtCGCGGGTTCGATCCCC | |
| | | | GTACTGGCCA | |
| 104 | CUA | tRNA- | GGGGGTATAGCTCAGTGGTAGAGCGCGTGCTT | 29 |
| | | Ala-AGC- | ctaATGCACGAGGtCCTGGGTTCGATCCCCAG | |
| | | 1-1 | TACCTCCA | |
| 105 | UCA | tRNA- | GCCGAGATAGCTCAGTTGGGAGAGCGTTAGAC | 30 |
| | | Phe-GAA- | TtcaGATCTAAAGGtCCCTGGTTCAATCCCGG | |
| | | 3-1 | GTTTCGGCA | |
| 106 | UUA | tRNA-Ile- | GGCCGGTTAGCTCAGTCGGCtAGAGCGTGGTG | 31 |
| | | AAT-8-1 | CTttaAACGCCAAGGtCGCGGGTTCGATCCCC | |
| | | | GTACGGGCCA | |
| 108 | CUA | tRNA- | GGCTCCGTGGCTTAGCTGGTLAAAGCGCCTGT | 32 |
| | | Thr-AGT- | CTctaAAACAGGAGAtCCTGGGTTCGAATCCC | |
| | | 2-1 | AGCGGGGCCT | |
| 109 | UUA | tRNA-Ile- | GGCTGGTTAGCTCAGTTGGTtAGAGCGTGGTG | 33 |
| | | AAT-3-1 | CTttaAACGCCAAGGtCGCGGGTTCGATCCCC | |
| | | | GTACTGGCCA | |
| 112 | CUA | tRNA- | GGGGAATTAGCTCAAATGGTAGAGCGCTCGCT | 34 |
| | | Ala-AGC- | TctaATGCGAGAGGtAGCGGGATCGATGCCCG | |
| | | 11-1 | CATTCTCCA | |
| 113 | UUA | tRNA-Ile- | GGCCGGTTAGCTCAGTTGGTCAGAGCGTGGTG | 35 |
| | | AAT-7-1 | CTttaAACGCCAAGGtCGCGGGTTCGATCCCC | |
| | | | GTACGGGCCA | |

The present invention in one embodiment includes compositions comprising tRNAs that can be used with higher effectiveness in order to treat a wide variety of nonsense mutation-associated diseases, such as depicted in Tables 2-14. In certain aspects, the tRNAs are stabilized.

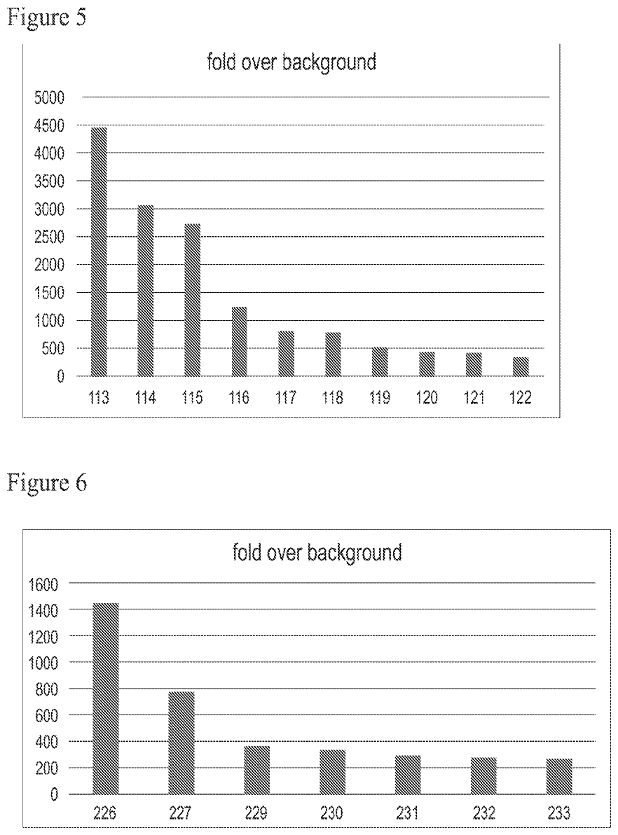

TABLE 2
Arginine (Arg) tRNAs (FIG. 5)

| | | fold | | SEQ |
|---|---|---|---|---|
| seq_tRNA | ACE | over | Rank | ID |
| GGCTCCGTGGCGCAATGGAtAGCGCATTGGACTctaA | TAG | 4422.24 | 20 | 113 |
| ATTCAAAGGtTCCGGGTTCGAGTCCCGGCGGAGTCG | | | | |
| GCCCCAGTGGCCTAATGGAtAAGGCATTGGCCTctaA | TAG | 3041.46 | 39 | 114 |
| AGCCAGGGAtTGTGGGTTCGAGTCCCATCTGGGGTG | | | | |
| GGCTCTGTGGCGCAATGGAtAGCGCATTGGACTctaA | TAG | 2719.18 | 43 | 115 |
| ATTCAAAGGtTGTGGGTTCGAGTCCCACCAGAGTCG | | | | |
| GCCCCGGTGGCCTAATGGAtAAGGCATTGGCCTctaA | TAG | 1227.07 | 79 | 116 |
| AGCCAGGGAtTGTGGGTTCGAGTCCCACCCGGGGTA | | | | |
| GGCTCTGTGGCGCAATGGAtAGCGCATTGGACTctaA | TAG | 791.34 | 97 | 117 |
| ATTCAAAGGtTGTGGGTTCGAATCCCACCAGAGTCG | | | | |
| GACCCAGTGGCCTAATGGAtAAGGCATCAGCCTctaG | TAG | 776.95 | 98 | 118 |
| AGCTGGGGAtTGTGGGTTCGAGTCCCATCTGGGTCG | | | | |
| GACCACGTGGCCTAATGGAtAAGGCGTCTGACTctaG | TAG | 505.44 | 127 | 119 |
| ATCAGAAGAtTGAGGGTTCGAATCCCTTCGTGGTTA | | | | |
| GGCCGCGTGGCCTAATGGAtAAGGCGTCTGACTctaG | TAG | 428.91 | 135 | 120 |
| ATCAGAAGAtTGCAGGTTCGAGTCCTGCCGCGGTCG | | | | |
| GGGCCAGTGGCGCAATGGAtAACGCGTCTGACTctaG | TAG | 399.39 | 138 | 121 |
| ATCAGAAGAtTCCAGGTTCGACTCCTGGCTGGCTCG | | | | |
| GGCTCTGTGGCGCAATGGAtAGCGCATTGGACTctaA | TAG | 326.35 | 151 | 122 |
| ATTCAAAGGtTGCGGGTTCGAGTCCCTCCAGAGTCG | | | | |

TABLE 3
Cysteine (Cys) tRNAs (FIG. 6)

| | | fold | | SEQ |
|---|---|---|---|---|
| seq_tRNA | ACE | over | Rank | ID |
| GGGGGTATAGCTCAGGGGTAGAGCATTTGACTctaGA | TAG | 1455.23 | 70 | 226 |
| TCAAGAGGtCCCTGGTTCAAATCCAGGTGCCCCCC | | | | |
| GGGGGTATAGCTCAGTGGGTAGAGCATTTGACTctaG | TAG | 773.95 | 100 | 227 |
| ATCAAGAGGtCCCCGGTTCAAATCCGGGTGCCCCCT | | | | |
| GGGGGTGTAGCTCAGTGGTAGAGCATTTGACTctaGA | TAG | 348.06 | 149 | 229 |
| TCAAGAGGtCCCTGGTTCAAATCCAGGTGCCCCCT | | | | |
| GGGGGTATAGCTCAGGGGTAGAGCATTTGACTctaGA | TAG | 329.27 | 150 | 230 |
| TCAAGAGGtCCCCGGTTCAAATCCGGGTGCCCCCT | | | | |
| GGGGGTATAGCTCAGGGGTAGAGCATTTGACTctaGA | TAG | 280.15 | 157 | 231 |
| TCAAGAGGtCCCTGGTTCAAATCCAGGTGCCCCCT | | | | |
| GGGGGCATAGCTCAGTGGTAGAGCATTTGACTctaGA | TAG | 266.50 | 161 | 232 |
| TCAAGAGGtCCCTGGTTCAAATCCAGGTGCCCCCT | | | | |
| GGGGGTATAGCTCAGGGGTAGAGCACTTGACTctaGA | TAG | 254.45 | 164 | 233 |
| TCAAGAGGtCCCTGGTTCAAATCCAGGTGCCCCCT | | | | |

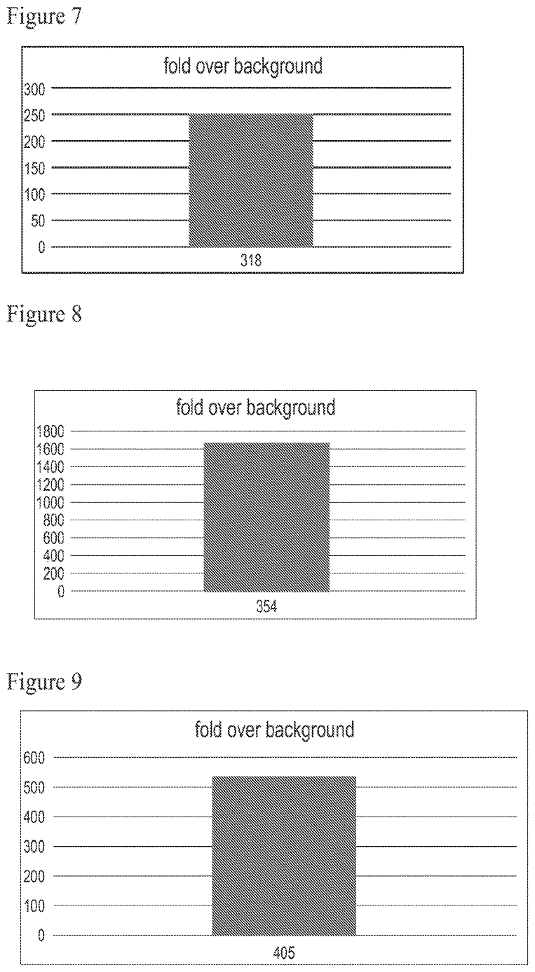

TABLE 4
Glutamine (Gln) tRNA (FIG. 7)

| | | fold | | SEQ |
|---|---|---|---|---|
| seq_tRNA | ACE | over | Rank | ID |
| GGCCCCATGGTGTAATGGTtAGCACTCTGGACTtcaAA | TGA | 252.02 | 165 | 318 |
| TCCAGCGaTCCGAGTTCAAATCTCGGTGGGACCT | | | | |

TABLE 5
Glutamate (Glu) tRNA (FIG. 8)

| | | fold | | SEQ |
|---|---|---|---|---|
| seq_tRNA | ACE | over | Rank | ID |
| TCCGTGGTGGTCTAGTGGCtAGGATTCGGCGCTctaAC | TAG | 1659.61 | 64 | 354 |
| CGCCTGCAGCTCGAGTTCGATTCCTGGTCAGGGAA | | | | |

TABLE 6
Glycine (Gly) tRNA (FIG. 9)

| | | fold | | SEQ |
|---|---|---|---|---|
| seq_tRNA | ACE | over | Rank | ID |
| GCGTCAGTGGTTTAGTGGTGGAATTCCTGCCTctaATG | TAG | 540.46 | 122 | 405 |
| CACGAGATCCGTGTTCAACTCCTGGTTGGTGCA | | | | |

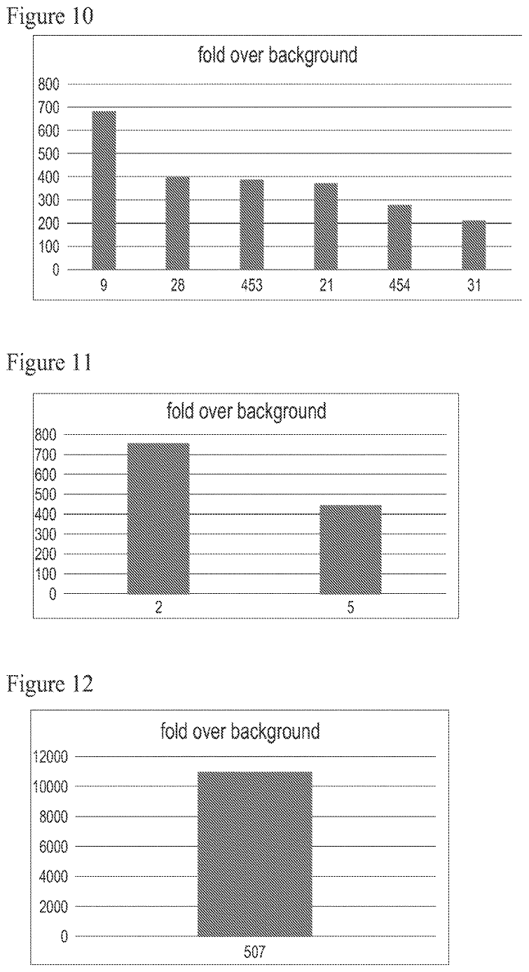

TABLE 7
Isoleucine (Ile) tRNAs (FIG. 10)

| | | fold | | SEQ |
|---|---|---|---|---|
| seq_tRNA | ACE | over | Rank | ID |
| GGCCGGTTAGCTCAGTTGGTtAGAGCGTGGTGCTttaA | TAA | 676.03 | 106 | 9 |
| ACGCTAAGGtCGCGGGTTCGATCCCCGTACTGGCCA | | | | |
| GGCCGGTTAGCTCAGTTGGTtAGAGCGTGGTGCTttaA | TAA | 392.98 | 141 | 28 |
| ACGCCAAGGtCGCGGGTTCGATCCCCGTACTGGCCA | | | | |
| GGCCGGTTAGCTCAGTTGGTtAGAGCGTGGTGCTttaA | TAA | 384.97 | 143 | 453 |
| ACGCCAAGGtCGCGGGTTCGATCCCCGTACGGGCCA | | | | |
| GGCCGGTTAGCTCAGTTGGTtAGAGCGTGGCGCTttaA | TAA | 364.35 | 148 | 21 |
| ACGCCAAGGtCGCGGGTTCGATCCCCGTACGGGCCA | | | | |
| GGCCGGTTAGCTCAGTTGGTcAGAGCGTGGTGCTttaA | TAA | 275.74 | 158 | 454 |
| ACGCCAAGGtCGCGGGTTCGATCCCCGTACGGGCCA | | | | |
| GGCCGGTTAGCTCAGTCGGCtAGAGCGTGGTGCTttaA | TAA | 206.52 | 175 | 31 |
| ACGCCAAGGtCGCGGGTTCGATCCCCGTACGGGCCA | | | | |

TABLE 8
Isoleucine (Ile) tRNAs (FIG. 11)

| | | fold | | SEQ |
|---|---|---|---|---|
| seq_tRNA | ACE | over | Rank | ID |
| GGCCGGTTAGCTCAGTTGGTtAGAGCGTGGCGCTctaA | TAG | 752.70 | 101 | 2 |
| ACGCCAAGGtCGCGGGTTCGATCCCCGTACGGGCCA | | | | |
| GGCCGGTTAGCTCAGTTGGTcAGAGCGTGGTGCTctaA | TAG | 443.62 | 134 | 5 |
| ACGCCAAGGtCGCGGGTTCGATCCCCGTACGGGCCA | | | | |

TABLE 9
Leucine (Leu) tRNA (FIG. 12)

| | | fold | | SEQ |
|---|---|---|---|---|
| seq_tRNA | ACE | over | Rank | ID |
| GGTAGCGTGGCCGAGCGGTctAAGGCGCTGGATTctaG | TAG | 10860.81 | 1 | 507 |
| CTCCAGTCTCTTCGGGGGCGTGGGTTCAAATCCCACC | | | | |
| GCTGCCA | | | | |

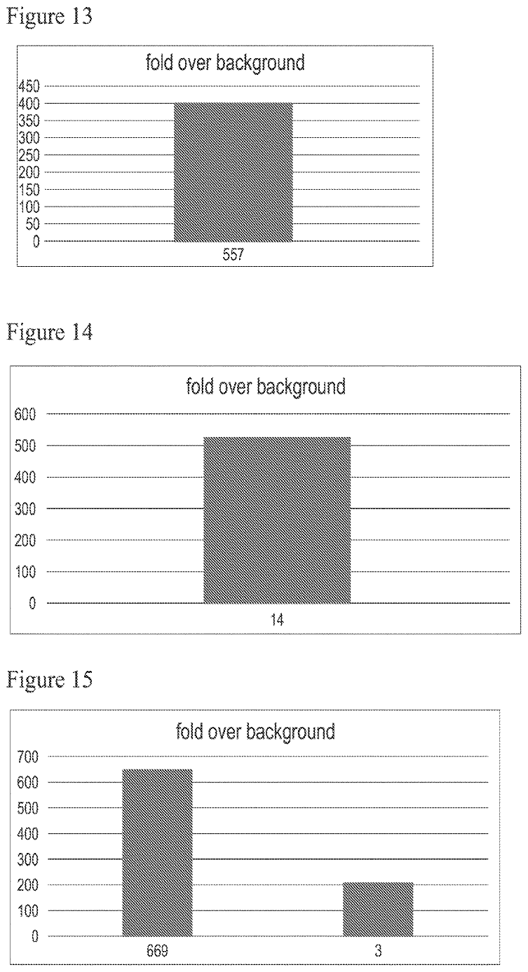

TABLE 10
Lysine (Lys) tRNA (FIG. 13)

| | | fold | | SEQ |
|---|---|---|---|---|
| seq_tRNA | ACE | over | Rank | ID |
| GCCTGGATAGCTCAGTTGGTAGAGCATCAGACTttaAA | TAA | 395.91 | 140 | 557 |
| TCTGAGGGtCCAGGGTTCAAGTCCCTGTTCAGGCG | | | | |

TABLE 11
Phenylalanine (Phe) tRNA (FIG. 14)

| | | fold | | SEQ |
|---|---|---|---|---|
| seq_tRNA | ACE | over | Rank | ID |
| GCCGAAATAGCTCAGTTGGGAGAGCGTTAGACTttaG | TAA | 523.85 | 125 | 14 |
| ATCTAAAGGtCCCTGGTTCGATCCCGGGTTTCGGCA | | | | |

TABLE 12
Phenylalanine (Phe) tRNA (FIG. 15)

| | | fold | | SEQ |
|---|---|---|---|---|
| seq_tRNA | ACE | over | Rank | ID |
| GCCGAAATAGCTCAGTTGGGAGAGCGTTAGACTctaG | TAG | 651.22 | 109 | 669 |
| ATCTAAAGGtCCCTGGTTCGATCCCGGGTTTCGGCA | | | | |
| GCCGAGATAGCTCAGTTGGGAGAGCGTTAGACTctaG | TAG | 208.17 | 174 | 3 |
| ATCTAAAGGtCCCTGGTTCAATCCCGGGTTTCGGCA | | | | |

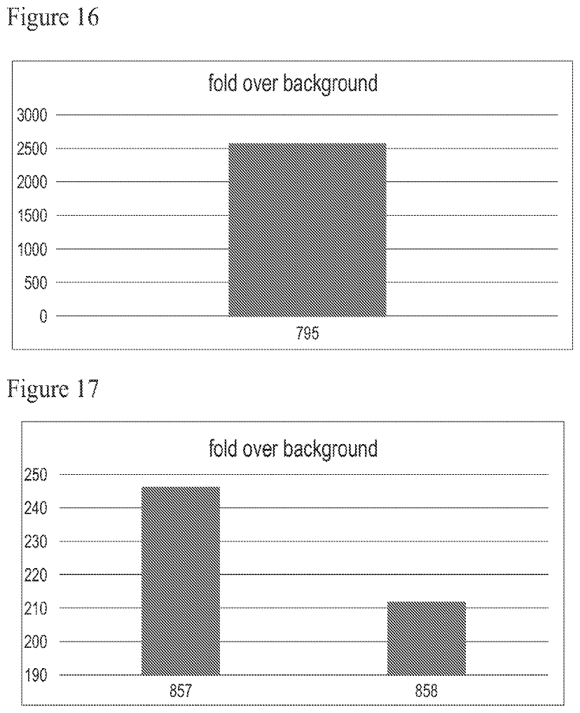

TABLE 13
Tryptophan (Trp) tRNAv (FIG. 16)

| | | fold | | SEQ |
|---|---|---|---|---|
| seq_tRNA | ACE | over | Rank | ID |
| GGCCTCATGGTGCAACAGTAGTGTGTCTGACTctaGA | TAG | 2550.4 | 49 | 795 |
| TCAGAAGGtTGTATGTTCAAATCACGTAGGGGTCA | | | | |

TABLE 14
Tyrosine (Tyr) tRNAs (FIG. 17)

| | | fold | | SEQ |
|---|---|---|---|---|
| seq_tRNA | ACE | over | Rank | ID |
| CCTTCGATAGCTCAGCTGGTAGAGCGGAGGACTtcaG | TGA | 246.5 | 166 | 857 |
| ATCCTTAGGtCGCTGGTTCGAATCCGGCTCGGAGGA | | | | |
| CCTTCGATAGCTCAGCTGGTAGAGCGGAGGACTtcaG | TGA | 211.5 | 172 | 858 |
| ATCCTTAGGtCGCTGGTTCGATTCCAGCTCGAAGGA | | | | |

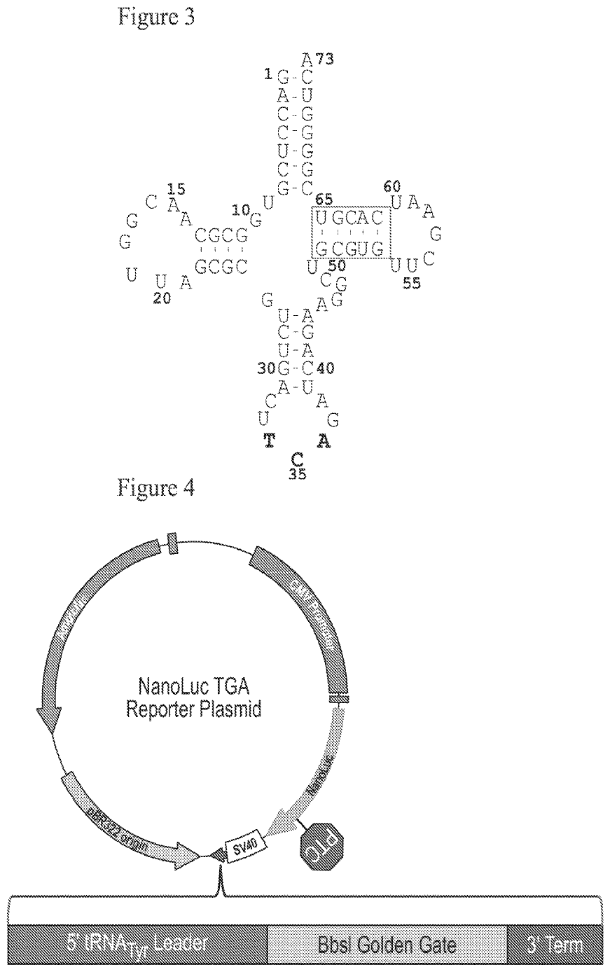

A depiction of an exemplary tRNA for nonsense suppression is as depicted in FIG. 3 (H. sapiens tRNA^{Trp}_{TGA}).

In certain aspects of the invention, human CUA, UCA UAA and UUA suppressor tRNAs have been designed. These tRNAs are approximately 100 nucleotides in length and can be introduced to cells to suppress nonsense codons mutations where the wild-type amino acid should be present. The tRNAs or oligonucleotides that encode them can be introduced directly to recipient cells or can be ligated in tandem to increase efficacy of the tRNAs or oligonucleotides.

In certain aspects the tRNAs described herein repair premature termination codons.

### Expression Cassettes and Vectors

In certain embodiments, the tRNA is encoded by an expression cassette. In yet another embodiment, the suppressor tRNA of the invention may be introduced to the cells using standard conventional genetic engineering techniques through use of vectors. Because of the internal promoter sequences of tRNA encoding sequences, the tRNA sequence need not be included in a separate transcription unit, although one may be provided.

In one embodiment of the present invention, the nucleotide expression system of the invention is included within an appropriate gene transfer vehicle which is then used to transduce cells to express the suppressor tRNA. The gene delivery vehicle can be any delivery vehicle known in the art, and can include naked DNA that is facilitated by a receptor and/or lipid mediated transfection, as well as any of a number of vectors. Such vectors include but are not limited to eukaryotic vectors, prokaryotic vectors (such as for example bacterial vectors) and viral vectors including, but not limited to, retroviral vectors, adenoviral vectors, adeno-associated viral vectors, lentivirus vectors (human and other including porcine), Herpes virus vectors, Epstein-Barr viral vectors, SV40 virus vectors, pox virus vectors, and pseudotyped viral vectors.

In certain embodiments, the tRNA is encoded in a vector. FIG. 4. In certain embodiments, the viral vector is a retroviral or adenoviral vector. Examples of retroviral vectors that may be employed include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus.

### Retroviruses; Retroviral Vectors

The term “retrovirus” is used in reference to RNA viruses that utilize reverse transcriptase during their replication cycle. The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules that encode the structural proteins and enzymes needed to produce new viral particles. At each end of the provirus are structures called “long terminal repeats” or “LTRs.” The LTR contains numerous regulatory signals including transcriptional control elements, polyadenylation signals and sequences needed for replication and integration of the viral genome. There are several genera included within the family Retroviridae, including Cisternavirus A, Oncovirus A, Oncovirus B, Oncovirus C, Oncovirus D, Lentivirus, and Spumavirus. Some of the retroviruses are oncogenic (i.e., tumorigenic), while others are not.

The oncoviruses induce sarcomas, leukemias, lymphomas, and mammary carcinomas in susceptible species. Retroviruses infect a wide variety of species, and may be transmitted both horizontally and vertically. They are integrated into the host DNA, and are capable of transmitting sequences of host DNA from cell to cell. This has led to the development of retroviruses as vectors for various purposes including gene therapy.

Retroviruses, including human foamy virus (HFV) and human immunodeficiency virus (HIV) have gained much recent attention, as their target cells are not limited to dividing cells and their restricted host cell tropism can be readily expanded via pseudotyping with vesicular stomatitis virus G (VSV-G) envelope glycoproteins.

Vector systems generally have a DNA vector containing a small portion of the retroviral sequence (the viral long terminal repeat or “LTR” and the packaging or “psi” signal) and a packaging cell line. The gene to be transferred is inserted into the DNA vector. The viral sequences present on the DNA vector provide the signals necessary for the insertion or packaging of the vector RNA into the viral particle and for the expression of the inserted gene. The packaging cell line provides the viral proteins required for particle. In one embodiment of the present invention, an FIV system employing a three-plasmid transfection production method in 293T cells was used. Replication incompetent virus was successfully produced.

The vector DNA is introduced into the packaging cell by any of a variety of techniques (e.g., calcium phosphate coprecipitation, lipofection, electroporation). The viral proteins produced by the packaging cell mediate the insertion of the vector sequences in the form of RNA into viral particles, which are shed into the culture supernatant.

For cells that are naturally dividing, or are stimulated to divide by growth factors, simple retroviruses like murine leukemia virus (MLV) vectors are suitable delivery systems. A major limitation in the use of many commonly used retroviral vectors in gene transfer, however, is that many of the vectors are restricted to dividing cells. If a non-dividing cell is the target cell, then a lentivirus, which is capable of infecting non-dividing cells, may be used.

As used herein, the term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, that causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells).

Lentiviruses including HIV, SIV, FIV and equine infectious anemia virus (EIAV) depend on several viral regulatory genes in addition to the simple structural gag-pol-env genes for efficient intracellular replication. Thus, lentiviruses use more complex strategies than classical retroviruses for gene regulation and viral replication, with the packaging signals apparently spreading across the entire viral genome. These additional genes display a web of regulatory functions during the lentiviral life cycle. For example, upon HIV-1 infection, transcription is up-regulated by the expression of Tat through interaction with an RNA target (TAR) in the LTR. Expression of the full-length and spliced mRNAs is then regulated by the function of Rev, which interacts with RNA elements present in the gag region and in the env region (RRE). Nuclear export of gag-pol and env mRNAs is dependent on the Rev function. In addition to these two essential regulatory genes, a list of accessory genes, including vif, vpr, vpx, vpu, and nef, are also present in the viral genome and their effects on efficient virus production and infectivity have been demonstrated, although they are not absolutely required for virus replication.

A “source” or “original” retrovirus is a wild-type retrovirus from which a pseudotyped retrovirus is derived, or is used as a starting point, during construction of the packaging or transgene vector, for the preparation of one or more of the genetic elements of the vector. The genetic element may be employed unchanged, or it may be mutated (but not beyond the point where it lacks a statistically significant sequence similarity to the original element). A vector may have more than one source retrovirus, and the different source retroviruses may be, e.g., MLV, FIV, HIV-1 and HIV-2, or HIV and SIV. The term “genetic element” includes but is not limited to a gene.

A cognate retrovirus is the wild-type retrovirus with which the vector in question has the greatest percentage sequence identity at the nucleic acid level. Normally, this will be the same as the source retrovirus. However, if a source retrovirus is extensively mutated, it is conceivable that the vector will then more closely resemble some other retrovirus. It is not necessary that the cognate retrovirus be the physical starting point for the construction; one may choose to synthesize a genetic element, especially a mutant element, directly, rather than to first obtain the original element and then modify it. The term “cognate” may similarly be applied to a protein, gene, or genetic element (e.g., splice donor site or packaging signal). When referring to a cognate protein, percentage sequence identities are determined at the amino acid level.

The term “cognate” retrovirus may be difficult to interpret in the extreme case, i.e., if all retroviral genetic elements have been replaced with surrogate non-lentiviral genetic elements. In this case, the source retrovirus strain mentioned previously is arbitrarily considered to be the cognate retrovirus.

The term “replication” as used herein in reference to a virus or vector, refers not to the normal replication of proviral DNA in a chromosome as a consequence of cell reproduction, or the autonomous replication of a plasmid DNA as a result of the presence of a functional origin of replication. Instead “replication” refers to the completion of a complete viral life cycle, wherein infectious viral particles containing viral RNA enter a cell, the RNA is reverse transcribed into DNA, the DNA integrates into the host chromosome as a provirus, the infected cell produces virion proteins and assembles them with full length viral genomic RNA into new, equally infectious particles.

The term “replication-competent” refers to a wild-type virus or mutant virus that is capable of replication, such that replication of the virus in an infected cell result in the production of infectious virions that, after infecting another, previously uninfected cell, causes the latter cell to likewise produce such infectious virions. The present invention contemplates the use of replication-defective virus.

As used herein, the term “attenuated virus” refers to any virus (e.g., an attenuated lentivirus) that has been modified so that its pathogenicity in the intended subject is substantially reduced. The virus may be attenuated to the point it is nonpathogenic from a clinical standpoint, i.e., that subjects exposed to the virus do not exhibit a statistically significant increased level of pathology relative to control subjects.

The present invention contemplates the preparation and use of a modified retrovirus. In some embodiments, the retrovirus is an mutant of murine leukemia virus, human immunodeficiency virus type 1, human immunodeficiency virus type 2, feline immunodeficiency virus, simian immunodeficiency virus, visna-maedi, caprine arthritis-encephalitis virus, equine infectious anemia virus, and bovine immune deficiency virus, or a virus comprised of portions of more than one retroviral species (e.g., a hybrid, comprised of portions of MLV, FIV, HIV-1 and HIV-2, or HIV-1 and/or SIV).

A reference virus is a virus whose genome is used in describing the components of a mutant virus. For example, a particular genetic element of the mutant virus may be said to differ from the cognate element of the reference virus by various substitutions, deletions or insertions. It is not necessary that the mutant virus actually be derived from the reference virus.

An exemplary reference virus is FIV. In certain embodiments, a three-plasmid transient transfection method can be used to produce replication incompetent pseudotyped retroviruses (e.g., FIV).

### Retroviral Vector System

The present invention contemplates a retroviral gene amplification and transfer system comprising a transgene vector, one or more compatible packaging vectors, an envelope vector, and a suitable host cell. The vectors used may be derived from a retrovirus (e.g., a lentivirus). Retrovirus vectors allow (1) transfection of the packaging vectors and envelope vectors into the host cell to form a packaging cell line that produces essentially packaging-vector-RNA-free viral particles, (2) transfection of the transgene vector into the packaging cell line, (3) the packaging of the transgene vector RNA by the packaging cell line into infectious viral particles, and (4) the administration of the particles to target cells so that such cells are transduced and subsequently express a transgene.

Either the particles are administered directly to the subject, in vivo, or the subject's cells are removed, infected in vitro with the particles, and returned to the body of the subject.

The packaging vectors and transgene vectors of the present invention will generate replication-incompetent viruses. The vectors chosen for incorporation into a given vector system of the present invention are such that it is not possible, without further mutation of the packaging vector(s) or transgene vector, for the cotransfected cells to generate a replication-competent virus by homologous recombination of the packaging vector(s) and transgene vector alone. The envelope protein used in the present system can be a retroviral envelope, a synthetic or chimeric envelope, or the envelope from a non-retroviral enveloped virus (e.g., baculovirus).

### Packaging Signal

As used herein, the term “packaging signal” or “packaging sequence” refers to sequences located within the retroviral genome or a vector that are required for, or at least facilitate, insertion of the viral or vector RNA into the viral capsid or particle. The packaging signals in an RNA identify that RNA as one that is to be packaged into a virion. The term “packaging signal” is also used for convenience to refer to a vector DNA sequence that is transcribed into a functional packaging signal. Certain packaging signals may be part of a gene, but are recognized in the form of RNA, rather than as a peptide moiety of the encoded protein.

The key distinction between a packaging vector and a transgene vector is that in the packaging vector, the major packaging signal is inactivated, and, in the transgene vector, the major packaging sign al is functional. Ideally, in the packaging vector, all packaging signals would be inactivated, and, in the transgene vector, all packaging signals would be functional. However, countervailing considerations, such as maximizing viral titer, or inhibiting homologous recombination, may lend such constructs less desirable.

### Packaging System; Packaging Vectors; Packaging Cell Line

A packaging system is a vector, or a plurality of vectors, which collectively provide in expressible form all of the genetic information required to produce a virion that can encapsidate suitable RNA, transport it from the virion-producing cell, transmit it to a target cell, and, in the target cell, cause the RNA to be reverse transcribed and integrated into the host genome in a such a manner that a transgene incorporated into the aforementioned RNA can be expressed. However, the packaging system must be substantially incapable of packaging itself. Rather, it packages a separate transgene vector.

In the present invention, the packaging vector will provide functional equivalents of the gag and pol genes (a “GP” vector). The env gene(s) will be provided by the envelope vector. In theory, a three vector system (“G”, “P”, and “E” vectors) is possible if one is willing to construct distinct gag and pol genes on separate vectors, and operably link them to different regulatable promoters (or one to a regulatable and the other to a constitutive promoter) such that their relative levels of expression can be adjusted appropriately.

A packaging cell line is a suitable host cell transfected by a packaging system that, under achievable conditions, produces viral particles. As used herein, the term “packaging cell lines” is typically used in reference to cell lines that express viral structural proteins (e.g., gag, pol and env), but do not contain a packaging signal. For example, a cell line has been genetically engineered to carry at one chromosomal site within its genome, a 5′-LTR-gag-pol-3′-LTR fragment that lacks a functional psi⁺ sequence (designated as Δ-psi), and a 5′-LTR-env-3′-LTR fragment that is also Δ-psi located at another chromosomal site. While both of these segments are transcribed constitutively, because the psi⁺ region is missing and the viral RNA molecules produced are less than full-size, empty viral particles are formed.

If a host cell is transfected by the packaging vector(s) alone, it produces substantially only viral particles without the full-length packaging vector. In one example, less than 10% of the viral particles produced by the packaging cell contain full length packaging vector-derived RNA. However, since the packaging vector lacks a functional primer-binding site, even if these particles infect a new cell, the packaging vector RNA will not be reverse transcribed back into DNA and therefore the new cell will not produce virion. Thus, by itself, the packaging vector is a replication-incompetent virus.

In some embodiments, the packaging cell and/or cell line contains a transgene vector. The packaging cell line will package the transgene vector into infectious particles. Such a cell line is referred to herein as a “transgenic virion production cell line.”

It is contemplated that packaging may be inducible, as well as non-inducible. In inducible packaging cells and packaging cell lines, retroviral particles are produced in response to at least one inducer. In non-inducible packaging cell lines and packaging cells, no inducer is required in order for retroviral particle production to occur.

The packaging vectors necessarily differ from wild-type, replication-competent retroviral genomes by virtue of the inactivation of at least one packaging signal of the cognate wild-type genome. More than one packaging signal may be inactivated. In one example, only the retroviral genes provided by the packaging vector are those encoding structural, or essential regulatory, proteins.

### Transgene Vectors

A transgene vector is an expression vector that bears an expressible non-retroviral gene of interest and includes at least one functional retroviral packaging signal, so that, after the transgene vector is transfected into a packaging cell line, the transgene vector is transcribed into RNA, and this RNA is packaged into an infectious viral particle. These particles, in turn, infect target cells, their RNA is reverse transcribed into DNA, and the DNA is incorporated into the host cell genome as a proviral element, thereby transmitting the gene of interest to the target cells.

As used herein, the term “transduction” refers to the delivery of a gene(s) using a viral or retroviral vector by means of infection rather than by transfection. In certain embodiments, retroviral vectors are transduced. Thus, a “transduced gene” is a gene that has been introduced into the cell via retroviral or vector infection and provirus integration. In certain embodiments, viral vectors (e.g., “transgene vectors”) transduce genes into “target cells” or host cells. The, present invention encompasses transgene vectors that are suitable for use in the present invention that are linked to any gene of interest (or a “marker gene” or “reporter gene,” used to indicate infection or expression of a gene).

As used herein, the term “long-term transduction” refers to vectors that are capable of remaining transduced in host or target cells for time periods that are longer than those observed with other vectors. For example, the present invention provides retroviral vectors that are capable of remaining transduced for at least 120 days, at least one year, or for the life of the subject or the necessary time course of treatment. The duration of expression is a function of the choice of promoter and the target cell type, more so than the choice of vector.

The term “stable transduction” or “stably transduced” refers to the introduction and integration of foreign DNA into the genome of the transducted cell. The term “stable transductant” refers to a cell that has stably integrated foreign DNA into the genomic DNA.

The term “transient transduction” or “transiently transduced” refers to the introduction of foreign DNA into a cell where the foreign DNA fails to integrate into the genome of the transducted cell. The foreign DNA persists in the nucleus of the transducted cell for several days. During this time the foreign DNA is subject to the regulatory controls that govern the expression of endogenous genes in the chromosomes. The term “transient transductant” refers to cells that have taken up foreign DNA but have failed to integrate this DNA.

In some embodiments, the target and/or host cells of the present invention are “non-dividing” cells. These cells include cells such as neuronal cells that do not normally divide. However, it is not intended that the present invention be limited to non-dividing cells (including, but not limited to muscle cells, white blood cells, spleen cells, liver cells, eye cells, epithelial cells).

In some embodiments, the vector and the vector progeny are capable of transducing a plurality of target cells so as to achieve vector titers of at least 10⁵ cfu/ml. The multiplicity of infection (MOI) may be at least one (i.e., one hit on average per cell), or even at least two.

### Expression Cassettes and Vectors

The present invention also provides an expression cassette comprising a sequence encoding tRNA.

In certain embodiments, the expression cassette further contains a promoter. In certain embodiments, the promoter is a regulatable promoter. In certain embodiments, the promoter is a constitutive promoter. In certain embodiments, the promoter is a PGK, CMV, RSV, H1 or U6 promoter (Pol II and Pol III promoters).

The present invention provides a vector containing the expression cassette described above. In certain embodiments, the vector is a viral vector. In certain embodiments, the viral vector is an adenoviral, lentiviral, adeno-associated viral (AAV), poliovirus, HSV, or murine Maloney-based viral vector.

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for a protein of interest. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

### Adeno Associated Virus (AAV)

Adeno associated virus (AAV) is a small nonpathogenic virus of the parvoviridae family. AAV is distinct from the other members of this family by its dependence upon a helper virus for replication. In the absence of a helper virus, AAV may integrate in a locus specific manner into the q arm of chromosome 19. The approximately 5 kb genome of AAV consists of one segment of single stranded DNA of either plus or minus polarity. The ends of the genome are short inverted terminal repeats that can fold into hairpin structures and serve as the origin of viral DNA replication. Physically, the parvovirus virion is non-enveloped and its icosohedral capsid is approximately 20 nm in diameter.

To date, numerous serologically distinct AAVs have been identified, and more than a dozen have been isolated from humans or primates. The genome of AAV2 is 4680 nucleotides in length and contains two open reading frames (ORFs). The left ORF encodes the non-structural Rep proteins, Rep 40, Rep 52, Rep 68 and Rep 78, which are involved in regulation of replication and transcription in addition to the production of single-stranded progeny genomes. Furthermore, two of the Rep proteins have been associated with the preferential integration of AAV genomes into a region of the q arm of human chromosome 19. Rep68/78 has also been shown to possess NTP binding activity as well as DNA and RNA helicase activities. The Rep proteins possess a nuclear localization signal as well as several potential phosphorylation sites. Mutation of one of these kinase sites resulted in a loss of replication activity.

The ends of the genome are short inverted terminal repeats (ITR) which have the potential to fold into T-shaped hairpin structures that serve as the origin of viral DNA replication. Within the ITR region two elements have been described which are central to the function of the ITR, a GAGC repeat motif and the terminal resolution site (trs). The repeat motif has been shown to bind Rep when the ITR is in either a linear or hairpin conformation. This binding serves to position Rep68/78 for cleavage at the trs, which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

The AAV virion is a non-enveloped, icosohedral particle approximately 25 nm in diameter, consisting of three related proteins referred to as VP1, VP2 and VP3. The right ORF encodes the capsid proteins VP1, VP2, and VP3. These proteins are found in a ratio of 1:1:10 respectively and are all derived from the right-hand ORE. The capsid proteins differ from each other by the use of alternative splicing and an unusual start codon. Deletion analysis has shown that removal or alteration of VP1 which is translated from an alternatively spliced message results in a reduced yield of infections particles. Mutations within the VP3 coding region result in the failure to produce any single-stranded progeny DNA or infectious particles. An AAV particle is a viral particle comprising an AAV capsid protein. An AAV capsid polypeptide can encode the entire VP1, VP2 and VP3 polypeptide. The particle can be a particle comprising AAV2 and other AAV capsid proteins (i.e., a chimeric protein, such as AAV1 and AAV2). Variations in the amino acid sequence of the AAV2 capsid protein are contemplated herein, as long as the resulting viral particle comprises the AAV2 capsid remains antigenically or immunologically distinct from AAV1, as can be routinely determined by standard methods. Specifically, for example, ELISA and Western blots can be used to determine whether a viral particle is antigenically or immunologically distinct from AAV1. Furthermore, the AAV2 viral particle preferably retains tissue tropism distinct from AAV1.

An AAV2 particle is a viral particle comprising an AAV2 capsid protein. An AAV2 capsid polypeptide encoding the entire VP1, VP2, and VP3 polypeptide can overall have at least about 63% homology (or identity) to the polypeptide having the amino acid sequence encoded by nucleotides set forth in NC_001401 (nucleotide sequence encoding AAV2 capsid protein). The capsid protein can have about 70% homology, about 75% homology, 80% homology, 85% homology, 90% homology, 95% homology, 98% homology, 99% homology, or even 100% homology to the protein encoded by the nucleotide sequence set forth in NC_001401. The capsid protein can have about 70% identity, about 75% identity, 80% identity, 85% identity, 90% identity, 95% identity, 98% identity, 99% identity, or even 100% identity to the protein encoded by the nucleotide sequence set forth in NC_001401. The particle can be a particle comprising another AAV and AAV2 capsid protein, i.e., a chimeric protein. Variations in the amino acid sequence of the AAV2 capsid protein are contemplated herein, as long as the resulting viral particle comprising the AAV2 capsid remains antigenically or immunologically distinct from AAV4, as can be routinely determined by standard methods. Specifically, for example, ELISA and Western blots can be used to determine whether a viral particle is antigenically or immunologically distinct from AAV1. Furthermore, the AAV2 viral particle preferably retains tissue tropism distinction from AAV1, such as that exemplified in the examples herein, though an AAV2 chimeric particle comprising at least one AAV2 coat protein may have a different tissue tropism from that of an AAV2 particle consisting only of AAV2 coat proteins.

In certain embodiments, the invention further provides an AAV2 particle containing, i.e., encapsidating, a vector comprising a pair of AAV2 inverted terminal repeats. The nucleotide sequence of AAV2 ITRs is known in the art. Furthermore, the particle can be a particle comprising both AAV1 and AAV2 capsid protein, i.e., a chimeric protein. Moreover, the particle can be a particle encapsidating a vector comprising a pair of AAV inverted terminal repeats from other AAVs (e.g., AAV1-AAV9 and AAVrh10). The vector encapsidated in the particle can further comprise an exogenous nucleic acid inserted between the inverted terminal repeats.

The following features of AAV have made it an attractive vector for gene transfer. AAV vectors have been shown in vitro to stably integrate into the cellular genome; possess a broad host range; transduce both dividing and non-dividing cells in vitro and in vivo and maintain high levels of expression of the transduced genes. Viral particles are heat stable, resistant to solvents, detergents, changes in pH, temperature, and can be concentrated on CsCl gradients or by other means. The present invention provides methods of administering AAV particles, recombinant AAV vectors, and recombinant AAV virions. For example, an AAV2 particle is a viral particle comprising an AAV2 capsid protein, or an AAV1 particle is a viral particle comprising an AAV1 capsid protein. A recombinant AAV2 vector is a nucleic acid construct that comprises at least one unique nucleic acid of AAV2. A recombinant AAV2 virion is a particle containing a recombinant AAV2 vector. To be considered within the term “AAV2 ITRs” the nucleotide sequence must retain one or both features described herein that distinguish the AAV2 ITR from the AAV1 ITR: (1) three (rather than four as in AAV1) “GAGC” repeats and (2) in the AAV2 ITR Rep binding site the fourth nucleotide in the first two “GAGC” repeats is a C rather than a T.

The promoter to drive expression of the sequence encoding the tRNA to be delivered can be any desired promoter, selected by known considerations, such as the level of expression of a nucleic acid functionally linked to the promoter and the cell type in which the vector is to be used. Promoters can be an exogenous or an endogenous promoter. Promoters can include, for example, known strong promoters such as SV40 or the inducible metallothionein promoter, or an AAV promoter, such as an AAV p5 promoter. Additional examples of promoters include promoters derived from actin genes, immunoglobulin genes, cytomegalovirus (CMV), adenovirus, bovine papilloma virus, adenoviral promoters, such as the adenoviral major late promoter, an inducible heat shock promoter, respiratory syncytial virus, Rous sarcomas virus (RSV), etc. Additional examples include regulated promoters.

The AAV vector can further comprise an exogenous (heterologous) nucleic acid functionally linked to the promoter. By “heterologous nucleic acid” is meant that any heterologous or exogenous nucleic acid can be inserted into the vector for transfer into a cell, tissue or organism. The nucleic acid can encode a tRNA, for example. By “functionally linked” is meant such that the promoter can promote expression of the heterologous nucleic acid, as is known in the art, such as appropriate orientation of the promoter relative to the heterologous nucleic acid. Furthermore, the heterologous nucleic acid preferably has all appropriate sequences for expression of the nucleic acid, as known in the art, to functionally encode, i.e., allow the nucleic acid to be expressed. The nucleic acid can include, for example, expression control sequences, such as an enhancer. The nucleic acid can encode more than one gene product, limited only by the size of nucleic acid that can be packaged.

An AAV1 particle is a viral particle comprising an AAV1 capsid protein. Variations in the amino acid sequence of the AAV1 capsid protein are contemplated herein, as long as the resulting viral particle comprising the AAV1 capsid remains antigenically or immunologically distinct from other AAV capsids, as can be routinely determined by standard methods. Specifically, for example, ELISA and Western blots can be used to determine whether a viral particle is antigenically or immunologically distinct from other AAV serotypes.

The term “polypeptide” as used herein refers to a polymer of amino acids and includes full-length proteins and fragments thereof. Thus, “protein” and “polypeptide” are often used interchangeably herein.

The present method provides a method of delivering a nucleic acid to a cell comprising administering to the cell an AAV particle containing a vector comprising the nucleic acid inserted between a pair of AAV inverted terminal repeats, thereby delivering the nucleic acid to the cell. Administration to the cell can be accomplished by any means, including simply contacting the particle, optionally contained in a desired liquid such as tissue culture medium, or a buffered saline solution, with the cells. The particle can be allowed to remain in contact with the cells for any desired length of time, and typically, the particle is administered and allowed to remain indefinitely. For such in vitro methods, the virus can be administered to the cell by standard viral transduction methods, as known in the art and as exemplified herein. Titers of virus to administer can vary, particularly depending upon the cell type, but will be typical of that used for AAV transduction in general. Additionally the titers used to transduce the particular cells in the present examples can be utilized. The cells can include any desired cell in humans as well as other large (non-rodent) mammals, such as primates, horse, sheep, goat, pig, and dog.

The present invention further provides a method of delivering a nucleic acid to a cell in a subject comprising administering to the subject an AAV particle comprising the nucleic acid inserted between a pair of AAV inverted terminal repeats, thereby delivering the nucleic acid to a cell in the subject.

Certain embodiments of the present disclosure provide a cell comprising a viral vector as described herein.

### AAV Vectors

In one embodiment, a viral vector of the disclosure is an AAV vector. An “AAV” vector refers to an adeno-associated virus, and may be used to refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV, which is identified by, and distinguished from other AAVs based on capsid protein reactivity with defined antisera, e.g., there are eight known serotypes of primate AAVs, AAV-1 to AAV-9 and AAVrh10. For example, serotype AAV2 is used to refer to an AAV, which contains capsid proteins encoded from the cap gene of AAV2 and a genome containing 5′ and 3′ ITR sequences from the same AAV2 serotype. As used herein, for example, rAAV1 may be used to refer an AAV having both capsid proteins and 5′-3′ ITRs from the same serotype or it may refer to an AAV having capsid proteins from one serotype and 5′-3′ ITRs from a different AAV serotype, e.g., capsid from AAV serotype 2 and ITRs from AAV serotype 5. For each example illustrated herein, the description of the vector design and production describes the serotype of the capsid and 5′-3′ ITR sequences. The abbreviation “rAAV” refers to recombinant adeno-associated virus, also referred to as a recombinant AAV vector (or “rAAV vector”).

An “AAV virus” or “AAV viral particle” refers to a viral particle composed of at least one AAV capsid protein (preferably by all of the capsid proteins of a wild-type AAV) and an encapsidated polynucleotide. If the particle comprises heterologous polynucleotide (i.e., a polynucleotide other than a wild-type AAV genome such as a transgene to be delivered to a mammalian cell), it is typically referred to as “rAAV”.

In one embodiment, the AAV expression vectors are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is flanked (5′ and 3′) with functional AAV ITR sequences.

By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. As used herein, an “AAV ITR” need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV7, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

In one embodiment, AAV ITRs can be derived from any of several AAV serotypes, including without limitation, AAV1, AAV2, AAV3, AAV4, AAV5, AAV7, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

In one embodiment, AAV capsids can be derived from AAV2. Suitable DNA molecules for use in AAV vectors will be less than about 5 kilobases (kb), less than about 4.5 kb, less than about 4 kb, less than about 3.5 kb, less than about 3 kb, less than about 2.5 kb in size and are known in the art.

In one embodiment, the selected nucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Such promoter sequences are commercially available from, e.g., Stratagene (San Diego, Calif).

In one embodiment, both heterologous promoters and other control elements, such as tissue-specific and inducible promoters, enhancers and the like, will be of particular use. Examples of heterologous promoters include the CMV promoter. Examples of inducible promoters include DNA responsive elements for ecdysone, tetracycline, hypoxia and aufin.

In one embodiment, the AAV expression vector that harbors the DNA molecule of interest bounded by AAV ITRs, can be constructed by directly inserting the selected sequence(s) into an AAV genome, which has had the major AAV open reading frames (“ORFs”), excised therefrom. Other portions of the AAV genome can also be deleted, so long as sufficient portions of the ITRs remain to allow for replication and packaging functions. Such constructs can be designed using techniques well known in the art.

Alternatively, AAV ITRs can be excised from the viral genome or from an AAV vector containing the same and fused 5′ and 3′ of a selected nucleic acid construct that is present in another vector using standard ligation techniques. For example, ligations can be accomplished in 20 mM Tris-Cl pH 7.5, 10 mM MgCl2, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration). AAV vectors which contain ITRs.

Additionally, chimeric genes can be produced synthetically to include AAV ITR sequences arranged 5′ and 3′ of one or more selected nucleic acid sequences. The complete chimeric sequence is assembled from overlapping oligonucleotides prepared by standard methods.

In order to produce rAAV virions, an AAV expression vector is introduced into a suitable host cell using known techniques, such as by transfection. A number of transfection techniques are generally known in the art. See, e.g., Sambrook et al. (1989) Molecular Cloning, a laboratory manual, Cold Spring Harbor Laboratories, New York. Particularly suitable transfection methods include calcium phosphate co-precipitation, direct micro-injection into cultured cells, electroporation, liposome mediated gene transfer, lipid-mediated transduction, and nucleic acid delivery using high-velocity microprojectiles.

In one embodiment, suitable host cells for producing rAAV virions include microorganisms, yeast cells, insect cells, and mammalian cells, that can be, or have been, used as recipients of a heterologous DNA molecule. The term includes the progeny of the original cell that has been transfected. Thus, a “host cell” as used herein generally refers to a cell that has been transfected with an exogenous DNA sequence. Cells from the stable human cell line, 293 (readily available through, e.g., the American Type Culture Collection under Accession Number ATCC CRL1573) can be used in the practice of the present disclosure. Particularly, the human cell line 293 is a human embryonic kidney cell line that has been transformed with adenovirus type-5 DNA fragments, and expresses the adenoviral E1a and E1b genes. The 293 cell line is readily transfected, and provides a particularly convenient platform in which to produce rAAV virions.

By “AAV rep coding region” is meant the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. Suitable homologues of the AAV rep coding region include the human herpesvirus 6 (HHV-6) rep gene which is also known to mediate AAV-2 DNA replication.

By “AAV cap coding region” is meant the art-recognized region of the AAV genome that encodes the capsid proteins VP1, VP2, and VP3, or functional homologues thereof. These Cap expression products supply the packaging functions, which are collectively required for packaging the viral genome.

In one embodiment, AAV helper functions are introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 that encode both Rep and Cap expression products. A number of other vectors have been described that encode Rep and/or Cap expression products.

Methods of delivery of viral vectors include injecting the AAV into the subject. Generally, rAAV virions may be introduced into cells using either in vivo or in vitro transduction techniques. If transduced in vitro, the desired recipient cell will be removed from the subject, transduced with rAAV virions and reintroduced into the subject. Alternatively, syngeneic or xenogeneic cells can be used where those cells will not generate an inappropriate immune response in the subject.

Suitable methods for the delivery and introduction of transduced cells into a subject have been described. For example, cells can be transduced in vitro by combining recombinant AAV virions with cells e.g., in appropriate media, and screening for those cells harboring the DNA of interest can be screened using conventional techniques such as Southern blots and/or PCR, or by using selectable markers. Transduced cells can then be formulated into pharmaceutical compositions, described more fully below, and the composition introduced into the subject by various techniques, such as by grafting, intramuscular, intravenous, subcutaneous and intraperitoneal injection.

In one embodiment, pharmaceutical compositions will comprise sufficient genetic material to produce a therapeutically effective amount of the nucleic acid of interest, i.e., an amount sufficient to reduce or ameliorate symptoms of the disease state in question or an amount sufficient to confer the desired benefit. The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., N.J. 1991).

It should be understood that more than one transgene could be expressed by the delivered viral vector. Alternatively, separate vectors, each expressing one or more different transgenes, can also be delivered to the subject as described herein. Furthermore, it is also intended that the viral vectors delivered by the methods of the present disclosure be combined with other suitable compositions and therapies.

As is apparent to those skilled in the art in view of the teachings of this specification, an effective amount of viral vector that must be added can be empirically determined. Administration can be in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

In certain embodiments, the rAAV is administered at a dose of about 0.3-2 ml of 1×10⁵-1×10¹⁶ vg/ml. In certain embodiments, the rAAV is administered at a dose of about 1-3 ml of 1×10⁷-1×10¹⁴ vg/ml. In certain embodiments, the rAAV is administered at a dose of about 1-2 ml of 1×10⁸-1×10³ vg/ml.

Formulations containing the rAAV particles will contain an effective amount of the rAAV particles in a vehicle, the effective amount being readily determined by one skilled in the art. The rAAV particles may typically range from about 1% to about 95% (w/w) of the composition, or even higher or lower if appropriate. The quantity to be administered depends upon factors such as the age, weight and physical condition of the animal or the human subject considered for treatment. Effective dosages can be established by one of ordinary skill in the art through routine trials establishing dose response curves. The subject is treated by administration of the rAAV particles in one or more doses. Multiple doses may be administered as is required to maintain adequate enzyme activity.

Vehicles including water, aqueous saline, artificial CSF, or other known substances can be employed with the subject invention. To prepare a formulation, the purified composition can be isolated, lyophilized and stabilized. The composition may then be adjusted to an appropriate concentration, optionally combined with an anti-inflammatory agent, and packaged for use.

The present invention provides a method of increasing the level of a target protein in a cell by introducing a nucleic acid molecule or tRNA described above into a cell in an amount sufficient to increase the level of the target protein in the cell. In certain embodiments, the accumulation of target protein is increased by at least 10%. The accumulation of target protein is increased by at least 10%, 20%, 30%, 40%, 50%, 60%, 70%, 80%, 90% 95%, or 99%.

### Nucleic Acids Encoding Therapeutic Agents

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides.

A “nucleic acid fragment” is a portion of a given nucleic acid molecule. The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. tRNAs with any one of the foregoing percent identities of the tRNA sequences provided herein are also provided. Nucleic acid molecules that encode such tRNA sequences are also provided.

Methods for Introducing Genetic Material into Cells

The exogenous genetic material (e.g., a DNA encoding one or more therapeutic tRNAs) is introduced into the cell in vivo by genetic transfer methods, such as transfection or transduction, to provide a genetically modified cell. Various expression vectors (i.e., vehicles for facilitating delivery of exogenous genetic material into a target cell) are known to one of ordinary skill in the art.

As used herein, “transfection of cells” refers to the acquisition by a cell of new genetic material by incorporation of added DNA. Thus, transfection refers to the insertion of nucleic acid into a cell using physical or chemical methods. Several transfection techniques are known to those of ordinary skill in the art including: calcium phosphate DNA co-precipitation; DEAE-dextran; electroporation; cationic liposome-mediated transfection; and tungsten particle-facilitated microparticle bombardment. Strontium phosphate DNA co-precipitation is another possible transfection method.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced cell. A cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

Typically, the exogenous genetic material includes a nucleic acid that encodes a tRNA (usually in the form of a cDNA) optionally together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion, an “enhancer” is simply any non-translated DNA sequence that works contiguous with the coding sequence (in cis) to change the basal transcription level dictated by the promoter. The exogenous genetic material may be introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes that encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eucaryotic cells. These include the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by intraperitoneal injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous genetic material that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may include a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence (described below) is deemed to be within the scope of one of ordinary skill in the art without undue experimentation.

### Disease Conditions and Methods of Treatment

The present invention in one embodiment includes compositions and methods for treating a genetic disease, such as cystic fibrosis, by reversing the effects of mutations present that are associated with nonsense mutations through introduction of the tRNAs or nucleic acids encoding the tRNAs of the invention.

Certain embodiments of the present disclosure provide a method of treating a disease in a mammal comprising administering a tRNA as provided herein or a nucleic acid (e.g., a vector) encoding the tRNA as described herein to the mammal. In certain embodiments, the mammal is human.

Certain embodiments of the present disclosure provide a use of a tRNA or nucleic acid (e.g., a vector) encoding the tRNA as described herein to prepare a medicament useful for treating disease in a mammal. In certain embodiments, the disease is cystic fibrosis.

The present disclosure also provides a mammalian cell containing a tRNA or nucleic acid encoding the tRNA (e.g., a vector) described herein. The cell may be human.

Certain aspects of the disclosure relate to polynucleotides, polypeptides, vectors, and genetically engineered cells (modified in vivo), and the use of them. In particular, the disclosure relates to a method for gene therapy that is capable of both systemic delivery of a therapeutically effective dose of the therapeutic agent.

According to one aspect, a cell expression system for expressing a therapeutic agent in a mammalian recipient is provided. The expression system (also referred to herein as a “genetically modified cell”) comprises a cell and an expression vector for expressing the therapeutic agent. Expression vectors include, but are not limited to, viruses, plasmids, and other vehicles for delivering heterologous genetic material to cells. Accordingly, the term “expression vector” as used herein refers to a vehicle for delivering heterologous genetic material to a cell. In particular, the expression vector is a recombinant adenoviral, adeno-associated virus, or lentivirus or retrovirus vector.

The expression vector can further include a promoter for controlling transcription of the heterologous gene. The promoter may be an inducible promoter (described herein). The expression system is suitable for administration to the mammalian recipient. The expression system may comprise a plurality of non-immortalized genetically modified cells, each cell containing at least one recombinant gene encoding at least one therapeutic agent.

The cell expression system is formed in vivo. According to yet another aspect, a method for treating a mammalian recipient in vivo is provided. The method includes introducing an expression vector for expressing a tRNA into a cell of the patient in situ, such as via intravenous administration. To form the expression system in vivo, an expression vector for expressing the therapeutic agent is introduced in vivo into the mammalian recipient i.v.

According to yet another aspect, a method for treating a mammalian recipient in vivo is provided. The method includes introducing the target therapeutic agent into the patient in vivo.

The expression vector for expressing the tRNA may include an inducible promoter for controlling transcription of the heterologous gene product. Accordingly, delivery of the therapeutic agent in situ is controlled by exposing the cell in situ to conditions, which induce transcription of the tRNA.

The present disclosure provides methods of treating a disease in a mammal by administering an expression vector to a cell or patient. For the gene therapy methods, a person having ordinary skill in the art of molecular biology and gene therapy would be able to determine, without undue experimentation, the appropriate dosages and routes of administration of the expression vector used in the novel methods of the present disclosure.

According to one embodiment, the cells are transformed or otherwise genetically modified in vivo. The cells from the mammalian recipient are transformed (i.e., transduced or transfected) in vivo with a vector containing exogenous genetic material for expressing a heterologous (e.g., recombinant) gene encoding a therapeutic agent and the therapeutic agent is delivered in situ.

As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into a tRNA.

The above-disclosed therapeutic agents and conditions amenable to gene therapy are merely illustrative and are not intended to limit the scope of the instant disclosure. The selection of a suitable therapeutic agent for treating a known condition is deemed to be within the scope of one of ordinary skill of the art without undue experimentation.

In certain embodiments, the therapy has potential use for the treatment/management of diseases that are caused by Premature Termination Codons (PTCs), including, but not limited to, cystic fibrosis, muscular dystrophy, β-thalassemia and Liddle's syndrome. This therapy is advantageous in that it provides improved stop codon suppression specificity. The therapeutic tRNAs of the present invention target a specific stop-codon, TGA for instance, thus reducing off-target effects at stop-codons unrelated to disease. The present therapy is also advantageous in that it provides amino-acid specificity. The expressed tRNA can be engineered to specifically replace the amino acid that was lost via insertion of the disease stop codon, thus negating any spurious effects on protein stability, folding and trafficking.

In certain embodiments, the present system is modular, and thus can be “personalized”. For instance, there are nine individual tryptophan tRNAs in the human genome that are recognized by the Trp synthetase, all of which suppress the mRNA UGG codon. Thus, each of these nine Trp tRNA provides an opportunity for codon re-editing tolerance (UGG→UGA). Additionally, given their proximity to stop codons in the genetic code, the mutation of arginine codons to PTC nonsense codons are common in disease. There are over thirty Arg tRNA that can be used for codon editing tolerance and suppression efficacy.

A further advantage of the present invention is that it provides facile expression and cell specific delivery, because an entire system (tRNA+promoter sequence, optionally) can be compact.

### Dosages, Formulations and Routes of Administration of the Agents of the Invention

The agents of the invention are administered so as to result in a reduction in at least one symptom associated with a disease, such as genetic disease (e.g., cystic fibrosis). The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems that are well known to the art.

The present invention envisions treating disease, such as a genetic disease (e.g., cystic fibrosis) by the administration of an agent, e.g., tRNA, an expression vector, or a viral particle of the invention. Administration of the therapeutic agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated.

One or more suitable unit dosage forms having the therapeutic agent(s) of the invention, which, as discussed below, may optionally be formulated for sustained release (for example using microencapsulation), can be administered by a variety of routes including parenteral, including by intravenous and intramuscular routes, as well as by direct injection into the diseased tissue. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well-known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0 and water.

### Definitions

Disease state: For the purposes of the present invention, a “disease state” or “disease phenotype” is a characteristic of a mammalian cell that results from a stop codon within the coding region of a gene inside the cell (e.g., that results from a nonsense mutation). For example, an increasing number of human genetic diseases are thought to be caused by nonsense mutations. To give but a few examples, β-thalessemia, Duchenne muscular dystrophy, xeroderma pigmentosum, Fanconi's anemia, and cystic fibrosis can all be caused by nonsense mutations in identified genes.

Endogenous tRNA synthetase: A tRNA synthetase is considered to be “endogenous” to a cell if it is present in the cell into which a tRNA is introduced according to the present invention. As will be the apparent to those of ordinary skill in the art, a tRNA synthetase may be considered to be endogenous for these purposes whether it is naturally found in cells of the relevant type, or whether the particular cell at issue has been engineered or otherwise manipulated by the hand of man to contain or express it.

Nonsense suppressor tRNA: A “nonsense suppressor tRNA” is one whose anti-codon is complementary with a codon that would otherwise terminate translation, so that detectable read-through occurs under the conditions of the experiment. Standard termination codons are amber (UAG), ochre (UAA), and opal (UGA) codons. However, non-standard termination codons (e.g., 4-nucleotide codons) have also been employed in the literature.

The invention is now illustrated by the following non-limiting Examples.

### Example 1

### tRNA Screening

A library of human tRNA sequences was produced where the tRNA sequences were engineered at the anti-codon loop region. All of the synthetic tRNA sequences are non-native and do not occur in nature. tRNAs specific for all 20 amino acids were used as starting materials, and were engineered to include one of the three stop codon types in the anti-codon loop region.

Over 900 human tRNA sequences from the library were screened for tolerance of the anti-codon engineering. The resulting engineered suppressor tRNA were individually examined for their ability to rescue an in-frame stop codon in a nano-luciferase reporter expressed in HEK293t cells.

The results are provided in Tables 15-34. In each case, “rank” refers to activity rank of the sequence in the full screen of all tRNAs. “Fold over” is the fold-over-background signal for rescued luciferase activity. The tRNA sequence and anti-codon edited (ACE) is provided.

TABLE 15A
Alanine (Ala) tRNA

| | | Fold | | SEQ |
|---|---|---|---|---|
| seq_tRNA | ACE | over | Rank | ID |
| GGGGATGTAGCTCAGTGGTAGAGCGCATGCTTttaATG | TAA | 132 | 199 | 36 |
| CATGAGGTCCCGGGTTCGATCCCCAGCATCTCCA | | | | |
| GGGGATGTAGCTCAGTGGTAGAGCGCGCGCTTttaATG | TAA | 118.3 | 206 | 37 |
| TGTGAGGtCCCGGGTTCAATCCCCGGCATCTCCA | | | | |
| GGGGGTGTAGCTCAGTGGTAGAGCGCGTGCTTttaATG | TAA | 115.9 | 208 | 38 |
| CACGAGGCCCCGGGTTCAATCCCTGGCACCTCCA | | | | |
| GGGGATGTAGCTCAGTGGTAGAGCGCATGCTTttaATG | TAA | 72.50 | 237 | 39 |
| TATGAGGcCCCGGGTTCGATCCCCGGCATCTCCA | | | | |
| GGGGGTGTAGCTCAGTGGTAGAGCGCGTGCTTttaATG | TAA | 65.54 | 242 | 40 |
| TACGAGGtCCCGGGTTCAATCCCCGGCACCTCCA | | | | |
| GGGGATGTAGCTCAGTGGTAGAGCGCATGCTTttaATG | TAA | 64.46 | 244 | 41 |
| TATGAGGtCCCGGGTTCGATCCCCGGCATCTCCA | | | | |
| GGGGGTGTAGCTCAGTGGTAGAGCGCGTGCTTttaATG | TAA | 61.02 | 248 | 42 |
| CACGAGGcCCCGGGTTCAATCCCCGGCACCTCCA | | | | |
| GGGGAATTAGCTCAAATGGTAGAGCGCTCGCTTttaAT | TAA | 58.8 | 249 | 43 |
| GCGAGAGGtAGCGGGATCGATGCCCGCATTCTCCA | | | | |
| GGGGGTATAGCTCAGTGGTAGAGCGCGTGCTTttaATG | TAA | 50.81 | 260 | 44 |
| CACGAGGtCCTGGGTTCGATCCCCAGTACCTCCA | | | | |
| GGGGGATTAGCTCAAATGGTAGAGCGCTCGCTTttaAT | TAA | 45.09 | 271 | 45 |
| GCGAGAGGtAGCGGGATCGATGCCCGCATCCTCCA | | | | |
| GGGGAATTAGCTCAAGTGGTAGAGCGCTCGCTTttaAT | TAA | 32.5 | 302 | 46 |
| GCGAGAGGLAGTGGGATCGATGCCCGCATTCTCCA | | | | |
| GGGGGTGTAGCTCAGTGGTAGAGCGCGTGCTTttaATG | TAA | 29 | 314 | 47 |
| CACGAGGcCCTGGGTTCAATCCCCAGCACCTCCA | | | | |
| GGGGAATTAGCTCAGGCGGTAGAGCGCTCGCTTttaAT | TAA | 28.00 | 317 | 48 |
| GCGAGAGGtAGCGGGATCGACGCCCGCATTCTCCA | | | | |
| GGGGGTATAGCTCAGCGGTAGAGCGCGTGCTTttaATG | TAA | 8.95 | 417 | 49 |
| CACGAGGtCCTGGGTTCAATCCCCAATACCTCCA | | | | |
| GGGGAATTAGCTCAAGTGGTAGAGCGCTTGCTTttaAC | TAA | 8.40 | 424 | 50 |
| GCAAGAGGtAGTGGGATCGATGCCCACATTCTCCA | | | | |
| GGGGAATTAGCTCAAGTGGTAGAGCGCTTGCTTttaAT | TAA | 7.17 | 444 | 51 |
| GCAAGAGGtAGTGGGATCGATGCCCACATTCTCCA | | | | |
| GGGGAATTAGCTCAAGCGGTAGAGCGCTTGCTTttaAT | TAA | 5.31 | 482 | 52 |
| GCAAGAGGtAGTGGGATCGATGCCCACATTCTCCA | | | | |
| GGGGAATTAGCTCAAGTGGTAGAGCGCTTGCTTttaAT | TAA | 3.91 | 532 | 53 |
| GCAAGAGGtAGTGGGATCAATGCCCACATTCTCCA | | | | |
| GGGGAATTAGCCCAAGTGGTAGAGCGCTTGCTTttaAT | TAA | 2.34 | 654 | 54 |
| GCAAGAGGtAGTGGGATCGATGCCCACATTCTCCA | | | | |
| GGGGGTGTAGCTCAGTGGTAGAGCGCGTGCTTttaATG | TAA | 0.01 | 920 | 55 |
| TACGAGGCCCCGGGTTCGACCCCCGGCTCCTCCA | | | | |

TABLE 15B
Alanine (Ala) tRNA

| | | fold | | SEQ |
|---|---|---|---|---|
| seq_tRNA | ACE | over | Rank | ID |
| GGGGATGTAGCTCAGTGGTAGAGCGCGCGCTTctaATG | TAG | 172 | 186 | 8 |
| TGTGAGGtCCCGGGTTCAATCCCCGGCATCTCCA | | | | |
| GGGGATGTAGCTCAGTGGTAGAGCGCATGCTTctaATG | TAG | 127 | 201 | 12 |
| TATGAGGcCCCGGGTTCGATCCCCGGCATCTCCA | | | | |
| GGGGGTGTAGCTCAGTGGTAGAGCGCGTGCTTctaATG | TAG | 115 | 209 | 56 |
| TACGAGGtCCCGGGTTCAATCCCCGGCACCTCCA | | | | |
| GGGGGTGTAGCTCAGTGGTAGAGCGCGTGCTTctaATG | TAG | 104 | 216 | 57 |
| TACGAGGCCCCGGGTTCGACCCCCGGCTCCTCCA | | | | |
| GGGGGTGTAGCTCAGTGGTAGAGCGCGTGCTTctaATG | TAG | 100 | 220 | 58 |
| CACGAGGCCCCGGGTTCAATCCCTGGCACCTCCA | | | | |
| GGGGATGTAGCTCAGTGGTAGAGCGCATGCTTctaATG | TAG | 88.25 | 228 | 23 |
| CATGAGGtCCCGGGTTCGATCCCCAGCATCTCCA | | | | |
| GGGGGATTAGCTCAAATGGTAGAGCGCTCGCTTctaAT | TAG | 84.16 | 234 | 59 |
| GCGAGAGGtAGCGGGATCGATGCCCGCATCCTCCA | | | | |
| GGGGAATTAGCTCAGGCGGTAGAGCGCTCGCTTctaAT | TAG | 48.91 | 263 | 60 |
| GCGAGAGGtAGCGGGATCGACGCCCGCATTCTCCA | | | | |
| GGGGGTGTAGCTCAGTGGTAGAGCGCGTGCTTctaATG | TAG | 40.66 | 282 | 61 |
| CACGAGGCCCTGGGTTCAATCCCCAGCACCTCCA | | | | |
| GGGGGTGTAGCTCAGTGGTAGAGCGCGTGCTTctaATG | TAG | 38.37 | 287 | 62 |
| CACGAGGcCCCGGGTTCAATCCCCGGCACCTCCA | | | | |
| GGGGAATTAGCTCAAGTGGTAGAGCGCTCGCTTctaAT | TAG | 35.4 | 291 | 63 |
| GCGAGAGGLAGTGGGATCGATGCCCGCATTCTCCA | | | | |
| GGGGAATTAGCTCAAGTGGTAGAGCGCTTGCTTctaAC | TAG | 34.92 | 293 | 64 |
| GCAAGAGGtAGTGGGATCGATGCCCACATTCTCCA | | | | |
| GGGGAATTAGCTCAAGTGGTAGAGCGCTTGCTTctaAT | TAG | 8.96 | 416 | 65 |
| GCAAGAGGtAGTGGGATCGATGCCCACATTCTCCA | | | | |
| GGGGAATTAGCTCAAGCGGTAGAGCGCTTGCTTctaAT | TAG | 7.78 | 433 | 66 |
| GCAAGAGGtAGTGGGATCGATGCCCACATTCTCCA | | | | |
| GGGGAATTAGCTCAAGTGGTAGAGCGCTTGCTTctaAT | TAG | 5.78 | 469 | 67 |
| GCAAGAGGtAGTGGGATCAATGCCCACATTCTCCA | | | | |
| GGGGAATTAGCCCAAGTGGTAGAGCGCTTGCTTctaAT | TAG | 3.25 | 571 | 68 |
| GCAAGAGGtAGTGGGATCGATGCCCACATTCTCCA | | | | |
| GGGGGTATAGCTCAGCGGTAGAGCGCGTGCTTctaATG | TAG | 2.40 | 644 | 69 |
| CACGAGGtCCTGGGTTCAATCCCCAATACCTCCA | | | | |
| GGGGGTATAGCTCAGTGGTAGAGCGCGTGCTTctaATG | TAG | 0.12 | 909 | 29 |
| CACGAGGtCCTGGGTTCGATCCCCAGTACCTCCA | | | | |
| GGGGAATTAGCTCAAATGGTAGAGCGCTCGCTTctaAT | TAG | 0.03 | 915 | 34 |
| GCGAGAGGTAGCGGGATCGATGCCCGCATTCTCCA | | | | |
| GGGGATGTAGCTCAGTGGTAGAGCGCATGCTTctaATG | TAG | 0.00 | 924 | 70 |
| TATGAGGtCCCGGGTTCGATCCCCGGCATCTCCA | | | | |

TABLE 15C
Alanine (Ala) tRNA

| | | fold | | SEQ |
|---|---|---|---|---|
| seq_tRNA | ACE | over | Rank | ID |
| GGGGGTGTAGCTCAGTGGTAGAGCGCGTGCTTtcaATG | TGA | 12.99 | 374 | 71 |
| TACGAGGCCCCGGGTTCGACCCCCGGCTCCTCCA | | | | |
| GGGGATGTAGCTCAGTGGTAGAGCGCGCGCTTtcaATG | TGA | 12.79 | 375 | 72 |
| TGTGAGGtCCCGGGTTCAATCCCCGGCATCTCCA | | | | |
| GGGGATGTAGCTCAGTGGTAGAGCGCATGCTTtcaATG | TGA | 11.55 | 391 | 73 |
| CATGAGGtCCCGGGTTCGATCCCCAGCATCTCCA | | | | |
| GGGGGTGTAGCTCAGTGGTAGAGCGCGTGCTTtcaATG | TGA | 7.37 | 441 | 74 |
| CACGAGGcCCCGGGTTCAATCCCTGGCACCTCCA | | | | |
| GGGGATGTAGCTCAGTGGTAGAGCGCATGCTTtcaATG | TGA | 7.03 | 449 | 75 |
| TATGAGGcCCCGGGTTCGATCCCCGGCATCTCCA | | | | |
| GGGGGTGTAGCTCAGTGGTAGAGCGCGTGCTTtcaATG | TGA | 6.36 | 459 | 76 |
| TACGAGGtCCCGGGTTCAATCCCCGGCACCTCCA | | | | |
| GGGGGATTAGCTCAAATGGTAGAGCGCTCGCTTtcaAT | TGA | 5.81 | 468 | 77 |
| GCGAGAGGtAGCGGGATCGATGCCCGCATCCTCCA | | | | |
| GGGGAATTAGCTCAAATGGTAGAGCGCTCGCTTtcaAT | TGA | 4.95 | 492 | 78 |
| GCGAGAGGAGCGGGATCGATGCCCGCATTCTCCA | | | | |
| GGGGGTGTAGCTCAGTGGTAGAGCGCGTGCTTtcaATG | TGA | 4.95 | 493 | 79 |
| CACGAGGcCCTGGGTTCAATCCCCAGCACCTCCA | | | | |
| GGGGGTGTAGCTCAGTGGTAGAGCGCGTGCTTtcaATG | TGA | 4.94 | 494 | 80 |
| CACGAGGcCCCGGGTTCAATCCCCGGCACCTCCA | | | | |
| GGGGGTATAGCTCAGTGGTAGAGCGCGTGCTTtcaATG | TGA | 4.90 | 496 | 81 |
| CACGAGGTCCTGGGTTCGATCCCCAGTACCTCCA | | | | |
| GGGGATGTAGCTCAGTGGTAGAGCGCATGCTTtcaATG | TGA | 4.38 | 514 | 82 |
| TATGAGGtCCCGGGTTCGATCCCCGGCATCTCCA | | | | |
| GGGGAATTAGCTCAAGTGGTAGAGCGCTCGCTTtcaAT | TGA | 3.67 | 544 | 83 |
| GCGAGAGGLAGTGGGATCGATGCCCGCATTCTCCA | | | | |
| GGGGGTATAGCTCAGCGGTAGAGCGCGTGCTTtcaATG | TGA | 3.01 | 591 | 84 |
| CACGAGGtCCTGGGTTCAATCCCCAATACCTCCA | | | | |
| GGGGAATTAGCTCAAGCGGTAGAGCGCTTGCTTtcaAT | TGA | 2.56 | 625 | 85 |
| GCAAGAGGtAGTGGGATCGATGCCCACATTCTCCA | | | | |
| GGGGAATTAGCTCAGGCGGTAGAGCGCTCGCTTtcaAT | TGA | 2.07 | 699 | 86 |
| GCGAGAGGtAGCGGGATCGACGCCCGCATTCTCCA | | | | |
| GGGGAATTAGCCCAAGTGGTAGAGCGCTTGCTTtcaAT | TGA | 1.82 | 733 | 87 |
| GCAAGAGGtAGTGGGATCGATGCCCACATTCTCCA | | | | |
| GGGGAATTAGCTCAAGTGGTAGAGCGCTTGCTTtcaAT | TGA | 1.77 | 748 | 88 |
| GCAAGAGGtAGTGGGATCGATGCCCACATTCTCCA | | | | |
| GGGGAATTAGCTCAAGTGGTAGAGCGCTTGCTTtcaAC | TGA | 1.66 | 765 | 89 |
| GCAAGAGGtAGTGGGATCGATGCCCACATTCTCCA | | | | |
| GGGGAATTAGCTCAAGTGGTAGAGCGCTTGCTTtcaAT | TGA | 1.39 | 817 | 90 |
| GCAAGAGGtAGTGGGATCAATGCCCACATTCTCCA | | | | |

TABLE 16A
Arginine (Arg) tRNA

| | | Fold | | SEQ |
|---|---|---|---|---|
| seq_tRNA | ACE | over | Rank | ID |
| GGCTCCGTGGCGCAATGGAtAGCGCATTGGACTttaAAT | TAA | 121.36 | 203 | 91 |
| TCAAAGGtTCCGGGTTCGAGTCCCGGCGGAGTCG | | | | |
| GACCCAGTGGCCTAATGGAtAAGGCATCAGCCTttaGA | TAA | 93.18 | 224 | 92 |
| GCTGGGGAtTGTGGGTTCGAGTCCCATCTGGGTCG | | | | |
| GCCCCAGTGGCCTAATGGAtAAGGCATTGGCCTttaAAG | TAA | 92.32 | 225 | 93 |
| CCAGGGAtTGTGGGTTCGAGTCCCATCTGGGGTG | | | | |
| GGGCCAGTGGCGCAATGGAtAACGCGTCTGACTttaGA | TAA | 70.38 | 239 | 94 |
| TCAGAAGAtTCCAGGTTCGACTCCTGGCTGGCTCG | | | | |
| GGGCCAGTGGCGCAATGGAtAACGCGTCTGACTttaGA | TAA | 66.91 | 241 | 95 |
| TCAGAAGAtTCTAGGTTCGACTCCTGGCTGGCTCG | | | | |
| GCCCCAGTGGCCTAATGGAtAAGGCACTGGCCTttaAA | TAA | 63.63 | 247 | 96 |
| GCCAGGGAtTGTGGGTTCGAGTCCCACCTGGGGTG | | | | |
| GTCTCTGTGGCGCAATGGAcgAGCGCGCTGGACTttaAA | TAA | 51.68 | 257 | 97 |
| TCCAGAGGtTCCGGGTTCGAGTCCCGGCAGAGATG | | | | |
| GGCTCTGTGGCGCAATGGAtAGCGCATTGGACTttaAAT | TAA | 51.08 | 258 | 98 |
| TCAAAGGTGTGGGTTCGAGTCCCACCAGAGTCG | | | | |
| GGCCGCGTGGCCTAATGGAtAAGGCGTCTGACTttaGAT | TAA | 48.33 | 264 | 99 |
| CAGAAGAtTGCAGGTTCGAGTCCTGCCGCGGTCG | | | | |
| GCCCCGGTGGCCTAATGGAtAAGGCATTGGCCTttaAAG | TAA | 47.14 | 266 | 100 |
| CCAGGGAtTGTGGGTTCGAGTCCCACCCGGGGTA | | | | |
| GACCGCGTGGCCTAATGGAtAAGGCGTCTGACTttaGAT | TAA | 44.92 | 272 | 101 |
| CAGAAGALTGAGGGTTCGAGTCCCTTCGTGGTCG | | | | |
| GCCCCAGTGGCCTAATGGAtAAGGCACTGGCCTttaAA | TAA | 34.21 | 296 | 102 |
| GCCAGGGAtTGTGGGTTCGAGTCCCACCTGGGGTA | | | | |
| GGCTCTGTGGCGCAATGGAtAGCGCATTGGACTttaAAT | TAA | 30.60 | 309 | 103 |
| TCAAAGGTGTGGGTTCGAGTCCCACCAGAGTCG | | | | |
| GGCTCTGTGGCGCAATGGAtAGCGCATTGGACTttaAAT | TAA | 26.99 | 320 | 104 |
| TCAAAGGTGTGGGTTCGAATCCCACCAGAGTCG | | | | |
| GACCACGTGGCCTAATGGAtAAGGCGTCTGACTttaGAT | TAA | 17.79 | 353 | 105 |
| CAGAAGATGAGGGTTCGAATCCCTCCGTGGTTA | | | | |
| GACCACGTGGCCTAATGGAtAAGGCGTCTGACTttaGAT | TAA | 12.50 | 376 | 106 |
| CAGAAGALTGAGGGTTCGAATCCCTTCGTGGTTA | | | | |
| GACCACGTGGCCTAATGGAtAAGGCGTCTGACTttaGAT | TAA | 12.19 | 381 | 107 |
| CAGAAGALTGAGGGTTCGAATCCCTTCGTGGTTG | | | | |
| GGCTCTGTGGCGCAATGGAtAGCGCATTGGACTttaAAT | TAA | 11.56 | 390 | 108 |
| TCAAAGGtTGCGGGTTCGAGTCCCTCCAGAGTCG | | | | |
| GGCCGCGTGGCCTAATGGAtAAGGCGTCTGATTttaGAT | TAA | 8.87 | 418 | 109 |
| CAGAAGALTGAGGGTTCGAGTCCCTTCGTGGTCG | | | | |
| GGCAGCATAGCAGAGTGGTtCAGGTTACAGGTttaAGA | TAA | 2.06 | 702 | 110 |
| TGTAAACTGAGTTCAAATCCCAGTTCTGCCA | | | | |
| GCCCCAGTGGCCTGATGGAtAAGGTACTGGCCTttaAAG | TAA | 2.02 | 707 | 111 |
| CCAGGGAtTGTGGGTTCGAGTTCCACCTGGGGTA | | | | |
| GGCCGTGTGGCCTAATGGAtAAGGCGTCTGACTttaGAT | TAA | 1.73 | 753 | 112 |
| CAAAAGAtTGCAGGTTTGAGTTCTGCCACGGTCG | | | | |

TABLE 16B
Arginine (Arg) tRNA

| | | fold | | SEQ |
|---|---|---|---|---|
| seq_tRNA | ACE | over | Rank | ID |
| GGCTCCGTGGCGCAATGGAtAGCGCATTGGACTcta | TAG | 4422.24 | 20 | 113 |
| AATTCAAAGGtTCCGGGTTCGAGTCCCGGCGGAGT | | | | |
| CG | | | | |
| GCCCCAGTGGCCTAATGGAtAAGGCATTGGCCTcta | TAG | 3041.46 | 39 | 114 |
| AAGCCAGGGAtTGTGGGTTCGAGTCCCATCTGGGG | | | | |
| TG | | | | |
| GGCTCTGTGGCGCAATGGAtAGCGCATTGGACTcta | TAG | 2719.18 | 43 | 115 |
| AATTCAAAGGETGTGGGTTCGAGTCCCACCAGAGT | | | | |
| CG | | | | |
| GCCCCGGTGGCCTAATGGAtAAGGCATTGGCCTcta | TAG | 1227.07 | 79 | 116 |
| AAGCCAGGGAtTGTGGGTTCGAGTCCCACCCGGGG | | | | |
| TA | | | | |
| GGCTCTGTGGCGCAATGGAtAGCGCATTGGACTcta | TAG | 791.34 | 97 | 117 |
| AATTCAAAGGTGTGGGTTCGAATCCCACCAGAGT | | | | |
| CG | | | | |
| GACCCAGTGGCCTAATGGAtAAGGCATCAGCCTcta | TAG | 776.95 | 98 | 118 |
| GAGCTGGGGAtTGTGGGTTCGAGTCCCATCTGGGT | | | | |
| CG | | | | |
| GACCACGTGGCCTAATGGAtAAGGCGTCTGACTcta | TAG | 505.44 | 127 | 119 |
| GATCAGAAGALTGAGGGTTCGAATCCCTTCGTGGT | | | | |
| TA | | | | |
| GGCCGCGTGGCCTAATGGAtAAGGCGTCTGACTcta | TAG | 428.91 | 135 | 120 |
| GATCAGAAGAtTGCAGGTTCGAGTCCTGCCGCGGT | | | | |
| CG | | | | |
| GGGCCAGTGGCGCAATGGAtAACGCGTCTGACTcta | TAG | 399.39 | 138 | 121 |
| GATCAGAAGATTCCAGGTTCGACTCCTGGCTGGCT | | | | |
| CG | | | | |
| GGCTCTGTGGCGCAATGGAtAGCGCATTGGACTcta | TAG | 326.35 | 151 | 122 |
| AATTCAAAGGETGCGGGTTCGAGTCCCTCCAGAGT | | | | |
| CG | | | | |
| GCCCCAGTGGCCTAATGGAtAAGGCACTGGCCTctaAA | TAG | 209.08 | 173 | 123 |
| GCCAGGGAtTGTGGGTTCGAGTCCCACCTGGGGTA | | | | |
| GACCGCGTGGCCTAATGGAtAAGGCGTCTGACTctaGA | TAG | 184.56 | 183 | 124 |
| TCAGAAGAtTGAGGGTTCGAGTCCCTTCGTGGTCG | | | | |
| GCCCCAGTGGCCTAATGGAtAAGGCACTGGCCTctaAA | TAG | 151.36 | 194 | 125 |
| GCCAGGGAtTGTGGGTTCGAGTCCCACCTGGGGTG | | | | |
| GACCACGTGGCCTAATGGAtAAGGCGTCTGACTctaGA | TAG | 128.84 | 200 | 126 |
| TCAGAAGAtTGAGGGTTCGAATCCCTTCGTGGTTG | | | | |
| GGCCGCGTGGCCTAATGGAtAAGGCGTCTGATTctaGA | TAG | 113.16 | 211 | 127 |
| TCAGAAGAtTGAGGGTTCGAGTCCCTTCGTGGTCG | | | | |
| GGGCCAGTGGCGCAATGGAtAACGCGTCTGACTctaGA | TAG | 103.79 | 217 | 128 |
| TCAGAAGATCTAGGTTCGACTCCTGGCTGGCTCG | | | | |
| GTCTCTGTGGCGCAATGGAcgAGCGCGCTGGACTctaA | TAG | 103.47 | 218 | 129 |
| ATCCAGAGGTTCCGGGTTCGAGTCCCGGCAGAGATG | | | | |
| GACCACGTGGCCTAATGGAtAAGGCGTCTGACTctaGA | TAG | 103.02 | 219 | 130 |
| TCAGAAGAtTGAGGGTTCGAATCCCTCCGTGGTTA | | | | |
| GCCCCAGTGGCCTGATGGAtAAGGTACTGGCCTctaAA | TAG | 27.87 | 318 | 131 |
| GCCAGGGAtTGTGGGTTCGAGTTCCACCTGGGGTA | | | | |
| GGCAGCATAGCAGAGTGGTtCAGGTTACAGGTctaAGA | TAG | 19.26 | 346 | 132 |
| TGTAAACTGAGTTCAAATCCCAGTTCTGCCA | | | | |
| GGCCGTGTGGCCTAATGGAtAAGGCGTCTGACTctaGA | TAG | 5.65 | 473 | 133 |
| TCAAAAGAtTGCAGGTTTGAGTTCTGCCACGGTCG | | | | |

TABLE 16C
Arginine (Arg) tRNA

| | | fold | | SEQ |
|---|---|---|---|---|
| seq_tRNA | ACE | over | Rank | ID |
| GACCACGTGGCCTAATGGAtAAGGCGTCTGACTtcaGATC | TGA | 1221.16 | 80 | 134 |
| AGAAGAtTGAGGGTTCGAATCCCTTCGTGGTTG | | | | |
| GGCTCTGTGGCGCAATGGAtAGCGCATTGGACTtcaAATT | TGA | 893.12 | 93 | 135 |
| CAAAGGETGTGGGTTCGAATCCCACCAGAGTCG | | | | |
| GCCCCAGTGGCCTAATGGAtAAGGCACTGGCCTtcaAAGC | TGA | 848.90 | 94 | 136 |
| CAGGGAtTGTGGGTTCGAGTCCCACCTGGGGTA | | | | |
| GCCCCGGTGGCCTAATGGAtAAGGCATTGGCCTtcaAAGC | TGA | 800.94 | 96 | 137 |
| CAGGGAtTGTGGGTTCGAGTCCCACCCGGGGTA | | | | |
| GCCCCAGTGGCCTAATGGAtAAGGCACTGGCCTtcaAAGC | TGA | 776.35 | 99 | 138 |
| CAGGGATGTGGGTTCGAGTCCCACCTGGGGTG | | | | |
| GGCTCTGTGGCGCAATGGAtAGCGCATTGGACTtcaAATT | TGA | 750.16 | 102 | 139 |
| CAAAGGETGTGGGTTCGAGTCCCACCAGAGTCG | | | | |
| GTCTCTGTGGCGCAATGGAcgAGCGCGCTGGACTtcaAAT | TGA | 725.36 | 103 | 140 |
| CCAGAGGTTCCGGGTTCGAGTCCCGGCAGAGATG | | | | |
| GACCCAGTGGCCTAATGGAtAAGGCATCAGCCTtcaGAGC | TGA | 678.27 | 105 | 141 |
| TGGGGATGTGGGTTCGAGTCCCATCTGGGTCG | | | | |
| GCCCCAGTGGCCTGATGGAtAAGGTACTGGCCTtcaAAGC | TGA | 663.25 | 107 | 142 |
| CAGGGAtTGTGGGTTCGAGTTCCACCTGGGGTA | | | | |
| GGCTCCGTGGCGCAATGGAtAGCGCATTGGACTtcaAATT | TGA | 623.65 | 110 | 143 |
| CAAAGGTTCCGGGTTCGAGTCCCGGCGGAGTCG | | | | |
| GACCGCGTGGCCTAATGGAtAAGGCGTCTGACTtcaGATC | TGA | 598.87 | 114 | 144 |
| AGAAGAtTGAGGGTTCGAGTCCCTTCGTGGTCG | | | | |
| GGCTCTGTGGCGCAATGGAtAGCGCATTGGACTtcaAATT | TGA | 563.93 | 117 | 145 |
| CAAAGGTTGCGGGTTCGAGTCCCTCCAGAGTCG | | | | |
| GGGCCAGTGGCGCAATGGAtAACGCGTCTGACTtcaGATC | TGA | 560.54 | 118 | 146 |
| AGAAGAtTCTAGGTTCGACTCCTGGCTGGCTCG | | | | |
| GGCTCTGTGGCGCAATGGAtAGCGCATTGGACTtcaAATT | TGA | 515.18 | 126 | 147 |
| CAAAGGTGTGGGTTCGAGTCCCACCAGAGTCG | | | | |
| GGGCCAGTGGCGCAATGGAtAACGCGTCTGACTtcaGATC | TGA | 313.53 | 153 | 148 |
| AGAAGAtTCCAGGTTCGACTCCTGGCTGGCTCG | | | | |
| GACCACGTGGCCTAATGGAtAAGGCGTCTGACTtcaGATC | TGA | 241.70 | 168 | 149 |
| AGAAGAtTGAGGGTTCGAATCCCTTCGTGGTTA | | | | |
| GGCCGCGTGGCCTAATGGAtAAGGCGTCTGACTtcaGATC | TGA | 216.85 | 169 | 150 |
| AGAAGAtTGCAGGTTCGAGTCCTGCCGCGGTCG | | | | |
| GACCACGTGGCCTAATGGAtAAGGCGTCTGACTtcaGATC | TGA | 213.63 | 171 | 151 |
| AGAAGAtTGAGGGTTCGAATCCCTCCGTGGTTA | | | | |
| GGCCGTGTGGCCTAATGGAtAAGGCGTCTGACTtcaGATC | TGA | 198.99 | 178 | 152 |
| AAAAGAtTGCAGGTTTGAGTTCTGCCACGGTCG | | | | |
| GGCCGCGTGGCCTAATGGAtAAGGCGTCTGATTtcaGATC | TGA | 89.63 | 226 | 153 |
| AGAAGAtTGAGGGTTCGAGTCCCTTCGTGGTCG | | | | |
| GGCAGCATAGCAGAGTGGTtCAGGTTACAGGTtcaAGAT | TGA | 2.59 | 622 | 154 |
| GTAAACTGAGTTCAAATCCCAGTTCTGCCA | | | | |
| GCCCCAGTGGCCTAATGGAtAAGGCATTGGCCTtcaAAGC | TGA | 2.57 | 624 | 155 |
| CAGGGAtTGTGGGTTCGAGTCCCATCTGGGGTG | | | | |

TABLE 17A
Asparagine (Asn) RNA

| | | fold | | SEQ |
|---|---|---|---|---|
| seq_tRNA | ACE | over | Rank | ID |
| GTCTCTGTGGCGCAATCGGTtAGCGCGTTCGGCTttaAA | TAA | 89.49 | 227 | 156 |
| CCGAAAGGETGGTGGTTCGAGCCCACCCAGGGACG | | | | |
| GTCTCTGTGGCGCAATGGGTtAGCGCGTTCGGCTttaAA | TAA | 34.41 | 294 | 157 |
| CCGAAAGGETGGTGGTTCGAGCCCATCCAGGGACG | | | | |
| GTCTCTGTGGCGCAATCGGTtAGCGCGTTCGGCTttaAA | TAA | 33.30 | 298 | 158 |
| CCGAAAGGETGGTGGTTCGATCCCACCCAGGGACG | | | | |
| GTCTCTGTGGCGCAATCGGTtAGCGCATTCGGCTttaAA | TAA | 25.89 | 322 | 159 |
| CCGAAAGGETGGTGGTTCGAGCCCACCCAGGGACG | | | | |
| GTCTCTGTGGCGCAATCGGTtAGCGCGTTCGGCTttaAA | TAA | 23.87 | 330 | 160 |
| CTGAAAGGETGGTGGTTCGAGCCCACCCAGGGACG | | | | |
| GTCTCTGTGGCGCAATCGGTtAGCGCGTTCGGCTttaAA | TAA | 11.84 | 385 | 161 |
| CCGAAAGAtTGGTGGTTCGAGCCCACCCAGGGACG | | | | |
| GTCTCTGTGGCGCAATCGGCtAGCGCGTTTGGCTttaAA | TAA | 9.98 | 409 | 162 |
| CTAAAAGGETGGTGGTTCGAACCCACCCAGAGGCG | | | | |
| GTCTCTGTGGCGCAATCGGCtAGCGCGTTTGGCTttaAA | TAA | 2.07 | 698 | 163 |
| CTAAAAGGETGGCGGTTCGAACCCACCCAGAGGCG | | | | |
| GTCTCTGTGGCGCAATCGGCtAGCGCGTTTGGCTttaAA | TAA | 1.63 | 768 | 164 |
| CTAAAAAGTTGGTGGTTCGAACACACCCAGAGGCG | | | | |
| GTCTCTGTGGCGTAGTCGGTtAGCGCGTTCGGCTttaAA | TAA | 1.48 | 799 | 165 |
| CCGAAAAGtTGGTGGTTCGAGCCCACCCAGGAACG | | | | |
| GTCTCTGTGGTGCAATCGGTtAGCGCGTTCCGCTttaAA | TAA | 1.42 | 814 | 166 |
| CCGAAAGCTTGGTGGTTCGAGCCCACCCAGGGATG | | | | |
| GTCTCTGTGGCGCAATCGGTtAGCGCGTTCGGCTttaAA | TAA | 1.17 | 842 | 167 |
| CTGAAAGGtTAGTGGTTCGAGCCCACCCGGGGACG | | | | |

TABLE 17B
Asparagine (Asn) tRNA

| | | fold | | SEQ |
|---|---|---|---|---|
| seq_tRNA | ACE | over | Rank | ID |
| GTCTCTGTGGCGCAATGGGTtAGCGCGTTCGGCTctaAA | TAG | 4.88 | 497 | 168 |
| CCGAAAGGETGGTGGTTCGAGCCCATCCAGGGACG | | | | |
| GTCTCTGTGGCGCAATCGGCtAGCGCGTTTGGCTctaAA | TAG | 4.54 | 508 | 169 |
| CTAAAAGGETGGTGGTTCGAACCCACCCAGAGGCG | | | | |
| GTCTCTGTGGCGCAATCGGTtAGCGCGTTCGGCTctaAA | TAG | 3.13 | 580 | 170 |
| CCGAAAGGETGGTGGTTCGATCCCACCCAGGGACG | | | | |
| GTCTCTGTGGCGCAATCGGTtAGCGCATTCGGCTctaAA | TAG | 2.71 | 614 | 171 |
| CCGAAAGGTGGTGGTTCGAGCCCACCCAGGGACG | | | | |
| GTCTCTGTGGCGCAATCGGTtAGCGCGTTCGGCTctaAA | TAG | 2.31 | 660 | 172 |
| CCGAAAGGETGGTGGTTCGAGCCCACCCAGGGACG | | | | |
| GTCTCTGTGGCGCAATCGGTtAGCGCGTTCGGCTctaAA | TAG | 1.97 | 716 | 173 |
| CTGAAAGGETGGTGGTTCGAGCCCACCCAGGGACG | | | | |
| GTCTCTGTGGCGCAATCGGTtAGCGCGTTCGGCTctaAA | TAG | 1.91 | 723 | 174 |
| CCGAAAGAtTGGTGGTTCGAGCCCACCCAGGGACG | | | | |
| GTCTCTGTGGCGCAATCGGCtAGCGCGTTTGGCTctaAA | TAG | 1.56 | 781 | 175 |
| CTAAAAAGtTGGTGGTTCGAACACACCCAGAGGCG | | | | |
| GTCTCTGTGGCGCAATCGGCtAGCGCGTTTGGCTctaAA | TAG | 1.33 | 822 | 176 |
| CTAAAAGGTTGGCGGTTCGAACCCACCCAGAGGCG | | | | |
| GTCTCTGTGGCGTAGTCGGTtAGCGCGTTCGGCTctaAA | TAG | 1.21 | 836 | 177 |
| CCGAAAAGtTGGTGGTTCGAGCCCACCCAGGAACG | | | | |
| GTCTCTGTGGTGCAATCGGTtAGCGCGTTCCGCTctaAA | TAG | 0.95 | 868 | 178 |
| CCGAAAGCTTGGTGGTTCGAGCCCACCCAGGGATG | | | | |
| GTCTCTGTGGCGCAATCGGTtAGCGCGTTCGGCTctaAA | TAG | 0.48 | 894 | 179 |
| CTGAAAGGtTAGTGGTTCGAGCCCACCCGGGGACG | | | | |

TABLE 17C
Asparagine (Asn) tRNA

| | | fold | | SEQ |
|---|---|---|---|---|
| seq_tRNA | ACE | over | Rank | ID |
| GTCTCTGTGGCGCAATCGGTtAGCGCGTTCGGCTtcaAA | TGA | 23.02 | 335 | 180 |
| CTGAAAGGETGGTGGTTCGAGCCCACCCAGGGACG | | | | |
| GTCTCTGTGGCGCAATCGGTtAGCGCGTTCGGCTtcaAA | TGA | 7.72 | 434 | 181 |
| CCGAAAGALTGGTGGTTCGAGCCCACCCAGGGACG | | | | |
| GTCTCTGTGGCGCAATCGGTtAGCGCATTCGGCTtcaAA | TGA | 5.32 | 481 | 182 |
| CCGAAAGGETGGTGGTTCGAGCCCACCCAGGGACG | | | | |
| GTCTCTGTGGCGCAATCGGTtAGCGCGTTCGGCTtcaAA | TGA | 4.01 | 527 | 183 |
| CCGAAAGGETGGTGGTTCGATCCCACCCAGGGACG | | | | |
| GTCTCTGTGGCGCAATGGGTtAGCGCGTTCGGCTtcaAA | TGA | 3.87 | 536 | 184 |
| CCGAAAGGETGGTGGTTCGAGCCCATCCAGGGACG | | | | |
| GTCTCTGTGGCGCAATCGGTtAGCGCGTTCGGCTtcaAA | TGA | 3.18 | 576 | 185 |
| CCGAAAGGETGGTGGTTCGAGCCCACCCAGGGACG | | | | |
| GTCTCTGTGGCGCAATCGGCtAGCGCGTTTGGCTtcaAA | TGA | 2.71 | 613 | 186 |
| CTAAAAGGETGGTGGTTCGAACCCACCCAGAGGCG | | | | |
| GTCTCTGTGGCGCAATCGGCtAGCGCGTTTGGCTtcaAA | TGA | 2.66 | 618 | 187 |
| CTAAAAAGtTGGTGGTTCGAACACACCCAGAGGCG | | | | |
| GTCTCTGTGGTGCAATCGGTtAGCGCGTTCCGCTtcaAA | TGA | 2.12 | 690 | 188 |
| CCGAAAGCtTGGTGGTTCGAGCCCACCCAGGGATG | | | | |
| GTCTCTGTGGCGCAATCGGCtAGCGCGTTTGGCTtcaAA | TGA | 1.73 | 754 | 189 |
| CTAAAAGGETGGCGGTTCGAACCCACCCAGAGGCG | | | | |
| GTCTCTGTGGCGTAGTCGGTtAGCGCGTTCGGCTtcaAA | TGA | 1.53 | 787 | 190 |
| CCGAAAAGtTGGTGGTTCGAGCCCACCCAGGAACG | | | | |
| GTCTCTGTGGCGCAATCGGTtAGCGCGTTCGGCTtcaAA | TGA | 1.30 | 829 | 191 |
| CTGAAAGGtTAGTGGTTCGAGCCCACCCGGGGACG | | | | |

TABLE 18A
Aspartic acid (Asp) tRNA

| | | fold | | SEQ |
|---|---|---|---|---|
| seq_tRNA | ACE | over | Rank | ID |
| TCCTCGTTAGTATAGTGGTtAGTATCCCCGCCTttaACG | TAA | 160.19 | 190 | 192 |
| CGGGAGaCCGGGGTTCAATTCCCCGACGGGGAG | | | | |
| TCCTCGTTAGTATAGTGGTgAGTATCCCCGCCTttaACG | TAA | 13.06 | 371 | 193 |
| CGGGAGaCCGGGGTTCGATTCCCCGACGGGGAG | | | | |
| TCCTCGTTAGTATAGTGGTgAGTGTCCCCGTCTttaACG | TAA | 0.71 | 882 | 194 |
| CGGGAGaCCGGGGTTCGATTCCCCGACGGGGAG | | | | |

TABLE 18B
Aspartic acid (Asp) tRNA

| | | fold | | SEQ |
|---|---|---|---|---|
| seq_tRNA | ACE | over | Rank | ID |
| TCCTCGTTAGTATAGTGGTgAGTATCCCCGCCTctaACG | TAG | 159.14 | 191 | 195 |
| CGGGAGaCCGGGGTTCGATTCCCCGACGGGGAG | | | | |
| TCCTCGTTAGTATAGTGGTtAGTATCCCCGCCTctaACG | TAG | 158.93 | 192 | 196 |
| CGGGAGaCCGGGGTTCAATTCCCCGACGGGGAG | | | | |
| TCCTCGTTAGTATAGTGGTgAGTGTCCCCGTCTctaACG | TAG | 0.73 | 879 | 197 |
| CGGGAGaCCGGGGTTCGATTCCCCGACGGGGAG | | | | |

TABLE 18C
Aspartic acid (Asp) tRNA

| | | fold | | SEQ |
|---|---|---|---|---|
| seq_tRNA | ACE | over | Rank | ID |
| TCCTCGTTAGTATAGTGGTgAGTGTCCCCGTCTtcaACG | TGA | 1.18 | 841 | 198 |
| CGGGAGaCCGGGGTTCGATTCCCCGACGGGGAG | | | | |
| TCCTCGTTAGTATAGTGGTtAGTATCCCCGCCTtcaACG | TGA | 0.79 | 877 | 199 |
| CGGGAGaCCGGGGTTCAATTCCCCGACGGGGAG | | | | |
| TCCTCGTTAGTATAGTGGTgAGTATCCCCGCCTtcaACG | TGA | 0.46 | 896 | 200 |
| CGGGAGaCCGGGGTTCGATTCCCCGACGGGGAG | | | | |

TABLE 19A
Cysteine (Cys) tRNA

| | | fold | | SEQ |
|---|---|---|---|---|
| seq_tRNA | ACE | over | Rank | ID |
| GGGGGTATAGCTCAGTGGTAGAGCATTTGACTttaGAT | TAA | 50.62 | 261 | 201 |
| CAAGAGGTCCCTGGTTCAAATCCGGGTGCCCCCT | | | | |
| GGGGGTATAGCTCAGTGGGTAGAGCATTTGACTttaGA | TAA | 43.72 | 276 | 202 |
| TCAAGAGGtCCCCGGTTCAAATCCGGGTGCCCCCT | | | | |
| GGGGGTATAGCTCAGGGGTAGAGCATTTGACTttaGAT | TAA | 22.64 | 337 | 203 |
| CAAGAGGtCCCCGGTTCAAATCCGGGTGCCCCCT | | | | |
| GGGGGTATAGCTTAGCGGTAGAGCATTTGACTttaGAT | TAA | 20.98 | 339 | 204 |
| CAAGAGGtCCCCGGTTCAAATCCGGGTGCCCCCT | | | | |
| GGGGGTATAGCTCAGGGGTAGAGCATTTGACTttaGAT | TAA | 20.10 | 344 | 205 |
| CAAGAAGCCCCGGTTCAAATCCGGGTGCCCCCT | | | | |
| GGGGGTGTAGCTCAGTGGTAGAGCATTTGACTttaGAT | TAA | 18.01 | 352 | 206 |
| CAAGAGGTCCCTGGTTCAAATCCAGGTGCCCCCT | | | | |
| GGGGGTATAGCTCAGGGGTAGAGCATTTGACTttaGAT | TAA | 11.98 | 383 | 207 |
| CAAGAGGtCCCTGGTTCAAATCCAGGTGCCCCCT | | | | |
| GGGGGTATAGCTCAGGGGTAGAGCATTTGACTttaGAT | TAA | 10.36 | 401 | 208 |
| CAAGAGGtCTCTGGTTCAAATCCAGGTGCCCCCT | | | | |
| GGGGGTATAGCTCAGGGGTAGAGCATTTGACTttaGAT | TAA | 8.18 | 429 | 209 |
| CAAGAGGtCCCCAGTTCAAATCTGGGTGCCCCCT | | | | |
| GGGGGTATAGCTCAGGGGTAGAGCATTTGACTttaGAT | TAA | 7.14 | 446 | 210 |
| CAAGAGGtCCCTGGTTCAAATCCAGGTGCCCCCC | | | | |
| GGGGGTATAGCTCAGGGGTAGAGCACTTGACTttaGAT | TAA | 4.77 | 501 | 211 |
| CAAGAGGtCCCTGGTTCAAATCCAGGTGCCCCCT | | | | |
| GGGGGTATAGCTTAGGGGTAGAGCATTTGACTttaGAT | TAA | 4.64 | 504 | 212 |
| CAAAAGGtCCCTGGTTCAAATCCAGGTGCCCCTT | | | | |
| GGGGGTATAGCTCAGGGGTAGAGCACTTGACTttaGAT | TAA | 3.97 | 531 | 213 |
| CAAGAAGtCCTTGGTTCAAATCCAGGTGCCCCCT | | | | |
| GGTATATCTCAGGGGGCAGAGCATTTGACTttaGATCAA | TAA | 3.31 | 568 | 214 |
| GAGGtCCCCGGTTGAAATCCGGGTGCT | | | | |
| GGGGGCATAGCTCAGTGGTAGAGCATTTGACTttaGAT | TAA | 2.35 | 652 | 215 |
| CAAGAGGTCCCTGGTTCAAATCCAGGTGCCCCCT | | | | |
| GGGGGTATAGCTCACAGGTAGAGCATTTGACTttaGAT | TAA | 2.14 | 686 | 216 |
| CAAGAGGtCCCCGGTTCAAATCCGGTTACTCCCT | | | | |
| GGCAGAATGGTGCAGCGGTtcAGCACCCAGgCTCTttaGc | TAA | 2.13 | 688 | 217 |
| CAGCTGTTGCCTGGGCTCAAATCCCAGCTCTGCCA | | | | |
| GGGGGTATAGCTCACAGGTAGAGCATTTGACTttaGAT | TAA | 2.11 | 692 | 218 |
| CAAGAGGtCCCCGGTTCAAATCTGGGTGCCCCCT | | | | |
| GGGGGTAGGGCTCAGGGAtAGAGCATTTGACTttaGAT | TAA | 1.66 | 763 | 219 |
| CAAGAGGtCCCCGGTTCGAATCTAGGTGCCCCCT | | | | |
| GGGCGTATAGCTCAGGGGTAGAGCATTTGACTttaGAT | TAA | 1.40 | 815 | 220 |
| CAAGAGGtCCCCAGTTCAAATCTGGGTGCCCCCT | | | | |
| GGGGGTATAGTTCAGGGGTAGAGCATTTGACTttaGAT | TAA | 1.20 | 839 | 221 |
| CAAGAGGtCCCTGGTTCAAATCCAGGTGCCCCCT | | | | |
| GGCTGTATAGCTCAGTGGTAGAGCATTTGACTttaGCTT | TAA | 1.16 | 843 | 222 |
| TAAAGTCATAtGTAGCTGGGTTCAAATCCTGTTTCTGC | | | | |
| CA | | | | |
| GGGGGTATAGCTCAGGGGTAGAGCATTTGACTttaAAT | TAA | 1.13 | 846 | 223 |
| CAAGAGGtCCCTGATTCAAATCCAGGTGCCCCCT | | | | |
| GGGGATATAGCTCAGGGGTAGAGCATTTGACTttaGAT | TAA | 1.05 | 856 | 224 |
| CAAGAGGtCCCCGGTTCAAATCCGGGTGCCCCCC | | | | |
| GGGCGTATAGCTCAGGGGTAGAGCATTTGACTttaGAT | TAA | 0.94 | 869 | 225 |
| CAAGAGGTCCCCAGTTCAAATCTGGGTGCCCA | | | | |

TABLE 19B
Cysteine (Cys) tRNA

| | | fold | | SEQ |
|---|---|---|---|---|
| seq_tRNA | ACE | over | Rank | ID |
| GGGGGTATAGCTCAGGGGTAGAGCATTTGACTctaG | TAG | 1455.23 | 70 | 226 |
| ATCAAGAGGtCCCTGGTTCAAATCCAGGTGCCCCC | | | | |
| C | | | | |
| GGGGGTATAGCTCAGTGGGTAGAGCATTTGACTcta | TAG | 773.95 | 100 | 227 |
| GATCAAGAGGtCCCCGGTTCAAATCCGGGTGCCCC | | | | |
| CT | | | | |
| GGGGGTATAGCTCAGTGGTAGAGCATTTGACTctaGAT | TAG | 537.85 | 123 | 228 |
| CAAGAGGtCCCTGGTTCAAATCCGGGTGCCCCCT | | | | |
| GGGGGTGTAGCTCAGTGGTAGAGCATTTGACTctaG | TAG | 348.06 | 149 | 229 |
| ATCAAGAGGtCCCTGGTTCAAATCCAGGTGCCCCC | | | | |
| T | | | | |
| GGGGGTATAGCTCAGGGGTAGAGCATTTGACTctaG | TAG | 329.27 | 150 | 230 |
| ATCAAGAGGtCCCCGGTTCAAATCCGGGTGCCCCC | | | | |
| T | | | | |
| GGGGGTATAGCTCAGGGGTAGAGCATTTGACTctaG | TAG | 280.15 | 157 | 231 |
| ATCAAGAGGtCCCTGGTTCAAATCCAGGTGCCCCC | | | | |
| T | | | | |
| GGGGGCATAGCTCAGTGGTAGAGCATTTGACTctaG | TAG | 266.50 | 161 | 232 |
| ATCAAGAGGtCCCTGGTTCAAATCCAGGTGCCCCC | | | | |
| T | | | | |
| GGGGGTATAGCTCAGGGGTAGAGCACTTGACTcta | TAG | 254.45 | 164 | 233 |
| GATCAAGAGGtCCCTGGTTCAAATCCAGGTGCCCC | | | | |
| CT | | | | |
| GGGGGTATAGCTCAGGGGTAGAGCATTTGACTctaGAT | TAG | 204.11 | 177 | 234 |
| CAAGAGGtCCCCAGTTCAAATCTGGGTGCCCCCT | | | | |
| GGGGGTATAGCTCAGGGGTAGAGCATTTGACTctaGAT | TAG | 195.93 | 180 | 235 |
| CAAGAAGtCCCCGGTTCAAATCCGGGTGCCCCCT | | | | |
| GGGGGTATAGCTTAGCGGTAGAGCATTTGACTctaGAT | TAG | 162.72 | 189 | 236 |
| CAAGAGGtCCCCGGTTCAAATCCGGGTGCCCCCT | | | | |
| GGGGGTATAGCTCAGGGGTAGAGCATTTGACTctaGAT | TAG | 151.49 | 193 | 237 |
| CAAGAGGtCTCTGGTTCAAATCCAGGTGCCCCCT | | | | |
| GGGGGTATAGCTCAGGGGTAGAGCACTTGACTctaGAT | TAG | 75.99 | 236 | 238 |
| CAAGAAGtCCTTGGTTCAAATCCAGGTGCCCCCT | | | | |
| GGGGGTAGGGCTCAGGGAtAGAGCATTTGACTctaGAT | TAG | 34.25 | 295 | 239 |
| CAAGAGGtCCCCGGTTCGAATCTAGGTGCCCCCT | | | | |
| GGGGATATAGCTCAGGGGTAGAGCATTTGACTctaGAT | TAG | 24.61 | 327 | 240 |
| CAAGAGGtCCCCGGTTCAAATCCGGGTGCCCCCC | | | | |
| GGGGGTATAGTTCAGGGGTAGAGCATTTGACTctaGAT | TAG | 24.05 | 329 | 241 |
| CAAGAGGtCCCTGGTTCAAATCCAGGTGCCCCCT | | | | |
| GGGGGTATAGCTCACAGGTAGAGCATTTGACTctaGAT | TAG | 21.95 | 338 | 242 |
| CAAGAGGtCCCCGGTTCAAATCCGGTTACTCCCT | | | | |
| GGGGGTATAGCTCAGGGGTAGAGCATTTGACTctaAAT | TAG | 20.79 | 341 | 243 |
| CAAGAGGtCCCTGATTCAAATCCAGGTGCCCCCT | | | | |
| GGGCGTATAGCTCAGGGGTAGAGCATTTGACTctaGAT | TAG | 20.75 | 342 | 244 |
| CAAGAGGtCCCCAGTTCAAATCTGGGTGCCCA | | | | |
| GGGGGTATAGCTCACAGGTAGAGCATTTGACTctaGAT | TAG | 19.81 | 345 | 245 |
| CAAGAGGtCCCCGGTTCAAATCTGGGTGCCCCCT | | | | |
| GGGCGTATAGCTCAGGGGTAGAGCATTTGACTctaGAT | TAG | 18.74 | 349 | 246 |
| CAAGAGGtCCCCAGTTCAAATCTGGGTGCCCCCT | | | | |
| GGTATATCTCAGGGGGCAGAGCATTTGACTctaGATCA | TAG | 15.92 | 364 | 247 |
| AGAGGtCCCCGGTTGAAATCCGGGTGCT | | | | |
| GGCTGTATAGCTCAGTGGTAGAGCATTTGACTctaGCT | TAG | 4.20 | 520 | 248 |
| TTAAAGTCATAtGTAGCTGGGTTCAAATCCTGTTTCTG | | | | |
| CCA | | | | |
| GGGGGTATAGCTTAGGGGTAGAGCATTTGACTctaGAT | TAG | 3.46 | 558 | 249 |
| CAAAAGGtCCCTGGTTCAAATCCAGGTGCCCCTT | | | | |
| GGCAGAATGGTGCAGCGGTtcAGCACCCAGgCTCTctaG | TAG | 2.31 | 661 | 250 |
| cCAGCTGTTGCCTGGGCTCAAATCCCAGCTCTGCCA | | | | |

TABLE 19C
Cysteine (Cys) tRNA

| | | fold | | SEQ |
|---|---|---|---|---|
| seq_tRNA | ACE | over | Rank | ID |
| GGGGGTATAGCTCAGTGGGTAGAGCATTTGACTtcaGA | TGA | 70.51 | 238 | 251 |
| TCAAGAGGtCCCCGGTTCAAATCCGGGTGCCCCCT | | | | |
| GGGGGTATAGCTTAGCGGTAGAGCATTTGACTtcaGAT | TGA | 47.96 | 265 | 252 |
| CAAGAGGtCCCCGGTTCAAATCCGGGTGCCCCCT | | | | |
| GGGGGTATAGCTCAGGGGTAGAGCATTTGACTtcaGAT | TGA | 46.07 | 267 | 253 |
| CAAGAGGtCCCCGGTTCAAATCCGGGTGCCCCCT | | | | |
| GGGGGTATAGCTCAGGGGTAGAGCATTTGACTtcaGAT | TGA | 45.81 | 269 | 254 |
| CAAGAGGtCCCCAGTTCAAATCTGGGTGCCCCCT | | | | |
| GGGGGTATAGCTCAGTGGTAGAGCATTTGACTtcaGAT | TGA | 45.64 | 270 | 255 |
| CAAGAGGtCCCTGGTTCAAATCCGGGTGCCCCCT | | | | |
| GGGGGTATAGCTCAGGGGTAGAGCATTTGACTtcaGAT | TGA | 41.28 | 278 | 256 |
| CAAGAGGCTCTGGTTCAAATCCAGGTGCCCCCT | | | | |
| GGGGGCATAGCTCAGTGGTAGAGCATTTGACTTCAGA | TGA | 32.06 | 304 | 257 |
| TCAAGAGGtCCCTGGTTCAAATCCAGGTGCCCCCT | | | | |
| GGGGGTATAGCTCAGGGGTAGAGCATTTGACTtcaGAT | TGA | 29.89 | 311 | 258 |
| CAAGAGGtCCCTGGTTCAAATCCAGGTGCCCCCT | | | | |
| GGGGGTGTAGCTCAGTGGTAGAGCATTTGACTtcaGAT | TGA | 16.37 | 361 | 259 |
| CAAGAGGtCCCTGGTTCAAATCCAGGTGCCCCCT | | | | |
| GGGGGTATAGCTTAGGGGTAGAGCATTTGACTtcaGAT | TGA | 13.82 | 368 | 260 |
| CAAAAGGtCCCTGGTTCAAATCCAGGTGCCCCTT | | | | |
| GGGGGTATAGCTCAGGGGTAGAGCACTTGACTtcaGAT | TGA | 7.33 | 443 | 261 |
| CAAGAGGtCCCTGGTTCAAATCCAGGTGCCCCCT | | | | |
| GGGGGTATAGCTCAGGGGTAGAGCATTTGACTtcaGAT | TGA | 6.52 | 456 | 262 |
| CAAGAAGtCCCCGGTTCAAATCCGGGTGCCCCCT | | | | |
| GGGGGTATAGCTCAGGGGTAGAGCATTTGACTtcaGAT | TGA | 6.06 | 464 | 263 |
| CAAGAGGtCCCTGGTTCAAATCCAGGTGCCCCCC | | | | |
| GGGGGTATAGCTCAGGGGTAGAGCACTTGACTtcaGAT | TGA | 4.61 | 505 | 264 |
| CAAGAAGtCCTTGGTTCAAATCCAGGTGCCCCCT | | | | |
| GGTATATCTCAGGGGGcAGAGCATTTGACTtcaGATCA | TGA | 4.40 | 512 | 265 |
| AGAGGtCCCCGGTTGAAATCCGGGTGCT | | | | |
| GGCTGTATAGCTCAGTGGTAGAGCATTTGACTtcaGCT | TGA | 4.39 | 513 | 266 |
| TTAAAGTCATAtGTAGCTGGGTTCAAATCCTGTTTCTG | | | | |
| CCA | | | | |
| GGCAGAATGGTGCAGCGGTtcAGCACCCAGgCTCTtcaG | TGA | 3.18 | 578 | 267 |
| cCAGCTGTTGCCTGGGCTCAAATCCCAGCTCTGCCA | | | | |
| GGGGGTATAGCTCACAGGTAGAGCATTTGACTtcaGAT | TGA | 2.70 | 616 | 268 |
| CAAGAGGtCCCCGGTTCAAATCCGGTTACTCCCT | | | | |
| GGGGATATAGCTCAGGGGTAGAGCATTTGACTtcaGAT | TGA | 2.62 | 619 | 269 |
| CAAGAGGtCCCCGGTTCAAATCCGGGTGCCCCCC | | | | |
| GGGGGTATAGCTCACAGGTAGAGCATTTGACTtcaGAT | TGA | 2.50 | 631 | 270 |
| CAAGAGGtCCCCGGTTCAAATCTGGGTGCCCCCT | | | | |
| GGGCGTATAGCTCAGGGGTAGAGCATTTGACTtcaGAT | TGA | 2.06 | 703 | 271 |
| CAAGAGGtCCCCAGTTCAAATCTGGGTGCCCA | | | | |
| GGGCGTATAGCTCAGGGGTAGAGCATTTGACTtcaGAT | TGA | 1.71 | 758 | 272 |
| CAAGAGGtCCCCAGTTCAAATCTGGGTGCCCCCT | | | | |
| GGGGGTAGGGCTCAGGGAtAGAGCATTTGACTtcaGAT | TGA | 1.61 | 772 | 273 |
| CAAGAGGtCCCCGGTTCGAATCTAGGTGCCCCCT | | | | |
| GGGGGTATAGCTCAGGGGTAGAGCATTTGACTtcaAAT | TGA | 1.06 | 854 | 274 |
| CAAGAGGTCCCTGATTCAAATCCAGGTGCCCCCT | | | | |
| GGGGGTATAGTTCAGGGGTAGAGCATTTGACTtcaGAT | TGA | 0.12 | 910 | 275 |
| CAAGAGGtCCCTGGTTCAAATCCAGGTGCCCCCT | | | | |

TABLE 20A
Glutamine (Gln) tRNA

| | | fold | | SEQ |
|---|---|---|---|---|
| seq_tRNA | ACE | over | Rank | ID |
| GGTCCCATGGTGTAATGGTtAGCACTCTGGACTttaAAT | TAA | 5001.82 | 14 | 276 |
| CCAGCGaTCCGAGTTCAAATCTCGGTGGGACCT | | | | |
| GGTTCCATGGTGTAATGGTtAGCACTCTGGACTttaAAT | TAA | 4422.37 | 19 | 277 |
| CCAGCGaTCCGAGTTCAAATCTCGGTGGAACCT | | | | |
| GGCCCCATGGTGTAATGGTtAGCACTCTGGACTttaAAT | TAA | 4185.35 | 22 | 278 |
| CCAGCGaTCCGAGTTCAAATCTCGGTGGGACCT | | | | |
| GGCCCCATGGTGTAATGGTcAGCACTCTGGACTttaAAT | TAA | 4059.89 | 25 | 279 |
| CCAGCGaTCCGAGTTCAAATCTCGGTGGGACCC | | | | |
| GGTTCCATGGTGTAATGGTaAGCACTCTGGACTttaAAT | TAA | 3519.57 | 33 | 280 |
| CCAGCGaTCCGAGTTCGAGTCTCGGTGGAACCT | | | | |
| GGTCCCATGGTGTAATGGTtAGCACTCTGGACTttaAAT | TAA | 3114.14 | 37 | 281 |
| CCAGCAaTCCGAGTTCGAATCTCGGTGGGACCT | | | | |
| GGTTCCATGGTGTAATGGTtAGCACTCTGGACTttaAAT | TAA | 2143.94 | 58 | 282 |
| CCGGTAaTCCGAGTTCAAATCTCGGTGGAACCT | | | | |
| GGTTCCATGGTGTAATGGTgAGCACTCTGGACTttaAAT | TAA | 1622.00 | 66 | 283 |
| CCAGCGaTCCGAGTTCGAGTCTCGGTGGAACCT | | | | |
| GGTTCCATGGTGTAATGGTtAGCACTCTGGACTttaAAT | TAA | 1286.02 | 78 | 284 |
| CCAGCGaTCCGAGTTCAAGTCTCGGTGGAACCT | | | | |
| GGTCCCATGGTGTAATGGTtAGCACTCTGGGCTttaAAT | TAA | 504.01 | 128 | 285 |
| CCAGCAaTCCGAGTTCGAATCTTGGTGGGACCT | | | | |
| GGTACAGTGTTAAAGGGGagaAAAATTGCTGACTttaAA | TAA | 4.16 | 522 | 286 |
| TaCAGTAGaCCTAGGTTTGAATCCTGGCTTTACCA | | | | |
| GGCTGTGTACCTCAGTGGGCAAGGGTATGGACTttaAA | TAA | 3.59 | 548 | 287 |
| GCCAGACTaTTTGGGTTCAAATCCCAGCTTGGCCT | | | | |
| GGTTCCATGGTGTAATGGTgAGGGCTTTGGACTttaACT | TAA | 2.99 | 593 | 288 |
| ACAGTGaTCAGAGTTCAAGTCTCAGTGGGACCT | | | | |
| GGTTCCATGGTGTAATGGTgACCACTTTGGACTttaAAT | TAA | 2.78 | 603 | 289 |
| ACAGTGATCAGAGTTCAAGTCTCACTGGAACCT | | | | |
| GGTTCCATGGTGTAATGGTaAGCACCCTGGACTttaAAT | TAA | 2.74 | 609 | 290 |
| CCAGCAaCCAGAGTTCCAGTCTCAGCGtGGACCT | | | | |
| GTTTCCATGGTGTAATGGTgAGCACTCTGGACTttaAAT | TAA | 2.74 | 610 | 291 |
| CCAGAAATACATTCAAAGAATTAAGAACA | | | | |
| GGTTCCATGGTGTAATGGTgAGCACTTTGGACTttaAAT | TAA | 1.90 | 724 | 292 |
| ACAGTGATCAGAGTTCAAGTCTCACTGGGACCT | | | | |
| GGTTCCATGgGTTAATGGTgAGCACCCTGGACTttaAAT | TAA | 1.61 | 771 | 293 |
| CAAGCGaTCCGAGTTCAAATCTCGGTGGTACCT | | | | |
| GGTTCCATGGTGTAATGGTaAGCACTCTGGACTttaAAT | TAA | 1.32 | 825 | 294 |
| CCAGCCATCTGAGTTCGAGTCTCTGTGGAACCT | | | | |
| GGTAGTGTAGTCTACTGGTTAAACGCTTGGgCTttaACA | TAA | 1.19 | 840 | 295 |
| TTAAcGtCCTGGGTTCAAATCCCAGCTTTGTCA | | | | |
| GACCATGTGGCCTAAGGGAaAAGACATCTCACTttaGGT | TAA | 0.37 | 899 | 296 |
| CAGAAGALTGAGGGTTCAAGTCCTTTCATGGTCA | | | | |

TABLE 20B
Glutamine (Gln) tRNA

| | | fold | | SEQ |
|---|---|---|---|---|
| seq_tRNA | ACE | over | Rank | ID |
| GGTTCCATGGTGTAATGGTgAGCACTCTGGACTctaAAT | TAG | 4037.64 | 27 | 297 |
| CCAGCGaTCCGAGTTCGAGTCTCGGTGGAACCT | | | | |
| GGCCCCATGGTGTAATGGTcAGCACTCTGGACTctaAAT | TAG | 4034.34 | 28 | 298 |
| CCAGCGaTCCGAGTTCAAATCTCGGTGGGACCC | | | | |
| GGTTCCATGGTGTAATGGTaAGCACTCTGGACTctaAAT | TAG | 3876.76 | 29 | 299 |
| CCAGCGaTCCGAGTTCGAGTCTCGGTGGAACCT | | | | |
| GGTTCCATGGTGTAATGGTtAGCACTCTGGACTctaAAT | TAG | 3813.96 | 31 | 300 |
| CCGGTAaTCCGAGTTCAAATCTCGGTGGAACCT | | | | |
| GGTTCCATGGTGTAATGGTtAGCACTCTGGACTctaAAT | TAG | 3230.26 | 35 | 301 |
| CCAGCGaTCCGAGTTCAAGTCTCGGTGGAACCT | | | | |
| GGCCCCATGGTGTAATGGTtAGCACTCTGGACTctaAAT | TAG | 3153.49 | 36 | 302 |
| CCAGCGaTCCGAGTTCAAATCTCGGTGGGACCT | | | | |
| GGTCCCATGGTGTAATGGTtAGCACTCTGGACTctaAAT | TAG | 3084.06 | 38 | 303 |
| CCAGCAaTCCGAGTTCGAATCTCGGTGGGACCT | | | | |
| GGTCCCATGGTGTAATGGTtAGCACTCTGGACTctaAAT | TAG | 2084.16 | 59 | 304 |
| CCAGCGaTCCGAGTTCAAATCTCGGTGGGACCT | | | | |
| GGTTCCATGGTGTAATGGTtAGCACTCTGGACTctaAAT | TAG | 1940.85 | 60 | 305 |
| CCAGCGaTCCGAGTTCAAATCTCGGTGGAACCT | | | | |
| GGTCCCATGGTGTAATGGTtAGCACTCTGGGCTctaAAT | TAG | 409.72 | 136 | 306 |
| CCAGCAaTCCGAGTTCGAATCTTGGTGGGACCT | | | | |
| GGTTCCATGGTGTAATGGTgAGCACTTTGGACTctaAAT | TAG | 16.66 | 359 | 307 |
| ACAGTGATCAGAGTTCAAGTCTCACTGGGACCT | | | | |
| GGTTCCATGGTGTAATGGTgACCACTTTGGACTctaAAT | TAG | 13.03 | 372 | 308 |
| ACAGTGATCAGAGTTCAAGTCTCACTGGAACCT | | | | |
| GGTTCCATGGTGTAATGGTaAGCACTCTGGACTctaAAT | TAG | 12.33 | 379 | 309 |
| CCAGCCATCTGAGTTCGAGTCTCTGTGGAACCT | | | | |
| GGTTCCATGGTGTAATGGTaAGCACCCTGGACTctaAAT | TAG | 11.71 | 387 | 310 |
| CCAGCAaCCAGAGTTCCAGTCTCAGCGtGGACCT | | | | |
| GGTTCCATGGTGTAATGGTgAGGGCTTTGGACTctaACT | TAG | 10.92 | 395 | 311 |
| ACAGTGaTCAGAGTTCAAGTCTCAGTGGGACCT | | | | |
| GGTAGTGTAGTCTACTGGTTAAACGCTTGGgCTctaACA | TAG | 10.15 | 406 | 312 |
| TTAAcGtCCTGGGTTCAAATCCCAGCTTTGTCA | | | | |
| GGTTCCATGgGTTAATGGTgAGCACCCTGGACTctaAAT | TAG | 7.09 | 448 | 313 |
| CAAGCGaTCCGAGTTCAAATCTCGGTGGTACCT | | | | |
| GGCTGTGTACCTCAGTGGGCAAGGGTATGGACTctaAA | TAG | 5.13 | 485 | 314 |
| GCCAGACTaTTTGGGTTCAAATCCCAGCTTGGCCT | | | | |
| GGTACAGTGTTAAAGGGGagaAAAATTGCTGACTctaAA | TAG | 4.80 | 499 | 315 |
| TaCAGTAGaCCTAGGTTTGAATCCTGGCTTTACCA | | | | |
| GTTTCCATGGTGTAATGGTgAGCACTCTGGACTctaAAT | TAG | 4.56 | 507 | 316 |
| CCAGAAATACATTCAAAGAATTAAGAACA | | | | |
| GACCATGTGGCCTAAGGGAaAAGACATCTCACTctaGG | TAG | 3.77 | 540 | 307 |
| TCAGAAGAtTGAGGGTTCAAGTCCTTTCATGGTCA | | | | |

TABLE 20C
Glutamine (Gln) tRNA

| | | fold | | SEQ |
|---|---|---|---|---|
| seq_tRNA | ACE | over | Rank | ID |
| GGCCCCATGGTGTAATGGTtAGCACTCTGGACTtca | TGA | 252.02 | 165 | 318 |
| AATCCAGCGaTCCGAGTTCAAATCTCGGTGGGACCT | | | | |
| GGTTCCATGGTGTAATGGTaAGCACTCTGGACTtcaAAT | TGA | 198.72 | 179 | 319 |
| CCAGCGaTCCGAGTTCGAGTCTCGGTGGAACCT | | | | |
| GGTCCCATGGTGTAATGGTtAGCACTCTGGACTtcaAAT | TGA | 184.51 | 184 | 320 |
| CCAGCAaTCCGAGTTCGAATCTCGGTGGGACCT | | | | |
| GGTCCCATGGTGTAATGGTtAGCACTCTGGACTtcaAAT | TGA | 162.80 | 188 | 321 |
| CCAGCGaTCCGAGTTCAAATCTCGGTGGGACCT | | | | |
| GGTTCCATGGTGTAATGGTgAGCACTCTGGACTtcaAAT | TGA | 119.19 | 204 | 322 |
| CCAGCGaTCCGAGTTCGAGTCTCGGTGGAACCT | | | | |
| GGTTCCATGGTGTAATGGTtAGCACTCTGGACTtcaAAT | TGA | 93.48 | 223 | 323 |
| CCGGTAaTCCGAGTTCAAATCTCGGTGGAACCT | | | | |
| GGCCCCATGGTGTAATGGTcAGCACTCTGGACTtcaAAT | TGA | 85.36 | 231 | 324 |
| CCAGCGaTCCGAGTTCAAATCTCGGTGGGACCC | | | | |
| GGTTCCATGGTGTAATGGTtAGCACTCTGGACTtcaAAT | TGA | 84.76 | 233 | 325 |
| CCAGCGaTCCGAGTTCAAATCTCGGTGGAACCT | | | | |
| GGTCCCATGGTGTAATGGTtAGCACTCTGGGCTtcaAAT | TGA | 17.34 | 357 | 326 |
| CCAGCAaTCCGAGTTCGAATCTTGGTGGGACCT | | | | |
| GACCATGTGGCCTAAGGGAaAAGACATCTCACTtcaGG | TGA | 9.89 | 410 | 327 |
| TCAGAAGAtTGAGGGTTCAAGTCCTTTCATGGTCA | | | | |
| GGTTCCATGGTGTAATGGTtAGCACTCTGGACTtcaAAT | TGA | 5.01 | 491 | 328 |
| CCAGCGaTCCGAGTTCAAGTCTCGGTGGAACCT | | | | |
| GGTTCCATGGTGTAATGGTgAGCACTTTGGACTtcaAAT | TGA | 4.53 | 509 | 329 |
| ACAGTGATCAGAGTTCAAGTCTCACTGGGACCT | | | | |
| GGTACAGTGTTAAAGGGGagaAAAATTGCTGACTtcaAA | TGA | 4.53 | 510 | 330 |
| TaCAGTAGaCCTAGGTTTGAATCCTGGCTTTACCA | | | | |
| GGCTGTGTACCTCAGTGGGCAAGGGTATGGACTtcaAA | TGA | 3.83 | 537 | 331 |
| GCCAGACTaTTTGGGTTCAAATCCCAGCTTGGCCT | | | | |
| GGTTCCATGGTGTAATGGTaAGCACTCTGGACTtcaAAT | TGA | 3.64 | 546 | 332 |
| CCAGCCATCTGAGTTCGAGTCTCTGTGGAACCT | | | | |
| GGTTCCATGgGTTAATGGTgAGCACCCTGGACTtcaAAT | TGA | 3.46 | 559 | 333 |
| CAAGCGaTCCGAGTTCAAATCTCGGTGGTACCT | | | | |
| GGTTCCATGGTGTAATGGTgAGGGCTTTGGACTtcaACT | TGA | 3.39 | 564 | 334 |
| ACAGTGaTCAGAGTTCAAGTCTCAGTGGGACCT | | | | |
| GTTTCCATGGTGTAATGGTgAGCACTCTGGACTtcaAAT | TGA | 3.12 | 582 | 335 |
| CCAGAAATACATTCAAAGAATTAAGAACA | | | | |
| GGTTCCATGGTGTAATGGTaAGCACCCTGGACTtcaAAT | TGA | 2.96 | 596 | 336 |
| CCAGCAaCCAGAGTTCCAGTCTCAGCGtGGACCT | | | | |
| GGTTCCATGGTGTAATGGTgACCACTTTGGACTtcaAAT | TGA | 2.19 | 675 | 337 |
| ACAGTGATCAGAGTTCAAGTCTCACTGGAACCT | | | | |
| GGTAGTGTAGTCTACTGGTTAAACGCTTGGgCTtcaACA | TGA | 0.70 | 884 | 338 |
| TTAAcGtCCTGGGTTCAAATCCCAGCTTTGTCA | | | | |

TABLE 21A
Glutamate (Glu) tRNAs

| | | fold | | SEQ |
|---|---|---|---|---|
| seq_tRNA | ACE | over | Rank | ID |
| TCCCACATGGTCTAGCGGTtAGGATTCCTGGTTttaACC | TAA | 241.76 | 167 | 339 |
| CAGGCGGCCCGGGTTCGACTCCCGGTGTGGGAA | | | | |
| TCCCATATGGTCTAGCGGTtAGGATTCCTGGTTttaACC | TAA | 189.58 | 182 | 340 |
| CAGGTGGCCCGGGTTCGACTCCCGGTATGGGAA | | | | |
| TCCCTGGTGGTCTAGTGGCtAGGATTCGGCGCTttaACC | TAA | 58.21 | 250 | 341 |
| GCCGCGGCCCGGGTTCGATTCCCGGTCAGGGAA | | | | |
| TCCCTGGTGGTCTAGTGGTtAGGATTCGGCGCTttaACC | TAA | 57.73 | 251 | 342 |
| GCCGCGGCCCGGGTTCGATTCCCGGTCAGGGAA | | | | |
| TCCCTGGTGGTCTAGTGGCtAGGATTCGGCGCTttaACC | TAA | 44.49 | 273 | 343 |
| GCCGCGGCCCGGGTTCGATTCCCGGCCAGGGAA | | | | |
| TCCTTGATGTCTAGTGGTtAGGATTTGGTGCTttaACTGC | TAA | 2.73 | 611 | 344 |
| AGCAGCCTGGGTTCATTTCTCAGTCAGGGAA | | | | |
| TCCTTGGTGGTCTAGTGGCtAGGATTCGGTGCTttaACC | TAA | 2.48 | 633 | 345 |
| TGTGCGGCCCGGGTTCAATTCCCGATGAAGGAA | | | | |
| TCCCTGGTGGTCTAGTGGCtAGGATTCGGCGCTttaACC | TAA | 2.34 | 657 | 346 |
| GCCTGCAGCTCGAGTTCGATTCCTGGTCAGGGAA | | | | |
| GCGTTGGTGGTGTAGTGGTgAGCACAGCTGCCTttaAA | TAA | 2.25 | 668 | 347 |
| GCAGTTAaCGCGGGTTCGATTCCCGGGTAACGAA | | | | |
| TCCCTGGTGGTCTAGTGGTAGGATTCGGCGCTttaACC | TAA | 2.16 | 682 | 348 |
| GCCGCGGCCCGGGTTCGATTCCCGGTCAGGAAA | | | | |
| GCAATGGTGGTTCAGTGGTAGAATTCTCGCCTttaACA | TAA | 2.07 | 695 | 349 |
| CAGGAGaCCCGGGTTCAATTCCTGACCCATGTA | | | | |
| TGTCTGGTGGTCAAGTGGCtAGGATTTGGCGCTttaACT | TAA | 2.06 | 700 | 350 |
| GCCGCGGCCCGCGTTCGATTCCCGGTCAGGGAA | | | | |
| CCCTGTGGTCTAGTGGCtAAGACTTTGTGCTttaATTGCT | TAA | 1.76 | 749 | 351 |
| GCAtCCTAGGTTCAATTCCCAGTCAGGGA | | | | |
| CCCCTGGTGGTCTAGTGCTtAGGATTCGGTGCTttaACC | TAA | 1.53 | 786 | 352 |
| GCTGCTGCCTGCGTTCGATTCCCGGTCAGGGAA | | | | |
| TCCGTGGTGGTCTAGTGGCtAGGATTCGGCGCTttaACC | TAA | 1.13 | 847 | 353 |
| GCCTGCAGCTCGAGTTCGATTCCTGGTCAGGGAA | | | | |

TABLE 21B
Glutamate (Glu) tRNAs

| | | fold | | SEQ |
|---|---|---|---|---|
| seq_tRNA | ACE | over | Rank | ID |
| TCCGTGGTGGTCTAGTGGCtAGGATTCGGCGCTcta | TAG | 1659.61 | 64 | 354 |
| ACCGCCTGCAGCTCGAGTTCGATTCCTGGTCAGG | | | | |
| GAA | | | | |
| TCCCACATGGTCTAGCGGTtAGGATTCCTGGTTctaACC | TAG | 687.03 | 104 | 355 |
| CAGGCGGCCCGGGTTCGACTCCCGGTGTGGGAA | | | | |
| TCCCTGGTGGTCTAGTGGCtAGGATTCGGCGCTctaACC | TAG | 535.33 | 124 | 356 |
| GCCGCGGCCCGGGTTCGATTCCCGGTCAGGGAA | | | | |
| TCCCTGGTGGTCTAGTGGTtAGGATTCGGCGCTctaACC | TAG | 489.16 | 129 | 357 |
| GCCGCGGCCCGGGTTCGATTCCCGGTCAGGGAA | | | | |
| TCCCTGGTGGTCTAGTGGCtAGGATTCGGCGCTctaACC | TAG | 453.68 | 131 | 358 |
| GCCGCGGCCCGGGTTCGATTCCCGGCCAGGGAA | | | | |
| TCCCATATGGTCTAGCGGTtAGGATTCCTGGTTctaACC | TAG | 259.90 | 163 | 359 |
| CAGGTGGCCCGGGTTCGACTCCCGGTATGGGAA | | | | |
| GCGTTGGTGGTGTAGTGGTgAGCACAGCTGCCTctaAA | TAG | 12.31 | 380 | 360 |
| GCAGTTAaCGCGGGTTCGATTCCCGGGTAACGAA | | | | |
| TCCCTGGTGGTCTAGTGGTtAGGATTCGGCGCTctaACC | TAG | 11.86 | 384 | 361 |
| GCCGCGGCCCGGGTTCGATTCCCGGTCAGGAAA | | | | |
| CCCTGTGGTCTAGTGGCtAAGACTTTGTGCTctaATTGC | TAG | 11.81 | 386 | 362 |
| TGCAtCCTAGGTTCAATTCCCAGTCAGGGA | | | | |
| TCCTTGATGTCTAGTGGTtAGGATTTGGTGCTctaACTG | TAG | 8.58 | 422 | 363 |
| CAGCAGCCTGGGTTCATTTCTCAGTCAGGGAA | | | | |
| GCAATGGTGGTTCAGTGGTAGAATTCTCGCCTctaACA | TAG | 6.29 | 461 | 364 |
| CAGGAGaCCCGGGTTCAATTCCTGACCCATGTA | | | | |
| CCCCTGGTGGTCTAGTGCTtAGGATTCGGTGCTctaACC | TAG | 5.54 | 476 | 365 |
| GCTGCTGCCTGCGTTCGATTCCCGGTCAGGGAA | | | | |
| TCCTTGGTGGTCTAGTGGCtAGGATTCGGTGCTctaACC | TAG | 5.49 | 478 | 366 |
| TGTGCGGCCCGGGTTCAATTCCCGATGAAGGAA | | | | |
| TCCCTGGTGGTCTAGTGGCtAGGATTCGGCGCTctaACC | TAG | 4.32 | 517 | 367 |
| GCCTGCAGCTCGAGTTCGATTCCTGGTCAGGGAA | | | | |
| TGTCTGGTGGTCAAGTGGCtAGGATTTGGCGCTctaACT | TAG | 4.20 | 519 | 368 |
| GCCGCGGCCCGCGTTCGATTCCCGGTCAGGGAA | | | | |

TABLE 21C
Glutamate (Glu) tRNAs

| | | fold | | SEQ |
|---|---|---|---|---|
| seq_tRNA | ACE | over | Rank | ID |
| TCCCATATGGTCTAGCGGTtAGGATTCCTGGTTtcaACC | TGA | 38.81 | 286 | 369 |
| CAGGTGGCCCGGGTTCGACTCCCGGTATGGGAA | | | | |
| TCCCACATGGTCTAGCGGTtAGGATTCCTGGTTtcaACC | TGA | 32.64 | 301 | 370 |
| CAGGCGGCCCGGGTTCGACTCCCGGTGTGGGAA | | | | |
| TGTCTGGTGGTCAAGTGGCtAGGATTTGGCGCTtcaACT | TGA | 3.54 | 552 | 371 |
| GCCGCGGCCCGCGTTCGATTCCCGGTCAGGGAA | | | | |
| TCCTTGATGTCTAGTGGTtAGGATTTGGTGCTtcaACTG | TGA | 3.44 | 562 | 372 |
| CAGCAGCCTGGGTTCATTTCTCAGTCAGGGAA | | | | |
| GCAATGGTGGTTCAGTGGTAGAATTCTCGCCTtcaACA | TGA | 2.25 | 670 | 373 |
| CAGGAGaCCCGGGTTCAATTCCTGACCCATGTA | | | | |
| TCCGTGGTGGTCTAGTGGCtAGGATTCGGCGCTtcaACC | TGA | 2.00 | 713 | 374 |
| GCCTGCAGCTCGAGTTCGATTCCTGGTCAGGGAA | | | | |
| TCCCTGGTGGTCTAGTGGCtAGGATTCGGCGCTtcaACC | TGA | 1.87 | 727 | 375 |
| GCCGCGGCCCGGGTTCGATTCCCGGCCAGGGAA | | | | |
| TCCCTGGTGGTCTAGTGGTAGGATTCGGCGCTtcaACC | TGA | 1.51 | 792 | 376 |
| GCCGCGGCCCGGGTTCGATTCCCGGTCAGGAAA | | | | |
| TCCTTGGTGGTCTAGTGGCtAGGATTCGGTGCTtcaACC | TGA | 1.43 | 811 | 377 |
| TGTGCGGCCCGGGTTCAATTCCCGATGAAGGAA | | | | |
| TCCCTGGTGGTCTAGTGGCtAGGATTCGGCGCTtcaACC | TGA | 1.43 | 812 | 378 |
| GCCTGCAGCTCGAGTTCGATTCCTGGTCAGGGAA | | | | |
| CCCCTGGTGGTCTAGTGCTtAGGATTCGGTGCTtcaACC | TGA | 1.33 | 823 | 379 |
| GCTGCTGCCTGCGTTCGATTCCCGGTCAGGGAA | | | | |
| GCGTTGGTGGTGTAGTGGTgAGCACAGCTGCCTtcaAA | TGA | 1.32 | 826 | 380 |
| GCAGTTAaCGCGGGTTCGATTCCCGGGTAACGAA | | | | |
| TCCCTGGTGGTCTAGTGGCtAGGATTCGGCGCTtcaACC | TGA | 0.89 | 870 | 381 |
| GCCGCGGCCCGGGTTCGATTCCCGGTCAGGGAA | | | | |
| TCCCTGGTGGTCTAGTGGTtAGGATTCGGCGCTtcaACC | TGA | 0.23 | 904 | 382 |
| GCCGCGGCCCGGGTTCGATTCCCGGTCAGGGAA | | | | |
| CCCTGTGGTCTAGTGGCtAAGACTTTGTGCTtcaATTGC | TGA | 0.16 | 907 | 383 |
| TGCAtCCTAGGTTCAATTCCCAGTCAGGGA | | | | |

TABLE 22A
Glycine (Gly) tRNA

| | | fold | | SEQ |
|---|---|---|---|---|
| seq_tRNA | ACE | over | Rank | ID |
| GCGTTGGTGGTATAGTGGTtAGCATAGCTGCCTttaAAG | TAA | 23.13 | 333 | 384 |
| CAGTTGaCCCGGGTTCGATTCCCGGCCAACGCA | | | | |
| GCGTTGGTGGTATAGTGGTgAGCATAGCTGCCTttaAA | TAA | 18.56 | 350 | 385 |
| GCAGTTGaCCCGGGTTCGATTCCCGGCCAACGCA | | | | |
| GCGTTGGTGGTATAGTGGTaAGCATAGCTGCCTttaAAG | TAA | 17.47 | 356 | 386 |
| CAGTTGaCCCGGGTTCGATTCCCGGCCAACGCA | | | | |
| GCATTGGTGGTTCAGTGGTAGAATTCTCGCCTttaACGC | TAA | 2.60 | 621 | 387 |
| GGGAGaCCCGGGTTCAATTCCCGGCCAATGCA | | | | |
| GCCTTGGTGGTGCAGTGGTAGAATTCTCGCCTttaACGT | TAA | 2.39 | 647 | 378 |
| GGGAGaCCCGGGTTCAATTCCCGGCCAATGCA | | | | |
| GCGCCGCTGGTGTAGTGGTATCATGCAAGATTttaATT | TAA | 2.33 | 659 | 389 |
| CTTGCGaCCCGGGTTCGATTCCCGGGCGGCGCA | | | | |
| GCGTCAGTGGTTTAGTGGTGGAATTCCTGCCTttaATGC | TAA | 2.22 | 672 | 390 |
| ACGAGATCCGTGTTCAACTCCTGGTTGGTGCA | | | | |
| GCGTTGGTGGTATAGTGGTgAGCATAGTTGCCTttaAAG | TAA | 1.90 | 725 | 391 |
| CAGTTGaCCCGGGCTCGATTCCCGgCCAACGCA | | | | |
| GCATGGGTGATTCAGTGGTAGAATTTTCACCTttaATGC | TAA | 1.85 | 729 | 392 |
| AGGAGGTCCAGGTTCATTTCCTGGCCTATGCA | | | | |
| GCGTCAGTGgTTTTAGTGGTGGAATTCCTGCCTttaATG | TAA | 1.85 | 730 | 393 |
| CACGAGATCCGTGTTCAACTCCTGGTTGGTGCA | | | | |
| GCATTGGTGGTTCAGTGGTAGAATTCTCGCCTttaACGC | TAA | 1.78 | 747 | 394 |
| GGGAGGCCCGGGTTTGATTCCCGGCCAGTGCA | | | | |
| GTGGTCTAGTGGTTAGGATTCAGCGCTttaACCGCCGC | TAA | 1.75 | 751 | 385 |
| AGCCCGGGTTCGATTCCCGGtCA | | | | |
| GCGTTGGTGGTTTAGTGGTAGAATTCTCGCCTttaATGC | TAA | 1.59 | 774 | 396 |
| GGGAGaCCCGGGTTCAATTCCCGGCCACTGCA | | | | |
| GCATTGGTGGTTCAGTGGTAGAATTCTCGCCTttaATGC | TAA | 1.59 | 776 | 397 |
| GGGCGGCCGGGCTTCGATTCCTgGCCAATGCA | | | | |
| GCATAGGTGGTTCAGTGGTAGAATTCTTGCCTttaACG | TAA | 1.45 | 807 | 398 |
| CAGGAGGCCCAGGTTTGATTCCTGGCCCATGCA | | | | |
| GCGTTGGCAGTTCAGTGGTAGAATTCTCGCCTttaACCC | TAA | 1.42 | 813 | 399 |
| GGGAGaCCTGGATTCCATTTCCGGCAAATGCA | | | | |
| GCGTTGGTGGTATAGTGGTgAGCATAGTTGCCTttaAAG | TAA | 1.40 | 816 | 400 |
| CAGTTGaCCCGGGCTCGATTCCCGCCCAACGCA | | | | |
| GCATGGGTGGTTCAGTGGTAGAATTCTCGCCTttaACG | TAA | 0.96 | 865 | 40 |
| CGGGAGGCCCGGGTTCGATTCCCGGCCCATGCA | | | | |
| GCATTGGTGGTTCAGTGGTAGAATTCTCGCCTttaACGC | TAA | 0.88 | 872 | 402 |
| GGGAGGCCCGGGTTCGATTCCCGGCCAATGCA | | | | |
| GCATTGGTGGTTCAATGGTAGAATTCTCGCCTttaACGC | TAA | 0.46 | 895 | 403 |
| AGGAGaCCCAGGTTCGATTCCTGGCCAATGCA | | | | |
| GGTGGTTCAGTGGTAGAATTCTCGCCTttaACGCGGGA | TAA | 0.44 | 897 | 404 |
| GaCCCGGGTTTAATTCCCGGTCA | | | | |

TABLE 22B
Glycine (Gly) tRNA

| | | fold | | SEQ |
|---|---|---|---|---|
| seq_tRNA | ACE | over | Rank | ID |
| GCGTCAGTGGTTTAGTGGTGGAATTCCTGCCTctaA | TAG | 540.46 | 122 | 405 |
| TGCACGAGATCCGTGTTCAACTCCTGGTTGGTGCA | | | | |
| GCGTTGGTGGTATAGTGGTtAGCATAGCTGCCTctaAAG | TAG | 29.83 | 313 | 406 |
| CAGTTGaCCCGGGTTCGATTCCCGGCCAACGCA | | | | |
| GCGTTGGTGGTATAGTGGTgAGCATAGCTGCCTctaAA | TAG | 25.51 | 324 | 407 |
| GCAGTTGaCCCGGGTTCGATTCCCGGCCAACGCA | | | | |
| GCGTTGGTGGTATAGTGGTaAGCATAGCTGCCTctaAA | TAG | 19.01 | 348 | 408 |
| GCAGTTGaCCCGGGTTCGATTCCCGGCCAACGCA | | | | |
| GCATTGGTGGTTCAGTGGTAGAATTCTCGCCTctaACG | TAG | 9.34 | 414 | 409 |
| CGGGAGaCCCGGGTTCAATTCCCGGCCAATGCA | | | | |
| GCGCCGCTGGTGTAGTGGTATCATGCAAGATTctaATT | TAG | 7.41 | 440 | 410 |
| CTTGCGaCCCGGGTTCGATTCCCGGGCGGCGCA | | | | |
| GCATTGGTGGTTCAATGGTAGAATTCTCGCCTctaACG | TAG | 6.55 | 455 | 411 |
| CAGGAGaCCCAGGTTCGATTCCTGGCCAATGCA | | | | |
| GCGTTGGTGGTTTAGTGGTAGAATTCTCGCCTctaATG | TAG | 5.12 | 486 | 412 |
| CGGGAGaCCCGGGTTCAATTCCCGGCCACTGCA | | | | |
| GCCTTGGTGGTGCAGTGGTAGAATTCTCGCCTctaACG | TAG | 4.60 | 506 | 413 |
| TGGGAGaCCCGGGTTCAATTCCCGGCCAATGCA | | | | |
| GCATGGGTGGTTCAGTGGTAGAATTCTCGCCTctaACG | TAG | 4.37 | 515 | 414 |
| CGGGAGGCCCGGGTTCGATTCCCGGCCCATGCA | | | | |
| GCATTGGTGGTTCAGTGGTAGAATTCTCGCCTctaACG | TAG | 4.12 | 523 | 415 |
| CGGGAGGCCCGGGTTTGATTCCCGGCCAGTGCA | | | | |
| GCGTTGGTGGTATAGTGGTgAGCATAGTTGCCTctaAA | TAG | 4.09 | 524 | 416 |
| GCAGTTGaCCCGGGCTCGATTCCCGCCCAACGCA | | | | |
| GCATTGGTGGTTCAGTGGTAGAATTCTCGCCTctaACG | TAG | 3.98 | 530 | 417 |
| CGGGAGGCCCGGGTTCGATTCCCGGCCAATGCA | | | | |
| GCATGGGTGATTCAGTGGTAGAATTTTCACCTctaATG | TAG | 3.79 | 538 | 418 |
| CAGGAGGTCCAGGTTCATTTCCTGGCCTATGCA | | | | |
| GCGTTGGTGGTATAGTGGTgAGCATAGTTGCCTctaAA | TAG | 3.63 | 547 | 419 |
| GCAGTTGaCCCGGGCTCGATTCCCGgCCAACGCA | | | | |
| GTGGTCTAGTGGTTAGGATTCAGCGCTctaACCGCCGC | TAG | 3.59 | 549 | 420 |
| AGCCCGGGTTCGATTCCCGGtCA | | | | |
| GCATTGGTGGTTCAGTGGTAGAATTCTCGCCTctaATG | TAG | 2.47 | 636 | 421 |
| CGGGCGGCCGGGCTTCGATTCCTgGCCAATGCA | | | | |
| GCATAGGTGGTTCAGTGGTAGAATTCTTGCCTctaACG | TAG | 2.05 | 705 | 422 |
| CAGGAGGCCCAGGTTTGATTCCTGGCCCATGCA | | | | |
| GCGTTGGCAGTTCAGTGGTAGAATTCTCGCCTctaACC | TAG | 1.96 | 717 | 423 |
| CGGGAGaCCTGGATTCCATTTCCGGCAAATGCA | | | | |
| GGTGGTTCAGTGGTAGAATTCTCGCCTctaACGCGGGA | TAG | 1.45 | 804 | 424 |
| GaCCCGGGTTTAATTCCCGGTCA | | | | |
| GCGTCAGTGgTTTTAGTGGTGGAATTCCTGCCTctaATG | TAG | 1.05 | 857 | 425 |
| CACGAGATCCGTGTTCAACTCCTGGTTGGTGCA | | | | |

TABLE 22C
Glycine (Gly) tRNA

| | | fold | | SEQ |
|---|---|---|---|---|
| seq_tRNA | ACE | over | Rank | ID |
| GCGTTGGTGGTATAGTGGTgAGCATAGCTGCCTtcaAA | TGA | 165.10 | 187 | 426 |
| GCAGTTGaCCCGGGTTCGATTCCCGGCCAACGCA | | | | |
| GCGTTGGTGGTATAGTGGTaAGCATAGCTGCCTtcaAA | TGA | 85.00 | 232 | 427 |
| GCAGTTGaCCCGGGTTCGATTCCCGGCCAACGCA | | | | |
| GCGTTGGCAGTTCAGTGGTAGAATTCTCGCCTtcaACC | TGA | 3.88 | 535 | 428 |
| CGGGAGaCCTGGATTCCATTTCCGGCAAATGCA | | | | |
| GTGGTCTAGTGGTTAGGATTCAGCGCTtcaACCGCCGC | TGA | 3.49 | 556 | 429 |
| AGCCCGGGTTCGATTCCCGGICA | | | | |
| GCGTTGGTGGTATAGTGGTgAGCATAGTTGCCTtcaAA | TGA | 3.40 | 563 | 430 |
| GCAGTTGaCCCGGGCTCGATTCCCGCCCAACGCA | | | | |
| GCCTTGGTGGTGCAGTGGTAGAATTCTCGCCTtcaACG | TGA | 3.24 | 573 | 431 |
| TGGGAGaCCCGGGTTCAATTCCCGGCCAATGCA | | | | |
| GCATTGGTGGTTCAATGGTAGAATTCTCGCCTtcaACG | TGA | 3.05 | 587 | 432 |
| CAGGAGaCCCAGGTTCGATTCCTGGCCAATGCA | | | | |
| GCGCCGCTGGTGTAGTGGTATCATGCAAGATTtcaATT | TGA | 3.04 | 589 | 433 |
| CTTGCGaCCCGGGTTCGATTCCCGGGCGGCGCA | | | | |
| GCATAGGTGGTTCAGTGGTAGAATTCTTGCCTtcaACG | TGA | 2.97 | 595 | 434 |
| CAGGAGGCCCAGGTTTGATTCCTGGCCCATGCA | | | | |
| GCATGGGTGATTCAGTGGTAGAATTTTCACCTtcaATG | TGA | 2.78 | 604 | 435 |
| CAGGAGGTCCAGGTTCATTTCCTGGCCTATGCA | | | | |
| GCATTGGTGGTTCAGTGGTAGAATTCTCGCCTtcaACG | TGA | 2.48 | 634 | 436 |
| CGGGAGGCCCGGGTTTGATTCCCGGCCAGTGCA | | | | |
| GGTGGTTCAGTGGTAGAATTCTCGCCTtcaACGCGGGA | TGA | 2.35 | 653 | 437 |
| GaCCCGGGTTTAATTCCCGGTCA | | | | |
| GCATTGGTGGTTCAGTGGTAGAATTCTCGCCTtcaATG | TGA | 2.28 | 665 | 438 |
| CGGGCGGCCGGGCTTCGATTCCTgGCCAATGCA | | | | |
| GCGTCAGTGGTTTAGTGGTGGAATTCCTGCCTtcaATG | TGA | 1.92 | 721 | 439 |
| CACGAGATCCGTGTTCAACTCCTGGTTGGTGCA | | | | |
| GCGTCAGTGgTTTTAGTGGTGGAATTCCTGCCTtcaATG | TGA | 1.92 | 722 | 440 |
| CACGAGATCCGTGTTCAACTCCTGGTTGGTGCA | | | | |
| GCGTTGGTGGTATAGTGGTtAGCATAGCTGCCTtcaAAG | TGA | 1.90 | 724 | 441 |
| CAGTTGaCCCGGGTTCGATTCCCGGCCAACGCA | | | | |
| GCGTTGGTGGTATAGTGGTgAGCATAGTTGCCTtcaAA | TGA | 1.80 | 741 | 442 |
| GCAGTTGaCCCGGGCTCGATTCCCGgCCAACGCA | | | | |
| GCATTGGTGGTTCAGTGGTAGAATTCTCGCCTtcaACG | TGA | 1.75 | 752 | 443 |
| CGGGAGaCCCGGGTTCAATTCCCGGCCAATGCA | | | | |
| GCATTGGTGGTTCAGTGGTAGAATTCTCGCCTtcaACG | TGA | 1.46 | 800 | 444 |
| CGGGAGGCCCGGGTTCGATTCCCGGCCAATGCA | | | | |
| GCGTTGGTGGTTTAGTGGTAGAATTCTCGCCTtcaATG | TGA | 1.22 | 835 | 445 |
| CGGGAGaCCCGGGTTCAATTCCCGGCCACTGCA | | | | |
| GCATGGGTGGTTCAGTGGTAGAATTCTCGCCTtcaACG | TGA | 0.58 | 892 | 446 |
| CGGGAGGCCCGGGTTCGATTCCCGGCCCATGCA | | | | |

TABLE 23A
Histidine (His) tRNA

| | | fold | | SEQ |
|---|---|---|---|---|
| seq_tRNA | ACE | over | Rank | ID |
| GCCATGATCGTATAGTGGT | TAA | 0.98 | 864 | 447 |
| tAGTACTCTGCGCTttaGC | | | | |
| CGCAGCAaCCTCGGTTCGA | | | | |
| ATCCGAGTCACGGCA | | | | |
| GCCGTGATCGTATAGTGGT | TAA | 0.70 | 885 | 448 |
| tAGTACTCTGCGTTttaGC | | | | |
| CGCAGCAaCCTCGGTTCGA | | | | |
| ATCCGAGTCACGGCA | | | | |

TABLE 23B
Histidine (His) tRNA

| | | fold | | SEQ |
|---|---|---|---|---|
| seq_tRNA | ACE | over | Rank | ID |
| GCCATGATCGTATAGTGGT | TAG | 1.00 | 862 | 449 |
| tAGTACTCTGCGCTctaGC | | | | |
| CGCAGCAaCCTCGGTTCGA | | | | |
| ATCCGAGTCACGGCA | | | | |
| GCCGTGATCGTATAGTGGT | TAG | 0.53 | 893 | 450 |
| tAGTACTCTGCGTTctaGC | | | | |
| CGCAGCAaCCTCGGTTCGA | | | | |
| ATCCGAGTCACGGCA | | | | |

TABLE 23C
Histidine (His) tRNA

| | | fold | | SEQ |
|---|---|---|---|---|
| seq_tRNA | ACE | over | Rank | ID |
| GCCATGATCGTATAGTGGT | TGA | 2.14 | 687 | 451 |
| tAGTACTCTGCGCTtcaGC | | | | |
| CGCAGCAaCCTCGGTTCGA | | | | |
| ATCCGAGTCACGGCA | | | | |
| GCCGTGATCGTATAGTGGT | TGA | 1.20 | 837 | 452 |
| tAGTACTCTGCGTTtcaGC | | | | |
| CGCAGCAaCCTCGGTTCGA | | | | |
| ATCCGAGTCACGGCA | | | | |

TABLE 24A
Isoleucine (Ile) tRNA

| | | fold | | SEQ |
|---|---|---|---|---|
| seq_tRNA | ACE | over | Rank | ID |
| GGCCGGTTAGCTCAGTTGGTtAGAGCGTGGTGCTt | TAA | 676.03 | 106 | 9 |
| taAACGCTAAGGCGCGGGTTCGATCCCCGTACTG | | | | |
| GCCA | | | | |
| GGCCGGTTAGCTCAGTTGGTtAGAGCGTGGTGCTt | TAA | 392.98 | 141 | 28 |
| taAACGCCAAGGtCGCGGGTTCGATCCCCGTACTG | | | | |
| GCCA | | | | |
| GGCCGGTTAGCTCAGTTGGTtAGAGCGTGGTGCTt | TAA | 384.97 | 143 | 453 |
| taAACGCCAAGGtCGCGGGTTCGATCCCCGTACGG | | | | |
| GCCA | | | | |
| GGCCGGTTAGCTCAGTTGGTtAGAGCGTGGCGCTt | TAA | 364.35 | 148 | 21 |
| taAACGCCAAGGtCGCGGGTTCGATCCCCGTACGG | | | | |
| GCCA | | | | |
| GGCCGGTTAGCTCAGTTGGTcAGAGCGTGGTGCTt | TAA | 275.74 | 158 | 454 |
| taAACGCCAAGGtCGCGGGTTCGATCCCCGTACGG | | | | |
| GCCA | | | | |
| GGCCGGTTAGCTCAGTCGGCtAGAGCGTGGTGCTt | TAA | 206.52 | 175 | 31 |
| taAACGCCAAGGtCGCGGGTTCGATCCCCGTACGG | | | | |
| GCCA | | | | |
| GGCCGGTTAGCTCAGTTGGTAGAGCGTGGTGCTttaA | TAA | 54.78 | 253 | 455 |
| ACGCCAAGGICGCGGGTTCGAACCCCGTACGGGCCA | | | | |
| GGCCGGTTAGCTCAGTTGGTaAGAGCGTGGTGCTttaA | TAA | 52.84 | 255 | 456 |
| ACACCAAGGICGCGGGCTCGACTCCCGCACCGGCCA | | | | |
| GGCTGGTTAGCTCAGTTGGTtAGAGCGTGGTGCTttaA | TAA | 27.16 | 319 | 33 |
| ACGCCAAGGtCGCGGGTTCGATCCCCGTACTGGCCA | | | | |
| GCTCCAGTGGCGCAATCGGTtAGCGCGCGGTACTttaAt | TAA | 12.05 | 382 | 457 |
| ggcagtatgtgtgcgagtgATGCCGAGGtTGTGAGTTCG | | | | |
| AGCCTCACCTGGAGCA | | | | |
| GCTCCAGTGGCGCAATCGGTtAGCGCGCGGTACTttaA | TAA | 8.96 | 415 | 458 |
| cagcagtacatgcagagcaATGCCGAGGTGTGAGTTCGAGCC | | | | |
| TCACCTGGAGCA | | | | |
| GCTCCAGTGGCGCAATCGGTtAGCGCGCGGTACTttaA | TAA | 8.30 | 426 | 459 |
| caacagtatatgtgcgggtgATGCCGAGGtTGTGAGTTCGAGCC | | | | |
| TCACCTGGAGCA | | | | |
| GCTCCAGTGGCGCAATCGGTtAGCGCGCGGTACTttaAt | TAA | 7.80 | 433 | 460 |
| gacagtgcgagcggagcaATGCCGAGGtTGTGAGTTCGATCCT | | | | |
| CACCTGGAGCA | | | | |
| GCTCCAGTGGCGCAATCGGTtAGCGCGCGGTACTttaA | TAA | 5.72 | 470 | 461 |
| agacagtgcacctgtgagcaATGCCGAGGtTGTGAGTTCAAGCC | | | | |
| TCACCTGGAGCA | | | | |
| GGCTGGTTAGTTCAGTTGGTtAGAGCGTGGTGCTttaA | TAA | 1.58 | 777 | 462 |
| ACGCCAAGGtCGTGGGTTCGATCCCCATATCGGCCA | | | | |

TABLE 24B
Isoleucine (Ile) tRNA

| | | fold | | SEQ |
|---|---|---|---|---|
| seq_tRNA | ACE | over | Rank | ID |
| GGCCGGTTAGCTCAGTTGGTtAGAGCGTGGCGCTc | TAG | 752.70 | 101 | 2 |
| taAACGCCAAGGtCGCGGGTTCGATCCCCGTACGG | | | | |
| GCCA | | | | |
| GGCCGGTTAGCTCAGTTGGTcAGAGCGTGGTGCT | TAG | 443.62 | 134 | 5 |
| ctaAACGCCAAGGtCGCGGGTTCGATCCCCGTACG | | | | |
| GGCCA | | | | |
| GGCCGGTTAGCTCAGTTGGTtAGAGCGTGGTGCTctaA | TAG | 195.80 | 181 | 20 |
| ACGCCAAGGICGCGGGTTCGATCCCCGTACGGGCCA | | | | |
| GGCCGGTTAGCTCAGTTGGTtAGAGCGTGGTGCTctaA | TAG | 149.58 | 195 | 1 |
| ACGCCAAGGICGCGGGTTCGATCCCCGTACTGGCCA | | | | |
| GGCTGGTTAGCTCAGTTGGTtAGAGCGTGGTGCTctaA | TAG | 138.08 | 197 | 4 |
| ACGCCAAGGtCGCGGGTTCGATCCCCGTACTGGCCA | | | | |
| GGCCGGTTAGCTCAGTTGGTaAGAGCGTGGTGCTctaA | TAG | 118.95 | 205 | 463 |
| ACACCAAGGCGCGGGCTCGACTCCCGCACCGGCCA | | | | |
| GGCCGGTTAGCTCAGTTGGTtAGAGCGTGGTGCTctaA | TAG | 107.93 | 212 | 7 |
| ACGCCAAGGCGCGGGTTCGAACCCCGTACGGGCCA | | | | |
| GGCCGGTTAGCTCAGTTGGTtAGAGCGTGGTGCTctaA | TAG | 93.84 | 222 | 17 |
| ACGCTAAGGtCGCGGGTTCGATCCCCGTACTGGCCA | | | | |
| GCTCCAGTGGCGCAATCGGTtAGCGCGCGGTACTctaA | TAG | 20.80 | 340 | 464 |
| tggcagtatgtgtgcgagtgATGCCGAGGTGTGAGTTCGAGCC | | | | |
| TCACCTGGAGCA | | | | |
| GCTCCAGTGGCGCAATCGGTtAGCGCGCGGTACTctaA | TAG | 18.14 | 351 | 465 |
| agacagtgcacctgtgagcaATGCCGAGGTGTGAGTTCAAGCC | | | | |
| TCACCTGGAGCA | | | | |
| GCTCCAGTGGCGCAATCGGTtAGCGCGCGGTACTctaA | TAG | 17.48 | 355 | 466 |
| caacagtatatgtgcgggtgATGCCGAGGtTGTGAGTTCGAGCC | | | | |
| TCACCTGGAGCA | | | | |
| GCTCCAGTGGCGCAATCGGTtAGCGCGCGGTACTctaA | TAG | 15.89 | 365 | 467 |
| cagcagtacatgcagagcaATGCCGAGGtTGTGAGTTCGAGCC | | | | |
| TCACCTGGAGCA | | | | |
| GGCCGGTTAGCTCAGTCGGCtAGAGCGTGGTGCTctaA | TAG | 12.41 | 378 | 468 |
| ACGCCAAGGtCGCGGGTTCGATCCCCGTACGGGCCA | | | | |
| GCTCCAGTGGCGCAATCGGTtAGCGCGCGGTACTctaA | TAG | 7.35 | 442 | 469 |
| tgacagtgcgagcggagcaATGCCGAGGtTGTGAGTTCGATCC | | | | |
| TCACCTGGAGCA | | | | |
| GGCTGGTTAGTTCAGTTGGTtAGAGCGTGGTGCTctaA | TAG | 2.14 | 685 | 470 |
| ACGCCAAGGICGTGGGTTCGATCCCCATATCGGCCA | | | | |

TABLE 24C
Isoleucine (Ile) tRNA

| | | fold | | SEQ |
|---|---|---|---|---|
| seq_tRNA | ACE | over | Rank | ID |
| GGCCGGTTAGCTCAGTTGGTtAGAGCGTGGTGCTtcaA | TGA | 52.36 | 256 | 10 |
| ACGCCAAGGtCGCGGGTTCGATCCCCGTACTGGCCA | | | | |
| GGCCGGTTAGCTCAGTTGGTtAGAGCGTGGTGCTtcaA | TGA | 40.90 | 281 | 13 |
| ACGCTAAGGtCGCGGGTTCGATCCCCGTACTGGCCA | | | | |
| GGCCGGTTAGCTCAGTCGGCtAGAGCGTGGTGCTtcaA | TGA | 25.54 | 323 | 25 |
| ACGCCAAGGtCGCGGGTTCGATCCCCGTACGGGCCA | | | | |
| GGCCGGTTAGCTCAGTTGGTcAGAGCGTGGTGCTtcaA | TGA | 23.72 | 331 | 471 |
| ACGCCAAGGICGCGGGTTCGATCCCCGTACGGGCCA | | | | |
| GGCCGGTTAGCTCAGTTGGTtAGAGCGTGGTGCTtcaA | TGA | 10.51 | 399 | 472 |
| ACGCCAAGGICGCGGGTTCGAACCCCGTACGGGCCA | | | | |
| GGCCGGTTAGCTCAGTTGGTtAGAGCGTGGTGCTtcaA | TGA | 7.59 | 437 | 473 |
| ACGCCAAGGICGCGGGTTCGATCCCCGTACGGGCCA | | | | |
| GGCCGGTTAGCTCAGTTGGTtAGAGCGTGGCGCTtcaA | TGA | 3.51 | 555 | 474 |
| ACGCCAAGGtCGCGGGTTCGATCCCCGTACGGGCCA | | | | |
| GGCCGGTTAGCTCAGTTGGTaAGAGCGTGGTGCTtcaA | TGA | 3.49 | 557 | 475 |
| ACACCAAGGICGCGGGCTCGACTCCCGCACCGGCCA | | | | |
| GCTCCAGTGGCGCAATCGGTtAGCGCGCGGTACTtcaA | TGA | 2.81 | 602 | 476 |
| tggcagtatgtgtgcgagtgATGCCGAGGtTGTGAGTTCGAGCC | | | | |
| TCACCTGGAGCA | | | | |
| GCTCCAGTGGCGCAATCGGTtAGCGCGCGGTACTtcaA | TGA | 2.30 | 663 | 477 |
| cagcagtacatgcagagcaATGCCGAGGtTGTGAGTTCGAGCC | | | | |
| TCACCTGGAGCA | | | | |
| GCTCCAGTGGCGCAATCGGTtAGCGCGCGGTACTtcaA | TGA | 2.12 | 691 | 478 |
| agacagtgcacctgtgagcaATGCCGAGGTGTGAGTTCAAGCC | | | | |
| TCACCTGGAGCA | | | | |
| GGCTGGTTAGTTCAGTTGGTtAGAGCGTGGTGCTtcaA | TGA | 1.81 | 736 | 479 |
| ACGCCAAGGICGTGGGTTCGATCCCCATATCGGCCA | | | | |
| GGCTGGTTAGCTCAGTTGGTtAGAGCGTGGTGCTtcaA | TGA | 1.64 | 766 | 480 |
| ACGCCAAGGCGCGGGTTCGATCCCCGTACTGGCCA | | | | |
| GCTCCAGTGGCGCAATCGGTtAGCGCGCGGTACTtcaA | TGA | 1.20 | 838 | 481 |
| tgacagtgcgagcggagcaATGCCGAGGTGTGAGTTCGATCC | | | | |
| TCACCTGGAGCA | | | | |
| GCTCCAGTGGCGCAATCGGTtAGCGCGCGGTACTtcaA | TGA | 0.86 | 873 | 482 |
| caacagtatatgtgcgggtgATGCCGAGGTGTGAGTTCGAGCC | | | | |
| TCACCTGGAGCA | | | | |

TABLE 25A
Leucine (Leu) tRNA

| | | fold | | SEQ |
|---|---|---|---|---|
| seq_tRNA | ACE | over | Rank | ID |
| ACCAGGATGGCCGAGTGGTtAAGGCGTTGGACTttaGA | TAA | 1119.86 | 86 | 483 |
| TCCAATGGACATATGTCCGCGTGGGTTCGAACCCCAC | | | | |
| TCCTGGTA | | | | |
| ACCGGGATGGCCGAGTGGTtAAGGCGTTGGACTttaGA | TAA | 660.61 | 108 | 484 |
| TCCAATGGGCTGGTGCCCGCGTGGGTTCGAACCCCA | | | | |
| CTCTCGGTA | | | | |
| GTCAGGATGGCCGAGTGGTctAAGGCGCCAGACTttaGT | TAA | 597.44 | 115 | 485 |
| TCTGGTCTCCGCATGGAGGCGTGGGTTCGAATCCCAC | | | | |
| TTCTGACA | | | | |
| GTCAGGATGGCCGAGTGGTctAAGGCGCCAGACTttaGT | TAA | 552.14 | 119 | 486 |
| TCTGGTCTCCGTATGGAGGCGTGGGTTCGAATCCCAC | | | | |
| TTCTGACA | | | | |
| GTCAGGATGGCCGAGTGGTctAAGGCGCCAGACTttaGT | TAA | 544.00 | 121 | 487 |
| TCTGGTCTCCAATGGAGGCGTGGGTTCGAATCCCACT | | | | |
| TCTGACA | | | | |
| ACCAGGATGGCCAAGTAGTTaAAGGCACTGGACTttaG | TAA | 403.61 | 137 | 488 |
| AGCCAATGGACATATGTCTGTGTGGGTTTGAACCCCA | | | | |
| CTCCTGGTG | | | | |
| ACCGGGATGGCTGAGTGGTtAAGGCGTTGGACTttaGA | TAA | 396.34 | 139 | 489 |
| TCCAATGGACAGGTGTCCGCGTGGGTTCGAGCCCCA | | | | |
| CTCCCGGTA | | | | |
| GGTAGCGTGGCCGAGCGGTctAAGGCGCTGGATTttaGC | TAA | 69.36 | 240 | 490 |
| TCCAGTCTCTTCGGAGGCGTGGGTTCGAATCCCACCG | | | | |
| CTGCCA | | | | |
| GGTAGCGTGGCCGAGTGGTctAAGGCGCTGGATTttaGC | TAA | 63.91 | 245 | 491 |
| TCCAGTCATTTCGATGgCGTGGGTTCGAATCCCACCG | | | | |
| CTGCCA | | | | |
| GGTAGTGTGGCCGAGCGGTctAAGGCGCTGGATTttaGC | TAA | 63.70 | 246 | 492 |
| TCCAGTCTCTTCGGGGGCGTGGGTTCGAATCCCACCA | | | | |
| CTGCCA | | | | |
| GGTAGCGTGGCCGAGCGGTctAAGGCGCTGGATTttaGC | TAA | 45.94 | 268 | 493 |
| TCCAGTCTCTTCGGGGGCGTGGGTTCAAATCCCACCG | | | | |
| CTGCCA | | | | |
| ACCAGAATGGCCGAGTGGTtAAGGCGTTGGACTttaGA | TAA | 10.35 | 402 | 494 |
| TCCAATGGATTCATATCCGCGTGGGTTCGAACCCCAC | | | | |
| TTCTGGTA | | | | |
| GGTAGTGTGGTTGAATGGTctAAGGCACTGAATTttaGC | TAA | 7.44 | 439 | 495 |
| TCCAGTCTCTTTGGGGaCGTGGGTTTAAATCCCACTG | | | | |
| CTGCAA | | | | |
| GAGAAAGTCATCGTAGTTACGAAGTTGGCTttaACCCA | TAA | 3.72 | 541 | 496 |
| GTTTtGGGAGGTTCAATTCCTTCCTTTCTCT | | | | |
| ACTCATTTGGCTGAGTGGTtAAGGCATTGGACTttaGAT | TAA | 2.92 | 597 | 497 |
| CCAATGGAGTAGTGGCTGTGTGGGTTTAAACCCCACT | | | | |
| ACTGGTA | | | | |
| GCCTCCTTAGTGCAGTAGGTAGCGCATCAGTCTttaAA | TAA | 2.34 | 656 | 498 |
| TCTGAATGgtCCTGAGTTCAAGCCTCAGAGGGGGCA | | | | |
| GGTAGGGTGGCCGAGCGGTctAAGGCACTGTATTttaAC | TAA | 2.33 | 658 | 499 |
| TCCAGTCTCTTCAGAGGCATGGGTTTGAATCCCACTG | | | | |
| CTGCCA | | | | |
| GGTTCCATGGTGTAATGGTgAGCACTCTGGACTttaAAT | TAA | 2.23 | 671 | 500 |
| CCAGAAGtAGTgCTGGAACAA | | | | |
| GTCAGGATGGCCGAGCAGTcttAAGGCGCTGCGTTttaA | TAA | 2.15 | 683 | 501 |
| TCGCACCCTCCGCTGGAGGCGTGGGTTCGAATCCCAC | | | | |
| TTTTGACA | | | | |
| GCCGAGCGGTctAAGGCTCCGGATTttaGCGCCGGTGTC | TAA | 2.01 | 709 | 502 |
| TTCGGAGgCATGGGTTCGAATTCCAC | | | | |
| GGGCCAGTGGCTCAATGGAtAATGCGTCTGACTttaAA | TAA | 1.81 | 738 | 503 |
| TCAGAAGAtTCCAGCCTTGACTCCTGGCTGGCTCA | | | | |
| GGTAGCGTGGCCGAGTGGTctAAGACGCTGGATTttaGC | TAA | 1.59 | 773 | 504 |
| TCCAGTCTCTTCGGGGGCGTGGGTTTGAATCCCACCG | | | | |
| CTGCCA | | | | |
| GTCAGGATGGCCGAGTGGTctAAGGCGCCAGACTttaGt | TAA | 1.11 | 849 | 505 |
| TTCTGGTCTCCGGATGGAGGCGTGGGTTCGAATCCCA | | | | |
| CTTCTGACA | | | | |
| GTCAGGGTGGCTGAGCAGTctGAGGGGCTGCGTTttaGT | TAA | 0.28 | 903 | 506 |
| CGCAGTCTGCCCTGGAGGCGTGGGTTCGAATCCCACT | | | | |
| CCTGAAA | | | | |

TABLE 25B
Leucine (Leu) tRNA

| | | fold | | SEQ |
|---|---|---|---|---|
| seq_tRNA | ACE | over | Rank | ID |
| GGTAGCGTGGCCGAGCGGTctAAGGCGCTGGATTct | TAG | 10860.81 | 1 | 507 |
| aGCTCCAGTCTCTTCGGGGGCGTGGGTTCAAATCC | | | | |
| CACCGCTGCCA | | | | |
| GTCAGGATGGCCGAGTGGTctAAGGCGCCAGACTctaG | TAG | 7346.38 | 6 | 508 |
| TTCTGGTCTCCGCATGGAGGCGTGGGTTCGAATCCCA | | | | |
| CTTCTGACA | | | | |
| ACCAGAATGGCCGAGTGGTtAAGGCGTTGGACTctaGA | TAG | 6543.02 | 8 | 509 |
| TCCAATGGATTCATATCCGCGTGGGTTCGAACCCCAC | | | | |
| TTCTGGTA | | | | |
| ACCAGGATGGCCGAGTGGTtAAGGCGTTGGACTctaGA | TAG | 6450.21 | 9 | 510 |
| TCCAATGGACATATGTCCGCGTGGGTTCGAACCCCAC | | | | |
| TCCTGGTA | | | | |
| GTCAGGATGGCCGAGTGGTctAAGGCGCCAGACTctaG | TAG | 6139.78 | 10 | 511 |
| TTCTGGTCTCCAATGGAGGCGTGGGTTCGAATCCCAC | | | | |
| TTCTGACA | | | | |
| GTCAGGATGGCCGAGTGGTctAAGGCGCCAGACTctaG | TAG | 5517.76 | 12 | 512 |
| TTCTGGTCTCCGTATGGAGGCGTGGGTTCGAATCCCA | | | | |
| CTTCTGACA | | | | |
| ACCGGGATGGCTGAGTGGTtAAGGCGTTGGACTctaGA | TAG | 5245.68 | 13 | 513 |
| TCCAATGGACAGGTGTCCGCGTGGGTTCGAGCCCCA | | | | |
| CTCCCGGTA | | | | |
| GGTAGCGTGGCCGAGTGGTctAAGGCGCTGGATTctaG | TAG | 2304.00 | 55 | 514 |
| CTCCAGTCATTTCGATGgCGTGGGTTCGAATCCCACC | | | | |
| GCTGCCA | | | | |
| ACCGGGATGGCCGAGTGGTtAAGGCGTTGGACTctaGA | TAG | 2238.60 | 56 | 515 |
| TCCAATGGGCTGGTGCCCGCGTGGGTTCGAACCCCA | | | | |
| CTCTCGGTA | | | | |
| GGTAGTGTGGCCGAGCGGTctAAGGCGCTGGATTctaG | TAG | 1741.67 | 63 | 516 |
| CTCCAGTCTCTTCGGGGGCGTGGGTTCGAATCCCACC | | | | |
| ACTGCCA | | | | |
| GGTAGCGTGGCCGAGCGGTctAAGGCGCTGGATTctaG | TAG | 1170.92 | 85 | 517 |
| CTCCAGTCTCTTCGGAGGCGTGGGTTCGAATCCCACC | | | | |
| GCTGCCA | | | | |
| ACCAGGATGGCCAAGTAGTTaAAGGCACTGGACTctaG | TAG | 484.70 | 130 | 518 |
| AGCCAATGGACATATGTCTGTGTGGGTTTGAACCCCA | | | | |
| CTCCTGGTG | | | | |
| GTCAGGATGGCCGAGTGGTctAAGGCGCCAGACTctaGt | TAG | 41.13 | 279 | 519 |
| TTCTGGTCTCCGGATGGAGGCGTGGGTTCGAATCCCA | | | | |
| CTTCTGACA | | | | |
| GGTTCCATGGTGTAATGGTgAGCACTCTGGACTctaAA | TAG | 37.85 | 289 | 520 |
| TCCAGAAGtAGTgCTGGAACAA | | | | |
| GTCAGGGTGGCTGAGCAGTctGAGGGGCTGCGTTctaG | TAG | 31.54 | 305 | 521 |
| TCGCAGTCTGCCCTGGAGGCGTGGGTTCGAATCCCAC | | | | |
| TCCTGAAA | | | | |
| GTCAGGATGGCCGAGCAGTcttAAGGCGCTGCGTTctaA | TAG | 30.90 | 306 | 522 |
| TCGCACCCTCCGCTGGAGGCGTGGGTTCGAATCCCAC | | | | |
| TTTTGACA | | | | |
| ACTCATTTGGCTGAGTGGTtAAGGCATTGGACTctaGAT | TAG | 17.53 | 354 | 523 |
| CCAATGGAGTAGTGGCTGTGTGGGTTTAAACCCCACT | | | | |
| ACTGGTA | | | | |
| GGTAGTGTGGTTGAATGGTctAAGGCACTGAATTctaGC | TAG | 16.54 | 360 | 524 |
| TCCAGTCTCTTTGGGGaCGTGGGTTTAAATCCCACTG | | | | |
| CTGCAA | | | | |
| GGTAGCGTGGCCGAGTGGTctAAGACGCTGGATTctaG | TAG | 8.03 | 431 | 525 |
| CTCCAGTCTCTTCGGGGGCGTGGGTTTGAATCCCACC | | | | |
| GCTGCCA | | | | |
| GCCTCCTTAGTGCAGTAGGTAGCGCATCAGTCTctaAA | TAG | 6.17 | 463 | 526 |
| TCTGAATGgtCCTGAGTTCAAGCCTCAGAGGGGGCA | | | | |
| GAGAAAGTCATCGTAGTTACGAAGTTGGCTctaACCCA | TAG | 4.06 | 525 | 527 |
| GTTTtGGGAGGTTCAATTCCTTCCTTTCTCT | | | | |
| GGGCCAGTGGCTCAATGGAtAATGCGTCTGACTctaAA | TAG | 3.55 | 550 | 528 |
| TCAGAAGAtTCCAGCCTTGACTCCTGGCTGGCTCA | | | | |
| GCCGAGCGGTctAAGGCTCCGGATTctaGCGCCGGTGT | TAG | 3.11 | 583 | 529 |
| CTTCGGAGgCATGGGTTCGAATTCCAC | | | | |
| GGTAGGGTGGCCGAGCGGTctAAGGCACTGTATTctaA | TAG | 0.19 | 905 | 530 |
| CTCCAGTCTCTTCAGAGGCATGGGTTTGAATCCCACT | | | | |
| GCTGCCA | | | | |

TABLE 25C
Leucine (Leu) tRNA

| | | fold | | SEQ |
|---|---|---|---|---|
| seq_tRNA | ACE | over | Rank | ID |
| ACCAGGATGGCCGAGTGGTLAAGGCGTTGGACTtcaGA | TGA | 603.23 | 113 | 531 |
| TCCAATGGACATATGTCCGCGTGGGTTCGAACCCCAC | | | | |
| TCCTGGTA | | | | |
| ACCAGAATGGCCGAGTGGTtAAGGCGTTGGACTtcaGA | TGA | 594.17 | 116 | 522 |
| TCCAATGGATTCATATCCGCGTGGGTTCGAACCCCAC | | | | |
| TTCTGGTA | | | | |
| ACCGGGATGGCCGAGTGGTtAAGGCGTTGGACTtcaGA | TGA | 449.63 | 132 | 533 |
| TCCAATGGGCTGGTGCCCGCGTGGGTTCGAACCCCA | | | | |
| CTCTCGGTA | | | | |
| ACCGGGATGGCTGAGTGGTtAAGGCGTTGGACTtcaGA | TGA | 377.69 | 144 | 534 |
| TCCAATGGACAGGTGTCCGCGTGGGTTCGAGCCCCA | | | | |
| CTCCCGGTA | | | | |
| GTCAGGATGGCCGAGTGGTctAAGGCGCCAGACTtcaG | TGA | 292.41 | 156 | 535 |
| TTCTGGTCTCCGTATGGAGGCGTGGGTTCGAATCCCA | | | | |
| CTTCTGACA | | | | |
| GTCAGGATGGCCGAGTGGTctAAGGCGCCAGACTtcaG | TGA | 261.52 | 162 | 536 |
| TTCTGGTCTCCGCATGGAGGCGTGGGTTCGAATCCCA | | | | |
| CTTCTGACA | | | | |
| GTCAGGATGGCCGAGTGGTctAAGGCGCCAGACTtcaG | TGA | 114.98 | 210 | 537 |
| TTCTGGTCTCCAATGGAGGCGTGGGTTCGAATCCCAC | | | | |
| TTCTGACA | | | | |
| GGTAGTGTGGCCGAGCGGTctAAGGCGCTGGATTtcaG | TGA | 25.11 | 325 | 538 |
| CTCCAGTCTCTTCGGGGGCGTGGGTTCGAATCCCACC | | | | |
| ACTGCCA | | | | |
| GGTAGCGTGGCCGAGCGGTctAAGGCGCTGGATTtcaG | TGA | 23.29 | 332 | 539 |
| CTCCAGTCTCTTCGGGGGCGTGGGTTCAAATCCCACC | | | | |
| GCTGCCA | | | | |
| GGTAGCGTGGCCGAGCGGTctAAGGCGCTGGATTtcaG | TGA | 23.13 | 334 | 540 |
| CTCCAGTCTCTTCGGAGGCGTGGGTTCGAATCCCACC | | | | |
| GCTGCCA | | | | |
| GGTAGCGTGGCCGAGTGGTctAAGGCGCTGGATTtcaG | TGA | 7.12 | 447 | 541 |
| CTCCAGTCATTTCGATGgCGTGGGTTCGAATCCCACC | | | | |
| GCTGCCA | | | | |
| GTCAGGATGGCCGAGCAGTcttAAGGCGCTGCGTTtcaA | TGA | 3.46 | 560 | 542 |
| TCGCACCCTCCGCTGGAGGCGTGGGTTCGAATCCCAC | | | | |
| TTTTGACA | | | | |
| GGTAGTGTGGTTGAATGGTctAAGGCACTGAATTtcaGC | TGA | 3.10 | 585 | 543 |
| TCCAGTCTCTTTGGGGaCGTGGGTTTAAATCCCACTG | | | | |
| CTGCAA | | | | |
| GGTTCCATGGTGTAATGGTgAGCACTCTGGACTtcaAA | TGA | 3.01 | 590 | 544 |
| TCCAGAAGtAGTgCTGGAACAA | | | | |
| GCCTCCTTAGTGCAGTAGGTAGCGCATCAGTCTtcaAA | TGA | 2.76 | 606 | 545 |
| TCTGAATGgtCCTGAGTTCAAGCCTCAGAGGGGGCA | | | | |
| ACCAGGATGGCCAAGTAGTTaAAGGCACTGGACTtcaG | TGA | 2.51 | 630 | 546 |
| AGCCAATGGACATATGTCTGTGTGGGTTTGAACCCCA | | | | |
| CTCCTGGTG | | | | |
| ACTCATTTGGCTGAGTGGTAAGGCATTGGACTtcaGAT | TGA | 2.28 | 666 | 547 |
| CCAATGGAGTAGTGGCTGTGTGGGTTTAAACCCCACT | | | | |
| ACTGGTA | | | | |
| GTCAGGATGGCCGAGTGGTctAAGGCGCCAGACTtcaGt | TGA | 2.17 | 679 | 548 |
| TTCTGGTCTCCGGATGGAGGCGTGGGTTCGAATCCCA | | | | |
| CTTCTGACA | | | | |
| GGTAGCGTGGCCGAGTGGTctAAGACGCTGGATTtcaG | TGA | 1.79 | 743 | 549 |
| CTCCAGTCTCTTCGGGGGCGTGGGTTTGAATCCCACC | | | | |
| GCTGCCA | | | | |
| GCCGAGCGGTctAAGGCTCCGGATTtcaGCGCCGGTGT | TGA | 1.54 | 785 | 550 |
| CTTCGGAGgCATGGGTTCGAATTCCAC | | | | |
| GGTAGGGTGGCCGAGCGGTctAAGGCACTGTATTtcaA | TGA | 1.37 | 819 | 551 |
| CTCCAGTCTCTTCAGAGGCATGGGTTTGAATCCCACT | | | | |
| GCTGCCA | | | | |
| GAGAAAGTCATCGTAGTTACGAAGTTGGCTtcaACCCA | TGA | 1.27 | 832 | 552 |
| GTTTtGGGAGGTTCAATTCCTTCCTTTCTCT | | | | |
| GTCAGGGTGGCTGAGCAGTctGAGGGGCTGCGTTtcaG | TGA | 0.18 | 906 | 553 |
| TCGCAGTCTGCCCTGGAGGCGTGGGTTCGAATCCCAC | | | | |
| TCCTGAAA | | | | |
| GGGCCAGTGGCTCAATGGAtAATGCGTCTGACTtcaAA | TGA | 0.04 | 914 | 554 |
| TCAGAAGAtTCCAGCCTTGACTCCTGGCTGGCTCA | | | | |

TABLE 26A
Lysine (Lys) tRNA

| | | fold | | SEQ |
|---|---|---|---|---|
| seq_tRNA | ACE | over | Rank | ID |
| GCCTGGGTAGCTCAGTCGGTAGAGCATCAGACTttaAA | TAA | 623.21 | 111 | 555 |
| TCTGAGGGtCCAGGGTTCAAGTCCCTGTCCAGGCG | | | | |
| GCCTGGATAGCTCAGTTGGTAGAGCATCAGACTttaAA | TAA | 443.64 | 133 | 556 |
| TCTGAGGGtCCAGGGTTCAAGTCCCTGTTCAGGCA | | | | |
| GCCTGGATAGCTCAGTTGGTAGAGCATCAGACTtta | TAA | 395.91 | 140 | 557 |
| AATCTGAGGGtCCAGGGTTCAAGTCCCTGTTCAGG | | | | |
| CG | | | | |
| GCCCGGCTAGCTCAGTCGGTAGAGCATGGGACTttaAA | TAA | 107.24 | 213 | 558 |
| TCTCAGGGtCGTGGGTTCGAGCCCCACGTTGGGCG | | | | |
| GCGCTAGTCAGTAGAGCATGAGACTttaAATCTCAGGG | TAA | 85.48 | 230 | 559 |
| tCGTGGGTTCGAGCCCCACATCGGGCG | | | | |
| GCCCGGATAGCTCAGTCGGTAGAGCATCAGACTttaAA | TAA | 24.38 | 328 | 560 |
| TCTGAGGGtCCGGGGTTCAAGTCCCTGTTCGGGCG | | | | |
| GCCTGGATAGCTCAGTCGGTAGAGCATCAGACTttaAA | TAA | 10.91 | 396 | 561 |
| TCTGAGGGtCCAGGGTTCAAGTCCCTGTTCAGGCG | | | | |
| GCCCGGCTAGCTCAGTCGGTAGAGCATGAGACCttaAA | TAA | 5.94 | 465 | 562 |
| TCTCAGGGtCGTGGGTTCGAGCCCCACGTTGGGCG | | | | |
| CCCCGGCTGGCTCAGTCAGTAGATCATGAGACTttaAA | TAA | 3.54 | 551 | 563 |
| TCTCAGGGtCGTGGGTTCACGCCCCACACTGGGCG | | | | |
| GCCCGACTACCTCAGTCGGTgGAGCATGGGACTttaCA | TAA | 3.53 | 554 | 564 |
| TCCCAGGGTGTGGGTTCGAGCCCCACATTGGGCA | | | | |
| GCCTGGCTACCTCAGTTGGTAGAGCATGGGACTttaAA | TAA | 3.32 | 567 | 565 |
| TCCCAGAGtcAGTGGGTTCAAGCCTCACATTGAGTG | | | | |
| GCCTGGATAGCTCCTTCGGTAGAGCATCATcagACTtta | TAA | 3.18 | 577 | 566 |
| AATGTGAGGGCCAGGGTTCAAGTTCCTGTTTGGGCG | | | | |
| GCCTGGCTAGCTCAGTCGGCAAAGCATGAGACTttaAA | TAA | 2.20 | 674 | 567 |
| TCTCAGGGtCGTGGGCTCGAGCTCCATGTTGGGCG | | | | |
| GCCTGGGTAGCTCAGTCGGTAGAGCTaTCAGACTttaA | TAA | 2.01 | 708 | 568 |
| GCCTGAGGAtTCAGGGTTCAATCCCTTGCTGGGGCG | | | | |
| GCCAGGATAGTTCAGGTGGTAGAGCATCAGACTttaAA | TAA | 1.96 | 718 | 569 |
| CCTGAGGGUTCAGGGTTCAAGTCTCTGTTTGGGCG | | | | |
| ACCCAGATAGCTCAGTCAGTAGAGCATCAGACTttaAA | TAA | 1.64 | 767 | 570 |
| TCTGAGGGtCCAAGGTTCATGTCCCTTTTTGGGTG | | | | |
| GCCCAGCTAGCTCAGTCGGTAGAGCATAAGACTttaAA | TAA | 1.54 | 784 | 571 |
| TCTCAGGGTGTGGATTCGTGCCCCATGCTGGGTG | | | | |
| GTTGGGGTAACTCAGTTGGTAGAGTAGCAGACTttaCA | TAA | 1.53 | 788 | 572 |
| TCTGAGGGtCCAGGGTTTAAGTCCATGTCCAGGCA | | | | |
| GCCCAGCTAGCTCAGTCGGTAGAGCATGAGACTttaAA | TAA | 1.45 | 803 | 573 |
| TCTCAGGGtCATGGGTTTGAGCCCCACGTTTGGTG | | | | |
| ACCTGGGTAGCTTAGTTGGTAGAGCATTGGACTttaAA | TAA | 1.44 | 808 | 574 |
| TTTGAGGGcCCAGGTTTCAAGTCCCTGTTTGGGTG | | | | |
| GCCCGGAGAGCTCAGTGGGTAGAGCATCAGACTttaA | TAA | 1.44 | 809 | 575 |
| ATCTGAGGGtCCAGGGTTCAAGTCCTCGTTCGGGCA | | | | |
| GTCTAGCTAGATCAGTTGGTAGAGCATAAGACTttaAA | TAA | 1.44 | 810 | 576 |
| TCTCAGGGtCATGGGTTTGAGCCCTACGTTGGGCG | | | | |
| GCCTGGATAGCTCAGTTGGTAGAACATCAGACTttaAA | TAA | 1.31 | 827 | 577 |
| TCTGACGGtGCAGGGTTCAAGTCCCTGTTCAGGCG | | | | |
| GCCCGGCTAGCTCAGTCGATAGAGCATGAGACTttaAA | TAA | 0.95 | 867 | 568 |
| TCTCAGGGtCGTGGGTTCGAGCCGCACGTTGGGCG | | | | |
| CTGCAGCTAGCTCAGTCGGTAGAGCATGAGACTttaAA | TAA | 0.65 | 888 | 569 |
| TCTCAGGGtCATGGGTTCGTGCCCCATGTTGGG | | | | |
| CCGAATAGCTTAGTTGATgAAGCGTGAGACTttaAATC | TAA | 0.61 | 890 | 580 |
| TCAGGGLAGTGGGTTCAAGCCCCACATTGGA | | | | |
| GCCCAGCTAGCTCAGCCGGTAGAGCACAAGACTttaA | TAA | 0.40 | 898 | 5381 |
| ATCTCAGGGtCGTGGGTTTGAGCCCTGTGTTGAGCA | | | | |
| CCAGCATGTCTCAGTCGGTATAGTGTGAGACTttaAAT | TAA | 0.36 | 901 | 582 |
| CTCAGGGtCGTGGGTTCAAGCCCCACATTGGG | | | | |
| ACCTGGGTAGCTCAGTAGGTAGAACATCAGACTttaAA | TAA | 0.11 | 911 | 583 |
| TCTGAGGGtCTAGGGTTCAAGTCCCTGTCCAGGCG | | | | |
| GATAGCTCAGTTGATAGAGCATCAGACTttaAATCTGA | TAA | 0.01 | 918 | 584 |
| GGGtCCAGGGTTCATGTCCCTGTT | | | | |

TABLE 26B
Lysine (Lys) tRNA

| | | fold | | SEQ |
|---|---|---|---|---|
| seq_tRNA | ACE | over | Rank | ID |
| CCGAATAGCTTAGTTGATgAAGCGTGAGACTctaAATC | TAG | 4126.12 | 24 | 585 |
| TCAGGGtAGTGGGTTCAAGCCCCACATTGGA | | | | |
| ACCTGGGTAGCTTAGTTGGTAGAGCATTGGACTctaAA | TAG | 2888.19 | 40 | 586 |
| TTTGAGGGcCCAGGTTTCAAGTCCCTGTTTGGGTG | | | | |
| GCCTGGGTAGCTCAGTCGGTAGAGCATCAGACTctaAA | TAG | 1065.00 | 88 | 587 |
| TCTGAGGGtCCAGGGTTCAAGTCCCTGTCCAGGCG | | | | |
| GCCTGGATAGCTCAGTCGGTAGAGCATCAGACTctaAA | TAG | 1006.94 | 89 | 588 |
| TCTGAGGGtCCAGGGTTCAAGTCCCTGTTCAGGCG | | | | |
| GCCTGGATAGCTCAGTTGGTAGAGCATCAGACTctaAA | TAG | 973.50 | 91 | 589 |
| TCTGAGGGtCCAGGGTTCAAGTCCCTGTTCAGGCG | | | | |
| GCCTGGATAGCTCAGTTGGTAGAGCATCAGACTctaAA | TAG | 842.97 | 95 | 590 |
| TCTGAGGGtCCAGGGTTCAAGTCCCTGTTCAGGCA | | | | |
| GCCCGGATAGCTCAGTCGGTAGAGCATCAGACTctaA | TAG | 315.19 | 152 | 591 |
| ATCTGAGGGtCCGGGGTTCAAGTCCCTGTTCGGGCG | | | | |
| GCCCGGCTAGCTCAGTCGGTAGAGCATGGGACTctaA | TAG | 97.19 | 221 | 592 |
| ATCTCAGGGCGTGGGTTCGAGCCCCACGTTGGGCG | | | | |
| GCCCGGCTAGCTCAGTCGGTAGAGCATGAGACCctaA | TAG | 41.12 | 280 | 593 |
| ATCTCAGGGtCGTGGGTTCGAGCCCCACGTTGGGCG | | | | |
| CCAGCATGTCTCAGTCGGTATAGTGTGAGACTctaAAT | TAG | 22.79 | 336 | 594 |
| CTCAGGGtCGTGGGTTCAAGCCCCACATTGGG | | | | |
| GCCTGGGTAGCTCAGTCGGTAGAGCTaTCAGACTctaA | TAG | 16.03 | 363 | 595 |
| GCCTGAGGAtTCAGGGTTCAATCCCTTGCTGGGGCG | | | | |
| GCCCGACTACCTCAGTCGGTgGAGCATGGGACTctaCA | TAG | 8.20 | 428 | 596 |
| TCCCAGGGTGTGGGTTCGAGCCCCACATTGGGCA | | | | |
| GCCCAGCTAGCTCAGTCGGTAGAGCATAAGACTctaA | TAG | 7.90 | 432 | 597 |
| ATCTCAGGGtTGTGGATTCGTGCCCCATGCTGGGTG | | | | |
| GCCTGGATAGCTCCTTCGGTAGAGCATCATcagACTcta | TAG | 6.71 | 453 | 598 |
| AATGTGAGGGtCCAGGGTTCAAGTTCCTGTTTGGGCG | | | | |
| GCGCTAGTCAGTAGAGCATGAGACTctaAATCTCAGG | TAG | 5.89 | 466 | 589 |
| GtCGTGGGTTCGAGCCCCACATCGGGCG | | | | |
| GCCTGGATAGCTCAGTTGGTAGAACATCAGACTctaAA | TAG | 5.69 | 471 | 600 |
| TCTGACGGtGCAGGGTTCAAGTCCCTGTTCAGGCG | | | | |
| GTCTAGCTAGATCAGTTGGTAGAGCATAAGACTctaAA | TAG | 5.68 | 472 | 601 |
| TCTCAGGGtCATGGGTTTGAGCCCTACGTTGGGCG | | | | |
| GCCCAGCTAGCTCAGCCGGTAGAGCACAAGACTctaA | TAG | 5.49 | 479 | 602 |
| ATCTCAGGGtCGTGGGTTTGAGCCCTGTGTTGAGCA | | | | |
| CTGCAGCTAGCTCAGTCGGTAGAGCATGAGACTctaAA | TAG | 5.29 | 483 | 603 |
| TCTCAGGGtCATGGGTTCGTGCCCCATGTTGGG | | | | |
| GCCCGGAGAGCTCAGTGGGTAGAGCATCAGACTctaA | TAG | 5.09 | 487 | 604 |
| ATCTGAGGGtCCAGGGTTCAAGTCCTCGTTCGGGCA | | | | |
| CCCCGGCTGGCTCAGTCAGTAGATCATGAGACTctaAA | TAG | 4.52 | 511 | 605 |
| TCTCAGGGtCGTGGGTTCACGCCCCACACTGGGCG | | | | |
| ACCTGGGTAGCTCAGTAGGTAGAACATCAGACTctaA | TAG | 4.32 | 516 | 606 |
| ATCTGAGGGtCTAGGGTTCAAGTCCCTGTCCAGGCG | | | | |
| GATAGCTCAGTTGATAGAGCATCAGACTctaAATCTGA | TAG | 4.26 | 518 | 607 |
| GGGCCAGGGTTCATGTCCCTGTT | | | | |
| GCCTGGCTAGCTCAGTCGGCAAAGCATGAGACTctaA | TAG | 3.99 | 528 | 608 |
| ATCTCAGGGtCGTGGGCTCGAGCTCCATGTTGGGCG | | | | |
| GCCTGGCTACCTCAGTTGGTAGAGCATGGGACTctaAA | TAG | 3.89 | 533 | 609 |
| TCCCAGAGtcAGTGGGTTCAAGCCTCACATTGAGTG | | | | |
| ACCCAGATAGCTCAGTCAGTAGAGCATCAGACTctaA | TAG | 3.88 | 534 | 610 |
| ATCTGAGGGtCCAAGGTTCATGTCCCTTTTTGGGTG | | | | |
| GCCCAGCTAGCTCAGTCGGTAGAGCATGAGACTctaA | TAG | 3.68 | 543 | 611 |
| ATCTCAGGGtCATGGGTTTGAGCCCCACGTTTGGTG | | | | |
| GCCCGGCTAGCTCAGTCGATAGAGCATGAGACTctaA | TAG | 3.07 | 586 | 612 |
| ATCTCAGGGtCGTGGGTTCGAGCCGCACGTTGGGCG | | | | |
| GCCAGGATAGTTCAGGTGGTAGAGCATCAGACTctaA | TAG | 2.57 | 623 | 613 |
| ACCTGAGGGtTCAGGGTTCAAGTCTCTGTTTGGGCG | | | | |
| GTTGGGGTAACTCAGTTGGTAGAGTAGCAGACTctaCA | TAG | 2.21 | 673 | 614 |
| TCTGAGGGtCCAGGGTTTAAGTCCATGTCCAGGCA | | | | |

TABLE 26C
Lysine (Lys) tRNA

| | | fold | | SEQ |
|---|---|---|---|---|
| seq_tRNA | ACE | over | Rank | ID |
| GCCCGACTACCTCAGTCGGTgGAGCATGGGACTtcaCA | TGA | 80.98 | 235 | 615 |
| TCCCAGGGTGTGGGTTCGAGCCCCACATTGGGCA | | | | |
| GCCTGGGTAGCTCAGTCGGTAGAGCATCAGACTtcaAA | TGA | 5.53 | 477 | 616 |
| TCTGAGGGtCCAGGGTTCAAGTCCCTGTCCAGGCG | | | | |
| GCCTGGCTAGCTCAGTCGGCAAAGCATGAGACTtcaA | TGA | 4.94 | 495 | 617 |
| ATCTCAGGGtCGTGGGCTCGAGCTCCATGTTGGGCG | | | | |
| GCCCGGCTAGCTCAGTCGATAGAGCATGAGACTtcaA | TGA | 4.69 | 503 | 618 |
| ATCTCAGGGCGTGGGTTCGAGCCGCACGTTGGGCG | | | | |
| GCCCGGCTAGCTCAGTCGGTAGAGCATGGGACTtcaA | TGA | 3.27 | 569 | 619 |
| ATCTCAGGGtCGTGGGTTCGAGCCCCACGTTGGGCG | | | | |
| GCCCGGATAGCTCAGTCGGTAGAGCATCAGACTtcaA | TGA | 3.23 | 574 | 620 |
| ATCTGAGGGtCCGGGGTTCAAGTCCCTGTTCGGGCG | | | | |
| GCCTGGATAGCTCAGTCGGTAGAGCATCAGACTtcaAA | TGA | 3.11 | 584 | 621 |
| TCTGAGGGtCCAGGGTTCAAGTCCCTGTTCAGGCG | | | | |
| GCCCGGAGAGCTCAGTGGGTAGAGCATCAGACTtcaA | TGA | 2.75 | 607 | 622 |
| ATCTGAGGGtCCAGGGTTCAAGTCCTCGTTCGGGCA | | | | |
| GCCTGGATAGCTCAGTTGGTAGAGCATCAGACTtcaAA | TGA | 2.75 | 608 | 623 |
| TCTGAGGGtCCAGGGTTCAAGTCCCTGTTCAGGCA | | | | |
| GCCCAGCTAGCTCAGTCGGTAGAGCATAAGACTtcaA | TGA | 2.73 | 612 | 624 |
| ATCTCAGGGtTGTGGATTCGTGCCCCATGCTGGGTG | | | | |
| GTCTAGCTAGATCAGTTGGTAGAGCATAAGACTtcaAA | TGA | 2.44 | 639 | 625 |
| TCTCAGGGtCATGGGTTTGAGCCCTACGTTGGGCG | | | | |
| GCCTGGATAGCTCAGTTGGTAGAACATCAGACTtcaAA | TGA | 2.43 | 641 | 626 |
| TCTGACGGtGCAGGGTTCAAGTCCCTGTTCAGGCG | | | | |
| CTGCAGCTAGCTCAGTCGGTAGAGCATGAGACTtcaAA | TGA | 2.35 | 651 | 627 |
| TCTCAGGGtCATGGGTTCGTGCCCCATGTTGGG | | | | |
| ACCTGGGTAGCTTAGTTGGTAGAGCATTGGACTtcaAA | TGA | 2.17 | 680 | 628 |
| TTTGAGGGcCCAGGTTTCAAGTCCCTGTTTGGGTG | | | | |
| GCCTGGATAGCTCCTTCGGTAGAGCATCATcagACTtca | TGA | 2.16 | 681 | 629 |
| AATGTGAGGGtCCAGGGTTCAAGTTCCTGTTTGGGCG | | | | |
| ACCTGGGTAGCTCAGTAGGTAGAACATCAGACTtcaA | TGA | 2.05 | 704 | 630 |
| ATCTGAGGGtCTAGGGTTCAAGTCCCTGTCCAGGCG | | | | |
| CCAGCATGTCTCAGTCGGTATAGTGTGAGACTtcaAAT | TGA | 1.99 | 714 | 631 |
| CTCAGGGtCGTGGGTTCAAGCCCCACATTGGG | | | | |
| CCGAATAGCTTAGTTGATgAAGCGTGAGACTtcaAATC | TGA | 1.84 | 731 | 632 |
| TCAGGGtAGTGGGTTCAAGCCCCACATTGGA | | | | |
| GCCTGGGTAGCTCAGTCGGTAGAGCTaTCAGACTtcaA | TGA | 1.83 | 732 | 633 |
| GCCTGAGGAtTCAGGGTTCAATCCCTTGCTGGGGCG | | | | |
| GCCTGGATAGCTCAGTTGGTAGAGCATCAGACTtcaAA | TGA | 1.82 | 735 | 634 |
| TCTGAGGGtCCAGGGTTCAAGTCCCTGTTCAGGCG | | | | |
| GCGCTAGTCAGTAGAGCATGAGACTtcaAATCTCAGG | TGA | 1.79 | 745 | 635 |
| GtCGTGGGTTCGAGCCCCACATCGGGCG | | | | |
| GCCTGGCTACCTCAGTTGGTAGAGCATGGGACTtcaAA | TGA | 1.71 | 760 | 636 |
| TCCCAGAGtcAGTGGGTTCAAGCCTCACATTGAGTG | | | | |
| GTTGGGGTAACTCAGTTGGTAGAGTAGCAGACTtcaCA | TGA | 1.66 | 764 | 637 |
| TCTGAGGGtCCAGGGTTTAAGTCCATGTCCAGGCA | | | | |
| ACCCAGATAGCTCAGTCAGTAGAGCATCAGACTtcaA | TGA | 1.62 | 770 | 638 |
| ATCTGAGGGtCCAAGGTTCATGTCCCTTTTTGGGTG | | | | |
| GATAGCTCAGTTGATAGAGCATCAGACTtcaAATCTGA | TGA | 1.59 | 775 | 639 |
| GGGtCCAGGGTTCATGTCCCTGTT | | | | |
| GCCAGGATAGTTCAGGTGGTAGAGCATCAGACTtcaA | TGA | 1.28 | 831 | 640 |
| ACCTGAGGGtTCAGGGTTCAAGTCTCTGTTTGGGCG | | | | |
| GCCCAGCTAGCTCAGCCGGTAGAGCACAAGACTtcaA | TGA | 1.02 | 859 | 641 |
| ATCTCAGGGtCGTGGGTTTGAGCCCTGTGTTGAGCA | | | | |
| GCCCGGCTAGCTCAGTCGGTAGAGCATGAGACCtcaA | TGA | 0.36 | 900 | 642 |
| ATCTCAGGGtCGTGGGTTCGAGCCCCACGTTGGGCG | | | | |
| GCCCAGCTAGCTCAGTCGGTAGAGCATGAGACTtcaA | TGA | 0.16 | 908 | 643 |
| ATCTCAGGGtCATGGGTTTGAGCCCCACGTTTGGTG | | | | |
| CCCCGGCTGGCTCAGTCAGTAGATCATGAGACTtcaAA | TGA | 0.03 | 917 | 644 |
| TCTCAGGGtCGTGGGTTCACGCCCCACACTGGGCG | | | | |

TABLE 27A
Methionine (Met) tRNA

| | | fold | | SEQ |
|---|---|---|---|---|
| seq_tRNA | ACE | over | Rank | ID |
| GCCTCGTTAGCGCAGTAGGTAGCGCGTCAGTCTttaAAT | TAA | 3.00 | 590 | 645 |
| CTGAAGGtCGTGAGTTCGATCCTCACACGGGGCA | | | | |
| GCCTCGTTAGCGCAGTAGGCAGCGCGTCAGTCTttaAAT | TAA | 2.40 | 645 | 646 |
| CTGAAGGtCGTGAGTTCGAGCCTCACACGGGGCA | | | | |
| GCCCTCTTAGCGCAGCTGGCAGCGCGTCAGTCTttaAAT | TAA | 1.57 | 779 | 647 |
| CTGAAGGtCCTGAGTTCAAGCCTCAGAGAGGGCA | | | | |
| GCCCTCTTAGCGCAGTGGGcAGCGCGTCAGTCTttaAAT | TAA | 1.50 | 795 | 648 |
| CTGAAGGtCCTGAGTTCGAGCCTCAGAGAGGGCA | | | | |
| GCCCTCTTAGCGCAGCGGGCAGCGCGTCAGTCTttaAAT | TAA | 1.27 | 833 | 649 |
| CTGAAGGtCCTGAGTTCGAGCCTCAGAGAGGGCA | | | | |
| GCCTCCTTAGCGCAGTAGGCAGCGCGTCAGTCTttaAAT | TAA | 1.13 | 845 | 650 |
| CTGAAGGtCCTGAGTTCGAACCTCAGAGGGGGCA | | | | |
| GCCCTCTTAGTGCAGCTGGCAGCGCGTCAGTTTttaAAT | TAA | 1.06 | 855 | 651 |
| CTGAAAGtCCTGAGTTCAAGCCTCAGAGAGGGCA | | | | |

TABLE 27B
Methionine (Met) tRNA

| | | fold | | SEQ |
|---|---|---|---|---|
| seq_tRNA | ACE | over | Rank | ID |
| GCCTCGTTAGCGCAGTAGGCAGCGCGTCAGTCTctaAAT | TAG | 12.08 | 382 | 652 |
| CTGAAGGtCGTGAGTTCGAGCCTCACACGGGGCA | | | | |
| GCCTCGTTAGCGCAGTAGGTAGCGCGTCAGTCTctaAAT | TAG | 11.05 | 393 | 653 |
| CTGAAGGtCGTGAGTTCGATCCTCACACGGGGCA | | | | |
| GCCCTCTTAGCGCAGTGGGCAGCGCGTCAGTCTctaAAT | TAG | 2.41 | 643 | 654 |
| CTGAAGGtCCTGAGTTCGAGCCTCAGAGAGGGCA | | | | |
| GCCCTCTTAGCGCAGCGGGCAGCGCGTCAGTCTctaAAT | TAG | 1.62 | 769 | 655 |
| CTGAAGGtCCTGAGTTCGAGCCTCAGAGAGGGCA | | | | |
| GCCCTCTTAGTGCAGCTGGCAGCGCGTCAGTTTctaAAT | TAG | 1.58 | 778 | 656 |
| CTGAAAGtCCTGAGTTCAAGCCTCAGAGAGGGCA | | | | |
| GCCTCCTTAGCGCAGTAGGCAGCGCGTCAGTCTctaAAT | TAG | 1.04 | 858 | 657 |
| CTGAAGGtCCTGAGTTCGAACCTCAGAGGGGGCA | | | | |
| GCCCTCTTAGCGCAGCTGGCAGCGCGTCAGTCTctaAAT | TAG | 0.89 | 871 | 658 |
| CTGAAGGtCCTGAGTTCAAGCCTCAGAGAGGGCA | | | | |

TABLE 27C
Methionine (Met) tRNA

| | | fold | | SEQ |
|---|---|---|---|---|
| seq_tRNA | ACE | over | Rank | ID |
| GCCCTCTTAGTGCAGCTGGCAGCGCGTCAGTTTtcaAAT | TGA | 2.71 | 615 | 659 |
| CTGAAAGtCCTGAGTTCAAGCCTCAGAGAGGGCA | | | | |
| GCCCTCTTAGCGCAGTGGGCAGCGCGTCAGTCTtcaAAT | TGA | 2.60 | 620 | 660 |
| CTGAAGGtCCTGAGTTCGAGCCTCAGAGAGGGCA | | | | |
| GCCTCGTTAGCGCAGTAGGCAGCGCGTCAGTCTtcaAAT | TGA | 2.53 | 627 | 661 |
| CTGAAGGtCGTGAGTTCGAGCCTCACACGGGGCA | | | | |
| GCCCTCTTAGCGCAGCTGGCAGCGCGTCAGTCTtcaAAT | TGA | 2.09 | 693 | 662 |
| CTGAAGGtCCTGAGTTCAAGCCTCAGAGAGGGCA | | | | |
| GCCCTCTTAGCGCAGCGGGCAGCGCGTCAGTCTtcaAAT | TGA | 2.06 | 701 | 663 |
| CTGAAGGtCCTGAGTTCGAGCCTCAGAGAGGGCA | | | | |
| GCCTCCTTAGCGCAGTAGGCAGCGCGTCAGTCTtcaAAT | TGA | 1.80 | 739 | 664 |
| CTGAAGGtCCTGAGTTCGAACCTCAGAGGGGGCA | | | | |
| GCCTCGTTAGCGCAGTAGGTAGCGCGTCAGTCTtcaAAT | TGA | 1.46 | 801 | 665 |
| CTGAAGGtCGTGAGTTCGATCCTCACACGGGGCA | | | | |

TABLE 28A
Phenylalanine (Phe) tRNA

| | | fold | | SEQ |
|---|---|---|---|---|
| seq_tRNA | ACE | over | Rank | ID |
| GCCGAAATAGCTCAGTTGGGAGAGCGTTAGACTtt | TAA | 523.85 | 125 | 14 |
| aGATCTAAAGGtCCCTGGTTCGATCCCGGGTTTCG | | | | |
| GCA | | | | |
| GCCGAAATAGCTCAGTTGGGAGAGCGTTAGACTttaG | TAA | 364.65 | 147 | 22 |
| ATCTAAAGGtCCCTGGTTCAATCCCGGGTTTCGGCA | | | | |
| GCCGAGATAGCTCAGTTGGGAGAGCGTTAGACTttaG | TAA | 86.76 | 229 | 666 |
| ATCTAAAGGtCCCTGGTTCAATCCCGGGTTTCGGCA | | | | |
| GCCGAAATAGCTCAGTTGGGAGAGCGTTAGACCttaG | TAA | 2.13 | 689 | 667 |
| ATCTtAAAGGCCCTGGTTCAATCCCGGGTTTCGGCA | | | | |
| GCTGAAATAGCTCAGTTGGGAGAGCGTTAGACTttaG | TAA | 0.85 | 874 | 668 |
| ATCTtAAAGTtCCCTGGTTCAACCCTGGGTTTCAGCC | | | | |

TABLE 28B
Phenylalanine (Phe) tRNA

| | | fold | | SEQ |
|---|---|---|---|---|
| seq_tRNA | ACE | over | Rank | ID |
| GCCGAAATAGCTCAGTTGGGAGAGCGTTAGACTct | TAG | 651.22 | 109 | 669 |
| aGATCTAAAGGtCCCTGGTTCGATCCCGGGTTTCG | | | | |
| GCA | | | | |
| GCCGAAATAGCTCAGTTGGGAGAGCGTTAGACTctaG | TAG | 366.95 | 146 | 670 |
| ATCTAAAGGtCCCTGGTTCAATCCCGGGTTTCGGCA | | | | |
| GCCGAGATAGCTCAGTTGGGAGAGCGTTAGACTct | TAG | 208.17 | 174 | 3 |
| aGATCTAAAGGtCCCTGGTTCAATCCCGGGTTTCG | | | | |
| GCA | | | | |
| GCTGAAATAGCTCAGTTGGGAGAGCGTTAGACTctaG | TAG | 0.76 | 878 | 671 |
| ATCTtAAAGTtCCCTGGTTCAACCCTGGGTTTCAGCC | | | | |
| GCCGAAATAGCTCAGTTGGGAGAGCGTTAGACCctaG | TAG | 0.31 | 902 | 672 |
| ATCTtAAAGGtCCCTGGTTCAATCCCGGGTTTCGGCA | | | | |

TABLE 28C
Phenylalanine (Phe) tRNA

| | | fold | | SEQ |
|---|---|---|---|---|
| seq_tRNA | ACE | over | Rank | ID |
| GCCGAAATAGCTCAGTTGGGAGAGCGTTAGACTtcaG | TGA | 49.03 | 262 | 11 |
| ATCTAAAGGtCCCTGGTTCGATCCCGGGTTTCGGCA | | | | |
| GCCGAGATAGCTCAGTTGGGAGAGCGTTAGACTtcaG | TGA | 10.35 | 403 | 30 |
| ATCTAAAGGtCCCTGGTTCAATCCCGGGTTTCGGCA | | | | |
| GCCGAAATAGCTCAGTTGGGAGAGCGTTAGACTtcaG | TGA | 3.26 | 570 | 19 |
| ATCTAAAGGtCCCTGGTTCAATCCCGGGTTTCGGCA | | | | |
| GCTGAAATAGCTCAGTTGGGAGAGCGTTAGACTtcaG | TGA | 1.80 | 742 | 673 |
| ATCTtAAAGTtCCCTGGTTCAACCCTGGGTTTCAGCC | | | | |
| GCCGAAATAGCTCAGTTGGGAGAGCGTTAGACCtcaG | TGA | 1.51 | 790 | 674 |
| ATCTtAAAGGtCCCTGGTTCAATCCCGGGTTTCGGCA | | | | |

TABLE 29A
Proline (Pro) tRNA

| | | fold | | SEQ |
|---|---|---|---|---|
| seq_tRNA | ACE | over | Rank | ID |
| GGCTCGTTGGTCTAGGGGTATGATTCTCGGTTttaGTC | TAA | 5.05 | 489 | 675 |
| CGAGAGGICCCGGGTTCAAATCCCGGACGAGCCC | | | | |
| GGCTCGTTGGTCTAGGGGTATGATTCTCGCTTttaGTG | TAA | 2.45 | 638 | 676 |
| TGAGAGGtCCCGGGTTCAAATCCCGGACGAGCCC | | | | |
| GGCTCGTTGGTCTAGTGGTATGATTCTCGCTTttaGTGC | TAA | 1.79 | 746 | 677 |
| GAGAGGtCCCGGGTTCAAATCCCGGACGAGCCC | | | | |
| GGCTCGTTGGTCTAGGGGTATGATTCTCGCTTttaGTG | TAA | 1.68 | 762 | 678 |
| CGAGAGGtCCCGGGTTCAAATCCCGGACGAGCCC | | | | |
| GGCTCGTTGGTCTAGGGGTATGATTCTCGCTTttaATG | TAA | 1.45 | 806 | 679 |
| CGAGAGGtCCCGGGTTCAAATCCCGGACGAGCCC | | | | |

TABLE 29B
Proline (Pro) tRNA

| | | fold | | SEQ |
|---|---|---|---|---|
| seq_tRNA | ACE | over | Rank | ID |
| GGCTCGTTGGTCTAGGGGTATGATTCTCGGTTctaGTC | TAG | 3.70 | 542 | 680 |
| CGAGAGGtCCCGGGTTCAAATCCCGGACGAGCCC | | | | |
| GGCTCGTTGGTCTAGGGGTATGATTCTCGCTTctaGTG | TAG | 1.56 | 780 | 681 |
| TGAGAGGtCCCGGGTTCAAATCCCGGACGAGCCC | | | | |
| GGCTCGTTGGTCTAGTGGTATGATTCTCGCTTctaGTG | TAG | 1.51 | 791 | 682 |
| CGAGAGGtCCCGGGTTCAAATCCCGGACGAGCCC | | | | |
| GGCTCGTTGGTCTAGGGGTATGATTCTCGCTTctaGTG | TAG | 1.34 | 820 | 683 |
| CGAGAGGtCCCGGGTTCAAATCCCGGACGAGCCC | | | | |
| GGCTCGTTGGTCTAGGGGTATGATTCTCGCTTctaATG | TAG | 1.02 | 860 | 684 |
| CGAGAGGtCCCGGGTTCAAATCCCGGACGAGCCC | | | | |

TABLE 29C
Proline (Pro) tRNA

| | | fold | | SEQ |
|---|---|---|---|---|
| seq_tRNA | ACE | over | Rank | ID |
| GGCTCGTTGGTCTAGGGGTATGATTCTCGCTTtcaGTG | TGA | 1.94 | 719 | 685 |
| CGAGAGGtCCCGGGTTCAAATCCCGGACGAGCCC | | | | |
| GGCTCGTTGGTCTAGGGGTATGATTCTCGCTTtcaGTG | TGA | 1.93 | 720 | 686 |
| TGAGAGGtCCCGGGTTCAAATCCCGGACGAGCCC | | | | |
| GGCTCGTTGGTCTAGGGGTATGATTCTCGCTTtcaATG | TGA | 1.71 | 759 | 687 |
| CGAGAGGtCCCGGGTTCAAATCCCGGACGAGCCC | | | | |
| GGCTCGTTGGTCTAGTGGTATGATTCTCGCTTtcaGTG | TGA | 1.55 | 782 | 688 |
| CGAGAGGtCCCGGGTTCAAATCCCGGACGAGCCC | | | | |
| GGCTCGTTGGTCTAGGGGTATGATTCTCGGTTtcaGTC | TGA | 1.07 | 853 | 689 |
| CGAGAGGtCCCGGGTTCAAATCCCGGACGAGCCC | | | | |

TABLE 30A
Serine (Ser) tRNA

| | | fold | | SEQ |
|---|---|---|---|---|
| seq_tRNA | ACE | over | Rank | ID |
| GCTGTGATGGCCGAGTGGTtAAGGCGTTGGACTttaAA | TAA | 4838.23 | 15 | 690 |
| TCCAATGGGGTCTCCCCGCGCAGGTTCGAATCCTGC | | | | |
| TCACAGCG | | | | |
| GTCACGGTGGCCGAGTGGTtAAGGCGTTGGACTttaA | TAA | 4576.62 | 17 | 691 |
| ATCCAATGGGGTTTCCCCGCACAGGTTCGAATCCTG | | | | |
| TTCGTGACG | | | | |
| GACGAGGTGGCCGAGTGGTtAAGGCGATGGACTttaA | TAA | 4537.79 | 18 | 692 |
| ATCCATTGTGCTCTGCACGCGTGGGTTCGAATCCCA | | | | |
| CCTTCGTCG | | | | |
| GCAGCGATGGCCGAGTGGTtAAGGCGTTGGACTttaA | TAA | 4174.34 | 23 | 693 |
| ATCCAATGGGGTCTCCCCGCGCAGGTTCGAACCCTG | | | | |
| CTCGCTGCG | | | | |
| GTAGTCGTGGCCGAGTGGTtAAGGCGATGGACTttaA | TAA | 3855.01 | 30 | 694 |
| ATCCATTGGGGTCTCCCCGCGCAGGTTCGAATCCTG | | | | |
| CCGACTACG | | | | |
| GTAGTCGTGGCCGAGTGGTtAAGGCGATGGACTttaA | TAA | 2707.86 | 44 | 695 |
| ATCCATTGGGGTTTCCCCGCGCAGGTTCGAATCCTG | | | | |
| TCGGCTACG | | | | |
| GCTGTGATGGCCGAGTGGTtAAGGCGTTGGACTttaAA | TAA | 2689.19 | 45 | 696 |
| TCCAATGGGGTCTCCCCGCGCAGGTTCAAATCCTGC | | | | |
| TCACAGCG | | | | |
| GACGAGGTGGCCGAGTGGTtAAGGCGATGGACTttaA | TAA | 2665.73 | 46 | 697 |
| ATCCATTGTGCTCTGCACACGTGGGTTCGAATCCCA | | | | |
| TCCTCGTCG | | | | |
| GTAGTCGTGGCCGAGTGGTtAAGGCGATGGACTttaA | TAA | 2561.19 | 48 | 698 |
| ATCCATTGGGGTTTCCCCACGCAGGTTCGAATCCTG | | | | |
| CCGACTACG | | | | |
| GCTGTGATGGCCGAGTGGTtAAGGTGTTGGACTttaAA | TAA | 2215.62 | 57 | 699 |
| TCCAATGGGGGTTCCCCGCGCAGGTTCAAATCCTGC | | | | |
| TCACAGCG | | | | |
| GACGAGGTGGCCGAGTGGTtAAGGCGATGGACTttaA | TAA | 1879.18 | 61 | 700 |
| ATCCATTGTGCTCTGCACGCGTGGGTTCGAATCCCA | | | | |
| CCCTCGTCG | | | | |
| GTAGTCGTGGCCGAGTGGTtAAGGTGATGGACTttaA | TAA | 294.33 | 155 | 701 |
| ACCCATTGGGGTCTCCCCGCGCAGGTTCGAATCCTG | | | | |
| CCGACTACG | | | | |
| GGCCGGTTAGCTCAGTTGGTtAGAGCGTGCTttaACTA | TAA | 2.48 | 635 | 702 |
| ATGCCAGGGtCGAGGTTTCGATCCCCGTACGGGCCT | | | | |
| CAAGAAATTCATAGAGGTTATGGGATTGGCTttaAAC | TAA | 2.40 | 646 | 703 |
| CAGTTTcAGGAGGTTCGATTCCTTCCTTTTTGG | | | | |
| GAGGCCTGGCCGAGTGGTtAAGGCGATGGACTttaAA | TAA | 2.04 | 706 | 704 |
| TCCATTGTGCTCTGCACGCGTGGGTTCGAATCCCAT | | | | |
| CCTCG | | | | |
| GGCCGGTTAGCTCAGTTGGTtAGAGCGTGCTGCTttaA | TAA | 1.09 | 852 | 705 |
| ATGCCAGGGtCGAGGTTTCGATCCCCGTACGGGCCT | | | | |
| AGTTGTAGCTGAGTGGTtAAGGCAACGAGCTttaAATT | TAA | 0.71 | 883 | 706 |
| CGTTGGTTTCTCTCTgTGCAGGTTTGAATCCTGCTAA | | | | |
| TTA | | | | |
| GGGTGTATGGCTCAGGGGTAGAGAATTTGACTttaGA | TAA | 0.60 | 891 | 707 |
| TCAAGAGGtCCCTGGTTCAAATCCAGGTGCCCCCT | | | | |

TABLE 30B
Serine (Ser) tRNA

| | ACE | fold | | SEQ |
|---|---|---|---|---|
| seq_tRNA | | over | Rank | ID |
| GTAGTCGTGGCCGAGTGGTtAAGGCGATGGACTctaA | TAG | 6722.46 | 7 | 708 |
| ATCCATTGGGGTTTCCCCGCGCAGGTTCGAATCCTG | | | | |
| TCGGCTACG | | | | |
| GCTGTGATGGCCGAGTGGTtAAGGCGTTGGACTctaA | TAG | 6131.32 | 11 | 709 |
| ATCCAATGGGGTCTCCCCGCGCAGGTTCAAATCCTG | | | | |
| CTCACAGCG | | | | |
| GCAGCGATGGCCGAGTGGTtAAGGCGTTGGACTctaA | TAG | 4806.34 | 16 | 710 |
| ATCCAATGGGGTCTCCCCGCGCAGGTTCGAACCCTG | | | | |
| CTCGCTGCG | | | | |
| GACGAGGTGGCCGAGTGGTtAAGGCGATGGACTctaA | TAG | 4196.45 | 21 | 711 |
| ATCCATTGTGCTCTGCACACGTGGGTTCGAATCCCA | | | | |
| TCCTCGTCG | | | | |
| GACGAGGTGGCCGAGTGGTtAAGGCGATGGACTctaA | TAG | 4045.03 | 26 | 712 |
| ATCCATTGTGCTCTGCACGCGTGGGTTCGAATCCCA | | | | |
| CCTTCGTCG | | | | |
| GTCACGGTGGCCGAGTGGTtAAGGCGTTGGACTctaA | TAG | 3592.39 | 32 | 713 |
| ATCCAATGGGGTTTCCCCGCACAGGTTCGAATCCTG | | | | |
| TTCGTGACG | | | | |
| GACGAGGTGGCCGAGTGGTtAAGGCGATGGACTctaA | TAG | 3300.23 | 34 | 714 |
| ATCCATTGTGCTCTGCACGCGTGGGTTCGAATCCCA | | | | |
| CCCTCGTCG | | | | |
| GTAGTCGTGGCCGAGTGGTtAAGGCGATGGACTctaA | TAG | 2884.39 | 41 | 715 |
| ATCCATTGGGGTCTCCCCGCGCAGGTTCGAATCCTG | | | | |
| CCGACTACG | | | | |
| GGCCGGTTAGCTCAGTTGGTtAGAGCGTGCTctaACTA | TAG | 2536.06 | 50 | 716 |
| ATGCCAGGGtCGAGGTTTCGATCCCCGTACGGGCCT | | | | |
| GCTGTGATGGCCGAGTGGTtAAGGTGTTGGACTctaA | TAG | 2470.61 | 53 | 717 |
| ATCCAATGGGGGTTCCCCGCGCAGGTTCAAATCCTG | | | | |
| CTCACAGCG | | | | |
| GTAGTCGTGGCCGAGTGGTtAAGGCGATGGACTctaA | TAG | 2434.53 | 54 | 718 |
| ATCCATTGGGGTTTCCCCACGCAGGTTCGAATCCTG | | | | |
| CCGACTACG | | | | |
| GCTGTGATGGCCGAGTGGTtAAGGCGTTGGACTctaA | TAG | 1382.40 | 72 | 719 |
| ATCCAATGGGGTCTCCCCGCGCAGGTTCGAATCCTG | | | | |
| CTCACAGCG | | | | |
| GTAGTCGTGGCCGAGTGGTtAAGGTGATGGACTctaA | TAG | 1313.05 | 73 | 720 |
| ACCCATTGGGGTCTCCCCGCGCAGGTTCGAATCCTG | | | | |
| CCGACTACG | | | | |
| GAGGCCTGGCCGAGTGGTtAAGGCGATGGACTctaAA | TAG | 6.65 | 454 | 721 |
| TCCATTGTGCTCTGCACGCGTGGGTTCGAATCCCAT | | | | |
| CCTCG | | | | |
| GGCCGGTTAGCTCAGTTGGTtAGAGCGTGCTGCTctaA | TAG | 6.32 | 460 | 722 |
| ATGCCAGGGCGAGGTTTCGATCCCCGTACGGGCCT | | | | |
| GGGTGTATGGCTCAGGGGTAGAGAATTTGACTctaGA | TAG | 3.13 | 579 | 723 |
| TCAAGAGGtCCCTGGTTCAAATCCAGGTGCCCCCT | | | | |
| CAAGAAATTCATAGAGGTTATGGGATTGGCTctaAAC | TAG | 2.87 | 600 | 724 |
| CAGTTTcAGGAGGTTCGATTCCTTCCTTTTTGG | | | | |
| AGTTGTAGCTGAGTGGTtAAGGCAACGAGCTctaAAT | TAG | 2.08 | 694 | 725 |
| TCGTTGGTTTCTCTCTgTGCAGGTTTGAATCCTGCTA | | | | |
| ATTA | | | | |

TABLE 30C
Serine (Ser) tRNA

| | | fold | | SEQ |
|---|---|---|---|---|
| seq_tRNA | ACE | over | Rank | ID |
| GCTGTGATGGCCGAGTGGTtAAGGTGTTGGACTtcaA | TGA | 1618.12 | 67 | 726 |
| ATCCAATGGGGGTTCCCCGCGCAGGTTCAAATCCTG | | | | |
| CTCACAGCG | | | | |
| GTAGTCGTGGCCGAGTGGTtAAGGCGATGGACTtcaA | TGA | 1611.87 | 68 | 727 |
| ATCCATTGGGGTTTCCCCACGCAGGTTCGAATCCTG | | | | |
| CCGACTACG | | | | |
| GCTGTGATGGCCGAGTGGTtAAGGCGTTGGACTtcaA | TGA | 1502.68 | 69 | 728 |
| ATCCAATGGGGTCTCCCCGCGCAGGTTCGAATCCTG | | | | |
| CTCACAGCG | | | | |
| GCTGTGATGGCCGAGTGGTtAAGGCGTTGGACTtcaA | TGA | 1399.00 | 71 | 729 |
| ATCCAATGGGGTCTCCCCGCGCAGGTTCAAATCCTG | | | | |
| CTCACAGCG | | | | |
| GCAGCGATGGCCGAGTGGTtAAGGCGTTGGACTtcaA | TGA | 1300.07 | 74 | 730 |
| ATCCAATGGGGTCTCCCCGCGCAGGTTCGAACCCTG | | | | |
| CTCGCTGCG | | | | |
| GACGAGGTGGCCGAGTGGTtAAGGCGATGGACTtcaA | TGA | 1299.61 | 75 | 731 |
| ATCCATTGTGCTCTGCACGCGTGGGTTCGAATCCCA | | | | |
| CCTTCGTCG | | | | |
| GTAGTCGTGGCCGAGTGGTtAAGGCGATGGACTtcaA | TGA | 1299.53 | 76 | 732 |
| ATCCATTGGGGTCTCCCCGCGCAGGTTCGAATCCTG | | | | |
| CCGACTACG | | | | |
| GACGAGGTGGCCGAGTGGTtAAGGCGATGGACTtcaA | TGA | 1219.85 | 81 | 733 |
| ATCCATTGTGCTCTGCACGCGTGGGTTCGAATCCCA | | | | |
| CCCTCGTCG | | | | |
| GTCACGGTGGCCGAGTGGTtAAGGCGTTGGACTtcaA | TGA | 1193.55 | 83 | 734 |
| ATCCAATGGGGTTTCCCCGCACAGGTTCGAATCCTG | | | | |
| TTCGTGACG | | | | |
| GTAGTCGTGGCCGAGTGGTtAAGGCGATGGACTtcaA | TGA | 1187.29 | 84 | 735 |
| ATCCATTGGGGTTTCCCCGCGCAGGTTCGAATCCTG | | | | |
| TCGGCTACG | | | | |
| GACGAGGTGGCCGAGTGGTtAAGGCGATGGACTtcaA | TGA | 1113.35 | 87 | 736 |
| ATCCATTGTGCTCTGCACACGTGGGTTCGAATCCCA | | | | |
| TCCTCGTCG | | | | |
| GTAGTCGTGGCCGAGTGGTtAAGGTGATGGACTtcaA | TGA | 387.56 | 142 | 737 |
| ACCCATTGGGGTCTCCCCGCGCAGGTTCGAATCCTG | | | | |
| CCGACTACG | | | | |
| AGTTGTAGCTGAGTGGTtAAGGCAACGAGCTtcaAAT | TGA | 3.34 | 566 | 738 |
| TCGTTGGTTTCTCTCTgTGCAGGTTTGAATCCTGCTA | | | | |
| ATTA | | | | |
| GGGTGTATGGCTCAGGGGTAGAGAATTTGACTtcaGA | TGA | 2.99 | 592 | 739 |
| TCAAGAGGtCCCTGGTTCAAATCCAGGTGCCCCCT | | | | |
| GAGGCCTGGCCGAGTGGTtAAGGCGATGGACTtcaAA | TGA | 2.97 | 594 | 740 |
| TCCATTGTGCTCTGCACGCGTGGGTTCGAATCCCAT | | | | |
| CCTCG | | | | |
| CAAGAAATTCATAGAGGTTATGGGATTGGCTtcaAAC | TGA | 2.55 | 626 | 741 |
| CAGTTTcAGGAGGTTCGATTCCTTCCTTTTTGG | | | | |
| GGCCGGTTAGCTCAGTTGGTtAGAGCGTGCTtcaACTA | TGA | 2.52 | 628 | 742 |
| ATGCCAGGGtCGAGGTTTCGATCCCCGTACGGGCCT | | | | |
| GGCCGGTTAGCTCAGTTGGTtAGAGCGTGCTGCTtcaA | TGA | 2.39 | 648 | 743 |
| ATGCCAGGGtCGAGGTTTCGATCCCCGTACGGGCCT | | | | |

TABLE 31A
Threonine (The) tRNA

| | | fold | | SEQ |
|---|---|---|---|---|
| seq_tRNA | ACE | over | Rank | ID |
| GGCTCCATAGCTCAGTGGTtAGAGCACTGGTCTttaAA | TAA | 126.45 | 202 | 744 |
| ACCAGGGGtCGCGAGTTCGATCCTCGCTGGGGCCT | | | | |
| GGCGCCGTGGCTTAGCTGGTAAAGCGCCTGTCTttaA | TAA | 52.93 | 254 | 745 |
| AACAGGAGAtCCTGGGTTCGAATCCCAGCGGTGCCT | | | | |
| GGCTCCGTGGCTTAGCTGGTtAAAGCGCCTGTCTttaA | TAA | 51.01 | 259 | 746 |
| AACAGGAGAtCCTGGGTTCGAATCCCAGCGGGGCCT | | | | |
| GGCTCCGTAGCTTAGTTGGTtAAAGCGCCTGTCTttaA | TAA | 44.24 | 275 | 747 |
| AACAGGAGAtCCTGGGTTCGACTCCCAGCGGGGCCT | | | | |
| GGCTCTATGGCTTAGTTGGTtAAAGCGCCTGTCTttaA | TAA | 40.18 | 283 | 748 |
| AACAGGAGAtCCTGGGTTCGACTCCCAGTGGGGCCT | | | | |
| GGCCCTGTGGCTTAGCTGGTcAAAGCGCCTGTCTttaA | TAA | 40.07 | 284 | 749 |
| AACAGGAGAtCCTGGGTTCGAATCCCAGCGGGGCCT | | | | |
| GGCTCTATGGCTTAGTTGGTtAAAGCGCCTGTCTttaA | TAA | 38.09 | 288 | 750 |
| AACAGGAGAtCCTGGGTTCGAATCCCAGTAGAGCCT | | | | |
| GGCGCCGTGGCTTAGTTGGTtAAAGCGCCTGTCTttaA | TAA | 34.94 | 292 | 751 |
| AACAGGAGAtCCTGGGTTCGAATCCCAGCGGTGCCT | | | | |
| GGCTCCATAGCTCAGGGGTtAGAGCACTGGTCTttaAA | TAA | 33.58 | 297 | 752 |
| ACCAGGGGtCGCGAGTTCAAATCTCGCTGGGGCCT | | | | |
| GGCTCTGTGGCTTAGTTGGCtAAAGCGCCTGTCTttaA | TAA | 33.26 | 299 | 753 |
| AACAGGAGAtCCTGGGTTCGAATCCCAGCGGGGCCT | | | | |
| GGCTTCGTGGCTTAGCTGGTtAAAGCGCCTGTCTttaA | TAA | 32.41 | 303 | 754 |
| AACAGGAGAtCCTGGGTTCGAATCCCAGCGAGGCCT | | | | |
| GGCGCGGTGGCCAAGTGGTAAGGCGTCGGTCTttaAA | TAA | 30.81 | 307 | 755 |
| ACCGAAGAtCGCGGGTTCGAACCCCGTCCGTGCCT | | | | |
| GGCGCGGTGGCCAAGTGGTAAGGCGTCGGTCTttaAA | TAA | 28.06 | 316 | 756 |
| ACCGAAGAtCACGGGTTCGAACCCCGTCCGTGCCT | | | | |
| GGCCCTATAGCTCAGGGGTtAGAGCACTGGTCTttaAA | TAA | 10.71 | 397 | 757 |
| ACCAGGGGtCGCGAGTTCAAATCTCGCTGGGGCCT | | | | |
| GGCTCCATAGCTCAGGGGTtAGAGCGCTGGTCTttaAA | TAA | 8.12 | 430 | 758 |
| ACCAGGGGtCGCGAGTTCAATTCTCGCTGGGGCCT | | | | |
| GGCTCCATAGCTCAGGGGTtAGAGCACTGGTCTttaAA | TAA | 2.14 | 684 | 759 |
| ACCAGGGtCGCGAGTTCAAATCTCGCTGGGGCCT | | | | |
| GGCCCTGTAGCTCAGCGGTtGGAGCGCTGGTCTttaAA | TAA | 1.72 | 755 | 760 |
| ACCtAGGGGtCGTGAGTTCAAATCTCACCAGGGCCT | | | | |

TABLE 31B
Threonine (The) tRNA

| | | fold | | SEQ |
|---|---|---|---|---|
| seq_tRNA | ACE | over | Rank | ID |
| GGCTCCATAGCTCAGGGGTtAGAGCACTGGTCTctaAA | TAG | 44.31 | 274 | 761 |
| ACCAGGGGtCGCGAGTTCAAATCTCGCTGGGGCCT | | | | |
| GGCTCCATAGCTCAGTGGTtAGAGCACTGGTCTctaAA | TAG | 43.02 | 277 | 762 |
| ACCAGGGGtCGCGAGTTCGATCCTCGCTGGGGCCT | | | | |
| GGCCCTATAGCTCAGGGGTtAGAGCACTGGTCTctaAA | TAG | 39.34 | 285 | 763 |
| ACCAGGGGtCGCGAGTTCAAATCTCGCTGGGGCCT | | | | |
| GGCTCCGTGGCTTAGCTGGTtAAAGCGCCTGTCTctaA | TAG | 37.49 | 290 | 32 |
| AACAGGAGAtCCTGGGTTCGAATCCCAGCGGGGCCT | | | | |
| GGCTCTGTGGCTTAGTTGGCtAAAGCGCCTGTCTctaA | TAG | 32.99 | 300 | 15 |
| AACAGGAGAtCCTGGGTTCGAATCCCAGCGGGGCCT | | | | |
| GGCGCGGTGGCCAAGTGGTAAGGCGTCGGTCTctaAA | TAG | 30.75 | 308 | 764 |
| ACCGAAGAtCGCGGGTTCGAACCCCGTCCGTGCCT | | | | |
| GGCTCTATGGCTTAGTTGGTtAAAGCGCCTGTCTctaA | TAG | 30.57 | 310 | 26 |
| AACAGGAGAtCCTGGGTTCGAATCCCAGTAGAGCCT | | | | |
| GGCTCCGTAGCTTAGTTGGTtAAAGCGCCTGTCTctaA | TAG | 29.84 | 312 | 18 |
| AACAGGAGAtCCTGGGTTCGACTCCCAGCGGGGCCT | | | | |
| GGCCCTGTGGCTTAGCTGGTcAAAGCGCCTGTCTctaA | TAG | 28.92 | 315 | 765 |
| AACAGGAGAtCCTGGGTTCGAATCCCAGCGGGGCCT | | | | |
| GGCGCCGTGGCTTAGTTGGTtAAAGCGCCTGTCTctaA | TAG | 20.16 | 343 | 766 |
| AACAGGAGAtCCTGGGTTCGAATCCCAGCGGTGCCT | | | | |
| GGCGCGGTGGCCAAGTGGTAAGGCGTCGGTCTctaAA | TAG | 19.23 | 347 | 24 |
| ACCGAAGAtCACGGGTTCGAACCCCGTCCGTGCCT | | | | |
| GGCTCTATGGCTTAGTTGGTtAAAGCGCCTGTCTctaA | TAG | 13.63 | 370 | 767 |
| AACAGGAGAtCCTGGGTTCGACTCCCAGTGGGGCCT | | | | |
| GGCTTCGTGGCTTAGCTGGTAAAGCGCCTGTCTctaA | TAG | 10.95 | 394 | 768 |
| AACAGGAGAtCCTGGGTTCGAATCCCAGCGAGGCCT | | | | |
| GGCGCCGTGGCTTAGCTGGTtAAAGCGCCTGTCTctaA | TAG | 9.75 | 411 | 769 |
| AACAGGAGAtCCTGGGTTCGAATCCCAGCGGTGCCT | | | | |
| GGCTCCATAGCTCAGGGGTtAGAGCGCTGGTCTctaAA | TAG | 6.23 | 462 | 770 |
| ACCAGGGGtCGCGAGTTCAATTCTCGCTGGGGCCT | | | | |
| GGCCCTGTAGCTCAGCGGTtGGAGCGCTGGTCTctaAA | TAG | 1.33 | 821 | 771 |
| ACCtAGGGGtCGTGAGTTCAAATCTCACCAGGGCCT | | | | |
| GGCTCCATAGCTCAGGGGTAGAGCACTGGTCTctaAA | TAG | 1.31 | 828 | 772 |
| ACCAGGGtCGCGAGTTCAAATCTCGCTGGGGCCT | | | | |

TABLE 31C
Threonine (The) tRNA

| | | fold | | SEQ |
|---|---|---|---|---|
| seq_tRNA | ACE | over | Rank | ID |
| GGCGCGGTGGCCAAGTGGTAAGGCGTCGGTCTtcaAA | TGA | 26.39 | 321 | 773 |
| ACCGAAGAtCACGGGTTCGAACCCCGTCCGTGCCT | | | | |
| GGCGCGGTGGCCAAGTGGTAAGGCGTCGGTCTtcaAA | TGA | 16.95 | 358 | 27 |
| ACCGAAGAtCGCGGGTTCGAACCCCGTCCGTGCCT | | | | |
| GGCTCCGTGGCTTAGCTGGTtAAAGCGCCTGTCTtcaA | TGA | 16.20 | 362 | 774 |
| AACAGGAGAtCCTGGGTTCGAATCCCAGCGGGGCCT | | | | |
| GGCTCCATAGCTCAGTGGTtAGAGCACTGGTCTtcaAA | TGA | 15.71 | 366 | 775 |
| ACCAGGGGtCGCGAGTTCGATCCTCGCTGGGGCCT | | | | |
| GGCCCTGTGGCTTAGCTGGTcAAAGCGCCTGTCTtcaA | TGA | 13.00 | 373 | 776 |
| AACAGGAGAtCCTGGGTTCGAATCCCAGCGGGGCCT | | | | |
| GGCTCTGTGGCTTAGTTGGCtAAAGCGCCTGTCTtcaA | TGA | 10.14 | 407 | 777 |
| AACAGGAGAtCCTGGGTTCGAATCCCAGCGGGGCCT | | | | |
| GGCCCTATAGCTCAGGGGTtAGAGCACTGGTCTtcaAA | TGA | 8.84 | 419 | 778 |
| ACCAGGGGtCGCGAGTTCAAATCTCGCTGGGGCCT | | | | |
| GGCGCCGTGGCTTAGCTGGTtAAAGCGCCTGTCTtcaA | TGA | 8.76 | 420 | 779 |
| AACAGGAGAtCCTGGGTTCGAATCCCAGCGGTGCCT | | | | |
| GGCTCTATGGCTTAGTTGGTtAAAGCGCCTGTCTtcaA | TGA | 8.41 | 423 | 780 |
| AACAGGAGAtCCTGGGTTCGACTCCCAGTGGGGCCT | | | | |
| GGCTCTATGGCTTAGTTGGTtAAAGCGCCTGTCTtcaA | TGA | 7.65 | 435 | 781 |
| AACAGGAGAtCCTGGGTTCGAATCCCAGTAGAGCCT | | | | |
| GGCGCCGTGGCTTAGTTGGTtAAAGCGCCTGTCTtcaA | TGA | 7.59 | 438 | 782 |
| AACAGGAGAtCCTGGGTTCGAATCCCAGCGGTGCCT | | | | |
| GGCTTCGTGGCTTAGCTGGTtAAAGCGCCTGTCTtcaA | TGA | 7.15 | 445 | 783 |
| AACAGGAGAtCCTGGGTTCGAATCCCAGCGAGGCCT | | | | |
| GGCTCCGTAGCTTAGTTGGTtAAAGCGCCTGTCTtcaA | TGA | 6.87 | 451 | 784 |
| AACAGGAGAtCCTGGGTTCGACTCCCAGCGGGGCCT | | | | |
| GGCTCCATAGCTCAGGGGTtAGAGCACTGGTCTtcaAA | TGA | 5.82 | 467 | 785 |
| ACCAGGGGtCGCGAGTTCAAATCTCGCTGGGGCCT | | | | |
| GGCTCCATAGCTCAGGGGTtAGAGCGCTGGTCTtcaAA | TGA | 4.82 | 498 | 786 |
| ACCAGGGGtCGCGAGTTCAATTCTCGCTGGGGCCT | | | | |
| GGCCCTGTAGCTCAGCGGTtGGAGCGCTGGTCTtcaAA | TGA | 1.46 | 802 | 787 |
| ACCtAGGGGtCGTGAGTTCAAATCTCACCAGGGCCT | | | | |
| GGCTCCATAGCTCAGGGGTtAGAGCACTGGTCTtcaAA | TGA | 1.09 | 851 | 788 |
| ACCAGGGtCGCGAGTTCAAATCTCGCTGGGGCCT | | | | |

TABLE 32A
Tryptophan (Trp) tRNA

| | | fold | | SEQ |
|---|---|---|---|---|
| seq_tRNA | ACE | over | Rank | ID |
| GACCTCGTGGCGCAATGGTAGCGCGTCTGACTttaGAT | TAA | 135.39 | 198 | 789 |
| CAGAAGGtTGCGTGTTCAAGTCACGTCGGGGTCA | | | | |
| GACCTCGTGGCGCAACGGCAGCGCGTCTGACTttaGAT | TAA | 106.21 | 214 | 790 |
| CAGAAGGtGCGTGTTCAAATCACGTCGGGGTCA | | | | |
| GACCTCGTGGCGCAACGGTAGCGCGTCTGACTttaGAT | TAA | 105.08 | 215 | 791 |
| CAGAAGGtTGCGTGTTCAAATCACGTCGGGGTCA | | | | |
| GACCTCGTGGCGCAACGGTAGCGCGTCTGACTttaGAT | TAA | 65.12 | 243 | 792 |
| CAGAAGGcTGCGTGTTCGAATCACGTCGGGGTCA | | | | |
| GGCCTCGTGGCGCAACGGTAGCGCGTCTGACTttaGAT | TAA | 54.99 | 252 | 793 |
| CAGAAGGtGCGTGTTCAAATCACGTCGGGGTCA | | | | |
| GGCCTCATGGTGCAACAGTAGTGTGTCTGACTttaGATC | TAA | 3.38 | 565 | 794 |
| AGAAGGtTGTATGTTCAAATCACGTAGGGGTCA | | | | |

TABLE 32B
Tryptophan (Trp) tRNA

| | | fold | | SEQ |
|---|---|---|---|---|
| seq_tRNA | ACE | over | Rank | ID |
| GGCCTCATGGTGCAACAGTAGTGTGTCTGACTctaG | TAG | 2550.4 | 49 | 795 |
| ATCAGAAGGtTGTATGTTCAAATCACGTAGGGGTC | | | | |
| A | | | | |
| GACCTCGTGGCGCAACGGTAGCGCGTCTGACTctaGAT | TAG | 1296.17 | 77 | 796 |
| CAGAAGGcTGCGTGTTCGAATCACGTCGGGGTCA | | | | |
| GGCCTCGTGGCGCAACGGTAGCGCGTCTGACTctaGAT | TAG | 1204.7 | 82 | 797 |
| CAGAAGGtTGCGTGTTCAAATCACGTCGGGGTCA | | | | |
| GACCTCGTGGCGCAATGGTAGCGCGTCTGACTctaGAT | TAG | 615.12 | 112 | 797 |
| CAGAAGGtTGCGTGTTCAAGTCACGTCGGGGTCA | | | | |
| GACCTCGTGGCGCAACGGTAGCGCGTCTGACTctaGAT | TAG | 551.09 | 120 | 799 |
| CAGAAGGtTGCGTGTTCAAATCACGTCGGGGTCA | | | | |
| GACCTCGTGGCGCAACGGCAGCGCGTCTGACTctaGAT | TAG | 204.73 | 176 | 800 |
| CAGAAGGtTGCGTGTTCAAATCACGTCGGGGTCA | | | | |

TABLE 32C
Tryptophan (Trp) tRNA

| | | fold | | SEQ |
|---|---|---|---|---|
| seq_tRNA | ACE | over | Rank | ID |
| GGCCTCGTGGCGCAACGGTAGCGCGTCTGACTtcaGAT | TGA | 13.71 | 369 | 801 |
| CAGAAGGtTGCGTGTTCAAATCACGTCGGGGTCA | | | | |
| GACCTCGTGGCGCAATGGTAGCGCGTCTGACTtcaGAT | TGA | 11.58 | 389 | 802 |
| CAGAAGGtTGCGTGTTCAAGTCACGTCGGGGTCA | | | | |
| GACCTCGTGGCGCAACGGTAGCGCGTCTGACTtcaGAT | TGA | 8.64 | 421 | 803 |
| CAGAAGGcTGCGTGTTCGAATCACGTCGGGGTCA | | | | |
| GACCTCGTGGCGCAACGGTAGCGCGTCTGACTtcaGAT | TGA | 6.88 | 450 | 804 |
| CAGAAGGtTGCGTGTTCAAATCACGTCGGGGTCA | | | | |
| GACCTCGTGGCGCAACGGCAGCGCGTCTGACTtcaGAT | TGA | 5.34 | 480 | 805 |
| CAGAAGGtTGCGTGTTCAAATCACGTCGGGGTCA | | | | |
| GGCCTCATGGTGCAACAGTAGTGTGTCTGACTtcaGAT | TGA | 2.46 | 637 | 806 |
| CAGAAGGtTGTATGTTCAAATCACGTAGGGGTCA | | | | |

TABLE 33A
Tyrosine (Tyr) tRNA

| | | fold | | SEQ |
|---|---|---|---|---|
| seq_tRNA | ACE | over | Rank | ID |
| CCTTCGATAGCTCAGTTGGTAGAGCGGAGGACTttaG | TAA | 2744.1 | 42 | 807 |
| ATCCTTAGGtCGCTGGTTCGAATCCGGCTCGAAGGA | | | | |
| CCTTCGATAGCTCAGCTGGTAGAGCGGAGGACTttaG | TAA | 2586.0 | 47 | 808 |
| ATCCTTAGGtCGCTGGTTCGAATCCGGCTCGGAGGA | | | | |
| CCTTCGATAGCTCAGTTGGTAGAGCGGAGGACTttaG | TAA | 2522.5 | 51 | 809 |
| ATCCTTAGGtCGCTGGTTCGAATCCGGCTCGGAGGA | | | | |
| CCTTCGATAGCTCAGTTGGTAGAGCGGAGGACTttaG | TAA | 2497.0 | 52 | 810 |
| ATCCTTAGGtCGCTGGTTCGATTCCGGCTCGAAGGA | | | | |
| CCTTCGATAGCTCAGCTGGTAGAGCGGAGGACTttaG | TAA | 916.5 | 92 | 811 |
| ATCCTTAGGtCGCTGGTTCGATTCCAGCTCGAAGGA | | | | |
| CCTTCGATAGCTCAGCTGGTAGAGCGGAGGACTttaG | TAA | 273.9 | 159 | 812 |
| ATCCTTAGGtCGCTGGTTCAATTCCGGCTCGAAGGA | | | | |
| CTTTCGATAGCTCAGTTGGTAGAGCGGAGGACTttaG | TAA | 215.7 | 170 | 813 |
| ATCCTTAGGtCGCTGGTTCGAATCCGGCTCGAAGGA | | | | |
| GGTAAAATGACTGAGTAAGCATTAGACTttaAATCTA | TAA | 117.4 | 207 | 814 |
| AAGaCAGAGGTCAAGACCTCTTTTTACCA | | | | |
| GGTAAAATGGCTGAGTAAGCATTAGACTttaAATCTA | TAA | 11.1 | 392 | 815 |
| AAGaCAGAGGTCAAGGCCTCTTTTTTCCT | | | | |
| GTCAGTGTTGCACAACGGTtaAGTGAAGAGGCTttaAA | TAA | 8.2 | 427 | 816 |
| CCCAGACTGGATGGGTTCAATTCCCATCTCTGCCG | | | | |
| GGTAAAATGGCTGAGTAAAGCATTAGACTttaAATCT | TAA | 7.6 | 434 | 817 |
| AAGGaCAGAGGCTAAACCTCTTTTTACCA | | | | |
| GGTAAAATGGCTGAGTGAAGCATTGGACTttaAATCT | TAA | 5.6 | 474 | 818 |
| AAAGaCAGGGGTTAAGCCTCTTTTTACCA | | | | |
| TATAGCTCAGTGGTAGAGCATTTAACTttaGATCAAG | TAA | 5.6 | 475 | 819 |
| AGGtCCCTGGATCAACTCTGGGTG | | | | |
| GGTAAAATGGCTGAGTAAGCATTAGACTttaAATCTA | TAA | 5.2 | 484 | 820 |
| AAGaCAGAGGTCAAGGCCTTTTTACCA | | | | |
| GGTAAAATGGCTGAGCAAGCATTGGACTttaAATCTA | TAA | 3.9 | 529 | 821 |
| AAGaCAGATGTTGAGCCATCTTTTTAGCA | | | | |
| GGTAAAATGGCTGAGCAAGCATTAGACTttaAATCTG | TAA | 3.7 | 539 | 822 |
| AAAaCAGAGGTCAAAGgTCTCTTTTTACCA | | | | |
| GGTAAAATGGCTGAGCAAGCATTAGACTttaAATCTA | TAA | 3.5 | 553 | 823 |
| AAGaCAGAGGTTAAGGCCTCTTTTTACCA | | | | |
| GGTAAAATGGCTGAGTAAGCATTGGACTttaAATCTA | TAA | 2.4 | 632 | 824 |
| AAGaCAGAGGTCAAGACCTCTTTTTACCA | | | | |
| GGTAAAATGACTGAATAAGCCTTAGACTttaAATCTG | TAA | 2.3 | 662 | 825 |
| AAGaCAGAGGTCAAGGCCTCTTTTTACCA | | | | |
| GGTAAAATGGCTGAGTAAGCTTTAGACTttaAATCTA | TAA | 1.5 | 794 | 826 |
| AAGAGAGATTGAGCTCTCTTTTTACCA | | | | |
| GGGGGTATAGCTCAGGGCtAGAGCTtTTTGACTttaGA | TAA | 1.4 | 798 | 827 |
| GCAAGAGGtCCCTGGTTCAAATCCAGGTTCTCCCT | | | | |
| CCTTCAATAGTTCAGCTGGTAGAGCAGAGGACTttaG | TAA | 1.4 | 805 | 828 |
| GTCCTTAGGtTGCTGGTTCGATTCCAGCTTGAAGGA | | | | |
| GGTAAAATGGCTGAGTGAAGCATTGGACTttaAATCT | TAA | 0.83 | 875 | 829 |
| AAAGaCAGGGGCTAAGCCTCTTTTTACCA | | | | |
| TCTTCAATAGCTCAGCTGGTAGAGCGGAGGACTttaG | TAA | 0.06 | 912 | 830 |
| ATTCTTAGGTGCTGGTTTGATTCCGACTTGGAGAG | | | | |

TABLE 33B
Tyrosine (Tyr) tRNA

| | | fold | | SEQ |
|---|---|---|---|---|
| seq_tRNA | ACE | over | Rank | ID |
| CCTTCGATAGCTCAGTTGGTAGAGCGGAGGACTctaG | TAG | 9722.7 | 2 | 831 |
| ATCCTTAGGtCGCTGGTTCGAATCCGGCTCGGAGGA | | | | |
| CCTTCGATAGCTCAGCTGGTAGAGCGGAGGACTctaG | TAG | 8890.7 | 3 | 832 |
| ATCCTTAGGtCGCTGGTTCGATTCCAGCTCGAAGGA | | | | |
| CCTTCGATAGCTCAGCTGGTAGAGCGGAGGACTctaG | TAG | 8149.2 | 4 | 833 |
| ATCCTTAGGtCGCTGGTTCGAATCCGGCTCGGAGGA | | | | |
| CCTTCGATAGCTCAGCTGGTAGAGCGGAGGACTctaG | TAG | 7656.9 | 5 | 834 |
| ATCCTTAGGtCGCTGGTTCAATTCCGGCTCGAAGGA | | | | |
| CCTTCGATAGCTCAGTTGGTAGAGCGGAGGACTctaG | TAG | 1803.7 | 62 | 835 |
| ATCCTTAGGtCGCTGGTTCGATTCCGGCTCGAAGGA | | | | |
| CCTTCGATAGCTCAGTTGGTAGAGCGGAGGACTctaG | TAG | 1626.2 | 65 | 836 |
| ATCCTTAGGtCGCTGGTTCGAATCCGGCTCGAAGGA | | | | |
| CTTTCGATAGCTCAGTTGGTAGAGCGGAGGACTctaG | TAG | 373.8 | 145 | 837 |
| ATCCTTAGGtCGCTGGTTCGAATCCGGCTCGAAGGA | | | | |
| GGTAAAATGGCTGAGTGAAGCATTGGACTctaAATCT | TAG | 12.4 | 377 | 838 |
| AAAGaCAGGGGCTAAGCCTCTTTTTACCA | | | | |
| GGTAAAATGGCTGAGCAAGCATTAGACTctaAATCTA | TAG | 11.6 | 388 | 839 |
| AAGaCAGAGGTTAAGGCCTCTTTTTACCA | | | | |
| GGTAAAATGGCTGAGTGAAGCATTGGACTctaAATCT | TAG | 10.5 | 398 | 840 |
| AAAGaCAGGGGTTAAGCCTCTTTTTACCA | | | | |
| TATAGCTCAGTGGTAGAGCATTTAACTctaGATCAAG | TAG | 10.4 | 400 | 841 |
| AGGtCCCTGGATCAACTCTGGGTG | | | | |
| GGTAAAATGGCTGAGTAAGCATTAGACTctaAATCTA | TAG | 10.2 | 404 | 842 |
| AAGaCAGAGGTCAAGGCCTCTTTTTTCCT | | | | |
| GGTAAAATGACTGAATAAGCCTTAGACTctaAATCTG | TAG | 10.1 | 408 | 843 |
| AAGaCAGAGGTCAAGGCCTCTTTTTACCA | | | | |
| GGTAAAATGGCTGAGCAAGCATTGGACTctaAATCTA | TAG | 9.6 | 412 | 844 |
| AAGaCAGATGTTGAGCCATCTTTTTAGCA | | | | |
| GGTAAAATGGCTGAGTAAGCATTGGACTctaAATCTA | TAG | 9.4 | 413 | 845 |
| AAGaCAGAGGTCAAGACCTCTTTTTACCA | | | | |
| GGTAAAATGGCTGAGTAAAGCATTAGACTctaAATCT | TAG | 8.3 | 425 | 846 |
| AAGGaCAGAGGCTAAACCTCTTTTTACCA | | | | |
| TCTTCAATAGCTCAGCTGGTAGAGCGGAGGACTctaG | TAG | 7.6 | 436 | 847 |
| ATTCTTAGGTGCTGGTTTGATTCCGACTTGGAGAG | | | | |
| GGTAAAATGGCTGAGCAAGCATTAGACTctaAATCTG | TAG | 6.4 | 457 | 848 |
| AAAaCAGAGGTCAAAGgTCTCTTTTTACCA | | | | |
| GTCAGTGTTGCACAACGGTtaAGTGAAGAGGCTctaA | TAG | 4.1 | 521 | 849 |
| ACCCAGACTGGATGGGTTCAATTCCCATCTCTGCCG | | | | |
| CCTTCAATAGTTCAGCTGGTAGAGCAGAGGACTctaG | TAG | 4.0 | 526 | 850 |
| GTCCTTAGGtTGCTGGTTCGATTCCAGCTTGAAGGA | | | | |
| GGTAAAATGACTGAGTAAGCATTAGACTctaAATCTA | TAG | 2.8 | 599 | 851 |
| AAGaCAGAGGTCAAGACCTCTTTTTACCA | | | | |
| GGGGGTATAGCTCAGGGCtAGAGCTtTTTGACTctaGA | TAG | 2.4 | 640 | 852 |
| GCAAGAGGtCCCTGGTTCAAATCCAGGTTCTCCCT | | | | |
| GGTAAAATGGCTGAGTAAGCATTAGACTctaAATCTA | TAG | 1.5 | 789 | 853 |
| AAGaCAGAGGTCAAGGCCTTTTTACCA | | | | |
| GGTAAAATGGCTGAGTAAGCTTTAGACTctaAATCTA | TAG | 0.9 | 866 | 854 |
| AAGAGAGATTGAGCTCTCTTTTTACCA | | | | |

TABLE 33C
Tyrosine (Tyr) tRNA

| | | fold | | SEQ |
|---|---|---|---|---|
| seq_tRNA | ACE | over | Rank | ID |
| CCTTCGATAGCTCAGTTGGTAGAGCGGAGGACTtcaG | TGA | 313.3 | 154 | 855 |
| ATCCTTAGGtCGCTGGTTCGAATCCGGCTCGAAGGA | | | | |
| CCTTCGATAGCTCAGTTGGTAGAGCGGAGGACTtcaG | TGA | 269.8 | 160 | 856 |
| ATCCTTAGGtCGCTGGTTCGATTCCGGCTCGAAGGA | | | | |
| CCTTCGATAGCTCAGCTGGTAGAGCGGAGGACTt | TGA | 246.5 | 166 | 857 |
| caGATCCTTAGGtCGCTGGTTCGAATCCGGCTCGG | | | | |
| AGGA | | | | |
| CCTTCGATAGCTCAGCTGGTAGAGCGGAGGACTt | TGA | 211.5 | 172 | 858 |
| caGATCCTTAGGtCGCTGGTTCGATTCCAGCTCGA | | | | |
| AGGA | | | | |
| CCTTCGATAGCTCAGTTGGTAGAGCGGAGGACTtcaG | TGA | 172.6 | 185 | 859 |
| ATCCTTAGGtCGCTGGTTCGAATCCGGCTCGGAGGA | | | | |
| CCTTCGATAGCTCAGCTGGTAGAGCGGAGGACTtcaG | TGA | 140.1 | 196 | 860 |
| ATCCTTAGGtCGCTGGTTCAATTCCGGCTCGAAGGA | | | | |
| GGTAAAATGGCTGAGTAAGCATTGGACTtcaAATCTA | TGA | 24.9 | 326 | 861 |
| AAGaCAGAGGTCAAGACCTCTTTTTACCA | | | | |
| CTTTCGATAGCTCAGTTGGTAGAGCGGAGGACTtcaG | TGA | 6.4 | 458 | 862 |
| ATCCTTAGGtCGCTGGTTCGAATCCGGCTCGAAGGA | | | | |
| GGTAAAATGACTGAGTAAGCATTAGACTtcaAATCTA | TGA | 5.0 | 490 | 863 |
| AAGaCAGAGGTCAAGACCTCTTTTTACCA | | | | |
| GGTAAAATGGCTGAGTAAGCTTTAGACTtcaAATCTA | TGA | 4.7 | 502 | 864 |
| AAGAGAGATTGAGCTCTCTTTTTACCA | | | | |
| GGTAAAATGGCTGAGTAAGCATTAGACTtcaAATCTA | TGA | 3.6 | 545 | 865 |
| AAGaCAGAGGTCAAGGCCTCTTTTTTCCT | | | | |
| GGTAAAATGGCTGAGTAAGCATTAGACTtcaAATCTA | TGA | 3.4 | 561 | 866 |
| AAGaCAGAGGTCAAGGCCTTTTTACCA | | | | |
| TCTTCAATAGCTCAGCTGGTAGAGCGGAGGACTtcaG | TGA | 3.2 | 572 | 867 |
| ATTCTTAGGTGCTGGTTTGATTCCGACTTGGAGAG | | | | |
| GGTAAAATGGCTGAGCAAGCATTGGACTtcaAATCTA | TGA | 3.2 | 575 | 868 |
| AAGaCAGATGTTGAGCCATCTTTTTAGCA | | | | |
| GGTAAAATGGCTGAGTGAAGCATTGGACTtcaAATCT | TGA | 2.8 | 601 | 869 |
| AAAGaCAGGGGTTAAGCCTCTTTTTACCA | | | | |
| GTCAGTGTTGCACAACGGTtaAGTGAAGAGGCTtcaA | TGA | 2.6 | 617 | 870 |
| ACCCAGACTGGATGGGTTCAATTCCCATCTCTGCCG | | | | |
| GGTAAAATGGCTGAGCAAGCATTAGACTtcaAATCTG | TGA | 2.3 | 650 | 871 |
| AAAaCAGAGGTCAAAGTCTCTTTTTACCA | | | | |
| GGTAAAATGGCTGAGTAAAGCATTAGACTtcaAATCT | TGA | 2.3 | 655 | 872 |
| AAGGaCAGAGGCTAAACCTCTTTTTACCA | | | | |
| GGTAAAATGACTGAATAAGCCTTAGACTtcaAATCTG | TGA | 2.2 | 667 | 873 |
| AAGaCAGAGGTCAAGGCCTCTTTTTACCA | | | | |
| GGTAAAATGGCTGAGCAAGCATTAGACTtcaAATCTA | TGA | 2.1 | 676 | 874 |
| AAGaCAGAGGTTAAGGCCTCTTTTTACCA | | | | |
| GGGGGTATAGCTCAGGGCtAGAGCTtTTTGACTtcaGA | TGA | 2.1 | 677 | 875 |
| GCAAGAGGtCCCTGGTTCAAATCCAGGTTCTCCCT | | | | |
| CCTTCAATAGTTCAGCTGGTAGAGCAGAGGACTtcaG | TGA | 2.0 | 697 | 876 |
| GTCCTTAGGtTGCTGGTTCGATTCCAGCTTGAAGGA | | | | |
| TATAGCTCAGTGGTAGAGCATTTAACTtcaGATCAAG | TGA | 1.8 | 734 | 877 |
| AGGtCCCTGGATCAACTCTGGGTG | | | | |
| GGTAAAATGGCTGAGTGAAGCATTGGACTtcaAATCT | TGA | 0.9 | 863 | 878 |
| AAAGaCAGGGGCTAAGCCTCTTTTTACCA | | | | |

TABLE 34A
Valine (Val) tRNA

| | | fold | | SEQ |
|---|---|---|---|---|
| seq_tRNA | ACE | over | Rank | ID |
| GGTTCCATAGTGTAGCGGTtATCACGTCTGCTTttaAC | TAA | 5.06 | 488 | 879 |
| GCAGAAGGtCCTGGGTTCGAGCCCCAGTGGAACCA | | | | |
| GGTTCCATAGTGTAGTGGTtATCACGTCTGCTTttaACG | TAA | 2.30 | 664 | 880 |
| CAGAAGGtCCTGGGTTCGAGCCCCAGTGGAACCA | | | | |
| GTTTCCGTAGTGTAGTGGTtATCACGTTCGCCTttaACG | TAA | 2.01 | 710 | 881 |
| CGAAAGGtCCCCGGTTCGAAACCGGGCGGAAACA | | | | |
| GTTTCCGTGGTGTAGTGGTtATCACATTCGCCTttaACG | TAA | 1.97 | 715 | 882 |
| CGAAAGGtCCTCGGGTCGAAACCGAGCGGAAACA | | | | |
| GGTTCCATAGTGTAGTGGTtATCACATCTGCTTttaACG | TAA | 1.87 | 726 | 883 |
| CAGAAGGtCCTGGGTTCAAGCCCCAGTGGAACCA | | | | |
| GTTTCCGTAGTGTAGTGGTcATCACGTTCGCCTttaAC | TAA | 1.71 | 761 | 884 |
| GCGAAAGGtCCCCGGTTCGAAACCGGGCGGAAACA | | | | |
| GTTTCCGTAGTGTAGCGGTtATCACATTCGCCTttaACG | TAA | 1.51 | 793 | 885 |
| CGAAAGGtCCCCGGTTCGATCCCGGGCGGAAACA | | | | |
| GTTTCCGTAGTGTAGTGGTtATCACGTTCGCCTttaACG | TAA | 1.49 | 797 | 886 |
| CGAAAGGtCCCCGGTTCGAAACTGGGCGGAAACA | | | | |
| GCTTCTGTAGTGTAGTGGTtATCACGTTCGCCTttaACG | TAA | 1.14 | 844 | 887 |
| CGAAAGGtCCCCGGTTCGAAACCGGGCAGAAGCA | | | | |
| GTTTCCGTAGTGTAGTGGTtATCACGTTTGCCTttaACG | TAA | 0.73 | 880 | 888 |
| CGAAAGGtCCCCGGTTCGAAACCGGGCAGAAACA | | | | |
| GTTTCCGTAGTGTAGTGGTtATCACGTTCGCCTttaACG | TAA | 0.72 | 881 | 889 |
| CGtAAAGGtCCCCGGTTCGAAACCGGGCGGAAACA | | | | |
| GTTTCCGTAGTGTAGTGGTtATCACGTTCGCCTttaACG | TAA | 0.69 | 886 | 890 |
| CGAAAGGtCCCTGGATCAAAACCAGGCGGAAACA | | | | |
| GTTTCCGTAGTGGAGTGGTtATCACGTTCGCCTttaAC | TAA | 0.62 | 889 | 891 |
| GCGAAAGGtCCCCGGTTTGAAACCAGGCGGAAACA | | | | |
| GTTTCCGTAGTGTAGTGGTtATCACGTTCGCCTttaACG | TAA | 0.05 | 913 | 892 |
| CGAAAGGtCCGCGGTTCGAAACCGGGCGGAAACA | | | | |

TABLE 34B
Valine (Val) tRNA

| | | fold | | SEQ |
|---|---|---|---|---|
| seq_tRNA | ACE | over | Rank | ID |
| GGTTCCATAGTGTAGTGGTtATCACGTCTGCTTctaAC | TAG | 15.55 | 367 | 893 |
| GCAGAAGGtCCTGGGTTCGAGCCCCAGTGGAACCA | | | | |
| GGTTCCATAGTGTAGCGGTtATCACGTCTGCTTctaAC | TAG | 10.20 | 405 | 894 |
| GCAGAAGGtCCTGGGTTCGAGCCCCAGTGGAACCA | | | | |
| GGTTCCATAGTGTAGTGGTtATCACATCTGCTTctaAC | TAG | 6.77 | 452 | 895 |
| GCAGAAGGtCCTGGGTTCAAGCCCCAGTGGAACCA | | | | |
| GTTTCCGTAGTGTAGTGGTtATCACGTTCGCCTctaAC | TAG | 4.78 | 500 | 896 |
| GCGAAAGGtCCCCGGTTCGAAACCGGGCGGAAACA | | | | |
| GCTTCTGTAGTGTAGTGGTtATCACGTTCGCCTctaAC | TAG | 3.13 | 581 | 897 |
| GCGAAAGGtCCCCGGTTCGAAACCGGGCAGAAGCA | | | | |
| GTTTCCGTGGTGTAGTGGTtATCACATTCGCCTctaAC | TAG | 3.05 | 588 | 898 |
| GCGAAAGGtCCTCGGGTCGAAACCGAGCGGAAACA | | | | |
| GTTTCCGTAGTGTAGTGGTcATCACGTTCGCCTctaAC | TAG | 2.88 | 598 | 899 |
| GCGAAAGGtCCCCGGTTCGAAACCGGGCGGAAACA | | | | |
| GTTTCCGTAGTGTAGCGGTtATCACATTCGCCTctaAC | TAG | 2.52 | 629 | 900 |
| GCGAAAGGtCCCCGGTTCGATCCCGGGCGGAAACA | | | | |
| GTTTCCGTAGTGTAGTGGTtATCACGTTCGCCTctaAC | TAG | 1.79 | 744 | 901 |
| GCGAAAGGtCCGCGGTTCGAAACCGGGCGGAAACA | | | | |
| GTTTCCGTAGTGTAGTGGTtATCACGTTCGCCTctaAC | TAG | 1.38 | 818 | 902 |
| GCGtAAAGGtCCCCGGTTCGAAACCGGGCGGAAACA | | | | |
| GTTTCCGTAGTGGAGTGGTtATCACGTTCGCCTctaAC | TAG | 1.09 | 850 | 903 |
| GCGAAAGGtCCCCGGTTTGAAACCAGGCGGAAACA | | | | |
| GTTTCCGTAGTGTAGTGGTtATCACGTTCGCCTctaAC | TAG | 1.02 | 861 | 904 |
| GCGAAAGGtCCCTGGATCAAAACCAGGCGGAAACA | | | | |
| GTTTCCGTAGTGTAGTGGTtATCACGTTCGCCTctaAC | TAG | 0.80 | 876 | 905 |
| GCGAAAGGtCCCCGGTTCGAAACTGGGCGGAAACA | | | | |
| GTTTCCGTAGTGTAGTGGTtATCACGTTTGCCTctaAC | TAG | 0.65 | 887 | 906 |
| GCGAAAGGtCCCCGGTTCGAAACCGGGCAGAAACA | | | | |

TABLE 34C
Valine (Val) tRNA

| | | fold | | SEQ |
|---|---|---|---|---|
| seq_tRNA | ACE | over | Rank | ID |
| GTTTCCGTAGTGTAGTGGTcATCACGTTCGCCTtcaAC | TGA | 2.78 | 605 | 907 |
| GCGAAAGGtCCCCGGTTCGAAACCGGGCGGAAACA | | | | |
| GTTTCCGTAGTGTAGTGGTtATCACGTTCGCCTtcaAC | TGA | 2.43 | 642 | 908 |
| GCGAAAGGtCCCCGGTTCGAAACTGGGCGGAAACA | | | | |
| GTTTCCGTAGTGTAGTGGTtATCACGTTCGCCTtcaAC | TGA | 2.37 | 649 | 909 |
| GCGAAAGGtCCCCGGTTCGAAACCGGGCGGAAACA | | | | |
| GTTTCCGTAGTGGAGTGGTtATCACGTTCGCCTtcaAC | TGA | 2.25 | 669 | 910 |
| GCGAAAGGtCCCCGGTTTGAAACCAGGCGGAAACA | | | | |
| GTTTCCGTAGTGTAGTGGTtATCACGTTCGCCTtcaAC | TGA | 2.17 | 678 | 911 |
| GCGAAAGGtCCGCGGTTCGAAACCGGGCGGAAACA | | | | |
| GGTTCCATAGTGTAGCGGTtATCACGTCTGCTTtcaAC | TGA | 2.07 | 696 | 912 |
| GCAGAAGGtCCTGGGTTCGAGCCCCAGTGGAACCA | | | | |
| GTTTCCGTAGTGTAGTGGTtATCACGTTCGCCTtcaAC | TGA | 2.01 | 711 | 913 |
| GCGtAAAGGtCCCCGGTTCGAAACCGGGCGGAAACA | | | | |
| GTTTCCGTGGTGTAGTGGTtATCACATTCGCCTtcaAC | TGA | 2.00 | 712 | 914 |
| GCGAAAGGtCCTCGGGTCGAAACCGAGCGGAAACA | | | | |
| GTTTCCGTAGTGTAGTGGTATCACGTTCGCCTtcaAC | TGA | 1.81 | 737 | 915 |
| GCGAAAGGtCCCTGGATCAAAACCAGGCGGAAACA | | | | |
| GGTTCCATAGTGTAGTGGTtATCACATCTGCTTtcaAC | TGA | 1.80 | 740 | 916 |
| GCAGAAGGtCCTGGGTTCAAGCCCCAGTGGAACCA | | | | |
| GTTTCCGTAGTGTAGTGGTtATCACGTTTGCCTtcaAC | TGA | 1.75 | 750 | 917 |
| GCGAAAGGtCCCCGGTTCGAAACCGGGCAGAAACA | | | | |
| GTTTCCGTAGTGTAGCGGTtATCACATTCGCCTtcaAC | TGA | 1.50 | 796 | 918 |
| GCGAAAGGtCCCCGGTTCGATCCCGGGCGGAAACA | | | | |
| GGTTCCATAGTGTAGTGGTtATCACGTCTGCTTtcaAC | TGA | 1.32 | 824 | 919 |
| GCAGAAGGtCCTGGGTTCGAGCCCCAGTGGAACCA | | | | |
| GCTTCTGTAGTGTAGTGGTtATCACGTTCGCCTtcaAC | TGA | 1.30 | 830 | 920 |
| GCGAAAGGtCCCCGGTTCGAAACCGGGCAGAAGCA | | | | |

Thirty-five of the top-ranked tRNA sequences for overall suppressor activity were generated and screened. Amongst these, the following activity trends were noted:
(1) Unexpected suppression activity of isoleucine (Ile) suppressor tRNA posted the new top two activity values and more than a dozen in the top 100 sequence activities overall. While Ile is not a disease PTC, these could be used as a generic read-through tRNA for TGA premature termination codons.
(2) The screen validated that serine tRNA as potent suppressors for all three stop codon types.

It should be noted that one of the Leu tRNA sequences ranks extremely highly (over 10,000-fold over background). This tRNA sequence is labeled as SEQ ID NO: 497.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A transfer RNA (tRNA) comprising a T-arm, a D-arm, an anticodon-arm and an acceptor arm, wherein the anticodon-arm comprises a tri-nucleotide anticodon, wherein the anticodon is 5′-CUA-3′ and recognizes TAG-stop codons, optionally, wherein the tRNA is encoded by a sequence comprising a sequence selected from the group consisting of: SEQ ID NOs: 1-8, 12, 15-18, 20, 23, 24, 26, 29, 32, 34, 113-122, 226, 227, 229-233, 354, 405, 507, 669, and 795.

2. The tRNA of claim 1, wherein the tRNA is encoded by a sequence that comprises the sequence as set forth in SEQ ID NO: 1.

3-43. (canceled)

44. A transfer RNA (tRNA) comprising a T-arm, a D-arm, an anticodon-arm and an acceptor arm, wherein the anticodon-arm comprises a tri-nucleotide anticodon, wherein the anticodon is 5′-UCA-3′ and recognizes TGA stop codons, optionally, wherein the tRNA is encoded by a sequence comprising a sequence selected from the group consisting of: SEQ ID NOs: 10, 11, 13, 19, 25, 27, 30, 318, 857 and 858.

45-54. (canceled)

55. A transfer RNA (tRNA) comprising a T-arm, a D-arm, an anticodon-arm and an acceptor arm, wherein the anticodon-arm comprises a tri-nucleotide anticodon, wherein the anticodon is 5′-UAA-3′ and recognizes TTA-stop codons, optionally, wherein the tRNA is encoded by a sequence comprising a sequence selected from the group consisting of: SEQ ID NO: 22.

56. A transfer RNA (tRNA) comprising a T-arm, a D-arm, an anticodon-arm and an acceptor arm, wherein the anticodon-arm comprises a tri-nucleotide anticodon, wherein the anticodon is 5′-UUA-3′ and recognizes TAA stop codons, wherein the tRNA is encoded by a sequence comprising a sequence selected from the group consisting of: SEQ ID NOs: 9, 14, 21, 28, 31, 33, 35, 453, 454, and 557.

57-66. (canceled)

67. The tRNA of claim 1, wherein the acceptor arm is operably linked to an amino acid.

68. (canceled)

69. The tRNA of claim 67, wherein the amino acid is arginine.

70. The tRNA of claim 1, wherein the acceptor arm is not charged with an amino acid.

71. An oligonucleotide sequence that encodes the tRNA claim 1, wherein the oligonucleotide has a total length of less than 150 nucleotides.

72. An oligonucleotide comprising a first oligonucleotide sequence and a second oligonucleotide sequence, wherein the first and second oligonucleotide sequences independently encode a tRNA of claim 1 wherein the first and second oligonucleotides independently have a total length of less than 150 nucleotides, and wherein the two sequences are in tandem.

73. The oligonucleotide of claim 72, wherein the oligonucleotide is DNA.

74. An expression cassette comprising a promoter and a nucleic acid encoding the tRNA of claim 1.

75. A vector comprising the oligonucleotide of claim 71.

76. The vector of claim 75, wherein the vector is a viral or plasmid vector.

77. A composition comprising:
the tRNA of claim 1, and
a pharmaceutically acceptable carrier.

78. The composition of claim 77, wherein the carrier is a liposome.

79. A cell comprising the vector of claim 75.

80. A method of treating a stop-codon-associated genetic disease, comprising administering the composition of claim 77 to a patient in need thereof.

81. The method of claim 80, wherein the genetic disease associated with a premature stop codon is cystic fibrosis, muscular dystrophy, β-thalassemia or Liddle's syndrome.

82. A method of restoring translation to a nucleotide sequence that includes a nonsense mutation in a cell, comprising introducing to the cell the composition of claim 77, wherein tRNA restores translation to the nucleotide sequence that includes a nonsense mutation.

83-85. (canceled)
